SEQUENCE LISTING (77 sequences)

SEQ ID NO: 1 - DNA, 2115 bp, Zea mays
caagaggacagcaaccggcggccctcgcagccgcgcctcacgcgccacggaatatccctccagttccacggggggccacggcgtcagaactcagaaggccgcagggataagagagagcgggtcccgtccgagccgaggcagcccattcgccgtccgccccgcctcttcctgccgccgagcgccacgaggcccacgcccgcgatggcgactatcacgacgccgctccgctccgctctgttctctccggccgcctcgtccgcgggccgccaccgcgggggccggcgccgcgcgccctcctccgtgcgctgcgacgcctccccgccctcgcacgccgcggccgcctcgctcgacccggacttcgacaagaaggcgttccgccacaacctcacgcgcagcgacaactacaaccgcaaggggttcgggcacaagaaggagacgctcgagctcatgagccaggagtacaccagcaacgtcatcaagacgctcaaggagaacggcaaccagtacacctggggccccgtcaccgtgaagctcgcggaggcctacgggttctgctggggcgtcgagcgcgccgtgcagatcgcgtacgaggcgcgcaagcagttccccgaggagcgcatctggctcaccaacgaaatcatccacaaccccaccgtcaacaagaggttggatgagatgggtgtagaaatcattcctgttgacgcgggtatcaaggatttcaatgtcgtcgagcaaggtgatgttgttgtgttgcctgcatttggagctgctgtggaggaaatgtacacgctaaatgagaagaaggtgcagattgttgatacgacatgcccttgggtttcaaaggtctggaatatggtcgaaaaacacaagaagagtgaatatacttcaattattcatggaaagtattcccatgaagaaactgttgccactgcttcttttgcaggaaagtacatcattgtgaagaatatggcagaggcaacctatgtgtgtgattatatacttggtggccaacttgatgggtctagctcaacaaaagaggagttccttgagaaattcaagaaagctgtttctccagggtttgatcctgatgttcatcttgatatggtgggaattgcaaatcaaacaacaatgcttaaaggagaaactgaagaaattgggaagcttattgaaaagacgatgatgcaaaaatatggagttgaaaatgtaaacgatcacttcatggccttcaatactatttgtgatgccactcaggaaagacaagatgctatgtatcagctggtgaaagagaaagttgaccttattcttgttgttggaggatggaattcaagtaacacctctcatctgcaagaaatcggagaactcagtggaattccatcatactggattgacagtgaacaaaggattggaccaggaaacaggatcagctacaagttaaatcatggtgaactggttgagaaaaataactggttacccgaggggcctattaccattggtgttacttcaggtgcctcaactccagataaggttgttgaggatgctcttcagaaggtatttgagatcaagcgtcaggaaattttgcaggttgcataaattttaagcagagatttggtgaagagctgaatagttttggcttggcaaaggttactagaaacgttgcacaggcaaatgtttgtacagtagctaaggatgtaacgagttgggcacgaatactaccacgagtcactatccttgtgctggaatttacagtacggtggaaactaaaatggtgttatcattggcccgaataacatttgcatcagcctttctaaagtctaactttttgccacttaaattgatgtcagggaagacactcagatgtgtaagttaaagaatgcacagttcctatgtgtaaaaagcttagttccgaggaggagatggcgccccttcctttcccgcctgaccgttcctatcccctgaccctctcctctacccccgacgcctcttcaccttcctccaccaaccccacgccggagaccactccgatcccggcggccaatcacctctccttccccggcgtcggcggggccctctgtttgtggtcggtccaaggcgcagcggtggtgtgacgacagcgca

SEQ ID NO: 2 - DNA, 1961 bp, Zea mays
gcatgaccactgaattgctcgagtgcatatatgatcggatcctccagtgatctgattgatgctcaaagaatccatgcactatgcaggtaggtggatcgatagctgggaggcattaaagcggacgatgacgccttggcctcgctgcaatcttgcagattgctgctgcagcgcttctttaagagccaaccatccacatataccttgcttgacgccaagccaccggcattccactcccagcagcggtgggaacaaagagtgccagcgtctcacccctgaggccgcgcagagccactgcttgcgtctctgtctctctgtctgctcgtttttctccttctgcgactcgtaggagggtgaggtcttgcctcgcggaatggcggcgtccggggcatggctggcccgtgcgacggcgacggcggtgctgggtttcgtcctggcggtggcgtcagctgaggcggcatcgggggacgtggagatggtgttcctcaaggccgcggtggccaaaggcgcagtgtgcttggacggcagcccaccggtgtaccatttctctcccggctccggttctggcgccaataactgggtcgtccacatggagggaggagggtggtgcaggaatcctgatgagtgtgctgtccgcaagggcaacttcaggggctcctccaaatttatgaagccactctcgttttcagggatattaggcggcaaccaaaaatccaatcctgatttctacaactggaatagagtaaagatcagatactgtgatggttcatcatttactggtgacgttgaggctgtggacactgcgaaagatctccgttacagagggttcagagtctggcgtgccgtcatcgatgatctacttactgtgaggggaatgagcaaggcgcaaaatgctcttctttctggatgctcagccggaggtctagcagcaatactacactgtgacagattccatgatctgtttccagcgaaaacaaaggtcaagtgtttttctgatgctggatattttttcgatgggaaggatatctccgggaacttttacgctaggtcaatctataagagcgttgtgaatctacatggatcagccaaaaatttaccagcttcatgtacctcaaagccaaagcaatcacctgagctgtgtatgttcccacagtatgttgtcccgacaatgcgcacaccattgttcatacttaatgcagcctacgattcgtggcaggtcaagaacgtcctagcacctagtccagctgatccgaagaagacttgggcccaatgcaagcttgacatcaagagctgctccgccagccaactcacaaccttgcaaaatttcaggacagattttctggcagcactccctaaaacgcagtctgtaggcatgttcatcgactcatgcaatgctcactgccaatcaggatctcaagacacgtggctagccgatggttctcccacggttaacaagacgcaaattggcaaggcggtgggggactggtactacgatagggaggtccctcggcagattgattgcccgtatccctgcaacccaacttgcaagaaccgtgatgatgattgagcaattgtataagtagttcatgttatcgaaatgaaaacaataaaggatcacaacgcgcgcccgtagttgtagatgatgaattataaacacatatgactgagctcaaagttgtttaatcatcatctgttgcgaaatgaggaagacaattggtgtcttgaagctgtgttttcgactgtgtctaaagcgtaaatgtaacgtatattgtgtcttcgcctatgcttaagacattggactagttgattggtcaatttaatttattaaatgttttgattggtgtaatgaatataataagtcgtgcatgccgcatgactaggcttccagtcttccacttacaccggctaagcactgtctatatatatgtagtcactttggatcaatgaatcagctgtttttatca

SEQ ID NO: 3 - DNA, 2149 bp, Zea mays
gagcgagccaccacccaacctgaccccttcgccccgtatcgttcccttcctcctctcttttccccaccgccccctttcttggcgtctacccggcgcgaccaggaccgaatcctaaggccgccggccgccgccgccgccgcctgctcccgttggacacggtaaaacctcctccctcctcgtccgtgtcgccggggtccggggtccaagcggctgcgcgcgcggtctcgccgccggcgccgatctgggcgccgccggcgttgaccctgtccgattcgccccgggctgcgagacctctgcctccctgaccggttactcggaacttctactcgcctgtgggatcctccagcggatcagatgagcacatcgacttgagcaccacgctttttggttggaaggcgaatcgtagagctttcctgggtttctctgagggctcttctcagatgtggcgtcctccagagtctaccacatactgtttgaggagtcctggttcttttcggaatccggactaaccaagggctcctactgtgcacgactgcttgacaggatatttcggattattattatgtgcgtgcgagcgcgcgtgtgcgccttctagaggcatgattcttaaatcagagccttcgtgtttaaatccgagtttgccttcgtgttgaactatgagtgaatttcattttctgcgggagttgaagtcgatttagatcaggacagtgtttcttgcgatctgattaagcctttttttttcttgctattgtgatttcttttttcagagtttggagtaaagaaaccaaccctgcatctgtattctgtctgtctgtgctgcttcgaataagccttgcatctcgctgacttgggatataactatgccgaaggacaggagctcccgcgtttcctcttatgagagccgccgggctggtgcctccccatacttctcatcgtctcatggacagagcagttcttgtcgccggtccgaggagtcttgtggggcagcagcggcggcagcagcaaagcaagctgcagagtgggaggatgttcggtgcccggtgtgcatggaccacccgcacaacgccgtcctgctggtctgctcctcacacgagaagggctgccgccccttcatgtgcgacaccagctcgcggcactcgaactgctatgaccagtaccggaaggcctccaaggattcaaggacagagtgcagcgagtgccagcagcaggttcagctctcgtgcccactgtgccgtgggccggtcagcgattgcatcaaggactacagcgcgcggaggttcatgaacaccaaggtccggtcgtgcaccacggagtcgtgcgagttcaggggcgcctaccaggagctgaggaagcatgctagggtggagcatccaacaggaaggccaatggaggtagaccctgagcggcagcgggactggcgccggatggagcagcaacgggaccttggagacttgatgagcatgctgcgttcagggttcaacagcaatattgaggacgacagtggcgggcttggagacaccgaagaagggggagaggaagctgaaatgactccggcctccataaccatggtcttcatcatgccatctagaggctcaatcatgcagtacctatcggaacgcagcagaacgatcattctggtcagtcggaggcgagcaagcagcagcagcggtggcgacgctgaagccactgctccagacagcgaggaaggtgatgaccctatgccatcggcagaggcatctgctggttcacagcattcttccgaacaagaggaggctgacggtgaccctgcccaatgacgtaagtcagctggcaagaggtgtgccatggcatcttcttagcctgaagatcccgaccaacatggcaacatggtatgtggcgaagaaaaacatggatgcaggtgccaccaaggcgtagccaggacaatctgtctacgcaggaaaatcgaggggaatcagcatcgcgcgaaggcttcagaaacgtggtggcctctagtaccaattctaatgtttccgcggggttctgtggcaatggagagagagaaacaattgggtggtagctatcttgtctgaatggatttcattttccttgcattgtaattctctaatatatattatcatatgaaatagattcggccgtatttgcactgcgt

SEQ ID NO: 4 - DNA, 1747 bp, Zea mays
gtcagctaagcgaacgtctcgattcgtcaaagtctgaaattaagctggacaccgctcccgcaccaaaccaaactaattacctcttcccgcgacctcctcccccggcgtacctccggagatccaccccgacccaccaccgcgtgcctcggcaatggccgccgctccgaccaccgtaaccaagtccccgccgtccctggtcccgccggcggggcccacgccaggcggttccctcccgctctcctccatcgacaagactgccgccgtccgcgtctccgtcgacttcatccaggtcttccccgcccctacgtcggggaaggaggaccggagcccctcctccacgatcgcggctatgcgcgagggctttgccaaggcgctcgtgccgtactaccccgtcgccggccgcatcgccgagcccgttccgggggagcctgagattgagtgcacaggggaaggggtgtggttcgtggaggccgaggccagctgctccctcgaggaggcgcggaacctcgagcgcccgctgtgcatccccaaggaggagctgcttcctcgtccgccggccggggtgcgcgtggaggacaccctgctgctcgctcaggttacaaagttcacatgtggtggatttgctgtgggcatttgcttcagtcacttggtgttcgatgggcagggtgctgcacaatttctgaaagcggtgggtgagatggctaggggcctccctgagccatcgatcaagccaatctgggctcgtgatgccatccccaacccacctaagccacccctaggtccgccgccgtcattcaccgcattcaactttgagaaatcggttcttgagatctctccggacagcatcaagaacgtgaaggatcaggttgcaagtgaaaccaaccagaagtgttccactttcgacgtggtcactgccataatcttcaaatgccgcgccttggcagtcgacttcgcgcccgacgctgaggtccgcttgggcttcgcagccagcactcgccacctgctgagcaatgtgctgccctcggtcgaaggctactacgggaactgtgtgtacccaggtggtctcaccaagaccagccaggaggtgaaggaagcttcgcttgtggagatcgtgaccgtgatcagggaagccaaggaagctctgtcatcgaggttccttgactggttgagcggcggcgccaaggagaaccactacaacgtgtcgctagactatggcaccctcgtcgtgactgactggagccatgtgggcttcaacgaggtggactacgggttcggtgagccgagctacgtgttcaccctgaacgacgacgtgaacatcgtcccctccgttgtgtacctgaagccgcccaagccgaagcagggcatcaggctggtcctgcagtgcgtggaaggccatcactctgccgtgttcggcgaggagttgcagaagcatgcatagagtgagtgtattctacagtgggaatctgttgtattttatttgttgtgtcaaattgctgctcccggaatttgcttgcaataaggcagattggtcgtgtttatactttgtaccattatcagcacgttacattatacatgtgatgaatattgacagtgacgaaagaataataatgttcccatttggaacaaattatttcagattcgttggcctgctgtaggttcctggtgtctcgagttttaacgtgtgtaactgtgttatgtataagtataactctgacagtgtttgatgattgatcaacggcagaaagaa

SEQ ID NO: 5 - DNA, 2311 bp, Zea mays
tctcattcaagtgctgtaaacatataacccaaatatgatcatttttttgtgttctcattatttcttttctaagaagtaagacccaaccagggttttttgtccatcattaagtgattgttgctgattgaatacagaagttacagaagaagaagctgaagaaaaattgcaagacacaataagggagaggttttcgtcctttggtgaggattaccatgctgttgatattctattagcagagatgatgtgtatgaactttttgctttcaagcattgtgtgggaagaaggatacagcttgccctttgtaaagaacttgatgagaggatgcatgacctgaaaaaggaactggagggttacaatactggagattttgatgaaactaacaagaagaaagctcttgatgcactgaagagaatggaaagctggaacttattcagagatacttcagtggaacatcatagttacacagtggctcatgattcatttcttgcacaacttggatctatgttatggggctctatgaggcatgtaattgctccttctgcctctcatagagtgtaccattactatgagaagttatcgtttcagttgtattttgtgacacgagagaaagtcaggagtataaagcagttacctgttaatgtaaaatccatcagggagagcctgaattctgtgctattacatcatcaaaactccatgtttagccaaaacatgctgtcattgtcagaggatccatcattgatgatggcattttcaatggcacgtcgtgcagctgcggtgccgcttctattagtcaatggcacctataagtcaactgttagcacataccttgattctgctattctccaacatcagctacagaagctaaatgagcacaattcactgaaaggaaggcattcaaatcacaggtcaacattagaggtcccaatattctggttcatacataatgaacccatattattggacaaacattatcaagccaaggctctctcaaatatggtcgtagtagttcagtcagatgatgattcctgggaaagccatttgcagtgcaatggaagacccatcttatgggatttgaggaaaccggttaaagctgctattgctgcaactgctgagtatgtatctggtctacttcctccacatctggtttatagccatgctcatgaaactgcaattgaggactggacctggtctgtgggttgtaatccctcagctgtgacttctgaaggttcacaactttcagagttccagcaagatgtgattgctcgtaactatattattacttcagtggaggaatccattcaagtaatcaattcagcaattcagcaattggtaatagagcggactactgaaaaaggcttcaaaattttcaaggctcacgaaagtaagatggttgagaagtacaatgccgttgttagcttgtggagaagagtatcggctatgtccaagggattgcgatatggtgatgcagtaaaacttatgtcaatgcttgaggatgcttcaaatgggttttctagtgctgtgaactccaccatttcaagtctgcaccctgtccaatgcacccgcgaaaggaaggtcgacgtgcagctagacttgacaacacttcctgcttttctagctgtatttttgttgctttggtttcttctacgtccaaggagaccgaagcctaagatcaactgaacaccgagccaatgagcagcataggccatagagtttttgtgaatacgcgcatggattacagatggcgctggagcatggcccgggaattccaaaggtccaaaacaccgggtggcagggaacaaggtttcagaagattgcaatcctgacacatccccaagttgtagcagagttggaatgtcatgaaaactttaattcattcagtcctgtcctcgttccgggtttagccaattcttcctcgttccgggtaaggccttgttcgtttgtgtcggattggtgggtcggaacaattcccggccggattgcttctctaatttatataaactttgattagccggaacgattccgggtgcaatccgacgcaaacgaacaagccctaactgagattaatttgtccttgctgtaatgtttagccagtcctgccccgatccggggaactgagagattgtctttatcgcaacattaacggctagcggttagtatcatcttccagtcacctggaatgttactagtacaatccaattgtctgtttcctgccgcttacatgtaaaagtccatactcaagttttacagaaagaaacatgttctgtcatttattacaaaataaagccaaatagtaaaatgttatgtgtacgta

SEQ ID NO: 6 - DNA, 2397 bp, Zea mays
ggagtttctggtaacgatgctactatgggtccaaagcacacgcttccacctggtagtgttacctcttcagctgaattggcttctagcgttctgaaagggagcgaggattgggatgctgatgtaatggataagtattctattggaaaagaaggcaaatctaaaaatattgatccagttaggaaggatgattcagtagcaatcttagaacagttctttggcaatgttttatcgaaaagcggcagcaacctaccaacttatgttgagaaccagccattgaaaactgatgatgacatgatcacttctgtgccagaatcatccaaatttgctcattggtttcttgatgaagacttgaaacctgcagaagacttatcttcaaagagcctgctctccatgattgtcaaaaatgaaaatccaggtctagaaaatttaaaccatactcctttatctgatgctgctgcccagaatttatccccaagagcacctattgataaacttgattctgcatcagagcttatctcatttacatcctctacgcctgccaatggagttcttgaacaatgcatccattctgatgttccagaggcagttcctattatgacatgtgaggatcttgagcagacgatgttagcacaggttagcaatagcagctcaactcagataaatgctacaaaggagcaactgactgttatggatgaaccagttgccatgcagaaagtaactgtagataatcatgcatcacaacatcttctttcattgttgcaaaaaggaacagataataagggagcaccttccctgggtttccagagagaatcaactgatgaacctctgagtgttgacacaaatttaatggcaaatggtggaatatctggaagtgatccggttaacagtgttgaaaatgttcctacttctgggaaggacttgacattggaagcgttattcggggctgcatttatgaatgagctccactcgaaagatgcaccagtttctattcgaggagccacaactggtggtcctactgagtttgcagagatgggtaaaactctgttgtcatctagccatgaaggatactaccctgttgaacagaccgtacacttcaacaatactaaagatgctgctgtccgtagagaaccaggtattgagcattcagcagtacctggtctaagtcaggggagtgctagttttgacaagaaaggaatggaaattcatctgcctgaagaagataatttgtttaccatgagtgattctctgcttggtcaaaattctgatattttggcatcagtaggatccagcagggttgaagggctattgcctgaaaaggcacttgataacctcagctataggtttcaaagtcttgtgcctggtgatgcagaacacattcaagtatatggtcctgatgcacttggatctcatcctcgtgattctcagaatatgtatcatcttctacagggtaggcctcctatgatagcacctcaccctatgatggatcacattgttaataggaaacagccagctccatttgatatggcacagtcgatacaccatgattctcaccgttctttcccatctaatgtgaatcatatgcaacataatcttcatgggccaggggtccctcacttggaccctgctggacatattatgcgacaacacatgtccatgcctggaagatttcctccagaaggcttgccaagaggtgtccctccatctcagcctgtgcatcacatggctggttatagacctgaaatgggtaatgtaaataatttccatatgcaccctcgccagcccaactatggagaatttggattgatgatgccaggtccagaggtgaggggcaatcatccagaggcgttcgaaaggttgatccagatggagatgtcagccagatcgaagcaacagcaggtgcaccaccctgcaatggccgctggccgtgtgcctagtgggatgtacgggcacgagcttgatgcgaaattgagatacagatgatggatgcctggatgcttcactccgtacagaggacctggaggtgtggtttgttgtatgtgcgtggtcactctttgcccagactgctgtgtattatttctgctaacatggtttagcatcagccgtcggtcgcgactgattggaggcctgcctcacttgtagggttgtagcatgtacatctgaacgggtgatggaacggagtgggtctaagatctgtaggagcggaagtctaccgggaaaaggggttatggtgtgctgaatggaagacgtggcgtcgacgtcttagcagccacatgtgtaatgacgttttctgtctactgtttctgacgactatgcagtttccattttgtataagctctgttatgcaaaaggaaaaaaaaagaagaaaaaaaactgagtcagattaacagattggcgaca

SEQ ID NO: 7 - DNA, 1470 bp, Zea mays
ctatcctaaaccccaccgaccggataacaggacactggcactgccattcccgtttcctcgctcgaacacacaccgaagagagagacagagcgagagaggatggcgtccctggcgcagcacgtcgcgggcctaccgtgcccccctctatccggcgcgtcgcgccgtcgccccgcggcgcagaggcggcctccgtcggcgcttgtgtgcggtacctatgcgctgaccaaggacgagcgggagcgggagcggatgcgccaggtgttcgacgacgcctccgagcgctgccgcaccgcgcccatggagggcgtcgccttctcccccgacgacctcgacaccgccgtcgagtccaccgacatagacacggagatcggctcgctcattaaaggaacagtatttatgactacctcaaatggtgcatatatcgacatccaatccaagtctactgcttttttgcccttagatgaggcatgtcttcttgatatcgataatgttgaagaggctggcattcgtcctgggttagtagaagaattcatgataattgatgagaacccaggtgatgaaactttgattctaagtttgcaagcaattcagcaagaacttgcatgggaaaggtgccggcaacttcaggccgaagatgtcgttgtcacgggtaaagtaattggtggaaacaaaggaggtgtagtagctcttgtggatgggcttaagggtttcgttccattttcgcaagtgtcatcgaaaacaaccgccgaagagctgcttgagaaagaattgcctctgaagtttgtagaggtcgatgaggaacaaggcaggcttgtcctcagtaatcgcaaggcaatggcagatagtcaggcccagctaggtattggatcagttgtcttgggaactgtagagagcctaaaaccttatggcgccttcattgacatcggtggaatcaacggccttctccatgtgagccagattagtcatgaccgtgttgcagatatctcaacagttctgcaaccaggagataccctcaaggttatgatactgagccatgaccgtgaaagaggccgagtcagcctttctactaagaagcttgagccaacacctggtgacatgatccgcaatcccaagcttgtgtttgagaaggctgatgagatggctcagatattcaggcagagaatagctcaggcagaggctatggctcgtgctgacatgttgagattccagccagagagtggattgaccctcagttcagagggcatcttaggaccattgtcgtcggatgcaccttcggaggattctgaagatcgcacagatgaatagaggcagttgacgaagtgcaccgggtttgagatatgggatggcagttcgtcaagctcattttcaatcgggtgggggaggcatgacgagatcattttctgttcagatcgtgaggtccgttccagttattatccatttggattaggaaatagaaaaagtaacagggtt

SEQ ID NO: 8 - DNA, 1285 bp, Zea mays
caccagtccaccgcgccacaggctccaccctcccctctcggcccggctcgatggggtccggcgtgtcgtcgagcatggcgctggcgctggcgggcttctgcttcagcgtcctcttcatcgtcttcgtctgcacgcgcctcgcctgcgcgctcgtccgccggcgccggcgccaggcccgcgcccgcctcgcggccgccccgccgctcccgcactacgcccacggctacgccgaccccgaccctttcccgtcgttccgcgccgcccgccaccaccaccacgccccgggcctcgatcccgccgccttccccacccgcgcctacgccgccgcacaagcctccgactccgacgacggctcccagtgcgtcatctgtctggcggaatacgaagagggagacgagctccgcgtgctgcctccctgcagccacaccttccacacgggctgtatcagcctgtggctggcgcagaactcgacgtgcccggtctgtagagtctcgctgctcgtgcctgatactagtactacccctgaaagcgaacactctgcaccccatcctcctcctcctcctcatcatcatcatcatctgtccagcatagtgataataagcccaccaagctcccccgaaccgtcgagatcggacccgtgccgatgcctgttcgccagcggtggtgggcactcgtcaagggcggcagaggctcctcctcctcctcctcctcccagacacgagcccgaccaggtcgtatctggtccaccaccggcagcagatggggctagcggctacagctcgccgttgcctgaagttattcaccccgctcctgctcctgaaaccaacgggcagacagtacggaagcaggcgggcagcagatctactaccccgctaggcccctgcaaatagcggccgctcactctgtgtgggtgggtggggtgaacaggtggtgcgtggtaaaagcgaagtagagagaaacaagcgacttgaagaggcctgggttcgttcgtgtacatacgatcgagaaatcgtttcaggtcattcattcatccattcattcatccatgggcacatactgtggtattacggagtattacggtgtacgtgtagtgtgccgcaggagagacgacgcgacggcagcagtgcgttttccatatgcgacgggacgggagcattcgaggagatgatggcaatggcatggttttgtgtactgtacggtaacatttgtcgctgggaattaataataaaaaacccgtggctggctgatgcagcagcagctgtcttattatccagccacgcatgtg

SEQ ID NO: 9 - protein, 462 aa, Zea mays
MATITTPLRSALFSPAASSAGRHRGGRRRAPSSVRCDASPPSHAAAASLDPDFDKKAFRHNLTRSDNYNRKGFGHKKETLELMSQEYTSNVIKTLKENGNQYTWGPVTVKLAEAYGFCWGVERAVQIAYEARKQFPEERIWLTNEIIHNPTVNKRLDEMGVEIIPVDAGIKDFNVVEQGDVVVLPAFGAAVEEMYTLNEKKVQIVDTTCPWVSKVWNMVEKHKKSEYTSIIHGKYSHEETVATASFAGKYIIVKNMAEATYVCDYILGGQLDGSSSTKEEFLEKFKKAVSPGFDPDVHLDMVGIANQTTMLKGETEEIGKLIEKTMMQKYGVENVNDHFMAFNTICDATQERQDAMYQLVKEKVDLILVVGGWNSSNTSHLQEIGELSGIPSYWIDSEQRIGPGNRISYKLNHGELVEKNNWLPEGPITIGVTSGASTPDKVVEDALQKVFEIKRQEILQVA

SEQ ID NO: 10 - protein, 397 aa, Zea mays
MAASGAWLARATATAVLGFVLAVASAEAASGDVEMVFLKAAVAKGAVCLDGSPPVYHFSPGSGSGANNWVVHMEGGGWCRNPDECAVRKGNFRGSSKFMKPLSFSGILGGNQKSNPDFYNWNRVKIRYCDGSSFTGDVEAVDTAKDLRYRGFRVWRAVIDDLLTVRGMSKAQNALLSGCSAGGLAAILHCDRFHDLFPAKTKVKCFSDAGYFFDGKDISGNFYARSIYKSVVNLHGSAKNLPASCTSKPKQSPELCMFPQYVVPTMRTPLFILNAAYDSWQVKNVLAPSPADPKKTWAQCKLDIKSCSASQLTTLQNFRTDFLAALPKTQSVGMFIDSCNAHCQSGSQDTWLADGSPTVNKTQIGKAVGDWYYDREVPRQIDCPYPCNPTCKNRDDD

SEQ ID NO: 11 - protein, 311 aa, Zea mays
MPKDRSSRVSSYESRRAGASPYFSSSHGQSSSCRRSEESCGAAAAAAAKQAAEWEDVRCPVCMDHPHNAVLLVCSSHEKGCRPFMCDTSSRHSNCYDQYRKASKDSRTECSECQQQVQLSCPLCRGPVSDCIKDYSARRFMNTKVRSCTTESCEFRGAYQELRKHARVEHPTGRPMEVDPERQRDWRRMEQQRDLGDLMSMLRSGFNSNIEDDSGGLGDTEEGGEEAEMTPASITMVFIMPSRGSIMQYLSERSRTIILVSRRRASSSSGGDAEATAPDSEEGDDPMPSAEASAGSQHSSEQEEADGDPAQ

SEQ ID NO: 12 - protein, 428 aa, Zea mays
MAAAPTTVTKSPPSLVPPAGPTPGGSLPLSSIDKTAAVRVSVDFIQVFPAPTSGKEDRSPSSTIAAMREGFAKALVPYYPVAGRIAEPVPGEPEIECTGEGVWFVEAEASCSLEEARNLERPLCIPKEELLPRPPAGVRVEDTLLLAQVTKFTCGGFAVGICFSHLVFDGQGAAQFLKAVGEMARGLPEPSIKPIWARDAIPNPPKPPLGPPPSFTAFNFEKSVLEISPDSIKNVKDQVASETNQKCSTFDVVTAIIFKCRALAVDFAPDAEVRLGFAASTRHLLSNVLPSVEGYYGNCVYPGGLTKTSQEVKEASLVEIVTVIREAKEALSSRFLDWLSGGAKENHYNVSLDYGTLVVTDWSHVGFNEVDYGFGEPSYVFTLNDDVNIVPSVVYLKPPKPKQGIRLVLQCVEGHHSAVFGEELQKHA

SEQ ID NO: 13 - protein, 451 aa, Zea mays
MHDLKKELEGYNTGDFDETNKKKALDALKRMESWNLFRDTSVEHHSYTVAHDSFLAQLGSMLWGSMRHVIAPSASHRVYHYYEKLSFQLYFVTREKVRSIKQLPVNVKSIRESLNSVLLHHQNSMFSQNMLSLSEDPSLMMAFSMARRAAAVPLLLVNGTYKSTVSTYLDSAILQHQLQKLNEHNSLKGRHSNHRSTLEVPIFWFIHNEPILLDKHYQAKALSNMVVVVQSDDDSWESHLQCNGRPILWDLRKPVKAAIAATAEYVSGLLPPHLVYSHAHETAIEDWTWSVGCNPSAVTSEGSQLSEFQQDVIARNYIITSVEESIQVINSAIQQLVIERTTEKGFKIFKAHESKMVEKYNAVVSLWRRVSAMSKGLRYGDAVKLMSMLEDASNGFSSAVNSTISSLHPVQCTRERKVDVQLDLTTLPAFLAVFLLLWFLLRPRRPKPKIN

SEQ ID NO: 14 - protein, 975 aa, Zea mays
MKTRIVYSREFLLSLGELEHCKKLPPDFDAALLSELQELSAGVLERNKGYYNTSQGRPDGSVGYTYSSRGGNTGGRWDTRSSGSSDRDGEPDRESQTQAGRGANQYRRNWQNTEHDGLLGRGGFPRPSGYTGQLSSKDHGNAPQLNRTSERYQPPRPYKAAPFSRKDIDSINDETFGSSELSNEDRAEEERKRRASFELMRKEQHKAVLGKKSGPDILKENPSDDIFSKLQTSTAKANAKTKNEKLDGSVVSSYQEDTTKPSSVLLAPAARPLVPPGFANAFADKKLQSQSSNITHEPKLEDDQSATGFTSESKEKGVSGNDATMGPKHTLPPGSVTSSAELASSVLKGSEDWDADVMDKYSIGKEGKSKNIDPVRKDDSVAILEQFFGNVLSKSGSNLPTYVENQPLKTDDDMITSVPESSKFAHWFLDEDLKPAEDLSSKSLLSMIVKNENPGLENLNHTPLSDAAAQNLSPRAPIDKLDSASELISFTSSTPANGVLEQCIHSDVPEAVPIMTCEDLEQTMLAQVSNSSSTQINATKEQLTVMDEPVAMQKVTVDNHASQHLLSLLQKGTDNKGAPSLGFQRESTDEPLSVDTNLMANGGISGSDPVNSVENVPTSGKDLTLEALFGAAFMNELHSKDAPVSIRGATTGGPTEFAEMGKTLLSSSHEGYYPVEQTVHFNNTKDAAVRREPGIEHSAVPGLSQGSASFDKKGMEIHLPEEDNLFTMSDSLLGQNSDILASVGSSRVEGLLPEKALDNLSYRFQSLVPGDAEHIQVYGPDALGSHPRDSQNMYHLLQGRPPMIAPHPMMDHIVNRKQPAPFDMAQSIHHDSHRSFPSNVNHMQHNLHGPGVPHLDPAGHIMRQHMSMPGRFPPEGLPRGVPPSQPVHHMAGYRPEMGNVNNFHMHPRQPNYGEFGLMMPGPEVRGNHPEAFERLIQMEMSARSKQQQVHHPAMAAGRVPSGMYGHELDAKLRYR

SEQ ID NO: 15 - protein, 401 aa, Zea mays
MASLAQHVAGLPCPPLSGASRRRPAAQRRPPSALVCGTYALTKDERERERMRQVFDDASERCRTAPMEGVAFSPDDLDTAVESTDIDTEIGSLIKGTVFMTTSNGAYIDIQSKSTAFLPLDEACLLDIDNVEEAGIRPGLVEEFMIIDENPGDETLILSLQAIQQELAWERCRQLQAEDVVVTGKVIGGNKGGVVALVDGLKGFVPFSQVSSKTTAEELLEKELPLKFVEVDEEQGRLVLSNRKAMADSQAQLGIGSVVLGTVESLKPYGAFIDIGGINGLLHVSQISHDRVADISTVLQPGDTLKVMILSHDRERGRVSLSTKKLEPTPGDMIRNPKLVFEKADEMAQIFRQRIAQAEAMARADMLRFQPESGLTLSSEGILGPLSSDAPSEDSEDRTDE

SEQ ID NO: 16 - protein, 279 aa, Zea mays
MGSGVSSSMALALAGFCFSVLFIVFVCTRLACALVRRRRRQARARLAAAPPLPHYAHGYADPDPFPSFRAARHHHHAPGLDPAAFPTRAYAAAQASDSDDGSQCVICLAEYEEGDELRVLPPCSHTFHTGCISLWLAQNSTCPVCRVSLLVPDTSTTPESEHSAPHPPPPPHHHHHLSSIVIISPPSSPEPSRSDPCRCLFASGGGHSSRAAEAPPPPPPPRHEPDQVVSGPPPAADGASGYSSPLPEVIHPAPAPETNGQTVRKQAGSRSTTPLGPCK

SEQ ID NO: 17 - DNA, 801 bp, Zea mays
  misc_feature (26)..(31): n is a, c, g, or t
  misc_feature (174)..(174): n is a, c, g, or t
  misc_feature (260)..(260): n is a, c, g, or t
  misc_feature (790)..(790): n is a, c, g, or t
caagcaagctgtgtagraacaaacannnnnngttaaatcagtacagctcyaagtcaccttatctggagttgaggcacctgaagtaacaccaatggtaataggcccctcgggtaaccagttatttttctcaaccagttcaccatgctgttaaacaatccaattttcggttaaaantgagatcatatactttcaataawtaaatttaagctctttccagtttctaattaaaagaaggtcccttcaaaattctattatttttnaaaaaatgaatgctgtggaaggtatgagaaattacatttarcttgtagctgatcctgtttcctggtccaatcctttgttcactgtcaatccagtatgatggaattccactgagttctccratttcttgcagatgagaggtgttacttgaattccatcctccaacaacaagaataaggtcaactttctctttcaccagctgatacatagcatcttgtctttcctgaggaatcaaacatatggtcggatgtatataagtcaatgcagcatatgtcaaaacaattataaatctatcacycaaagagwcawtttctcaaaccaaaagtggagcaacaaatgkactaggatcctcttaactttgatccmaaataaaagtgccatctagttgyctcagtttcctttgcaggttgcatgtaaagggtgatggagattgtattataccatgaatggctaccrtctatcatgaattgtaygctagccttttctwgacrttacagtacagtagtgctaaatccatcattcccagtttttnaaatcatcaag

SEQ ID NO: 18 - DNA, 801 bp, Zea mays
cggtgggggactggtactacgatagggaggtccctcggcagattgattgcccgtatccctgcaacccaacttgcaagaaccgtgatgatgattgagcaattgtataagtagttcatgttatcgaaatgaaaacaataaaggatcacaacgcgcgcccgtagttgtagatgatgaattataaacacatatgactgagctcaaagttgtttaatcatcatctgttgcgaaatgaggaagacaattggtgtcttgaagctgtgttttcgactgtgtctaaagcgtaaatgtaacgtayattgtgtcttsgcctatgcttaagacatkggactagttgattggtcaatttaatttattaaatgttttgattggtgtaatgaatataataagtcgtgcatgccgcgtgactaggcttccagtcttccacttacaccggctaagcactgtctatatatatgtartcactttggatcaatgaatcagctgtttttatcagtttaggttttctttttcacttcttgttttgccatggctgagactggccgcagcgcttcccgccagtagtcctctcctctatcactgttcctgtttagcgtcactctactgctgcgtcagtcgtctctacacttgcctctacgcgctacagctctagaggaatacataggaccacgctagatgtggcgggctgcaagctgtcccctgccctcgaaaccagtgcacgacgtgggccctcttctaattgttagtataagaaaaattgataatagataaaaaatagtatatgaaatgatattttatggttgt

SEQ ID NO: 19 - DNA, 801 bp, Zea mays
agagtttggagtaaagaaaccaaccctgcatctgtattctgtctgtctgtgctgcttcgaataagccttgcatctcgctgacttgrgatataactatgccgaaggacaggagctcccgcgtttcctcttaygagagccgccgggctggtgcctccccatacttctcatcgtctcatggacagagcagttcttstcgccggtcmgaggagtcttgtgkggcagcagcggcggcrgcagcaaagcaagctgcagagtgggaggaygttcggtgcccggtgtgcatggaccacccgcacaacgccgtcctgctggtctgctcctcacacgagaagggctgccgccccttcatgtgcgacaccagctcgcggcactcgaactgctatgaccagtaccggaaggcatccaaggattcaaggacagagtgcagcgagtgccagcagcaggttcagctctcgtgcccactgtgccgtgggccggtcagcgattgcatcaaggactacagcgcgcggaggttcatgaacaccaaggtccggtcgtgcaccacggagtcgtgcgagttcaggggcgcctaccakgagctgaggaagcatgctagggtggagcatccaacaggaaggccaatggaggtagaccctgagcggcagcgggactggcgccggatggagcagcaacgggaccttggrgacttgatgagcatgctgcgttcagggttcamcagcaatattgaggacgacagtggcgggcttggagacaccgaagaagggggagaggaagctgaaatgactccggcctccataacca

SEQ ID NO: 20 - DNA, 801 bp, Zea mays
caggccaacgartctgaaataatttgttccaaatgggaacattattattcyttcgtcactgtcaatattcatcacatgtataatgtaacgtgctgataatggtacaaagtataaacacgaccaatctgccttattgsaagcaawttccgggagcagcaatttgacacaacaaataaaatacaacagattcccactgtrgaatacactcactctatgcatgcttctgcaactcctcgcygaacacrgcagagtgatggccttccacgcactgcagsaccagcctgatgccctgcttcggcttgggcggcttcaggtayacaacggaggggacgatgttcacgtcgtcgttcagggtgaacacgtagctcggctcaccgaacccgtagtcvacctcgttgaaacccacatggctccagtcagtcacgacgagggtgccrtagtctagcgacacrttgtagtggttctccttggcgccgccgctcaaccagtcaaggaacctygaygacagagcttccttggcttccctgatcrcggtcacratctccacaagcgaagcttccttcacctcctggctggtcttggtgagaccacctgggtacacacagttcccgtagtagccttcgacygagggcagcacattgctcagcaggtggcgagtgctggctgcgaagcccaagcgracctcagcgtcgggcgygaagtcgactgccaaggcgcggcatttgaagattatggcmgtgaccacgtcgaaagtggaacacttctggttggtttcactygcaacctgatccttcacrytct

SEQ ID NO: 21 - DNA, 801 bp, Zea mays
ggccgggaattgttccgacccaccaatccgacacaaacgaacaaggccttacccggaacgaggaagaattggctaaacccggaacgaggacaggactgratgaattaaagttttcatgacattccaactctgctacaacttggggawgtgtcaggattgyaatcttctgaaaccttgttccctgccacccggtgttttggacctttggaattcccgrgccatgctccagcgccatctgtaatccatgcgcgtattcacaaaaastctatggcctatgctgctcattggctcggtgttcagttgatcttaggcttcggtctccttggacgtagaagaaaccaaagcaacaaaaatacagctagaaaagcaggaagtgttgtcaagtctagctgcacgtcgagcttcctttcgcgggtgcattggacagggtgcagacttgaaatggtggagttcacagcactagaaaacctgaaaagaagtaacactataattcagtcaaagaagtacaatgaatcaggggccctattctagtcgcagatataactatgttttgttttctaagtcggacaacttgctggttgttatgattgaccgacttgggcgattaatcacgattagtcggacgacttgggcgattaatcttacgacttgaaaacagtatactatgttcacccggtaaaaaacctcatcccacgacaggattatctcccccaaggcatgcatgtatgaggagcggtaaccagygcggcacwgcctatgactggatcttaacggatacaagcacacagtgaagggttagaa

SEQ ID NO: 22 - DNA, 801 bp, Zea mays
  misc_feature (662)..(662): n is a, c, g, or t
aaaaggcacttgataacctcagctataggtttcaargtcttgtgcctggtgatgcagaacacattcaagtatatggtcctgatgcacttggatctcatcctcgtgattctcagaatatgtatcatcttctacagggtaggcctcctatgatrgcacctcaccctatgatggatcacattgttaataggaaacagccagctccatttgatatggcacagtcgatacaccatgattctcaccgttctttcccatctaatgtgaatcatatgcaacataatcttcatgggccaggggtccctcacttggaccctgctggacatattatgcgacaacacatgtccatgcctggaagatttcctccagaaggcttgccaagaggtgtccctccatctcagcctgtccatcacatggctggttatagacctgaaatgggtaatgtaaataatttccatatgcaccctcgccagcccaactatggagaatttggattgatgatgccaggcaagtctcaattgtcctaattctatttgttctattaactggcagattactttgtcattatttgcaagttcagacatgccatagtgccagactttctatcggtggcactgttaacttatcatactccctccgtcccaaaatatagttctttctagctcacnttttttttctgtccacattcttttaaatgataattaatatagatatacatgtaaactgcgttcatatgttacttaataaatgtgtgattagtctwaaaaaattatattttrggatggagggagtactggttttgcatat

SEQ ID NO: 23 - DNA, 801 bp, Zea mays
  misc_feature (787)..(787): n is a, c, g, or t
gtagaaaggctgactcggcctctttcacggtcatggctcagtatcataacctataagcaacakrattcattggtgttagctccattaagtatggatatggcataacaaccaagaaccacagccacaataccttgagggtatctcctggttgcagaactgttgagatatctgcaacacggtcatgactaatctggctcacatggagaaggccgttgattccrccgatgtcratgaaggcrccataaggttttaggctctcwacagttcccaagacaactgatccaatwcctagctgggcctgactatctgccattgccttgcgattactgaggacaagcctgccttgttcctcatcgacctctacaaacttcagaggcaattctttctcaagcagctcttcagcggttgttttctgatcaagatcaaacaaaagtttatcaaataaggaattgtaaagctcagctagagcaaagcatcaaacataaaatatggtaaatayatgaaaggcagctcatgcttgccaattaaacgtagaatataagaaacttctgtgaaaagatcaagaactaaccgatgacacttgcgaaaatggaacgaaacccttaagcccatccacaagagctacwacacctcctttgtttccaccaattacctgcaagcaaaattgaataaggttraagaaactaagataacctagaaaggctgaacaatgacataaaggtattcccaacagagaccagccatatataaagttataccttcccctactgttttatggtaaaaaaancactataaacaaat

SEQ ID NO: 24 - DNA, 801 bp, Zea mays
aggaaatggctacatgatttctcactaacattaagcacagggttggggatataaatacctaacacattgtacyagtagagattcaggcaatatgttggtactagctaacggtgcgtgaaattggctttgaggtgtccaaggaaacttgcatatgacgagaaccaccgtagaacttgtattagcctatagcactactgtctacagtgtccaaccaacttgtgawtgaaaacaactacattaagaaccttagcctcgcgcccagaatagmgagacctctgcatcagtgattaccctctatacctgtactctctgagagagcaccaaacgagcatgacagcgatatccggaagagcccaattattcccagtgaacagcgcgagcgaccccgggatgaagcagaccaccccatttccaatctggttccgtgcaccccccgsaatagatccccggcggtggacccggcccctccttcccygcaaacctcctccgagggccggccggccttcgcagctacgctagttgagtcggcatcgggatccgcaaaaccaccaacgaatcctgccggcagaaacgtgcgcgatcgcgtgacggcggcacagcctgcgactgcaagtctgaaatcggtggggtgggtacctagtggcgatggcgagagctagcccaccmcccgcgccggcaagcggtagcaggctccagttgttgtactgtacccacagcaacgggcaactccaccgaaccgcacgcgaaagcaggagatcggtgggggtggagcgggggtggggagggtgggttggattcgaa

SEQ ID NO: 25 - DNA, 25 bp, Artificial Sequence
  PRIMER
tgttcactgtcaatccagtatgatg

SEQ ID NO: 26 - DNA, 24 bp, Artificial Sequence
  PRIMER
cctcaggaaagacaagatgctatg

SEQ ID NO: 27 - DNA, 20 bp, Artificial Sequence
  PROBE
tgcagatgagaggtgttact

SEQ ID NO: 28 - DNA, 22 bp, Artificial Sequence
  PROBE
ttgcagatgagaggtattactt

SEQ ID NO: 29 - DNA, 20 bp, Artificial Sequence
  PRIMER
tagccggtgtaagtggaaga

SEQ ID NO: 30 - DNA, 24 bp, Artificial Sequence
  PRIMER
tcgactgtgtctaaagcgtaaatg

SEQ ID NO: 31 - DNA, 16 bp, Artificial Sequence
  PROBE
cctagtcatgcggcat

SEQ ID NO: 32 - DNA, 15 bp, Artificial Sequence
  PROBE
ctagtcacgcggcat

SEQ ID NO: 33 - DNA, 18 bp, Artificial Sequence
  PROBE
tcgctgcactctgtcctt

SEQ ID NO: 34 - DNA, 21 bp, Artificial Sequence
  PRIMER
cggcactcgaactgctatgac

SEQ ID NO: 35 - DNA, 17 bp, Artificial Sequence
  PROBE
atccttggatgccttcc

SEQ ID NO: 36 - DNA, 17 bp, Artificial Sequence
  PROBE
aatccttggaggccttc

SEQ ID NO: 37 - DNA, 19 bp, Artificial Sequence
  PRIMER
gctcaccgaacccgtagtc

SEQ ID NO: 38 - DNA, 17 bp, Artificial Sequence
  PRIMER
gcgccaaggagaaccac

SEQ ID NO: 39 - DNA, 16 bp, Artificial Sequence
  PROBE
tcgttgaagcccacat

SEQ ID NO: 40 - DNA, 17 bp, Artificial Sequence
  PROBE
tcgttgaaacccacatg

SEQ ID NO: 41 - DNA, 22 bp, Artificial Sequence
  PRIMER
gcaggaagtgttgtcaagtcta

SEQ ID NO: 42 - DNA, 20 bp, Artificial Sequence
  PRIMER
agtgctgtgaactccaccat

SEQ ID NO: 43 - DNA, 16 bp, Artificial Sequence
  PROBE
cacgtcgagcttcctt

SEQ ID NO: 44 - DNA, 16 bp, Artificial Sequence
  PROBE
cacgtcgaccttcctt

SEQ ID NO: 45 - DNA, 21 bp, Artificial Sequence
  PRIMER
ccaagaggtgtccctccatct

SEQ ID NO: 46 - DNA, 22 bp, Artificial Sequence
  PRIMER
gacttgcctggcatcatcaatc

SEQ ID NO: 47 - DNA, 15 bp, Artificial Sequence
  PROBE
cagcctgtccatcac

SEQ ID NO: 48 - DNA, 15 bp, Artificial Sequence
  PROBE
cagcctgtgcatcac

SEQ ID NO: 49 - DNA, 23 bp, Artificial Sequence
  PRIMER
gttcctcatcgacctctacaaac

SEQ ID NO: 50 - DNA, 25 bp, Artificial Sequence
  PRIMER
gctctagctgagctttacaattcct

SEQ ID NO: 51 - DNA, 17 bp, Artificial Sequence
  PROBE
agctcttcagcggttgt

SEQ ID NO: 52 - DNA, 16 bp, Artificial Sequence
  PROBE
agctcttcggcggttg

SEQ ID NO: 53 - DNA, 25 bp, Artificial Sequence
  PRIMER
gagcccaattattcccagtgaacag

SEQ ID NO: 54 - DNA, 18 bp, Artificial Sequence
  PRIMER
gggtgcacggaaccagat

SEQ ID NO: 55 - DNA, 16 bp, Artificial Sequence
  PROBE
aagcagagcaccccat

SEQ ID NO: 56 - DNA, 16 bp, Artificial Sequence
  PROBE
aagcagaccaccccat

SEQ ID NO: 57 - DNA, 801 bp, Zea mays
tacaagcaagytgtgtagraacaaacasttwaagttaratcagtacagctcyaagtcaccttrtctggagttgaggcacctgaagtaacaccaatrgtaataggcccctcgggtaaccagttatttttctcaacmagttcaccatgctgttaaacaatccaattttcrgttaaaatgagatcatatactttcaataawtaaatttaagctctttccagtttmtaaytaaaagaakgtcccttcaaaattctattatttttaaaaaatgaatgctgtkrarggtatkagaaattacrtttarcttgtagctgatcctgtttcctggtccaatcctttgttcactgtcaatccagtatgatggaattccactgagttctccratttcttgcagatgagargtrttacttgaattccatcctccaacaacaagaataaggtcaactttctcwttcaccagctgatacatagcatcttgtctttcctragraatcaaacatatrgtyrkatgtatataagtcaatgcagcatatgtcaaaacaattataaayctrtmacycaaagagwcawtttctcaaaccaaaagtggagmaacaaatgkaytaggatcctcttarctttgrtccmaaataaaagtgycatctagttgyctcagtttccttygcaggttgcatgtaaagggtratggagattgtattataccatgaatggctaccrtctatcrtgaattgtaykctagccttttctwgacrttacagtacagtagtgctaaatccatcattcccagtttttaaatcatcaaga

SEQ ID NO: 58 - DNA, 801 bp, Zea mays
acaaccataaaatatcatttcatataytattttttatctattatcaatttttcttmtactaacaattagaagagkkcccaygtcgtgcactggtttcgagggcaggggacagcttgcagcccgccacatctascgtggtcctatgtattcctctagagctgtagcgcktagagrcaagtgtagagacgactgacgcagcagtaragtgaygctaaacagraacagtgrtagaggagaggaytactggcgggaagcgctgcggccagtctcagccatggcaaaacaagaagtgaaaaagaraacmtaaactgataaaaacagctgattcattgatccaaagtgastacayatatatakacagtgcttagccggtgtragtggaagactggragcctagtcaygcrrmatgcacracttattatattcattacaycratyaaaayatttaataaattaaawtraycaatsaactartcymatgycttaagyatagrcsaagacacartrtaygttacatttacgctttagacayagtcraaaacayagcttcaagacaccrattgtcttyctcatttykcaacmgakgatgattaaacaactttgagctcagtcatatgtgtttataattcatcatctacaactacgsgcgcgcgttgtgatcctttattgttttcatttcgataacrtgaactacttatacwwttrctcaatcatcatcacggttcttgcaagttgggttgcagggatacgggcaatcaatctgccgagggacctccctatcgwagtaccagtcccccacyg

SEQ ID NO: 59 - DNA, 801 bp, Zea mays
tggttatggaggccggagtcatttcagcttcctctcccccttcttcggtgtctccaagcccgccactgycgtcctcaayaytgctgbtgaaccctgaacgcagcatgctcatcaagtcyccaaggtcycgttgctgctccatccggcgccartcccgctgccgctcagggtctacctccattggccttcctgttggatgctccaccctagcatgcttcctcagctcmtggtargcgcccctgaactcgcacgactccgtggtgcacgaccggaccttggtgttcatgaacctccgcgcgctgtagtccttgatgcaatcgctgaccggcccacggcacagtgggcacgagagctkaacctgctgctggcactcgctgcactctgtmcttgaatccttggakgccttccggtactggtcatagcagttcgagtgccgcgagctggtgtcgcacatgaaggggcggcagcccttctcgtgtgaggagcagaccagcaggacggcgttgtgcgggtggtccatgcacaccggrcaccgaacrtcctcccactctgcagcttgctttgctgcygccgccgctgctgccccacaagactcctckgaccggcgasaagaactgctctgtccatgmgacgatgagaagtatggrgaggcaccagcccggcggctctcrtaagaggaaacgcgggagctcctgtccttcggcatagttatatcycaagtcagcgagatgcaaggcttattcgaagcagcacagacagacagaatacagatgcagggttggtttctttactccaaactct

SEQ ID NO: 60 - DNA, 801 bp, Zea mays
caggccaacgartctgaaataatttgttccaaatgggaacaktattattcyttcgtcactgtcaatattcatcacatgtataatgtaacgtgcygataatggtacaaagtataaacaygaccaatmtgmcttattgsaagcaawttccrggagcagcaatttgacrcaacaaayaaaatacaacasattcccastgtwgaatacactcactctatgcatgcttctgcaactcctcgcygaacacrgcagagtgatggccttccacgcactgcagsaccagcctgatgccctgcttcggcttgggcggcttcaggtayacaacggaggggacgatgttcacgtcgtcgttcagggtgaacacrtagctcggctcaccgaacccgtagtcmacctcgttgaarcccacatggctccagtcagtcacgacgagggtgccrtagtctarcgacacrttgtagtgrttctccttggcgccgccgctcaaccagtcaaggaacctygaygacagagcttccttggcttccctgatcryggtcacratctccacaagcgaagcttccttcacctcctggctggtcttggtgasaccacctgggtacacacagttcccgtagtagccttcgacygagggcagcacrttgcycagcaggtggcgagtgctggctgcgaagcccarrcgracctcagcrtcgggygygaagtcgaytgccawggcrcggcatttgaagattatggcmgtgaccacrtcgaaagtggaacacttctggttggtttcactygcaacctgatccttcacrytct

SEQ ID NO: 61 - DNA, 801 bp, Zea mays
ttctaacccttcactrtgtgyttgtatccgttaagatccagtcataggcwgtgccgcrctggttaccgctcctcatacatgcwtgccttgggggagatratccygtcgtgggatgaggttttttaccgggtgaacatagtatactgttttcaagtcgtmwgattaatcgyccargtcgtmcgactaatcgtgattaatcgcccaagtcggtcaatcataacaaccagcaagtygtccgacttagaaaacaaarcatagttatatctgcgactagaataggrcccctgattcattgtacttctttkmctgaattatagtgttacwwcttttcaggttttctagtgctgtgaactccaccatttcaagtctrcaccctgtccaatgcacccgsgaaaggaagstcgacgtgcagctagacttgacaacacttcctgcttttctagctgtatttttgttgctttggtttcttctacgtccaaggagaccgaagcctaagatcaactgaacaccgagccaatgagcagcataggccatagastttttgtgaatrcgmgcatggattacagatggcgctggagcatggcycgggaattccaaaggtccaaaacaccgggtggcagggaacaaggtttcagaagattrcaatcctgacacwtccccaagttgtagyagagttggaatgtcatgaaaactttaattyatycagtcctgtccycgttccrggtttagccaattcttcctcgttccgggtaaggccttgttcgtttgtgtcggattggtgggtcggaacaattyccgrcc

SEQ ID NO: 62 - DNA, 801 bp, Zea mays
atatgcaaaaccagtactccmtccrtccyaaaatataattttttwagactaatcacacatttattaagtaacatatgaacgcagtttacatgtatatctatattaattatcatttaaaagaatgtggacagaaaaaaaargtragctagaaagaactatattttgggayrgagggagtatrrtaagttaacagtgccacygatagaaagkctggcactatggcatgtctgaacttgcaaataatgacaaagtaatctgccagttaatagaacwwatagaattwrgacaattgagacttgcctggcatcatcaatccaaattctccatagttgggctggcgagggtgcatatggaaattatttacattacccatytcaggtctataaccagccatgtgatgsayaggctgagatggagrgacacctcttggcaagccttctggaggaaatcttccaggcatggacatgtgttgtcgcataatatgtccagcagggtccaagtgagggacccctggcccatgaagattatgttgcatatgattyacattagatgggaaagaacggtgagaatcatggtgtatcgaytgtgccatatcaaatggagctggytgtttcctattaacaatgtgrtccatcatagggtgaggtgcyatcataggaggcctaccctgtagaagatgatacatattctgagaatcacgaggatgagatccaagtgcatcaggaccawrtacttgaatgtgttctgcatcaccaggcacaagacyttgaaacctatarctgaggtyatcaagtgcctttt

SEQ ID NO: 63 - DNA, 1001 bp, Zea mays
  misc_feature (103)..(103): n is a, c, g, or t
  misc_feature (264)..(264): n is a, c, g, or t
  misc_feature (309)..(309): n is a, c, g, or t
  misc_feature (321)..(321): n is a, c, g, or t
  misc_feature (330)..(330): n is a, c, g, or t
  misc_feature (339)..(339): n is a, c, g, or t
  misc_feature (360)..(360): n is a, c, g, or t
  misc_feature (387)..(387): n is a, c, g, or t
  misc_feature (414)..(414): n is a, c, g, or t
  misc_feature (429)..(429): n is a, c, g, or t
  misc_feature (675)..(675): n is a, c, g, or t
  misc_feature (696)..(696): n is a, c, g, or t
  misc_feature (717)..(717): n is a, c, g, or t
  misc_feature (726)..(726): n is a, c, g, or t
  misc_feature (741)..(741): n is a, c, g, or t
  misc_feature (907)..(907): n is a, c, g, or t
  misc_feature (909)..(909): n is a, c, g, or t
  misc_feature (991)..(991): n is a, c, g, or t
ttcagcacatgagaaaatgttgcttttgtttattcttaccttctcaaacacaagcttgggattgcggatcatgtcaccaggtgttggctcaagcttcttagtngaaaggctgactcggcctctttcacggtcatggctcagtatcataacctataagcaacagaattcattggtgttagctccattaagtatggatatggcataacaaccaagaaccacagccacaataccttgagggtatctcctggttgcagaactgttganatatctgcaacacggtcatgactaatctggctcacatggagaagnccgttgattccnccgatgtcnatgaaggcnccataaggttttaggctctcnacagttcccaagacaactgatccaatncctagctgggcctgactatctgccatngccttgcgattactnaggacaagcctgccttgttcctcatcgacctctacaaacttcagaggcaattctttctcaagcagctcttcrgcggttgttttctgatcaagatcaaacaaaagtttatcaaataaggaattgtaaagctcagctagagcaaagcatcaaacataaaatatggtaaatatatgaaaggcagctcatgcttgccaattaaacgtagaatataagaaacttctgtgaaaagatcaagaactaaccgangacacttgcgaaaatggaacnaaacccttaagcccatccacnagagctacnacacctcctttgttnccaccaattacctgcaagcaaaattgaataaggttaaagaaactaagataacctagaaaggctgaacaatgacataaaggtattcccaacagagaccagccatatataaagttataccttcccctactgttttatggtaaaaaaacactataaacaaatagacaananaggagcaaagatatataacaccttgaagacagatgacatgttctcataaactgactgatcctaacttcataagttcaataantgtagcacat

SEQ ID NO: 64 - DNA, 1001 bp, Zea mays
  misc_feature (3)..(3): n is a, c, g, or t
  misc_feature (47)..(47): n is a, c, g, or t
  misc_feature (88)..(88): n is a, c, g, or t
  misc_feature (90)..(90): n is a, c, g, or t
  misc_feature (104)..(104): n is a, c, g, or t
  misc_feature (107)..(107): n is a, c, g, or t
  misc_feature (112)..(112): n is a, c, g, or t
  misc_feature (125)..(125): n is a, c, g, or t
  misc_feature (225)..(225): n is a, c, g, or t
  misc_feature (241)..(241): n is a, c, g, or t
  misc_feature (260)..(260): n is a, c, g, or t
  misc_feature (264)..(264): n is a, c, g, or t
  misc_feature (266)..(266): n is a, c, g, or t
  misc_feature (317)..(317): n is a, c, g, or t
  misc_feature (319)..(319): n is a, c, g, or t
  misc_feature (368)..(368): n is a, c, g, or t
  misc_feature (445)..(445): n is a, c, g, or t
  misc_feature (987)..(987): n is a, c, g, or t
canagggcttcatgatttgtcagtaacactgagcaagaagaacaggnatgtcaaatggcttcatgatttctcaggaacaccaatagcnangcacaggcttaggnaanggctncatgatttctcantaacattaagcacagggttggggatataaatacctaacacattgtaccagtagagattcaggcaatatgttggtactagctaacggtgcgtgaaattggntttgaggtgtccaagnaaacttgcatatgacgagnaccncngtagaacttgtattagcctatagcactactgtctacagtgtccaaccaacntntgattgaaaacaactacattaagaaccttagcctcgcgcccagaatagngagacctctgcatcagtgattaccctctatacctgtactctctgagagagcaccaaacgagcatgacagcgatatcnggaagagcccaattattcccagtgaacagcgcgagcgaccccgggatgaagcagascaccccatttccaatctggttccgtgcaccccccgcaatagatccccggcggtggacccggcccctccttccctgcaaacctcctccgagggccggccggccttcgcagctacgctagttgagtcggcatcgggatccgcaaaaccaccaacgaatcctgccggcagaaacgtgcgcgatcgcgtgacggcggcacagcctgcgactgcaagtctgaaatcggtggggtgggtacctagtggcgatggcgagagctagcccaccacccgcgccggcaagcggtagcaggctccagttgttgtactgtacccacagcaacgggcaactccaccgaaccgcacgcgaaagcaggagatcggtgggggtggagcgggggtggggagggtgggttggattcgaagcttgcgttgcacgggacagatctacagctgagcggtggcgtgcgggcacagattggaatcgcccgccgacccagagcgcgggganggaggtgggcgaat

SEQ ID NO: 65 - DNA, 552 bp, Zea mays
  misc_feature (75)..(75): n is a, c, g, or t
  misc_feature (79)..(79): n is a, c, g, or t
  misc_feature (121)..(122): n is a, c, g, or t
  misc_feature (502)..(504): n is a, c, g, or t
  misc_feature (506)..(506): n is a, c, g, or t
  misc_feature (508)..(508): n is a, c, g, or t
tagggtcctgctacaagagatcgccacattttattgctacggaagtccagttgtgtctgtctgtttggtggtcantggnatatggttcggtttttactgctgtaaaaagggactsgggaannaaaaatgcaaactgacttggattttttgttctgttctgcatgaagatgaaatggtagggtcgtcggaggaggacgaagcatgctcgggaggagacacggaggcgacggagccggggcagcaggagcacagctcccgcctggcggaccrtgagctgaaggagatgctgctgaagaagtayagcgggtgcctgagccggctgcggtccgagttcctgaagaagaggaagaaagggaagctgcccaaggacgcgcggtcggcgctcatggactggtggaacacgcactaccgctggccgtaccctacggtaaccatgcatgcatcctggcaaacacgcagcagcagcatcgctcgctggaatgrcagatctgtgaccagcatnnncngncggtgcaggaggaggacaaggtgaggctggcggcggcgactggg

SEQ ID NO: 66 - DNA, 7437 bp, Zea mays
  misc_feature (4454)..(4454): n is a, c, g, or t
  misc_feature (4463)..(4463): n is a, c, g, or t
  misc_feature (4474)..(4474): n is a, c, g, or t
  misc_feature (4497)..(4498): n is a, c, g, or t
  misc_feature (4536)..(4536): n is a, c, g, or t
  misc_feature (4549)..(4601): n is a, c, g, or t
  misc_feature (5112)..(5112): n is a, c, g, or t
  misc_feature (5194)..(5194): n is a, c, g, or t
  misc_feature (5297)..(5299): n is a, c, g, or t
aatgtcaagtatatccatttaaatatcattaggtcccgtttgtttcctttcattttaaggaattggaatcttactaataaaataagctatttttttagaatacgagattccaccactttccaaagttatcagataagcctatctcaaattcatggggtgagagatggaaattgattctatagatttacatgttattttcccgatgtacaacttatatcatactctcctaattgcttcgctataacataaatgcactatataactatctctcttatatgatttaggataatatacaaatatattacatatataaatatattaacttaattagttttgtctaaattataattattaaaatggaattcaattccaacgaaacaaacgggcccttacaaaatttctagtatcatttaaccatctattcaacacaccaaagataattggataaaatagcaacactaggacaaatactacatagcacattacatgttccattatatggtcattaaattgtcgttaagccttctataacattatggcctaaaaggttgttaaatagtaaaaaaagatggatgactaaagtccaagttcatgcttgggctaagtttatattgggtattttataccacgggttaacgggtatgggtgaaggcggaacgttctgattcccgtttacttattgggtgaagatttttgcctaataatagacctacgggtgaatatttatcccacatatatatcctagtggagtcaatatccatcggatatcggtcgtgggtacccattgccatctctagatcgaagagtagaaatttacttcctaaacctctctctgtctcatgcagcacaatagacgctttgtttcgttgcaacagcttgtttctctttgacgtccaaattcgtctatctacggacccacggccgcccagattttgaaatttcaaaacggaacacaccccggggttcggagtatctgctgcccctgcggtcctggaaagcccggcccacctctcacttgcaccaccagctcaccggttccggtcaacagtctctcgcgggtgcctgtgccggtggtcctccgtcctggctctggctgccacccacctcccgattaccgttctcgcctcgacttcaaaacaagagcccagatctaaaccaagcacgcccatctttgccacaccacacccccagtattcgaatctctcgtgcccagatgcggcaaattaaaaacaacggacagacgcggaacccctccggccaacggatctcaccctctgcggcatgggtcccactcacgctcgggtccactcgacagcgtgtagggcagagaggcgagcggtaccagtaccataggcctcccgacgcagtccgggcagcgggccccgcggattggaccggtcaaaaggcgtggcccaaccaaaccccaagggatccggcgctttgtctgcacgtgaatggtgccaagatcgcctggttgacaggtgggacccgtgaggttgtagacccacatgtctgtggcgttaaaggaggggggagggatcggcgggcgggtggtgcgcgcgggcaggcgggcgtcgcgtggtggtggtggtggtgggtgctttgactgcaggcctcggcagcaggcagagaggactagaggagtcggggcctcggaggaggggagggagagggcgaagagtagggggaaccaaatcttgaagggtaaacggagagttctttcgtggaggaggaagggggggacagcaggaggagggtagaggtatgtgcgcacccatctgttcttgctcctgatttggctgtttgttttttctgtctgttcttcgctgttggtagtttgtgaccgtgaatgggcgttcctggtccatgttcgcgtgcgctgctgccgattctgggagctctctggtcgtccgtctcgctgggatctgccttttccccggtgagagccgcggaacgttcgccgccttttcttactcgcgggccagttatggtttctggagcgttttctctgttcttggcgaggtggtcatcgctctgagaacgatgcgctctttctccgagtttgtgctcaagttttcgtcagcctagaggctatagcgtttgctgcggatctcacgacttctctcttcctcttctctattggtgcatacgttttcatccgaaatccattagttagtgcccgagccgtcaattctttgtggatttgcttgttccccttcgttacaggctcggaaatgcccctgaacagattcacaggggtcctagattaggattattttctatgactttccaagagtcaggagcacgattgctttctctcggctgtctgcctggttcatgactcagccgggtttgcaagcctaggaagaacttgctcacgtttcttacatttatctagattcgagggacgggttgtactcgttaacaaagttcacctcgttagtcattaaagctccgctgttgtgaatgatgctgccattgcgatatctggaatcatcgctctgatcgatttggttgttaatccacttacaggtagctcaatagatctactgctctcgggggagttaatgcaaagctgagttgctgcacgttggctttcttcagagatggcttcagctggtgtagccccatctgggtacaaaaacagcagcagcactagcattggtgccgagaagttgcaagatcagatgaacgagctaaagattagagatgataaggtgaagatgccttgatatcttgtttcgggcttactgtaatttcctcaagattatgtgaaaaatgggactgtgatgtaacctttggtgtgaatgccaaatgcaggaagttgaagcaaccataattaatgggaaagggactgaaactgggcacataattgtcaccactactggtggcaagaatggtcaaccaaaacaggtgagtgctttactgcatttgatcatgatttatcaactattctacatgtttttagtgcatgtctgaatctaataattgagagtcaagaccataatttaatgtccttcttttgcatattgccaatatatccatgttgctaacttataagattgtggagttgttctgatcagttttgtcagattctttttgtataataatgtgtatttattggttgcatttgcagacagtgagctacatggctgagcgcattgtaggtcaaggttcttttgggatcgtcttccaggttatttgcaataacttgtgactgactttgatatgtactattatgtagccgcctgtggtgttgctttccacggcgctgcacatgttttagatcttcatatcttgcgtgctataaatcacctttcttaatcagatgccatttcacctgttcataggctaagtgtttggagacgggtgagactgttgccataaagaaggttcttcaggataagcgttacaagaaccgcgagttgcagaccatgcgccttcttgaccaccctaatgttgttgctttgaagcattgcttcttttcaactaccgagaaggatgagctttatctgaacttggtccttgagtatgtgccggagacagttcatcgagttgtgaagcatcacaacaagatgaaccaacgcatgccacttatttatgtgaagctgtatatgtaccaggtaatggtttgtcctgttcctttttgctgttgttttaattataccttaaagcttatgttttgggccctgtttgatgttgaaactaacaaacatatttcatttcgcctaaatattgtctgctccaatgaatgtgctagttctttttcaatatttgatattatattggattttggcagatatgtagggcattggcttacattcatggcactattggtgtctgccacagagatattaagccacaaaaccttctggtatgctggaaaatctgctattttgctactgtatctttttgtaaagaaatgatttgtactttgaaattgatgttcaaacttcactacaggtgaacccacacacccaccagcttaaactatgtgactttggcagtgcaaaagttctggtcaagggggaaccaaacatatcgtacatctgctcccgatactatagggctccagagctcatatttggtgccactgagtataccacagcgattgacatttggtctgctggatgtgttcttgctgagcttatgctagggcaggtaaggtgtctcaaatttttattgccattttaaaaaaggttttcaagccaacaaggtcctttcagttcacactgtcttacaagaactatttggacagcctttgtttccgggtgaaagtggtgtggaccaacttgttgaaatcatcaaggtaattgtcggttctacaagcttgtgaattgtcttctatagaagcataaaatctgatcacccctaaaatgattttgtatggcaggtcctcggtacgccaacaagggaagaaattaaatgcatgaacccaaattacacagagtttnaagttcccnacaaatcaaangcacacccatggcacaaggtgcnnaaatctktctacattttgttacaatactctaagaaaanctgttactgttgnnnnnnnnnnnnnnnnnnnnnnnnnnnnnnnnnnnnnnnnnnnnnnnnnnnnntgttactwatttactttttgtacattttatctttcaggtmttccacaaaaggatgccgccagaagctgttgatctggtctctcggctactccagtactccccaaatctgagatgcactgctgtaagtgcatgccattgtacattatacatgatggaaatacccctgttgactttggttttctaagatcttyatgaatgttttgtccagatggaggcacttgttcacccattcttygatgagcytcgagatcctaatactcgccttccaaatggtcgctttttgccaccactattcaatttcaagcctcacggtatgtttcatgcctacataattcaacatcgttatcatagctgctacaaccaggtakcagtgtagtwcyaagtttgttctttgtatatcaccaccttacatgctcgccacctctgttctgcagaacttaaaggagtcccatcagacattgtcgcgaaattgattccagaacatgcaaagaagcaatgctcctatgttggnattgtgaaatgaccgcgccttgagactggaacctgtggttgcaattgtgaatttcccctgggatgtttgacgatctgaggcnatgcgagcctgttgttgaagatgcaaggttacgtacttgtacgacaatgtgacctgtgtagctgagtagtctatgtcgcagtgacatgtaacggcaccccccnnnttcctactaactgacgcttactcgagattgccatagttgatcttgtaatttgttatagagcagtatgaatgtatttatggtagcttgaatctatgtatggattcacttcgtttttccatgtttccttgtctccagacccagattgctaccgtattgtttcagaattcctagctacctgttgcctattgagtattgactaccagcttgcacttgtctgttattgcactggctgtggaatcagctgttgatttttgccacaatattttagttcagatgtactccctattctaaaaagaatgtgaaatcttactaatagaatagactactttttttagaatttctttccattttgaggaattaaaatcttactaatagaatagactacttttttttagaatgtgacattacaccactttctaaagttatcatataagcctatctcatttatggggtgagagatgaaaattgattatatagatttacatactgtttttccgatgtacaatttatagcacacccttctacttgcttcgctataacataaatgtagtatataactatctctttcatgtgatttaagataatatataaatatattacatatataaatatatgaacttaattagttttatctaaattataactattaaaataaaattcaatttcaacgaaacaaacggggccttgattaattataaaatgtatttttgtaataagttgatttaaagctataatgtaaatactatttactagaaacttggttaaatatgaattagtttaactaacgagtttaattggcataccacttatagttatattctttgagacggagggacgagtacgttgttcgatcggtctggaagtatgctgacttgatcgttcttaccagaaagttgcattattgcagcgtttgagacgactgacgaggaaatgtgacacgcagatgctactcagtgcttggcaggactgcattccaagtggtccttctggggagagaggaatcatagactgtagctccggtttcttgaaaaaaaacggttcccgtgaaatggcaggtatggttctccggttcctttgaaaactactctttgtaaaatgaagtatgcttggctctatcgaagttagctgttgttaacagccataccagacaggttctttcagtgtccggttagattttgaggcgtcgagggttgtttggttgagaagtggagagttcctttagagtgtgtttagttgagaagtggaggaaaatggatcgactatattcctattttttttatgtttagtttccaagaaaagcggagcagagcggctcctgaagttttagaaatttaccataaatagtttaaatgctcccgctccgtcaaaacgaacatacacgagcgctctcctccctctacttccttctacaaccgtatgtctttccaatcaagcaaagaacggagtagctctgctctattctactcttaaccaaacaaaaaaatgaagtgactctgttctgcttgtcaaatgcgaaatagaatgattctattctaaaaaattggaatagagccgctccaaccaaactaacctcactcgagggactaaagtttagtctttactctatttgattccaaggactaaaagtattcataacatattaaatgacttgaaaactaaaatgttcttaacattcttccgccattagcataactaaaataaactagggataagtgaaattaatatggactaaaacaatttggtcgctgttttattcccatatttgacaatttagaaattaaataaaactaaaatagatggattaatttttagttcctcaaacaattttttctaacaattttcgatggactaagtttagtcatttttcataagaaattaatatggactaagggcctgtttgtttacccctcagattatataatctggattaaataatcctaagaggcaaacaaacagtctagcttatttgtcgagattatataatctaactcctggattatgataatccataagcaagtgaggaggtgcttatttcagattatttttttccacttctccactaccctttcaagtttcctagaaattacccaccattgccattataacccaccattggcattcttgtcttcctcatacaa

SEQ ID NO: 67 - DNA, 570 bp, Zea mays
aaggtgggaaaactgaaaaagacaatcatgaaggtgtaaattctttactatctgaggagttggaaaaactagctaatgggaatagcaaggtatgatataccctccatgattctgctttcatttattctttgttcatatggtatggttatttaacagtgctatgtattgccgtaaatgcagattcctggtacattagatgagtatagaaagcttgtcrttccaataattgaggagtattttagtacaggagatgtggaattggcagcttctgagctgaagtgtcttggatctgatcagtttcatcattactttgtgaagaagcttatatctatggcaatggatcgccatgacaaagaaaaagaaatggcatcgattctgttatcatctttrtatgctgatctactgagctcctacaggatcagtgaaggttttatgatgcttctggagtctacagaagatctaactgttgatataccrgatgctactgatgtattggcagtttttattgcacgggctattgttgatgaaattttgcctcctgttttcctcactcgagctagggcactactt

SEQ ID NO: 68 - DNA, 904 bp, Zea mays
  misc_feature (87)..(89): n is a, c, g, or t
  misc_feature (94)..(97): n is a, c, g, or t
  misc_feature (138)..(138): n is a, c, g, or t
  misc_feature (145)..(145): n is a, c, g, or t
  misc_feature (257)..(258): n is a, c, g, or t
  misc_feature (263)..(263): n is a, c, g, or t
  misc_feature (319)..(319): n is a, c, g, or t
  misc_feature (424)..(424): n is a, c, g, or t
  misc_feature (441)..(444): n is a, c, g, or t
  misc_feature (448)..(448): n is a, c, g, or t
  misc_feature (451)..(459): n is a, c, g, or t
  misc_feature (461)..(461): n is a, c, g, or t
  misc_feature (464)..(465): n is a, c, g, or t
  misc_feature (494)..(494): n is a, c, g, or t
  misc_feature (547)..(547): n is a, c, g, or t
  misc_feature (552)..(552): n is a, c, g, or t
  misc_feature (557)..(557): n is a, c, g, or t
  misc_feature (602)..(607): n is a, c, g, or t
  misc_feature (634)..(637): n is a, c, g, or t
  misc_feature (647)..(647): n is a, c, g, or t
  misc_feature (652)..(654): n is a, c, g, or t
  misc_feature (657)..(657): n is a, c, g, or t
  misc_feature (659)..(659): n is a, c, g, or t
  misc_feature (665)..(665): n is a, c, g, or t
  misc_feature (682)..(682): n is a, c, g, or t
  misc_feature (691)..(691): n is a, c, g, or t
  misc_feature (693)..(693): n is a, c, g, or t
  misc_feature (746)..(746): n is a, c, g, or t
  misc_feature (806)..(806): n is a, c, g, or t
  misc_feature (842)..(842): n is a, c, g, or t
atgcgcccttgggtacatagcaggagactggaaaggtaacaaatggaatacggacacgtagataaccatggaggcccagcaatgttnnngaggnnnntattgtttggtcactgcatagccgatgatgatcaccacgcnctcgtgnatgcctcatgtcttaacagcagccagatgatctctgrgcccggttgccacctaccacatccacggtatctggcacataccttcaagaacaaatcgataaggtcaaaaaaaannggggnacggcttttacatagataataaaggaccagcacagggaaacaaatraaggaaatcnaaaagtgattratgattttacatagatataacactgaaaacgagaccagcagaagaagctagtcttattgcagcagctaatgatgccaaccctggtacacccccnagaaagaggatcaacannnngaanagnnnnnnnnntngsnncttattttgctgatgactaacaactggangcaaaaagaccaacagaaggaccggttgatattaaaaatgtaattacttttancagcnagcgnctccggttttctagtacttgcagcaactaatgtcacataaaaacnnnnnntgtcaaatgccaatcaaacacacaaannnngaaacgaganatcannnagngntgtgcnttacaattcttcctccnggttcattncnttcagagcattctttacaatacactggaaggcatcttccacgttcgttccatnccttagcagacgtctcaaagtacgggatgttccctttagaggcgcaccatgcctttgccntttttctccgacacctaaaaccaaagcagattatantttctaagcagctcgtgatccagttcaagagaagaatttattcagagaacaaatcatgtagc

SEQ ID NO: 69 - DNA, 426 bp, Zea mays
  misc_feature (310)..(310): n is a, c, g, or t
  misc_feature (316)..(324): n is a, c, g, or t
  misc_feature (348)..(348): n is a, c, g, or t
  misc_feature (396)..(396): n is a, c, g, or t
  misc_feature (405)..(407): n is a, c, g, or t
tcttcagtctccaccctgattcaacaacagactctgacagcttgcacggtagctgccccgycatcgaaggccagaagtggtccgcgactaaatggatccatgtgaggtcatttgacctcaccgtcaagcagccgggtccctctgatggatgtgaggacgacaatgtcctctgcccccagtgggcggccgtgggcgagtgygccaagaaccctaactacatggtggggaccaaggaagcacctggcttctgccggaagagctgcaaagtatgcgcagagtaaggtatcggtcctctgcgtctgatgagtantcgtgnnnnnnnnnttacgtagttgctgtcaccatttnaccagggtttagatacgaccgagtacagcatgtataagacagtacaancccggaagnnngagtcgtaagagttagggg

SEQ ID NO: 70 - DNA, 595 bp, Zea mays
  misc_feature (201)..(204): n is a, c, g, or t
  misc_feature (458)..(461): n is a, c, g, or t
  misc_feature (569)..(576): n is a, c, g, or t
cgtccgcggcagcggccgcgtccaggtggtcgggcccgacggggtgcgcgtcctggagacgcgsgtcgagggcggcttcctcttcatcgtgccccgcttccacgtcgtctccaagatcgccgacgcgtccggcatggagtggttctccatcatcaccacccccaagtaatttgttgtctcgatcgatcgatccatcgatcnnnntttttttattgcgaattgcactggagatttgattgcacgtgaattaatgyttgcattgcattgcagcccgatcttcagccacctggccgggaagacgtcggtgtggaaggccatctcggcggaggtgctgcaggcgtcgttcaacaccacgccggagatggagaagctgttccggtccaagaggctcgactcggagatcttcttcgctcccccatccaactgagaaaataggccggaagccccacggtggagtnnnncctctcgttaggtcgtcgtgcttagattaggttagctagcttgcctttaataaaaagagagtggtggtcgtcggcgtcggcttcggcggtctgcttcttcttcattcnnnnnnnnagtgcgtcggtcggtttag

SEQ ID NO: 71 - DNA, 558 bp, Zea mays
  misc_feature (39)..(39): n is a, c, g, or t
  misc_feature (58)..(58): n is a, c, g, or t
  misc_feature (60)..(60): n is a, c, g, or t
  misc_feature (67)..(67): n is a, c, g, or t
  misc_feature (80)..(80): n is a, c, g, or t
  misc_feature (82)..(82): n is a, c, g, or t
  misc_feature (97)..(97): n is a, c, g, or t
  misc_feature (104)..(104): n is a, c, g, or t
  misc_feature (111)..(111): n is a, c, g, or t
  misc_feature (116)..(116): n is a, c, g, or t
  misc_feature (138)..(138): n is a, c, g, or t
  misc_feature (150)..(150): n is a, c, g, or t
  misc_feature (155)..(155): n is a, c, g, or t
  misc_feature (157)..(157): n is a, c, g, or t
  misc_feature (159)..(159): n is a, c, g, or t
  misc_feature (166)..(166): n is a, c, g, or t
  misc_feature (172)..(172): n is a, c, g, or t
  misc_feature (174)..(174): n is a, c, g, or t
  misc_feature (186)..(186): n is a, c, g, or t
  misc_feature (191)..(191): n is a, c, g, or t
  misc_feature (193)..(193): n is a, c, g, or t
  misc_feature (196)..(196): n is a, c, g, or t
  misc_feature (205)..(205): n is a, c, g, or t
  misc_feature (208)..(208): n is a, c, g, or t
  misc_feature (212)..(212): n is a, c, g, or t
  misc_feature (215)..(215): n is a, c, g, or t
  misc_feature (217)..(217): n is a, c, g, or t
  misc_feature (219)..(219): n is a, c, g, or t
  misc_feature (228)..(228): n is a, c, g, or t
  misc_feature (231)..(231): n is a, c, g, or t
  misc_feature (233)..(233): n is a, c, g, or t
  misc_feature (247)..(247): n is a, c, g, or t
  misc_feature (255)..(255): n is a, c, g, or t
  misc_feature (258)..(258): n is a, c, g, or t
  misc_feature (261)..(261): n is a, c, g, or t
  misc_feature (265)..(265): n is a, c, g, or t
  misc_feature (278)..(278): n is a, c, g, or t
  misc_feature (280)..(280): n is a, c, g, or t
  misc_feature (292)..(292): n is a, c, g, or t
  misc_feature (321)..(321): n is a, c, g, or t
  misc_feature (323)..(323): n is a, c, g, or t
  misc_feature (329)..(329): n is a, c, g, or t
  misc_feature (331)..(331): n is a, c, g, or t
  misc_feature (335)..(335): n is a, c, g, or t
  misc_feature (348)..(348): n is a, c, g, or t
  misc_feature (370)..(370): n is a, c, g, or t
  misc_feature (393)..(393): n is a, c, g, or t
  misc_feature (395)..(395): n is a, c, g, or t
  misc_feature (403)..(403): n is a, c, g, or t
  misc_feature (413)..(413): n is a, c, g, or t
  misc_feature (432)..(432): n is a, c, g, or t
  misc_feature (447)..(447): n is a, c, g, or t
  misc_feature (467)..(467): n is a, c, g, or t
  misc_feature (472)..(472): n is a, c, g, or t
  misc_feature (474)..(474): n is a, c, g, or t
  misc_feature (477)..(477): n is a, c, g, or t
  misc_feature (494)..(494): n is a, c, g, or t
  misc_feature (505)..(505): n is a, c, g, or t
  misc_feature (508)..(508): n is a, c, g, or t
  misc_feature (526)..(526): n is a, c, g, or t
  misc_feature (529)..(529): n is a, c, g, or t
  misc_feature (550)..(550): n is a, c, g, or t
ccgtgttggttggatctatggctcggtgacggaggatgntggtcactgggtaccggantngcacaanccggggttggaangntcggtgtactgtgtnyaccaangcgtgancgccnttccgcggcaccgcgcccatcnaacctgacyganccgtncntnccaccanggtgcntnccggtgggctanctggnantcnagtggagantcnttcnttncntncccgmaacnaancngcgctgctggcgangccgcagnaantgnaagnttcttgcagaggnantcgcgtacctgnaacgtgggtatctacccgttcacgtccantncttccntngatncgtctactgcttncctgccggcgctgtcgctgttnctcggggcagttcatcgtgaagnancgctgaancgtgacgttncctgacgtacctgctggtngatcacgctgacgcntgtgcctgctggcggtgctnggagnantcnaagtggtcggggatcangyctggaggangtnggtggcggaacgagcagnttnctggctgatcggcggcacgangcgcgcac

SEQ ID NO: 72 - DNA, 1193 bp, Zea mays
  misc_feature (8)..(8): n is a, c, g, or t
  misc_feature (15)..(15): n is a, c, g, or t
  misc_feature (25)..(25): n is a, c, g, or t
  misc_feature (79)..(86): n is a, c, g, or t
  misc_feature (88)..(88): n is a, c, g, or t
  misc_feature (100)..(102): n is a, c, g, or t
  misc_feature (124)..(126): n is a, c, g, or t
  misc_feature (135)..(135): n is a, c, g, or t
  misc_feature (239)..(242): n is a, c, g, or t
  misc_feature (357)..(357): n is a, c, g, or t
  misc_feature (365)..(365): n is a, c, g, or t
  misc_feature (379)..(379): n is a, c, g, or t
  misc_feature (470)..(470): n is a, c, g, or t
  misc_feature (496)..(503): n is a, c, g, or t
  misc_feature (506)..(507): n is a, c, g, or t
  misc_feature (512)..(513): n is a, c, g, or t
  misc_feature (519)..(524): n is a, c, g, or t
  misc_feature (571)..(579): n is a, c, g, or t
  misc_feature (623)..(625): n is a, c, g, or t
  misc_feature (670)..(670): n is a, c, g, or t
  misc_feature (691)..(691): n is a, c, g, or t
  misc_feature (806)..(808): n is a, c, g, or t
  misc_feature (841)..(841): n is a, c, g, or t
  misc_feature (847)..(848): n is a, c, g, or t
  misc_feature (850)..(850): n is a, c, g, or t
  misc_feature (968)..(975): n is a, c, g, or t
  misc_feature (1016)..(1018): n is a, c, g, or t
  misc_feature (1083)..(1083): n is a, c, g, or t
  misc_feature (1108)..(1108): n is a, c, g, or t
gtgaatgnacagggngactcctggnacagcctactcggcaggaacaagcacgacgaccaggagaagaagaaccagcagnnnnnnnngncggaggaggagnnnctggcgaccggcatggagaagnnngtcacggtnggccgagcccgaccacaaggaggagggacacgaggccgccgagaagaaggacagccttctcgccaagctgcaccgcaccagctccagttccagctcggtgagtnnnntcgtcgtaaaacatgayctgctgctagctagtttaattgactccgccttcggawcagtaagctaataaaccggcttctcactgcgatcgtggtgcctgcgcgcatgcagtcgagncgacgacnaggaagaggaggtngatcgatgagaacggcgarattgtcaagaggaagaagaagaagaagggccttaaggagaaggtcaaggagaagctggcggcccacaaggcnccacgatgagggcgaccaccaccagnnnnnnnnacnngcccnngcgccnnnnnngcccgtggtggtggacacgcatgctcaccaccaggagggagagcacnnnnnnnnnttcccggcgccggcgcctcccccgcacgtggagacgcaccaccnnnccgtcgtcgtccacaagatcgaggacgacgacacgaagattcagnaccccaccacaggcaccggangaggagaagaaaggcctgctggacaagatcaaggagaagcttcccggtggccacaagaagccggaagacgctgctgccgccgccgccgcgccggccgtccacgcgccaccgccgnnngcgccgcacgccgaggtcgacgtcagcagcccncgatgnngncaagaagggcttgctgggcaagatcatggacaagataccccgctaccacaagagctcgggtgaagaagaccgcaaggacgccgccggcgagcacaagaccagctcctaaggtcgcagnnnnnnnncgtgtgcgtgtccgtcgtacgttctggccggccgggccttnnngggcgcgcgatcagaagcgttgcgttggcgtgtgtgtgsttctggtttgctttaattttaccaangtttgtttcaaggtggatcgcgtgngtcaaggtccgtgtgctttaaagacccaccggcactggcagtgagtgttgctgcttgtgtaggctttggtacgtatgggctttat

SEQ ID NO: 73 - DNA, 774 bp, Zea mays
  misc_feature (281)..(283): n is a, c, g, or t
  misc_feature (328)..(328): n is a, c, g, or t
  misc_feature (652)..(652): n is a, c, g, or t
agcatcatggagtacggtcagcaggggcagcgcggccacggcgccacgggccatgtcgaccagtacggcaacccagtcggcggcgtcgagcacggcaccggcggcatgaggcacggcacgggaaccaccggcggcatgggccagctgggtgagcacggcggcgctggcatgggtggcgggcagttccagcctgcgagggaggagcacaagaccggcggcatcctgcatcgctccggcagctccagctccagctcggtaattacgactctggatacttcttnnntcttttgtgtgcgcgctgcttcgtcctatatataataatacatgnagttaggcttagtaataatcaattaatttaatccgtgggtttcgtgtttaagtcggaggacgacggcatgggcggaaggaggaagaagggaatcaaggagaagatcaaagagaagctgcccggaggccacaaggacgaccagcacgccacggcgacgaccggcggcgcctaygggcagcagggacacaccggcagcgcctacgggcagcagggacacaccggcggcgcctacgccaccgrcaccgagggcaccggcgagaagaaaggcattatggacaagatcaaggagaagctgcccggacagcactgagcggcgcctatacntggctgtgctgtgctgtgctggcgcgtcaaagccgtactcttcagygttccatagataataagataaacccatgaataagtgtccctaccctttgatcatgtgacagggacagggacaggga

SEQ ID NO: 74 - DNA, 885 bp, Zea mays
  misc_feature (140)..(141): n is a, c, g, or t
  misc_feature (211)..(212): n is a, c, g, or t
  misc_feature (662)..(663): n is a, c, g, or t
  misc_feature (675)..(675): n is a, c, g, or t
taggtattgtacacgctctagcttgacaaatggtcagccgttgatctctgctatttgcaaccagagcttaagttcatcttggattcatgcaggtcctggtgctccaaccatattcaactgcgtaaggccaccctgtcatnngcttgactggtcctcttgtgatatgttcatgttaatagcatratgtcttttgttctattggaaaataaanngtctccctggactctaaaatcaatgcctgtgaacacatgaactgtttgtgtcacccatgttcctctgctccttggcactttctgatgcatgctcaaatgcttaagaaagactcatagaagcgactcctattcctatgccaggtcattgagataccaaggggcagcaaggttaaatatgaacttgacaagaaaactggactgatcaaggtaaagcaatgttgttttcctcccgctgaagtcttattgtgaagctatatttcttgccagttctaatatttactcctttccgtttcaatctgtgtgcatgtgcaggtggaccgtgtgctgtattcatcagttgtttaccctcacaactatggattcattcctcgcacgctttgtgaagacagtgatcctttggatgtactggttataatgcaggtatgcttcttttttatatatatcattggnngattcacaaaantggtacatcagtagtgatctgagtatccttgggcataagttgagctaattttcaaatcttgtcattttccatttctgsgaatggtcgagaacatgtctataaactgttacttccaagcatgtaggagccagtcattttccatttctgtttatagttgcctagtcgggaacatgtatgtaaactgttacttccgtgcatgcaggagcctgt

SEQ ID NO: 75 - DNA, 935 bp, Zea mays
  misc_feature (84)..(84): n is a, c, g, or t
  misc_feature (219)..(221): n is a, c, g, or t
  misc_feature (303)..(305): n is a, c, g, or t
  misc_feature (783)..(783): n is a, c, g, or t
  misc_feature (804)..(804): n is a, c, g, or t
atcaagagcagcagctgctttgctgagaaacaggctgaccccgcatttgcacagttgcaggcctactagcaccacttgcagctnggttgacttgccatcctatccaacaaggagaatgaagaatgattaggtgctcccgtatacagataaacaatagcaaacatastgatcatgggattcatggcttatttttcactttgaatcatatgcaatattatnnngtwgcacagtgttctttgtttgtactcagtccctcaataaaagagggcctccatatgttgacatactatacttgatgactcnnnaaggaatgagaaaatgctgccacaaaaaagtctacaacacaaatgatctagttacctgttctttatctcccctgccatggtcatgaatgccttctccacatttgttgcatccttggcactagtctcaaggaatggtattccgatgtcatcagcaagggcctatgatgacaagcaacatgcagccaatttaactatcatcccggttgaaagaagcatgtccagtaaaagtaattaatgcagagaaatattaccttgccagcctcgtaagaaactactctgttctcagccaggtcacacttgttccccaccaaaagcttgttcacattttcactggcatacctatcratttcattcagccactgcttgacattgttaaagctctcctggtcagttacatcatacacaacctatagaaatacaaaagtttaaacaagactcagattaacaaagatgagataatagcagataggaaaaaaancgaaataagaaaaagaaagcntcacaataatgccatgagctccacggtagtagctgcttgtgatggtcctaaagcgttcttggccagcagtatcccactaaatcagaaraatgtggagaaacataagtgtcaaagcttctaactgttaggaa

SEQ ID NO: 76 - DNA, 710 bp, Zea mays
  misc_feature (593)..(596): n is a, c, g, or t
  misc_feature (598)..(600): n is a, c, g, or t
  misc_feature (606)..(607): n is a, c, g, or t
  misc_feature (610)..(617): n is a, c, g, or t
agcatsaccagaagcagagcctgatggacaaggcgaaggggttcgtygyggagaagatcgcgcacatccccaagcccgaggcsacgctggacggcgtgacgttcaagggcctgagccgggagtgcatcacgctgcacagcagcgtgaacgtgtccaacccctacgaccaccgcctccccatctgcgaggtgacctacacgctccggtgcgccggcaaggaggtggcgtccggcaccatgccggaccccggctggatcgccgccagcggctccaccgcgctggagatccccgccaaggtgccctacgacttcctcgtctccctcgtcagggacgtcggccgggactgggacatcgactacgagctccaggtcggrctcaccgtcgacctccccatcgtcggcaacttcaccatcccgctctccacctcyggcgagttcaagctccccaccctcaaggacttgttctgatctagtagtagctcgcttgccttstgttctgtgcgggcgcgcaccagcgatctgtacgacgascttttgcaaataaamgamgcagctcctctgttctatatatctmagkgratgsmtrrkytaaknnnntnnntgrytnnrynnnnnnnnaaataaagagctggatttcrttcaggttcctgtctcyaagctggattycattsgggcatccaccrtgatstggatgtgcctgccgcgtccgtc

SEQ ID NO: 77 - DNA, 663 bp, Zea mays
  misc_feature (24)..(24): n is a, c, g, or t
  misc_feature (101)..(101): n is a, c, g, or t
  misc_feature (159)..(159): n is a, c, g, or t
  misc_feature (229)..(229): n is a, c, g, or t
  misc_feature (249)..(249): n is a, c, g, or t
  misc_feature (252)..(252): n is a, c, g, or t
  misc_feature (264)..(264): n is a, c, g, or t
  misc_feature (267)..(267): n is a, c, g, or t
  misc_feature (294)..(294): n is a, c, g, or t
  misc_feature (439)..(439): n is a, c, g, or t
  misc_feature (462)..(462): n is a, c, g, or t
  misc_feature (588)..(588): n is a, c, g, or t
  misc_feature (594)..(594): n is a, c, g, or t
  misc_feature (616)..(618): n is a, c, g, or t
  misc_feature (641)..(641): n is a, c, g, or t
ttcctctataagtacccgccccanatctgcgccattttctcatcgcagaaatcctccgcacttcacagcgtatcatcgttttycatcgctcctactcctancatccagaaaatctgagmggtattgatggcgcccaaggcggagaagaagccggcggcnaagaaggtggcggaggaggagccctcggagaaggcggctccggcggagaaggcccccgcggggaagaagnccaaggcggagaagcggctnccngcgggcaagtcngcnggcaaggagggcggcgacaagaagggnaggaagaaggcgaagaagagcgtggagacctacaagatctacatcttcaaggtcctgaagcaggtgcaccccgacatcggcatctcctccaaggccatgtccatcatgaactccttcatcaacgacatcttcgagaagctcgccncggaggccgccaagctcgcccgntacaacaagaagcccaccatcacctcccgcgagatccagacctccgtccgcctcgtcctccccggcgagctcgccaagcacgccgtctcggagggtaccaaggccgtcaccaagttcacctcgtcntagccnccttgtwgtaggcgtcgttgtnnnctgcttctcaagcaagcactgtnatgtgccgcttctcatggcagt

(19) United States
(12) Patent Application Publication
(10) Pub. No.: US 2020/0263262 A1
(43) Pub. Date: Aug. 20, 2020

(54) GENETIC REGIONS & GENES ASSOCIATED WITH INCREASED YIELD IN PLANTS

(71) Applicant: Syngenta Participations AG, Basel (CH)

(72) Inventors: Allison Lynn Weber, Wildwood, MO (US); Elhan Sultan Ersoz, Johnston, IA (US); Robert John Bensen, Northfield, MN (US); Todd Lee Warner, Stanton, MN (US); Michael Mahlon Magwire, Research Triangle Park, NC (US)

(73) Assignee: Syngenta Participations AG, Basel (CH)

(21) Appl. No.: 16/061,249

(22) PCT Filed: Dec. 14, 2016
(86) PCT No.: PCT/US2016/066543

§ 371 Date: Jun. 11, 2018

Related U.S. Application Data
(60) Provisional application No. 62/268,158, filed on Dec. 16, 2015.

(51) Int. Cl. C12Q 1/6895; A01H 1/04; A01H 6/46

(52) U.S. Cl. CPC C12Q 1/6895; C12Q 2600/13; A01H 6/4684; A01H 1/04

(57) ABSTRACT

The present invention relates to methods and compositions for identifying, selecting and/or producing a plant or germplasm having root increased drought tolerance and/or increased yield under non-drought conditions as compared to a control plant. A maize plant, part thereof and/or germplasm, including any progeny and/or seeds derived from a maize plant or germplasm identified, selected and/or produced by any of the methods of the present invention is also provided.

### RELATED APPLICATIONS

This application claims the benefit of U.S. Provisional Application No. 62/268,158, filed 16 Dec. 2015, the contents of which are incorporated herein by reference.

### STATEMENT REGARDING ELECTRONIC FILING OF A SEQUENCE LISTING

A Sequence Listing in ASCII text format, is submitted, entitled 80955 SEQ LIST_ST25.txt and 122 kilobytes in size, generated on Dec. 5, 2016 and an electronic sequence listing is filed in conjunction with this application. This Sequence Listing is hereby incorporated by reference into the specification for its disclosures.

### FIELD OF THE INVENTION

The present invention relates to compositions and methods for introducing into a plant alleles, genes and/or chromosomal intervals that confer in said plant the traits of increased drought tolerance and/or increased yield under water stressed conditions and/or increased yield in the absence of water stress.

### BACKGROUND

Drought is one of the major limitations to maize production worldwide. Around 15% of the world's maize crop is lost every year due to drought. Periods of drought stress can occur at any time during the growing season. Maize is particularly sensitive to drought stress in the period just before and during flowering. When drought stress occurs during this critical period, a significant decrease in grain yield can result.

Identifying genes that enhance the drought tolerance of crops could lead to more efficient crop production practices by allowing for the identification, selection and production of crop plants with increased drought tolerance.

As such, a goal of plant breeding is to combine, in a single plant, various desirable traits. For field crops such as corn, soybean, etc. these traits can include greater yield and better agronomic quality. However, genetic loci that influence yield and agronomic quality are not always known, and even if known, their contributions to such traits are frequently unclear. Thus, new loci that can positively influence such desirable traits need to be identified and/or the abilities of known loci to do so need to be discovered.

Once discovered, these desirable loci can be selected for as part of a breeding program in order to generate plants that carry desirable traits. An exemplary embodiment of a method for generating such plants includes the transfer by introgression of nucleic acid sequences from plants that have desirable genetic information into plants that do not by crossing the plants using traditional breeding techniques. Further, one may use newly invented genome editing capabilities to edit a plant genome to comprise desirable genes or genetic allelic forms.

Desirable loci can be introduced into commercially available plant varieties using marker-assisted selection (MAS), marker-assisted breeding (MAB), transgenic expression of gene(s) and/or through recent gene editing technologies such as, for example CRISPR, TALEN, and etc.

What are needed, then, are new methods and compositions for introducing into a plant a gene or genomic region that may result in drought tolerant crops and/or crops that have increased yield in both well-watered and water stressed conditions.

### SUMMARY OF THE INVENTION

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

Compositions and methods for identifying, selecting and/or producing plants with increased yield under drought conditions are provided. As described herein, a genomic regions (interchangeably—“chromosome intervals”) may comprise, consist essentially of or consist of gene(s), a single allele or a combination of alleles at one or more genetic loci associated with increased drought tolerance and/or increased yield.

All disclosed maize chromosome positions herein correspond with the maize “B73 reference genome version 2”. The “B73 reference genome, version 2” is a publically available physical and genetic framework of the maize B73 genome. It is the result of a sequencing effort utilizing a minimal tiling path of approximately 19,000 mapped BAC clones, and focusing on producing high-quality sequence coverage of all identifiable gene-containing regions of the maize genome. These regions were ordered, oriented, and along with all of the intergenic sequences, anchored to the extant physical and genetic maps of the maize genome. It can be accessed using a genome browser, the Maize Genome Browser that is publicly available on the internet can facilitate user interaction with sequence and map data.

The present invention has identified eight causative loci within the maize genome that are highly associated with increased drought tolerance (e.g. increased bushels of corn per acre under drought conditions) and with increased yield (e.g. increased bushels of corn per acre under non-drought, normal or well-watered conditions), these eight loci collectively referred to herein as (‘yield alleles’). Specifically, the invention discloses the following eight yield alleles which demark the center highly associated yield loci, these alleles including: (1) SM2987 (herein, (‘yield allele 1’) or (‘SM2987’)) located on maize chromosome 1 corresponding to a G allele at position 272937870; (2) SM2991 (herein, (‘yield allele 2’) or (‘SM2991’)) located on maize chromosome 2 corresponding to a G allele at position 12023706; (3) SM2995 (herein, (‘yield allele 3’) or (‘SM2995’)) located on maize chromosome 3 corresponding to a A allele at position 225037602; (4) SM2996 (herein, (‘yield allele 4’) or (‘SM2996’)) located on maize chromosome 3 corresponding to a A allele at position 225340931; (5) SM2973 (herein, (‘yield allele 5’) or (‘SM2973’)) located on maize chromosome 5 corresponding to a G allele at position 159121201; (6) SM2980 (herein, (‘yield allele 6’) or (‘SM2980’)) located on maize chromosome 9 corresponding to a C allele at position 12104936; (7) SM2982 (herein, (‘yield allele 7’) or (‘SM2982’)) located on maize chromosome 9 corresponding to a A allele at position 133887717; and (8) SM2984 (herein, (‘yield allele 8’) or (‘SM2984’)) located on maize chromosome 10 corresponding to a G allele at position 4987333 (see Tables 1-7). Not to be limited by theory, it is believed that each of these yield alleles fall within or near a gene(s) that are causative for the given phenotype (e.g. yield either under drought or non-drought conditions). It is well known in the art that markers within the causative gene and all closely associated markers may be used in marker assisted breeding to select for, identify and assist in producing plants having the trait associated with the given marker (e.g. in this case, increased drought tolerance and/or yield, See Tables 1-7 demonstrating yield alleles and examples of closely associated markers that may be used to identify or produce maize lines having increased drought tolerance for each respective loci or chromosomal interval). Accordingly, in one aspect of the invention is disclosed a method of selecting or identifying a maize line or germplasm having increased drought tolerance and or increased yield (i.e. increases bushels per acre as compared to control plants) wherein the method comprises the steps of; (a) isolating a nucleic acid from a maize plant part; (b) detecting in the nucleic acid of (a) a molecular marker that is associated with drought tolerance and/or increased yield wherein the molecular marker is closely associated with any one of “Yield alleles 1-8” wherein closely associated means the marker is within 50 cM, 40 cM, 30 cM, 20 cM, 15 cM, 10 cM, 9 cM, 8 cM, 7 cM, 6 cM, 5 cM, 4 cM, 3 cM, 2 cM, 1 cM or 0.5 cM of the said Yield allele; and (c) selecting or identifying a maize plant on the basis of the presence of said marker in (b). In some embodiments the marker of (b) selected is any marker or closely associated marker described in Tables 1-7. In other embodiments the marker of (b) can be used to produce maize plants having increased drought tolerance or increased yield by selecting a maize plant according to the method described in steps (a)-(c) above and further comprising the steps of (d) crossing the plant of (c) with a second maize plant not comprising the marker identified in (b); and (d) producing a progeny plant comprising in its genome the marker of (b) wherein said progeny plant has increased drought tolerance and/or yield as compared to a control plant. In another instance, one may also wish to use the same marker identified in (b) to select progeny plants produced in (d).

In some embodiments of the present invention, is a method of identifying and/or selecting a drought tolerant maize plant, maize germplasm or plant part thereof, the method comprising: detecting, in said maize plant, maize germplasm or plant part thereof, at least one allele of a marker locus that is associated with drought tolerance in maize, wherein said at least one marker locus is located within a chromosomal interval selected from the group consisting of: a chromosomal interval flaked by and including markers IIM56014 and IIM48939 on chromosome 1 physical positions 248150852-296905665 (herein “interval 1”), IIM39140 and IIM40144 on chromosome 3 physical positions 201538048-230992107 (herein “interval 2”), IIM6931 and IIM7657 on chromosome 9 physical positions 121587239-145891243 (herein “interval 3”), IIM40272 and IIM41535 on chromosome 2 physical positions 1317414-36929703 (herein “interval 4”), IIM25303 and IIM48513 on chromosome 5 physical positions 139231600-183321037 (herein “interval 5”), IIM4047 and IIM4978 on chromosome 9 physical positions 405220-34086738 (herein “interval 6”), IIM19 and IIM818 on chromosome 10 physical positions 1285447-29536061 (herein “interval 7”), and any combinations thereof (See Tables 1-7 showing SNPs within said chromosome intervals that associate with increased drought tolerance. Allele positions bracketed with ‘***’ as well as bolded and underlined indicate “yield alleles” that are located within or in close proximity to the causative gene for drought tolerance and/or increased yield)

TABLE 1
Markers linked to SM2987 (“Interval 1”)

| | | | | | Un- |
|---|---|---|---|---|---|
| | | | | Favor- | favor- |
| | | | | able | able |
| Marker | Chr | Position | NegLogP | Allele | Allele |
| IIM56027 | chr1 | 248737375 | 1.50433144 | A | G |
| IIM56795 | chr1 | 274410772 | 1.52032289 | A | G |
| IIM56256 | chr1 | 256567682 | 1.52408159 | C | A |
| IIM57609 | chr1 | 296657535 | 1.54279066 | C | A |
| IIM56470 | chr1 | 264596664 | 1.5480698 | G | A |
| IIM57589 | chr1 | 296023245 | 1.55241175 | A | G |
| IIM56097 | chr1 | 251387901 | 1.56656654 | T | A |
| IIM56014 | chr1 | 248150852 | 1.56691816 | G | A |
| IIM56462 | chr1 | 264502708 | 1.58657142 | G | A |
| IIM56962 | chr1 | 278623502 | 1.59060728 | G | A |
| IIM56483 | chr1 | 264954421 | 1.61714921 | A | G |
| IIM56176 | chr1 | 253901998 | 1.62261833 | G | A |
| IIM57611 | chr1 | 296658712 | 1.64140263 | C | A |
| IIM56705 | chr1 | 272220484 | 1.64893408 | A | G |
| IIM56731 | chr1 | 272939276 | 1.65358638 | A | G |
| IIM56611 | chr1 | 269026864 | 1.6576665 | A | G |
| IIM48891 | chr1 | 277849479 | 1.6612574 | A | G |
| IIM48892 | chr1 | 277850711 | 1.66326748 | A | G |
| IIM56145 | chr1 | 250068693 | 1.66992884 | G | A |
| IIM57051 | chr1 | 281204729 | 1.67817169 | A | G |
| IIM56167 | chr1 | 253717214 | 1.71655968 | G | A |
| IIM57586 | chr1 | 296016145 | 1.72825308 | A | G |
| IIM56112 | chr1 | 251041358 | 1.74676025 | A | G |
| IIM56772 | chr1 | 273849481 | 1.74901932 | A | G |
| IIM56250 | chr1 | 256489660 | 1.75217508 | A | G |
| IIM56399 | chr1 | 261890581 | 1.75916206 | A | C |
| IIM56602 | chr1 | 268814080 | 1.78515615 | A | C |
| IIM56246 | chr1 | 256469982 | 1.79602001 | G | A |
| IIM57340 | chr1 | 289476630 | 1.79747313 | G | A |
| IIM57612 | chr1 | 296658750 | 1.80523315 | G | A |
| IIM48880 | chr1 | 271346409 | 1.87014894 | G | A |
| IIM56166 | chr1 | 253716559 | 1.87167124 | G | A |
| IIM57620 | chr1 | 296847731 | 1.89860828 | G | A |
| IIM56261 | chr1 | 256797371 | 1.9194226 | A | G |
| IIM57626 | chr1 | 296904553 | 1.94907352 | A | G |
| IIM56918 | chr1 | 277325614 | 1.95413295 | A | C |
| IIM57605 | chr1 | 296576237 | 1.95462281 | G | A |
| IIM56965 | chr1 | 278667820 | 1.95467702 | G | A |
| IIM48939 | chr1 | 296905665 | 1.98049851 | G | A |
| IIM56658 | chr1 | 270705505 | 2.01356053 | A | G |
| IIM56526 | chr1 | 266310270 | 2.03016871 | A | G |
| IIM56700 | chr1 | 271973928 | 2.04323513 | A | G |
| IIM59860 | chr1 | 264224945 | 2.04575749 | G | A |
| IIM61006 | chr1 | 269328951 | 2.08092191 | C | G |
| IIM56578 | chr1 | 267888703 | 2.08510624 | A | C |
| IIM56610 | chr1 | 269022807 | 2.10975909 | A | G |
| IIM59859 | chr1 | 264224831 | 2.13076828 | A | G |
| IIM56746 | chr1 | 273299369 | 2.14095739 | A | C |
| IIM56472 | chr1 | 264799902 | 2.14743404 | T | A |
| IIM56770 | chr1 | 273787354 | 2.30160506 | A | G |
| IIM56910 | chr1 | 276982100 | 2.30198971 | G | A |
| IIM56748 | chr1 | 273326817 | 2.31513285 | A | G |
| IIM56626 | chr1 | 269496695 | 2.68054642 | C | A |
| IIM58395 | chr1 | 271110745 | 2.7447275 | A | G |
| IIM48879 | chr1 | 271114177 | 2.86569589 | G | A |
| ***SM2987*** | chr1 | 272937870 | 2.88605665 | G | A |
| IIM56759 | chr1 | 273677932 | 3.09691001 | A | G |
| IIM69670 | chr1 | 277310887 | 3.30103 | G | A |
| IIM59541 | chr1 | 277311204 | 4 | C | A |

TABLE 2
Markers linked to SM2995 and SM2996 (“interval 2”)

| | | | | | Un- |
|---|---|---|---|---|---|
| | | | | Favor- | favor- |
| | | | | able | able |
| Marker | Chr | Position | NegLogP | Allele | Allele |
| IIM39140 | chr3 | 201538048 | 1.9773088 | A | G |
| IIM39142 | chr3 | 201541112 | 1.9773088 | A | G |
| IIM39334 | chr3 | 207552021 | 1.5761566 | A | G |
| IIM39347 | chr3 | 208056164 | 1.5499955 | A | G |
| IIM39377 | chr3 | 209127383 | 1.6079142 | A | G |
| IIM39378 | chr3 | 209127601 | 1.6079142 | G | A |
| IIM39380 | chr3 | 209130133 | 1.9656038 | A | G |
| IIM39381 | chr3 | 209130483 | 1.6339539 | A | G |
| IIM39383 | chr3 | 209130518 | 1.6339539 | G | A |
| IIM39384 | chr3 | 209137452 | 1.6896011 | A | G |
| IIM39385 | chr3 | 209137558 | 1.9363858 | C | A |
| IIM39386 | chr3 | 209137712 | 1.8206902 | A | G |
| IIM39390 | chr3 | 209492822 | 1.6522923 | A | G |
| IIM39485 | chr3 | 212548418 | 1.7750362 | A | G |
| SM2994 | chr3 | 213657163 | — | A | G |
| IIM39527 | chr3 | 213787190 | 2.2445931 | A | G |
| IIM39715 | chr3 | 219371525 | 1.8722122 | G | A |
| IIM39716 | chr3 | 219373746 | 1.7634839 | A | C |
| IIM39725 | chr3 | 219488538 | 1.7931088 | A | G |
| IIM39726 | chr3 | 219579336 | 2.638273 | A | G |
| IIM39731 | chr3 | 219693372 | 1.6675217 | G | A |
| IIM39729 | chr3 | 219695215 | 2.500178 | G | A |
| IIM39728 | chr3 | 219695311 | 1.5995023 | C | A |
| IIM39732 | chr3 | 219714084 | 1.8590536 | A | G |
| IIM39771 | chr3 | 220750715 | 1.5919602 | C | A |
| IIM39784 | chr3 | 221239625 | 1.5391511 | A | C |
| IIM39783 | chr3 | 221240133 | 1.5434047 | A | G |
| IIM39787 | chr3 | 221308065 | 2.1413076 | G | A |
| IIM39802 | chr3 | 221560239 | 2.1951791 | G | A |
| IIM39856 | chr3 | 223309596 | 1.6562343 | G | A |
| IIM39870 | chr3 | 223681739 | 1.9534217 | A | G |
| IIM39873 | chr3 | 223683787 | 1.6416155 | A | G |
| IIM39877 | chr3 | 223741777 | 2.0054997 | A | C |
| IIM39883 | chr3 | 223842795 | 2.1156788 | A | C |
| IIM39900 | chr3 | 224502756 | 1.7101472 | G | A |
| IIM39914 | chr3 | 224687774 | 1.7446215 | C | A |
| ***SM2995*** | chr3 | 225037602 | 3.81 | A | C |
| ***SM2996*** | chr3 | 225340931 | 4.07 | A | G |
| IIM39935 | chr3 | 225454492 | 1.867438 | C | A |
| IIM39941 | chr3 | 225584567 | 1.7513472 | A | G |
| IIM39976 | chr3 | 226624315 | 1.6116138 | A | G |
| IIM39990 | chr3 | 227083822 | 1.8538532 | A | G |
| IIM39994 | chr3 | 227191973 | 1.5543445 | A | G |
| IIM40032 | chr3 | 228093802 | 1.9344464 | G | A |
| IIM40033 | chr3 | 228250735 | 2.1393386 | A | G |
| IIM40045 | chr3 | 228451330 | 1.753483 | G | A |
| IIM40046 | chr3 | 228451397 | 1.6663862 | A | G |
| IIM40047 | chr3 | 228547395 | 2.0200519 | G | A |
| IIM48771 | chr3 | 228720304 | 1.6996651 | C | A |
| IIM40055 | chr3 | 228999318 | 2.9802561 | C | A |
| IIM40060 | chr3 | 229131539 | 2.1015707 | G | A |
| IIM40061 | chr3 | 229153740 | 2.1532103 | A | G |
| IIM40062 | chr3 | 229192335 | 1.6650966 | A | G |
| IIM40064 | chr3 | 229241840 | 2.4160972 | C | A |
| IIM40094 | chr3 | 230016344 | 2.0844205 | A | G |
| IIM40095 | chr3 | 230016843 | 2.1287806 | G | A |
| IIM40096 | chr3 | 230017436 | 2.3739704 | G | A |
| IIM40099 | chr3 | 230159751 | 1.7522514 | G | A |
| IIM40144 | chr3 | 230992107 | 2.0250472 | G | C |

TABLE 3
Markers linked to SM2982 (Chromosome interval 3)

| | | | | | Un- |
|---|---|---|---|---|---|
| | | | | Favor- | favor- |
| | | | | able | able |
| Marker | Chr | Position | NegLogP | Allele | Allele |
| IIM6931 | chr9 | 121587239 | 2.4152966 | A | G |
| IIM6934 | chr9 | 121602328 | 1.7866655 | C | A |
| IIM6946 | chr9 | 122220390 | 1.5737392 | A | G |
| IIM6961 | chr9 | 122956699 | 1.5099794 | G | A |
| IIM7041 | chr9 | 125899355 | 1.5343446 | A | G |
| IIM7054 | chr9 | 126400936 | 1.8302675 | G | A |
| IIM7055 | chr9 | 126401198 | 1.8302675 | G | A |
| IIM7086 | chr9 | 127696098 | 1.8539401 | G | A |
| IIM7101 | chr9 | 128301095 | 1.5660632 | G | A |
| IIM7104 | chr9 | 128542456 | 1.8172872 | A | G |
| IIM7105 | chr9 | 128542462 | 2.2254661 | G | A |
| IIM7109 | chr9 | 128617535 | 1.9931737 | A | G |
| IIM7110 | chr9 | 128645142 | 2.0915709 | G | A |
| IIM7114 | chr9 | 128653793 | 2.0565009 | G | A |
| IIM7117 | chr9 | 128726984 | 2.2321071 | G | A |
| IIM7141 | chr9 | 129514761 | 2.1321032 | A | G |
| IIM7151 | chr9 | 130015036 | 1.6408661 | A | G |
| IIM7151 | chr9 | 130015036 | 2.7621932 | A | G |
| IIM7163 | chr9 | 130488854 | 2.334145 | G | A |
| IIM7168 | chr9 | 130523091 | 3.0780195 | A | G |
| IIM7166 | chr9 | 130526677 | 1.6566408 | G | A |
| IIM7178 | chr9 | 130784212 | 2.2491198 | G | A |
| IIM7184 | chr9 | 130873209 | 1.7936628 | G | A |
| IIM7183 | chr9 | 130884382 | 1.6142931 | G | A |
| IIM7204 | chr9 | 131523248 | 1.7478445 | A | G |
| IIM7231 | chr9 | 132005716 | 1.8100293 | A | G |
| IIM7235 | chr9 | 132814746 | 1.5555701 | A | G |
| IIM7249 | chr9 | 133549736 | 1.5460904 | A | G |
| ***SM2982*** | chr9 | 133887717 | 2.31 | A | G |
| IIM7272 | chr9 | 134284675 | 1.6088131 | A | G |
| IIM7273 | chr9 | 134285829 | 2.2233117 | A | G |
| IIM7275 | chr9 | 134289176 | 2.5406828 | A | G |
| IIM7284 | chr9 | 134504060 | 1.7825381 | G | A |
| IIM7283 | chr9 | 134544459 | 1.5180161 | A | G |
| IIM7285 | chr9 | 134569704 | 1.7815149 | C | A |
| IIM7318 | chr9 | 135891509 | 1.5427136 | G | A |
| IIM7319 | chr9 | 135897300 | 1.6597074 | A | G |
| IIM7351 | chr9 | 136828552 | 1.7655136 | A | G |
| IIM7354 | chr9 | 136867832 | 2.5639781 | A | G |
| IIM7384 | chr9 | 137413358 | 1.6001204 | G | A |
| IIM7386 | chr9 | 137421864 | 2.3691795 | A | G |
| IIM7388 | chr9 | 137424404 | 1.653031 | G | A |
| IIM7397 | chr9 | 137846999 | 2.6465223 | A | C |
| IIM7417 | chr9 | 138615643 | 1.5495106 | C | A |
| IIM7427 | chr9 | 138892323 | 1.8512733 | A | G |
| IIM7463 | chr9 | 139961409 | 1.6971348 | A | G |
| IIM7480 | chr9 | 140345720 | 2.2204193 | C | A |
| IIM7481 | chr9 | 140348142 | 1.669621 | A | G |
| IIM7548 | chr9 | 142202674 | 2.2741955 | G | A |
| IIM7616 | chr9 | 144307969 | 1.8380075 | C | A |
| IIM48034 | chr9 | 144308202 | 1.526927 | G | A |
| IIM7636 | chr9 | 145336391 | 1.870377 | A | G |
| IIM7653 | chr9 | 145771250 | 1.8295507 | G | A |
| IIM7657 | chr9 | 145891243 | 1.9924887 | G | A |

TABLE 4
Markers linked to SM2991 (“Interval 4”)

| | | | | | Un- |
|---|---|---|---|---|---|
| | | | | Favor- | favor- |
| | | | | able | able |
| Marker | Chr | Position | NegLogP | Allele | Allele |
| IIM40272 | chr2 | 1317414 | 2.1857338 | A | G |
| IIM40279 | chr2 | 1560595 | 1.9656757 | A | G |
| IIM40301 | chr2 | 1824359 | 1.7788805 | G | A |
| IIM40310 | chr2 | 2041151 | 2.3921399 | A | C |
| IIM40311 | chr2 | 2041283 | 2.2924444 | A | G |
| SM2990 | chr2 | 5069026 | — | G | — |
| IIM40440 | chr2 | 5379267 | 2.0379023 | G | A |
| IIM40442 | chr2 | 5379504 | 1.8314561 | G | A |
| IIM40463 | chr2 | 5824493 | 1.608778 | A | C |
| IIM40486 | chr2 | 6154706 | 2.3494015 | A | G |
| IIM40522 | chr2 | 7191765 | 1.5761147 | A | C |
| IIM40627 | chr2 | 9274354 | 2.0637719 | A | C |
| IIM40646 | chr2 | 9973084 | 2.1681313 | C | A |
| IIM40709 | chr2 | 11053622 | 1.5216061 | G | A |
| IIM40719 | chr2 | 11369240 | 1.814594 | G | A |
| ***SM2991*** | chr2 | 12023706 | 1.5176435 | G | A |
| ***SM2991*** | chr2 | 12023706 | 2.03 | G | A |
| IIM40768 | chr2 | 12352131 | 2.3463621 | C | A |
| IIM40771 | chr2 | 12685114 | 1.5093959 | A | G |
| IIM40775 | chr2 | 12801930 | 2.7533596 | G | A |
| IIM40788 | chr2 | 13301971 | 1.8605552 | C | A |
| IIM40789 | chr2 | 13308210 | 1.644831 | G | A |
| IIM40790 | chr2 | 13308222 | 1.5935647 | G | A |
| IIM40795 | chr2 | 13382024 | 1.9498919 | G | A |
| IIM40802 | chr2 | 13783137 | 1.716527 | G | A |
| IIM40804 | chr2 | 13784730 | 1.7393164 | G | A |
| IIM40837 | chr2 | 14880624 | 1.9769813 | G | A |
| IIM40839 | chr2 | 14891011 | 1.6620462 | G | A |
| IIM40848 | chr2 | 15129464 | 1.8567125 | C | G |
| IIM47120 | chr2 | 15580132 | 2.2051284 | A | G |
| IIM40862 | chr2 | 15969866 | 1.8412728 | G | A |
| IIM40863 | chr2 | 15972959 | 2.1076789 | A | G |
| IIM40888 | chr2 | 16532267 | 1.6967631 | C | A |
| IIM40893 | chr2 | 16776017 | 1.5728762 | A | G |
| IIM40909 | chr2 | 17154478 | 1.6400482 | G | A |
| IIM40928 | chr2 | 17904412 | 2.2083971 | G | A |
| IIM40931 | chr2 | 17997157 | 1.7663092 | A | G |
| IIM40932 | chr2 | 18002381 | 2.8017619 | G | A |
| IIM40940 | chr2 | 18131285 | 2.206648 | A | G |
| IIM47155 | chr2 | 18132241 | 1.8400941 | A | G |
| IIM40936 | chr2 | 18134248 | 2.4939932 | A | G |
| IIM47156 | chr2 | 18204855 | 1.6913651 | A | G |
| IIM40991 | chr2 | 19361220 | 2.2006584 | A | C |
| IIM40998 | chr2 | 19832410 | 1.7103352 | C | A |
| IIM41001 | chr2 | 19918031 | 1.7178692 | A | G |
| IIM41008 | chr2 | 20018130 | 1.6649729 | G | A |
| IIM41013 | chr2 | 20205707 | 1.5762741 | G | A |
| IIM41064 | chr2 | 21794826 | 2.679845 | A | G |
| IIM41153 | chr2 | 24735926 | 1.5429672 | G | A |
| IIM41229 | chr2 | 27562776 | 1.8184282 | A | C |
| IIM41230 | chr2 | 27564732 | 1.7804251 | G | A |
| IIM41247 | chr2 | 28006625 | 1.5067883 | G | A |
| IIM41259 | chr2 | 28402733 | 1.9235509 | A | G |
| IIM41261 | chr2 | 28404853 | 3.0111113 | C | A |
| IIM41263 | chr2 | 28405435 | 3.0102334 | A | G |
| IIM41283 | chr2 | 28703638 | 2.8718985 | A | G |
| IIM41287 | chr2 | 28894630 | 2.0602923 | G | C |
| IIM41310 | chr2 | 29544066 | 1.674683 | G | A |
| SM2985 | ch2 | 30233543 | — | G | C |
| IIM41321 | chr2 | 30260710 | 2.0019004 | A | G |
| IIM41359 | chr2 | 30872159 | 2.5061276 | C | A |
| IIM41357 | chr2 | 30874237 | 2.6366301 | G | A |
| IIM41366 | chr2 | 31154060 | 1.7125946 | C | A |
| IIM41377 | chr2 | 31594230 | 1.5644559 | G | A |
| IIM46720 | chr2 | 32522416 | 1.7639852 | A | G |
| IIM41412 | chr2 | 33037195 | 1.9417919 | G | A |
| IIM41430 | chr2 | 33499665 | 1.6721862 | G | A |
| IIM41448 | chr2 | 33727735 | 1.5441876 | A | C |
| IIM41456 | chr2 | 34222566 | 1.7385048 | C | A |
| IIM49103 | chr2 | 34700898 | 1.6320133 | G | A |
| IIM41479 | chr2 | 35272010 | 1.8484383 | G | A |
| IIM41509 | chr2 | 36605493 | 2.3639798 | A | G |
| IIM41535 | chr2 | 36929703 | 1.5899451 | G | A |

TABLE 5
Markers linked to SM2973 (“Interval 5”)

| | | | | | Un- |
|---|---|---|---|---|---|
| | | | | Favor- | favor- |
| | | | | able | able |
| Marker | Chr | Position | NegLogP | Allele | Allele |
| IIM25303 | chr5 | 139231600 | 1.5145926 | A | G |
| IIM25304 | chr5 | 139232274 | 1.6219585 | G | A |
| IIM25320 | chr5 | 139811946 | 1.8272476 | G | A |
| IIM25350 | chr5 | 141129999 | 1.6203994 | G | A |
| IIM25391 | chr5 | 142579539 | 1.5993338 | A | G |
| IIM25399 | chr5 | 142826085 | 2.0932768 | A | G |
| IIM25400 | chr5 | 142854837 | 1.6700915 | C | A |
| IIM25402 | chr5 | 143010005 | 1.891167 | G | A |
| IIM25407 | chr5 | 143163659 | 1.5429971 | G | A |
| IIM25414 | chr5 | 143197473 | 1.9987973 | A | G |
| IIM25429 | chr5 | 143971282 | 1.6684186 | G | A |
| IIM25442 | chr5 | 144176066 | 1.8282413 | A | C |
| IIM25449 | chr5 | 144574260 | 2.148713 | G | A |
| IIM25526 | chr5 | 147629967 | 1.6416016 | A | G |
| IIM25543 | chr5 | 148226517 | 1.6058567 | A | G |
| IIM25545 | chr5 | 148304095 | 1.5077863 | G | A |
| IIM25600 | chr5 | 151166589 | 2.1164145 | A | G |
| IIM25688 | chr5 | 154482401 | 1.8649207 | C | A |
| IIM25694 | chr5 | 154995048 | 2.0894606 | C | A |
| IIM25731 | chr5 | 155962380 | 2.0494173 | A | G |
| IIM25740 | chr5 | 156888309 | 2.9498972 | C | G |
| IIM25799 | chr5 | 159104587 | 1.57457 | A | G |
| IIM25800 | chr5 | 159109882 | 1.776678 | G | A |
| ***SM2973*** | chr5 | 159121201 | 2.47 | G | C |
| IIM25805 | chr5 | 159233574 | 1.7102116 | G | A |
| IIM25806 | chr5 | 159233808 | 2.2163922 | A | G |
| IIM25819 | chr5 | 159929251 | 1.8231616 | A | C |
| IIM25820 | chr5 | 159929284 | 1.6497217 | C | A |
| IIM25821 | chr5 | 159929345 | 1.9306803 | A | G |
| IIM25823 | chr5 | 159946905 | 1.7860221 | G | A |
| IIM25824 | chr5 | 159946929 | 1.71639 | G | A |
| IIM25828 | chr5 | 160079236 | 2.5122424 | A | G |
| IIM25830 | chr5 | 160088883 | 1.6140322 | G | A |
| IIM25856 | chr5 | 161197310 | 2.2441902 | G | A |
| IIM25864 | chr5 | 161557626 | 1.5512714 | A | G |
| IIM25870 | chr5 | 162008437 | 2.4576417 | G | A |
| IIM25905 | chr5 | 163289969 | 1.5527557 | G | A |
| IIM25921 | chr5 | 163834981 | 2.8649125 | C | A |
| IIM25938 | chr5 | 164481571 | 1.8954276 | A | G |
| IIM25939 | chr5 | 164531662 | 1.7887783 | G | A |
| IIM25945 | chr5 | 164658024 | 1.8762444 | A | C |
| IIM25965 | chr5 | 165320944 | 2.0473961 | A | G |
| IIM25966 | chr5 | 165321089 | 2.1556521 | A | G |
| IIM25968 | chr5 | 165516174 | 1.5203399 | G | A |
| IIM25975 | chr5 | 165860488 | 1.6022312 | A | G |
| IIM25978 | chr5 | 165979358 | 1.7078799 | A | C |
| IIM25983 | chr5 | 166302411 | 1.5188686 | A | G |
| IIM25984 | chr5 | 166302435 | 1.923457 | A | G |
| IIM25987 | chr5 | 166332322 | 1.9495422 | G | A |
| IIM25999 | chr5 | 166576990 | 1.5848507 | A | G |
| IIM25999 | chr5 | 166576990 | 1.9472188 | A | G |
| IIM26009 | chr5 | 167120764 | 1.5012315 | A | G |
| IIM26023 | chr5 | 167584724 | 1.7957114 | A | T |
| IIM26084 | chr5 | 169578102 | 1.9345068 | C | A |
| IIM26119 | chr5 | 170828221 | 1.8751832 | A | G |
| IIM26132 | chr5 | 171363180 | 1.5448205 | C | A |
| IIM26133 | chr5 | 171456415 | 1.5141995 | G | A |
| IIM26145 | chr5 | 171964318 | 2.2128945 | G | A |
| IIM26151 | chr5 | 172023565 | 2.302314 | A | G |
| IIM48428 | chr5 | 172294403 | 2.4958518 | G | A |
| IIM26170 | chr5 | 172477144 | 1.611221 | G | A |
| IIM26175 | chr5 | 172810787 | 2.4460352 | A | C |
| IIM26226 | chr5 | 174308410 | 1.9455886 | G | A |
| IIM26263 | chr5 | 175663600 | 1.6526181 | A | G |
| IIM26264 | chr5 | 175684872 | 1.6041878 | A | C |
| IIM26267 | chr5 | 175688745 | 1.5085936 | C | A |
| IIM26268 | chr5 | 175689408 | 2.3466903 | G | A |
| IIM26271 | chr5 | 175731290 | 1.6002639 | C | A |
| IIM26272 | chr5 | 175731649 | 1.5240736 | A | G |
| IIM26273 | chr5 | 175731823 | 1.5240736 | G | A |
| IIM26274 | chr5 | 175731857 | 1.5240736 | G | A |
| IIM26291 | chr5 | 176115205 | 2.2782434 | A | G |
| IIM26383 | chr5 | 179014380 | 1.9056247 | G | A |
| IIM26402 | chr5 | 179855228 | 1.6946571 | G | A |
| IIM26493 | chr5 | 183319499 | 1.7054321 | A | G |
| IIM26495 | chr5 | 183319662 | 1.8167617 | G | C |
| IIM48513 | chr5 | 183321037 | 1.721518 | A | G |

TABLE 6
Markers linked to SM2980 (“Interval 6”)

| | | | | | Un- |
|---|---|---|---|---|---|
| | | | | Favor- | favor- |
| | | | | able | able |
| Marker | Chr | Position | NegLogP | Allele | Allele |
| IIM4047 | chr9 | 405220 | 1.5064877 | G | A |
| IIM4046 | chr9 | 415627 | 2.0155796 | G | A |
| IIM4044 | chr9 | 479709 | 1.6120201 | G | A |
| IIM4038 | chr9 | 572218 | 1.5126752 | C | A |
| IIM4109 | chr9 | 2333656 | 1.5703597 | C | A |
| IIM4121 | chr9 | 2625102 | 1.6988197 | A | G |
| IIM4143 | chr9 | 3859664 | 1.8797829 | A | G |
| IIM4177 | chr9 | 4841629 | 1.7875945 | G | A |
| IIM4203 | chr9 | 6055174 | 1.5092519 | G | A |
| IIM4212 | chr9 | 6300466 | 2.1020642 | A | G |
| IIM4214 | chr9 | 6468616 | 1.7263303 | A | G |
| IIM4214 | chr9 | 6468616 | 1.7974659 | G | A |
| IIM4215 | chr9 | 6491517 | 2.1369729 | A | C |
| IIM4219 | chr9 | 6887520 | 2.2296565 | T | A |
| IIM4226 | chr9 | 7177627 | 2.3343678 | G | A |
| IIM4227 | chr9 | 7177720 | 2.5636449 | G | A |
| IIM4229 | chr9 | 7178752 | 2.3162885 | G | A |
| IIM4231 | chr9 | 7190557 | 2.3726563 | G | A |
| IIM4232 | chr9 | 7190777 | 2.361686 | G | A |
| IIM4233 | chr9 | 7191269 | 2.4320888 | G | A |
| IIM4235 | chr9 | 7191887 | 2.6000164 | A | G |
| IIM4236 | chr9 | 7202656 | 2.4448816 | A | C |
| IIM4237 | chr9 | 7202660 | 2.5210319 | A | G |
| IIM4239 | chr9 | 7308398 | 1.9493314 | A | G |
| IIM4239 | chr9 | 7308398 | 2.4988801 | G | A |
| IIM4240 | chr9 | 7311899 | 2.1735336 | C | A |
| IIM4241 | chr9 | 7312169 | 2.4036344 | G | A |
| IIM4242 | chr9 | 7312304 | 2.1606746 | C | G |
| IIM4244 | chr9 | 7314063 | 2.5739783 | A | G |
| IIM4255 | chr9 | 7660637 | 1.6965989 | G | A |
| IIM4263 | chr9 | 7959566 | 1.6777851 | A | G |
| IIM4264 | chr9 | 7959809 | 1.9638743 | G | A |
| IIM4265 | chr9 | 7960037 | 1.8329034 | A | G |
| IIM4308 | chr9 | 8432871 | 3.2817951 | G | A |
| IIM4295 | chr9 | 8779931 | 2.306936 | A | C |
| IIM4289 | chr9 | 8783918 | 1.6023467 | A | C |
| IIM4280 | chr9 | 8972061 | 1.5209009 | G | A |
| IIM4345 | chr9 | 10596644 | 1.7279538 | G | A |
| IIM4387 | chr9 | 11562960 | 1.7874442 | A | G |
| IIM4387 | chr9 | 11562960 | 2.6563637 | A | G |
| IIM4388 | chr9 | 11628485 | 2.6146287 | A | G |
| IIM4388 | chr9 | 11628485 | 2.7447304 | A | G |
| IIM4389 | chr9 | 11711337 | 2.5496102 | C | A |
| IIM4390 | chr9 | 11711659 | 1.5659169 | G | A |
| IIM4390 | chr9 | 11711659 | 2.2327542 | G | A |
| IIM4392 | chr9 | 11743722 | 2.4608175 | A | G |
| IIM4395 | chr9 | 11922053 | 2.2960734 | A | G |
| ***SM2980*** | chr9 | 12104936 | 1.38 | C | G |
| IIM4458 | chr9 | 13911977 | 1.7564944 | G | A |
| IIM4469 | chr9 | 14020866 | 2.1290449 | A | G |
| IIM4482 | chr9 | 14535102 | 1.512826 | A | C |
| IIM4607 | chr9 | 18894260 | 1.6887963 | G | A |
| IIM4608 | chr9 | 18894276 | 1.6830963 | A | G |
| IIM4609 | chr9 | 19015745 | 1.5739462 | A | G |
| IIM4613 | chr9 | 19163650 | 1.511585 | G | A |
| IIM4614 | chr9 | 19230857 | 2.1372864 | A | G |
| IIM4674 | chr9 | 21723713 | 1.5696618 | A | G |
| IIM4681 | chr9 | 21872777 | 1.9222131 | A | C |
| IIM4682 | chr9 | 21875158 | 2.0384372 | A | G |
| IIM4738 | chr9 | 23754586 | 1.5802904 | G | A |
| IIM4755 | chr9 | 24197681 | 1.5176253 | G | A |
| IIM4756 | chr9 | 24198120 | 1.5572086 | G | A |
| IIM4768 | chr9 | 24511976 | 2.8603943 | G | A |
| IIM4777 | chr9 | 25257190 | 1.9010474 | G | A |
| IIM4816 | chr9 | 26939945 | 1.544776 | G | A |
| IIM4818 | chr9 | 26946314 | 1.599011 | A | C |
| IIM4822 | chr9 | 27092723 | 1.7489679 | A | G |
| IIM4831 | chr9 | 27222601 | 1.6716351 | G | A |
| IIM4851 | chr9 | 28017219 | 1.5155081 | A | C |
| IIM4856 | chr9 | 28399075 | 1.7607202 | G | A |
| IIM47276 | chr9 | 28399313 | 1.6053862 | A | G |
| IIM4857 | chr9 | 28399852 | 1.7112239 | A | G |
| IIM4858 | chr9 | 28399876 | 1.9082411 | A | G |
| IIM4859 | chr9 | 28400535 | 1.5147582 | C | A |
| IIM4860 | chr9 | 28402016 | 1.7689672 | G | A |
| IIM4875 | chr9 | 28620000 | 1.6193674 | A | G |
| IIM4878 | chr9 | 29232071 | 1.7046519 | G | A |
| IIM4967 | chr9 | 33712769 | 1.6855973 | G | A |
| IIM4974 | chr9 | 33842125 | 2.0037492 | A | G |
| IIM4978 | chr9 | 34086738 | 1.8369235 | A | G |

TABLE 7
Markers linked to SM2984 (“Interval 7”)

| | | | | | Un- |
|---|---|---|---|---|---|
| | | | | Favor- | favor- |
| | | | | able | able |
| Marker | Chr | Position | NegLogP | Allele | Allele |
| IIM19 | chr10 | 1285447 | 1.705682176 | G | A |
| IIM26 | chr10 | 1631939 | 1.136776407 | G | A |
| IIM32 | chr10 | 1947358 | 1.109774953 | A | G |
| IIM43 | chr10 | 2255896 | 1.329573503 | A | C |
| IIM66 | chr10 | 2479844 | 1.109488841 | C | G |
| IIM72 | chr10 | 2659080 | 1.048740325 | A | G |
| IIM78 | chr10 | 2792381 | 1.790597272 | A | G |
| IIM77 | chr10 | 2792533 | 1.409845658 | G | A |
| IIM84 | chr10 | 3017675 | 1.05328797 | A | G |
| IIM108 | chr10 | 3170298 | 1.280015583 | C | A |
| IIM121 | chr10 | 4064242 | 1.211598989 | A | G |
| IIM46822 | chr10 | 4072690 | 1.62535345 | G | A |
| ***SM2984*** | chr10 | 4987333 | 1.87 | G | C |
| IIM193 | chr10 | 6072208 | 1.252588192 | C | A |
| IIM211 | chr10 | 6935300 | 1.191744777 | G | A |
| IIM236 | chr10 | 8094983 | 1.050166695 | A | G |
| IIM274 | chr10 | 9220030 | 1.132162785 | C | A |
| IIM275 | chr10 | 9370054 | 1.075976942 | A | C |
| IIM291 | chr10 | 9586995 | 1.157618367 | A | G |
| IIM638 | chr10 | 22996187 | 1.009978053 | A | C |
| IIM738 | chr10 | 26138181 | 1.002208052 | A | C |
| IIM739 | chr10 | 26138274 | 1.266861847 | G | A |
| IIM818 | chr10 | 29536061 | 1.150314943 | A | C |

In some embodiments, methods of producing a drought tolerant maize plant are provided. Such methods can comprise detecting, in a maize germplasm or maize plant, the presence of a marker associated with increased drought tolerance (e.g. a marker within any chromosomal interval or combination thereof comprising at least one chromosome interval 1-15 as herein defined, any marker or combination thereof of a marker listed in Tables 1-7 or any of yield alleles 1-8 or closely associated markers to yield alleles 1-8) and producing a progeny plant from said maize germplasm or plant wherein said progeny plant comprises said marker associated with increased drought tolerance and said progeny plant further demonstrates increased drought tolerance as compared to a control plant not comprising said marker. The invention also provides seed produced from said progeny plant.

In some embodiments, is provided a maize seed produced by two parental maize lines wherein at least one parental line was identified or selected for increased yield under drought stress or increased yield under non-drought conditions and further wherein yield is increased bushels of corn per acre as compared to a control plant and wherein the at least on parental line was selected according to the method comprising the steps of: (a) isolating a nucleic acid from a maize parental line plant part; (b) detecting in the nucleic acid of (a) a molecular marker that is associated with drought tolerance and/or increased yield wherein the molecular marker is closely associated with any one of “Yield alleles 1-8” wherein closely associated means the marker is within 50 cM, 40 cM, 30 cM, 20 cM, 15 cM, 10 cM, 9 cM, 8 cM, 7 cM, 6 cM, 5 cM, 4 cM, 3 cM, 2 cM, 1 cM or 0.5 cM of the said Yield allele; and (c) selecting or identifying a maize plant on the basis of the presence of said marker in (b). In some aspects of the embodiment the molecular marker of (b) is within a chromosomal interval selected from any one of chromosomal intervals 1-15 as defined herein.

In some embodiments, the presence of a marker associated with increased drought tolerance is detected using a marker probe. In some such embodiments, the presence of a marker associated with increased drought tolerance is detected in an amplification product from a nucleic acid sample isolated from a maize plant or germplasm. In some embodiments, the marker comprises a haplotype, and a plurality of probes is used to detect the alleles that make up the haplotype. In some such embodiments, the alleles that make up the haplotype are detected in a plurality of amplification products from a nucleic acid sample isolated from a maize plant or germplasm.

In some embodiments, methods of selecting a drought tolerant maize plant or germplasm are provided. Such methods can comprise crossing a first maize plant or germplasm with a second maize plant or germplasm, wherein the first maize plant or germplasm comprises a marker associated with increased drought tolerance, and selecting a progeny plant or germplasm that possesses the marker (e.g. a marker located 50 cM, 20 cM, 10 cM, 5 cM, 2 cM or 1 cM from any one of chromosome intervals 1-15, a marker located within a chromosomal interval or combination thereof comprising at least one interval 1-15 as herein defined, or any marker or combination thereof of a marker listed in Tables 1-7 or yield alleles 1-8) that have been demonstrated to associate with increased drought tolerance and/or yield.

In some embodiments, methods of introgressing an allele associated with increased drought tolerance into a maize plant or maize germplasm are provided. Such methods can comprise crossing a first maize plant or germplasm comprising an allele associated with increased drought tolerance (e.g. any allele as identified in Tables 1-7) with a second maize plant or germplasm that lacks said allele and repeatedly backcrossing progeny plants comprising said allele with the second maize plant or germplasm to produce a drought tolerant maize plant or germplasm comprising the allele associated with increased drought tolerance. Progeny comprising the allele associated with increased drought tolerance can be identified by detecting, in their genomes, the presence of a marker associated with said allele; for example a marker located within a chromosomal interval (e.g. any of chromosome intervals 1-15 or a portion thereof or within 50 cM, 20 cM, 10 cM or less from yield alleles 1-8) or combination thereof comprising at least one chromosome interval 1-15 as herein defined, or any marker or combination thereof of a marker listed in Tables 1-7.

Plants and/or germplasms identified, produced or selected by any of the methods of the invention are also provided, as are any progeny or seeds derived from a plant or germplasm identified, produced or selected by these methods described herein.

Non-naturally occurring maize plants and/or germplasms having introgressed (e.g. through plant breeding, transgenic expression or genome editing) into its genome any one of chromosome intervals 1-15 comprising one or more markers associated with increased drought tolerance are also provided. In some embodiments the non-naturally occurring maize plant and/or germplasm is a progeny plant of a maize plant that has been selected for breeding purposes on the basis of the presence of a marker that associates with increased drought tolerance and/or increased yield under well watered conditions and wherein said marker is located within a chromosomal interval that corresponds to any one or more of chromosome interval 1, 2, 3, 4, 5, 6, 7 or portions thereof. In other embodiments, a non-naturally occurring plant is created by editing within a plant's genome a allelic change corresponding to any one of yield alleles 1-8 or favorable alleles as identified in any one of Tables 1-7, wherein the allelic change results in a plant having increased drought and/or increase yield as compared to a control plant.

Methods of employing markers associated with increased drought tolerance are also provided. Such markers can comprise a nucleotide sequence having at least 85%, 90%, 95%, or 99% sequence identity to any one of SEQ ID NOs: 1-8, 17-66; the reverse complement thereof, or an informative or functional fragment thereof.

Compositions comprising a primer pair capable of amplifying a nucleic acid sample isolated from a maize plant or germplasm to generate a marker associated with increased drought tolerance are also provided. Such compositions can comprise, consist essentially of, or consist of one of the amplification primer pairs identified in Table 8.

TABLE 8
SEQ ID NOs. of Exemplary Oligonucleotide Primers
and Probes that can be Employed for Analyzing Water
Optimization Loci, Alleles, and Haplotypes

| Genomic | | |
|---|---|---|
| Locus SEQ ID NO | Exemplary | Exemplary Assay |
| (Associated Locus): | Amplification Primers | Probes |
| 17 (SM2987) | 25, 26 | 27, 28 |
| 18 (SM2991) | 29, 30 | 31, 32 |
| 19 (SM2995) | 33, 34 | 35, 36 |
| 20 (SM2996) | 37, 38 | 39, 40 |
| 21 (SM2973) | 41, 42 | 43, 44 |
| 22 (SM2980) | 45, 46 | 47, 48 |
| 23 (SM2982) | 49, 50 | 51, 52 |
| 24 (SM2984) | 53, 54 | 55, 56 |

A marker associated with increased drought tolerance can comprise, consist essentially of, and/or consist of a single allele or a combination of alleles at one or more genetic loci (e.g. a genetic loci comprising any one of SEQ ID NOs: 1-8, 17-65 and/or yield alleles 1-8, as defined herein).

Another embodiment of the invention is a method of selecting or identifying a maize plant having increased drought tolerance as compared to a control plant wherein increased drought tolerance is increased yield in bushels per acre as compared to a control plant, the method comprises the steps of: a) isolating a nucleic acid from a maize plant; b) detecting in the nucleic acid of a) a molecular marker that is closely linked and associated with drought tolerance (e.g. any marker from Tables 1-7); and c) identifying or selecting a maize line having increased drought tolerance as compared to a control plant based on the molecular marker detected in b). In some embodiments the marker detected in b) is within a chromosome interval selected from any one of chromosome intervals 1-15 as defined herein. In another embodiment the marker detected in b) comprises any one of SEQ ID Nos: 17-24 wherein the sequence comprises any favorable allele as described in Tables 1-7. Further embodiments include a chromosome interval wherein any one of the primer pairs in Table 8 anneal to the said interval and PCR amplification creates an amplicon diagnostic for associating a given marker with increased drought tolerance.

In another embodiment, the genes, chromosomal intervals, markers and genetic loci of the invention may be combined with the markers described in U.S. Patent Application 2011-0191892, herein incorporated in its entirety by reference. For example, genetic loci comprising any one of SEQ ID NOs: 1-8; 17-77 or alleles comprised therein that associate with increased drought tolerance and/or increased yield under well-watered conditions in maize may be combined with any one or more of Haplotypes A-M wherein haplotypes A-M are defined as follows:

i. Haplotype A comprises a G nucleotide at the position that corresponds to position 115 of SEQ ID NO: 65, an A nucleotide at the position that corresponds to position 270 of SEQ ID NO: 65, a T nucleotide at the position that corresponds to position 301 of SEQ ID NO: 65, and an A nucleotide at the position that corresponds to position 483 of SEQ ID NO: 1 on chromosome 8 in the first plant's genome;

ii. Haplotype B comprises a deletion at positions 4497-4498 of SEQ ID NO: 66, a G nucleotide at the position that corresponds to position 4505 of SEQ ID NO: 66, a T nucleotide at the position that corresponds to position 4609 of SEQ ID NO: 66, an A nucleotide at the position that corresponds to position 4641 of SEQ ID NO: 66, a T nucleotide at the position that corresponds to position 4792 of SEQ ID NO: 66, a T nucleotide at the position that corresponds to position 4836 of SEQ ID NO: 66, a C nucleotide at the position that corresponds to position 4844 of SEQ ID NO: 66, a G nucleotide at the position that corresponds to position 4969 of SEQ ID NO: 66, and a TCC trinucleotide at the position that corresponds to positions 4979-4981 of SEQ ID NO: 66 on chromosome 8 in the first plant's genome;

iii. Haplotype C comprises an A nucleotide at the position that corresponds to position 217 of SEQ ID NO: 67, a G nucleotide at the position that corresponds to position 390 of SEQ ID NO: 67, and an A nucleotide at the position that corresponds to position 477 of SEQ ID NO: 67 on chromosome 2 in the first plant's genome;

iv. Haplotype D comprises a G nucleotide at the position that corresponds to position 182 of SEQ ID NO: 68, an A nucleotide at the position that corresponds to position 309 of SEQ ID NO: 68, a G nucleotide at the position that corresponds to position 330 of SEQ ID NO: 68, and a G nucleotide at the position that corresponds to position 463 of SEQ ID NO: 68 on chromosome 8 in the first plant's genome;

v. Haplotype E comprises a C nucleotide at the position that corresponds to position 61 of SEQ ID NO: 69, a C nucleotide at the position that corresponds to position 200 of SEQ ID NO: 69, and a deletion of nine nucleotides at the positions that corresponds to positions 316-324 of SEQ ID NO: 69 on chromosome 5 in the first plant's genome;

vi. Haplotype F comprises a G nucleotide at the position that corresponds to position 64 of SEQ ID NO: 70 and a T nucleotide at the position that corresponds to position 254 of SEQ ID NO: 70 on chromosome 8 in the first plant's genome;

vii. Haplotype G comprises an C nucleotide at the position that corresponds to position 98 of SEQ ID NO: 71, a T nucleotide at the position that corresponds to position 147 of SEQ ID NO: 71, a C nucleotide at the position that corresponds to position 224 of SEQ ID NO: 71, and a T nucleotide at the position that corresponds to position 496 of SEQ ID NO: 71 on chromosome 9 in the first plant's genome;

viii. Haplotype H comprises a T nucleotide at the position that corresponds to position 259 of SEQ ID NO: 72, a T nucleotide at the position that corresponds to position 306 of SEQ ID NO: 72, an A nucleotide at the position that corresponds to position 398 of SEQ ID NO: 72, and a C nucleotide at the position that corresponds to position 1057 of SEQ ID NO: 72 on chromosome 4 in the first plant's genome;

ix. Haplotype I comprises a C nucleotide at the position that corresponds to position 500 of SEQ ID NO: 73, a G nucleotide at the position that corresponds to position 568 of SEQ ID NO: 73, and a T nucleotide at the position that corresponds to position 698 of SEQ ID NO: 73 on chromosome 6 in the first plant's genome;

x. Haplotype J comprises an A nucleotide at the position that corresponds to position 238 of SEQ ID NO: 74, a deletion of the nucleotides that correspond to positions 266-268 of SEQ ID NO: 74, and a C nucleotide at the position that corresponds to position 808 of SEQ ID NO: 74 in the first plant's genome;

xi. Haplotype K comprises a C nucleotide at the position that corresponds to position 166 of SEQ ID NO: 75, and A nucleotide at the position that corresponds to position 224 of SEQ ID NO: 75, a G nucleotide at the position that corresponds to position 650 of SEQ ID NO: 75, and a G nucleotide at the position that corresponds to position 892 of SEQ ID NO: 75 on chromosome 8 in the first plant's genome;

xii. Haplotype L comprises a C nucleotide at the positions that correspond to positions 83, 428, 491, and 548 of SEQ ID NO: 76 on chromosome 9 in the first plant's genome; and

xiii. Haplotype M comprises a C nucleotide at the position that corresponds to position 83 in SEQ ID NO: 77, an A nucleotide at the position that corresponds to position 119 of SEQ ID NO: 77, and a T nucleotide at the position that corresponds to position 601 of SEQ ID NO: 77.

Thus, in some embodiments the presently disclosed subject matter provides a method of stacking a haplotype selected from the group comprised of any one of Haplotypes A, B, C, D, E, F, G, H, I, J, K, L, and M with a marker selected from the group comprising and closely associated with SM2987, SM2991, SM2995, SM2996, SM2973, SM2980, SM2982, and SM2984 such as those in tables 1-7; or markers closely linked to of SM2987, SM2991, SM2995, SM2996, SM2973, SM2980, SM2982, and SM2984 or markers comprising any one of SEQ ID Nos: 17-24. Further provided are maize plants comprising in their genome stacks of haplotypes and or loci that are not present in nature wherein the stacks comprise any one of Haplotypes A-M, as defined in combination with any one of SM2987, SM2991, SM2995, SM2996, SM2973, SM2980, SM2982, and SM2984. In some instances maize plants comprising these unique stacks not present in nature (e.g. comprising a combination of Haplotypes A-M or loci SM2987, SM2991, SM2995, SM2996, SM2973, SM2980, SM2982, and SM2984) are hybrid maize plants and in some instances the hybrid maize plant comprises in its genome an active transgene for either herbicide resistance and/or insect resistance.

Thus, in some embodiments the presently disclosed subject matter provides methods for producing a hybrid plant with increased drought tolerance. In some embodiments, the method comprise (a) providing a first plant comprising a first genotype comprising any one of haplotypes A-M: (b) providing a second plant comprising a second genotype comprising any one from the group comprised of SM2987, SM2991, SM2995, SM2996, SM2973, SM2980, SM2982, and SM2984, wherein the second plant comprises at least one marker from the group comprised of SM2987, SM2991, SM2995, SM2996, SM2973, SM2980, SM2982, and SM2984 that is not present in the first plant; (c) crossing the first plant and the second maize plant to produce an F1 generation; identifying one or more members of the F1 generation that comprises a desired genotype comprising any combination of haplotypes A-M and any markers from the group comprised of SM2987, SM2991, SM2995, SM2996, SM2973, SM2980, SM2982, and SM2984, wherein the desired genotype differs from both the first genotype of (a) and the second genotype of (b), whereby a hybrid plant with increased drought is produced. In some aspects of the embodiment the hybrid plant of (b) further comprises within its genome a transgene for herbicide resistance and/or insect resistance. In some aspects the hybrid plant of (b) is an elite maize line.

In another embodiment, the presently disclosed subject matter discloses a method to produce a maize plant having increased drought tolerance as compared to a control plant wherein yield is increased bushels per acre (in some embodiments YGSMN), the method comprising the steps of: a) isolating a nucleic acid from a first maize plant; b) detecting in the nucleic acid of a) a molecular marker associated with increased drought tolerance (e.g. any of the markers described in Tables 1-7 or closely associate markers) wherein the marker is located within a chromosomal interval 1-15; or wherein the chromosome interval is defined as 50 cM, 40 cM, 30 cM, 20 cM, 10 cM, 9 cM, 8 cM, 7 cM, 6 cM, 5 cM, 4 cM, 3 cM, 2 cM, 1 cM or 0.5 cM or less from any one of yield alleles 1-8; or the chromosomal interval comprises any one of SEQ ID Nos 17-24; or the marker is closely associated to a respective marker described in Tables 1-7; c) selecting a first maize plant on the basis of the marker detected in b); d) crossing the first maize plant with a second maize plant not comprising the marker of b); e) producing a progeny plant from the crossing of d) wherein the progeny plant has introgressed into its genome the marker of b) thereby producing a maize plant having increased drought tolerance as compared to a control plant. In some aspects seed produced by the embodiment wherein the seed comprises the marker of b) in its genome.

In another embodiment, the presently disclosed subject matter discloses a method to produce a plant having increased drought tolerance, increased yield under drought or increased yield under non-drought conditions as compared to a control plant, the method comprising the steps of a) in a plant cell, editing a plant's genome (i.e. through CRISPR, TALEN or Meganucleases) to comprise a molecular marker (e.g. SNP) associated with increased drought tolerance, increased yield under drought or increased yield under non-drought conditions wherein the molecular marker is any marker (e.g. favorable allele) as described in Tables 1-7 and further wherein the plant genome did not have said molecular marker previously; b) producing a plant or plant callus from the plant cell of a). In particular the editing comprises any one of yield alleles 1-8 or closely associated alleles thereof. In another aspect of the embodiment the editing is to a gene having 70%, 80%, 85%, 90%, 92%, 95%, 98%, 99% or 100% sequence homology or sequence identity to a gene comprising SEQ ID Nos: 1-8.

In some embodiments, the hybrid plant with increased drought tolerance comprises each of haplotypes A-M that are present in the first plant as well as at least one additional loci selected from the group comprised of SM2987, SM2991, SM2995, SM2996, SM2973, SM2980, SM2982, and SM2984 (or a marker within any one of chromosome intervals 1-15 that associates with either increased drought tolerance and/or increased yield under well-watered conditions, wherein yield is increased bushels per acre, or a marker comprising SEQ ID Nos 17-24) that is present in the second plant. In some embodiments, the first plant is a recurrent parent comprising at least one of haplotypes A-M and the second plant is a donor that comprises at least one marker from the group comprised of SM2987, SM2991, SM2995, SM2996, SM2973, SM2980, SM2982, or SM2984 that is not present in the first plant. In some embodiments, the first plant is homozygous for at least two, three, four, or five of haplotypes A-M. In some embodiments, the hybrid plant comprises at least three, four, five, six, seven, eight, or nine of haplotypes A-M and markers from the group comprised of SM2987, SM2991, SM2995, SM2996, SM2973, SM2980, SM2982, or SM2984 or any one of yield alleles 1-8.

In some embodiments, one may identify a drought tolerant maize plant by genotyping one or more members of an F1 generation produced by crossing the first plant and the second plant with respect to each of the haplotypes A-M and markers from the group comprised of SM2987, SM2991, SM2995, SM2996, SM2973, SM2980, SM2982, and SM2984 present in either the first plant or the second plant. In some embodiments, the first plant and the second plant are Zea mays plants and in other instances the first and second plant are inbred Zea mays plants.

In some embodiments, “increased water optimization” confers increased or stabilized yield in a water stressed environment as compared to a control plant. Maize plants having enhance water optimization may be selected, identified or produced using any of the markers listed in Tables 1-7 or a marker within chromosome intervals 1-15. In some embodiments, the hybrid with increased water optimization can be planted at a higher crop density. In some embodiments, the hybrid with increased water optimization confers no yield drag when under favorable moisture levels. In yet another embodiment the plants comprising any of the markers or chromosome intervals identified in Tables 1-7 may confer any one of increased drought tolerance or increased yield as compared to a control plant or further increased yield under non-drought or well-watered conditions wherein yield is increased bushels of corn per acre (i.e. YGSMN).

The presently disclosed subject matter also provides in some embodiments hybrid Zea mays plants produced by the presently disclosed methods, or a cell, tissue culture, seed, or plant part thereof.

The presently disclosed subject matter also provides in some embodiments inbred Zea mays plants produced by backcrossing and/or selfing and/or producing double haploids from the hybrid Zea mays plants disclosed herein, or a cell, tissue culture, seed, or part thereof.

In some embodiments, maize plants having increased drought tolerance are identified by genotyping one or more members of an F1 generation produced by crossing the first plant and the second plant with respect to each of any chromosomal intervals, markers and/or combination thereof displayed in Tables 1-7 or comprised in any one of or combination of SEQ ID NOs: 1-8; 17-65 present in either the first plant or the second plant. In some embodiments, the first plant and the second plant are Zea mays plants. In other embodiments the first plant or second plant is either a Zea mays inbred or a Zea mays hybrid or an elite Zea mays line.

The presently disclosed subject matter also provides in some embodiments, hybrid or inbred Zea mays plants that have been modified to include a transgene. In some embodiments, the transgene encodes a gene product that provides resistance to a herbicide selected from among glyphosate, Sulfonylurea, imidazolinione, dicamba, glufisinate, phenoxy proprionic acid, cycloshexome, traizine, benzonitrile, and broxynil. For example, any hybrid or inbred Zea mays plant having comprised in its genome a transgene encoding any one of glyphosate, Sulfonylurea, imidazolinione, dicamba, glufisinate, phenoxy proprionic acid, cycloshexome, traizine, benzonitrile, and broxynil resistance transgene and further wherein said plant has introduced via plant breeding, transgenic expression or genome editing into its genome any one of SEQ ID Nos 1-8 or any of Yield alleles 1-8.

The presently disclosed subject matter also provides in some embodiments methods for identifying Zea mays plants comprising at least one allele associated with increased drought tolerance as disclosed herein (e.g. any marker closely associated with alleles described in Tables 1-7). In some embodiments, the methods comprise (a) genotyping and identifying at least one Zea mays plant with at least one nucleic acid marker comprising any one of SEQ ID NOs: 1-8; 17-60; and (b) selecting at least one Zea mays plant comprising an allele associated with drought tolerance identified in b).

The presently disclosed subject matter also provides in some embodiments Zea mays plants produced by introgressing an allele of interest of a locus associated with increased drought tolerance into a Zea mays germplasm. In some embodiments, the introgressing comprises (a) selecting a Zea mays plant that comprises an allele of interest of a locus associated with increased drought tolerance, wherein the locus associated with increased drought tolerance comprises a nucleotide sequence that is at least 80%, 85%, 90%, 95%, 98% or 100% identical to any of SEQ ID NOs: 1-8; 17-60 or wherein the nucleotide sequence comprises any one of yield alleles 1-7 or a combination thereof; and (b) introgressing the allele of interest into Zea mays germplasm that lacks the allele.

In another embodiment, the invention provides maize germplasm that has been enriched with any one of chromosome intervals 1-15 or yield alleles 1-7, wherein enrichment comprises the steps of identifying or selecting lines having the said chromosome intervals or yield alleles and crossing these lines with lines not having said intervals or portions thereof and backcrossing to create inbred lines with said intervals or yield alleles then employing said inbred lines into a plant breeding system to create a commercial maize population enriched for said interval or yield alleles thereof (e.g. a commercial hybrid maize population having greater than 30%, 40% or over 50% of its hybrids enriched with said interval or yield alleles as compared to a 5 year historical pedigree of said hybrid maize population having <30% enrichment of said interval or yield alleles.

In some embodiments, a method of identifying and/or selecting a maize plant or plant part having increased yield under non-drought conditions, increased yield stability under drought conditions, and/or increased drought tolerance, comprising: detecting, in a maize plant or plant part, an allele of at least one marker locus that is associated with increased yield under non-drought conditions, increased yield stability under drought conditions, and/or increased drought tolerance in a plant, wherein said at least one marker locus is located within a chromosomal interval selected from the group consisting of:

(a) a chromosome interval on maize chromosome 1 defined by and including base pair (bp) position 272937470 to base pair (bp) position 272938270 (herein “interval 8”);

(b) a chromosome interval on maize chromosome 2 defined by and including base pair (bp) position 12023306 to base pair (bp) position 12024104 (herein “interval 9”);

(c) a chromosome interval on maize chromosome 3 defined by and including base pair (bp) position 225037202 to base pair (bp) position 225038002 (herein “interval 10”);

(d) a chromosome interval on maize chromosome 3 defined by and including base pair (bp) position 225340531 to base pair (bp) position 225341331 (herein “interval 11”);

(e) a chromosome interval on maize chromosome 5 defined by and including base pair (bp) position 159,120,801 to base pair (bp) position 159,121,601 (herein “interval 12”);

(f) a chromosome interval on maize chromosome 9 defined by and including base pair (bp) position 12104536 to base pair (bp) position 12105336 (herein “interval 13”); (g) a chromosome interval on maize chromosome 9 defined by and including base pair (bp) position 225343590 to base pair (bp) position 225340433 (herein “interval 14”);

(h) a chromosome interval on maize chromosome 10 defined by and including base pair (bp) position 14764415 to base pair (bp) position 14765098 (herein “interval 15”); is contemplated. In a preferred embodiment chromosome intervals 8-14 further comprise a respective yield allele 1-7 as defined herein.

In further embodiments, a method of identifying and/or selecting a maize plant or plant part having increased yield under non-drought conditions, increased yield stability under drought conditions, and/or increased drought tolerance, comprising: detecting, in a maize plant or plant part, an allele of at least one marker locus that is associated with increased yield under non-drought conditions, increased yield stability under drought conditions, and/or increased drought tolerance in a plant, wherein said at least one marker is selected from the group or a marker located within 50 cM, 40 cM, 30 cM, 20 cM, 15 cM, 10 cM, 9 cM, 8 cM, 7 cM, 6 cM, 5 cM, 4 cM, 3 cM, 2 cM, 1 cM or 0.5 cM of the following causative alleles:

Chromosome 1 bp position 272937870 comprises a G allele;

Chromosome 2 bp position 12023706 comprises a G allele;

Chromosome 3 bp position 225037602 comprises a A allele;

Chromosome 3 bp position 225340931 comprises an A allele;

Chromosome 5 bp position 159121201 comprises a G allele;

Chromosome 9 bp position 12104936 comprises a C allele;

Chromosome 9 bp position 133887717 comprises an A allele; and

Chromosome 10 bp position 4987333 comprises a G allele; or any combination thereof.

In another embodiment, a method for selecting a drought tolerant maize plant the method comprising the steps of: a) isolating a nucleic acid from a plant cell; b) detecting in said nucleic acid a molecular marker associated with increased drought tolerance wherein said marker is within a chromosome interval comprising any one of chromosome intervals 1-15, as defined herein; and c) selecting or identifying a maize plant having increased drought tolerance based on the detection of the marker in b). Some further embodiments, wherein the respective chromosomal interval comprises any one of the following alleles:

Chromosome 1 bp position 272937870 comprises a G allele;

Chromosome 2 bp position 12023706 comprises a G allele;

Chromosome 3 bp position 225037602 comprises a A allele;

Chromosome 3 bp position 225340931 comprises an A allele;

Chromosome 5 bp position 159121201 comprises a G allele;

Chromosome 9 bp position 12104936 comprises a C allele;

Chromosome 9 bp position 133887717 comprises an A allele; and

Chromosome 10 bp position 4987333 comprises a G allele;

any allele listed in Tables 1-7; or any combination thereof.

In some embodiments, the invention provides methods for producing a hybrid maize plant with increased yield, wherein increased yield in either drought or non-drought conditions and increased yield is increased bushels per acre of corn as compared to a control, the method comprising the steps of: (a) identifying a first maize plant comprising a first genotype by identifying any one of markers SM2987, SM2996, SM2982, SM2991, SM2995, SM2973, SM2980, or SM2984, yield alleles 1-8 or any closely associated markers thereof (e.g. any markers in Tables 1-7); (b) identifying a second maize plant comprising a second genotype by identifying anyone of markers SM2987, SM2996, SM2982, SM2991, SM2995, SM2973, SM2980, or SM2984 or yield alleles 1-8 not comprised in the first maize plant, c) crossing the first maize plant and the second maize plant to produce an F1 generation; and (d) selecting one or more members of the F1 generation that comprises a desired genotype comprising any combination of markers SM2987, SM2996, SM2982, SM2991, SM2995, SM2973, SM2980, or SM2984, wherein the desired genotype differs from both the first genotype of (a) and the second genotype of (b), whereby a hybrid maize plant with increased yield in bushels per acre is produced.

In one embodiment, the present invention provides a non-natural hybrid plant comprising a nucleic acid molecule selected from the group consisting of SEQ ID NO: 17-24 or fragments thereof, yield alleles 1-8 or complements thereof.

The present invention also provides a plant comprising alleles of SM2987, SM2996, SM2982, SM2991, SM2995, SM2973, SM2980, or SM2984 or fragments and complements thereof as well as any plant comprising any combination of one or more drought tolerance loci selected from the group consisting of SEQ ID NOs: 17-24 wherein said drought tolerance loci associate with increased drought tolerance. Such alleles may be homozygous or heterozygous.

In another embodiment, the invention provides methods of introducing into a plant genome a gene that confers increased drought tolerance or increased yield in said plant. It is contemplated that genes may be introduced via conventional plant breeding methods, transgenic expression, via mutation such as by Ethyl methanesulfonate (ESM), or through gene editing approaches such as TALEN, CRISPR, meganuclease, or etc. In some embodiments, not to be limited by theory, a nucleotide sequence comprising any one or more of the gene models listed in Table 9 below, or SEQ ID Nos 1-8 may be introduced into a plant's genome to create plants having increased yield and/or increased drought tolerance as compared to a control plant. Also it is contemplated that one may likewise introduce a causative allele for increased yield wherein the causative allele is selected from the alleles listed in any one of Tables 1-7.

TABLE 9
Summary of putative gene models causative for increased drought tolerance and/or increased yield in plants:

| SNP Name | Assay | Gene_Model | Gene_Charatertization |
|---|---|---|---|
| PZE01271951242 | SM2987 | GRMZM2G027059 | Gene involved in the plastid nonmevalonate pathway of isoprenoid |
| | | | biosynthesis. Isoprene emission protects photosynthesis but reduces plant |
| | | | productivity during drought in transgenic tobacco. |
| PZE0211924330 | SM2991 | GRMZM2G156365 | Pectinacetylesterase, involved in homogalacturonan degradation. Similar to |
| | | | Protein kinase APK1A, chloroplast precursor (EC 2.7.1.—). |
| PZE03223368820 | SM2995 | GRMZM2G134234 | Protein of unknown function with zion ion binding. In Arabidopsis the gene is |
| | | | involved in pollen tube growth and response to vial pathogen. |
| PZE03223703236 | SM2996 | GRMZM2G094428 | Transferase; Chloramphenicol acetyltransferase-like domain |
| PZE05158466685 | SM2973 | GRMZM2G416751 | Uncharacterized protein, hypothesized to be involved in pollen exine |
| | | | formation in Arabidopsis |
| PZE0911973339 | SM2980 | GRMZM2G467169 | Uncharacterized protein, hypothesized to be involved in actin nucleation, root |
| | | | hair cell differentation, and trichome morphogenesis in Arabidopsis |
| S_18791654 | SM2982 | GRMZM5G862107 | Ribosomal protein S1, RNA-binding domain, hypothesized to be involved in |
| | | | translation initiation of many mRNAs and might also play a role in translation |
| | | | elongation. A heat-responsive protein that funcitons in protein biosynthesis in |
| | | | the chloroplast in Arabidopsis, when knocked down leads to loss of heat |
| | | | tolerance |
| S_20808011 | SM2984 | GRMZM2G050774 | Zinc finger (C3HC4-type RING finger) family; |

In one embodiment, compositions and methods for producing plants having increased drought tolerance may be produced using any of the molecular markers as described in Tables 1-7 are contemplated. For example a maize plant can be identified, selected or produced through the identification and/or selection of an allele that associates with increased drought tolerance as displayed in Tables 1-7.

In another aspect of the invention transgenic plants having increased tolerance to drought and/or increased yield may be produced by operably linking any one of the genes in Table 9, or SEQ ID Nos: 1-8, or homologs/orthologs thereof to a plant promoter (constitutive or tissue specific) and expressing said gene in plant. For example, it is contemplated that said genes may be expressed either by constitutive or by tissue specific/preferred expression. Not to be limited by example, but it is contemplated that one could target expression to, for example, the corn ear, the shank, reproductive tissue, fruit, seed, or other plant parts to produce transgenic plants having increased yield and/or drought tolerance.

These and other aspects of the invention are set forth in more detail in the description of the invention below.

### BRIEF DESCRIPTION OF THE DRAWINGS

FIG. 1 is a bar chart demonstrating that transgenic plants expressing GRMZM2G027059 (construct 23294) have significantly more total chlorophyll as compared to a control (CK) plants.

FIG. 2 is a bar chart demonstrating that transgenic plants expression GRMZM2G156365 T show increased sugars involved in pectin formation (Event data relative to increase over controls).

FIG. 3 is a metabolite profile of transgenic T1 plants overexpressing GRMZM2G094428 (Columns to the right are wild type controls: overexpression of this gene in Arabidopsis decreased two major substrates for lignin formation and increased the ester receptor spermidine.)

FIG. 4 is a metabolite profile of transgenic T1 plants overexpressing GRMZM2G416751 (controls are on the right; overexpression of this gene in Arabidopsis decreased expression of glucoronate, 3-deoxyoctulosonate and sinapate).

FIG. 5 is a bar chart demonstrating that transgenic plants expressing GRMZM2G467169 (construct 23403) have significantly more total chlorophyll as compared to a control (CK) plants.

FIG. 6 is a bar chart demonstrating that transgenic plants expressing GRMZM5G862107 (construct 23292) have significantly higher expression of HsfA2 in 2 events as compared to wild type controls indicating possible role in heat stress tolerance.

### BRIEF DESCRIPTION OF THE SEQUENCES

The instant disclosure includes a plurality of nucleotide and/or amino acid sequences. Throughout the disclosure and the accompanying sequence listing, the WIPO Standard ST.25 (1998; hereinafter the “ST.25 Standard”) is employed to identify nucleotides. This nucleotide identification standard is summarized below:

Nucleotide Naming Conventions in WIPO Standard ST.25

| Symbol | Meaning | Symbol | Meaning |
|---|---|---|---|
| a | a | k | g or t/u |
| c | c | s | g or c |
| g | g | w | a or t/u |
| t | t | b | g or c or t/u |
| u | u | d | a or g or t/u |
| r | g or a | h | a or c or t/u |
| v | t/u or c | v | a or g or c |
| m | a or c | n | a or g or c or t/u, |
| | | | unknown, other, |
| | | | or absent |

Additionally, whether specifically noted or not, for each recitation of “n” in the Sequence Listing, it is understood that any individual “n” (including some or all n's in a sequence of consecutive n's) can represent a, c, g, t/u, unknown, or other, or can be absent. Thus, unless specifically defined to the contrary in the Sequence Listing, an “n” can in some embodiments represent no nucleotide.

SEQ ID NO: 1 is a nucleotide sequence of the cDNA of the water optimization gene GRMZM2G027059 located on Zm chromosome 1 within chromosome intervals 1 and 8;
SEQ ID NO: 2 is a nucleotide sequence of the cDNA of the water optimization gene GRMZM2G156366 located on Zm chromosome 2 within chromosome intervals 4 and 9.
SEQ ID NO: 3 is a nucleotide sequence of the cDNA of the water optimization gene GRMZM2G134234 located on Zm chromosome 3 within chromosome intervals 2 and 10.
SEQ ID NO: 4 is a nucleotide sequence of the cDNA of the water optimization gene GRMZM2G094428 located on Zm chromosome 3 within chromosome intervals 2 and 11.
SEQ ID NO: 5 is a nucleotide sequence of the cDNA of the water optimization gene GRMZM2G416751 located on Zm chromosome 5 within chromosome intervals 5 and 12.
SEQ ID NO: 6 is a nucleotide sequence of the cDNA of the water optimization gene GRMZM2G467169 located on Zm chromosome 9 within chromosome intervals 6 and 13.
SEQ ID NO: 7 is a nucleotide sequence of the cDNA of the water optimization gene GRMZM5G862107 located on Zm chromosome 9 within chromosome intervals 3 and 14.
SEQ ID NO: 8 is a nucleotide sequence of the cDNA of the water optimization gene GRMZM2G050774 located on Zm chromosome 10 within chromosome intervals 7 and 15.
SEQ ID NO: 9 is a protein sequence of the water optimization gene GRMZM2G027059.
SEQ ID NO: 10 is a protein sequence of the water optimization gene GRMZM2G156365.
SEQ ID NO: 11 is a protein sequence of the water optimization gene GRMZM2G134234.
SEQ ID NO: 12 is a protein sequence of the water optimization gene GRMZM2G094428.
SEQ ID NO: 13 is a protein sequence of the water optimization gene GRMZM2G416751.
SEQ ID NO: 14 is a protein sequence of the water optimization gene GRMZM2G467169.
SEQ ID NO: 15 is a protein sequence of the water optimization gene GRMZM5G862107.
SEQ ID NO: 16 is a protein sequence of the water optimization gene GRMZM2G050774.
SEQ ID NO: 17 is a nucleotide sequence that is associated with the water optimization locus SM2987, subsequences of which can be amplified from chromosome 1 of the Zea mays genome using the polymerase chain reaction with amplification primers as set forth in Table 8.
SEQ ID NO: 18 is a nucleotide sequence that is associated with the water optimization locus SM2991, subsequences of which can be amplified from chromosome 2 of the Zea mays genome using the polymerase chain reaction with amplification primers as set forth in Table 8.
SEQ ID NO: 19 is a nucleotide sequence that is associated with the water optimization locus SM2995, subsequences of which can be amplified from chromosome 3 of the Zea mays genome using the polymerase chain reaction with amplification primers as set forth in Table 8.
SEQ ID NO: 20 is a nucleotide sequence that is associated with the water optimization locus SM2996, subsequences of which can be amplified from chromosome 3 of the Zea mays genome using the polymerase chain reaction with amplification primers as set forth in Table 8.
SEQ ID NO: 21 is a nucleotide sequence that is associated with the water optimization locus SM2973, subsequences of which can be amplified from chromosome 5 of the Zea mays genome using the polymerase chain reaction with amplification primers as set forth in Table 8.
SEQ ID NO: 22 is a nucleotide sequence that is associated with the water optimization locus SM2980, subsequences of which can be amplified from chromosome 9 of the Zea mays genome using the polymerase chain reaction with amplification primers as set forth in Table 8.
SEQ ID NO: 23 is a nucleotide sequence that is associated with the water optimization locus SM2982, subsequences of which can be amplified from chromosome 9 of the Zea mays genome using the polymerase chain reaction with amplification primers as set forth in Table 8.
SEQ ID NO: 24 is a nucleotide sequence that is associated with the water optimization locus SM2984, subsequences of which can be amplified from chromosome 10 of the Zea mays genome using the polymerase chain reaction with amplification primers as set forth in Table 8.
SEQ ID NO: 25 is a primer for amplifying SM2987
SEQ ID NO: 26 is a primer for amplifying SM2987
SEQ ID NO: 27 is a probe for SM2987
SEQ ID NO: 28 is a probe for SM2987
SEQ ID NO: 29 is a primer for amplifying SM2991
SEQ ID NO: 30 is a primer for amplifying SM2991
SEQ ID NO: 31 is a probe for SM2991
SEQ ID NO: 32 is a probe for SM2991
SEQ ID NO: 33 is a primer for amplifying SM2995
SEQ ID NO: 34 is a primer for amplifying SM2995
SEQ ID NO: 35 is a probe for SM2995
SEQ ID NO: 36 is a probe for SM2995
SEQ ID NO: 37 is a primer for amplifying SM2996
SEQ ID NO: 38 is a primer for amplifying SM2996
SEQ ID NO: 39 is a probe for SM2996
SEQ ID NO: 40 is a probe for SM2996
SEQ ID NO: 41 is a primer for amplifying SM2973
SEQ ID NO: 42 is a primer for amplifying SM2973
SEQ ID NO: 43 is a probe for SM2973
SEQ ID NO: 44 is a probe for SM2973
SEQ ID NO: 45 is a primer for amplifying SM2980
SEQ ID NO: 46 is a primer for amplifying SM2980
SEQ ID NO: 47 is a probe for SM2980
SEQ ID NO: 48 is a probe for SM2980
SEQ ID NO: 49 is a primer for amplifying SM2982
SEQ ID NO: 50 is a primer for amplifying SM2982
SEQ ID NO: 51 is a probe for SM2982
SEQ ID NO: 52 is a probe for SM2982
SEQ ID NO: 53 is a primer for amplifying SM2984
SEQ ID NO: 54 is a primer for amplifying SM2984
SEQ ID NO: 55 is a probe for SM2984
SEQ ID NO: 56 is a probe for SM2984
SEQ ID NO: 57 is a nucleotide sequence that is associated with the water optimization locus PZE01271951242 maize Chromosome 1 272,937,470 bp-272,938,270 bp (interval 8)
SEQ ID NO: 58 is a nucleotide sequence that is associated with the water optimization locus PZE0211924330 maize Chromosome 2 12,023,306 bp-12,024,104 bp (interval 9).
SEQ ID NO: 59 is a nucleotide sequence that is associated with the water optimization locus PZE03223368820 maize Chromosome 3 225,037,202 bp-225,038,002 bp (interval 10).
SEQ ID NO: 60 is a nucleotide sequence that is associated with the water optimization locus PZE03223703236 maize Chromosome 3 225,340,531 bp-225,341,331 bp (interval 11).
SEQ ID NO: 61 is a nucleotide sequence that is associated with the water optimization locus PZE05158466685 maize Chromosome 5 159,120,801 bp-159,121,601 bp (interval 12).
SEQ ID NO: 62 is a nucleotide sequence that is associated with the water optimization locus PZE0911973339 maize Chromosome 9 12,104,536 bp-12,105,336 bp (interval 13).
SEQ ID NO: 63 is a nucleotide sequence that is associated with the water optimization locus S_18791654 maize Chromosome 9 from bp 225343590-225340433 (interval 14).
SEQ ID NO: 64 is a nucleotide sequence that is associated with the water optimization locus S_20808011 maize Chromosome 9 from bp 14764415-14765098 (interval 15).
SEQ ID NO. 65 is a nucleotide sequence that is associated with water optimization locus Haplotype A.
SEQ ID NO. 66 is a nucleotide sequence that is associated with water optimization locus Haplotype B.
SEQ ID NO. 67 is a nucleotide sequence that is associated with water optimization locus Haplotype C.
SEQ ID NO. 68 is a nucleotide sequence that is associated with water optimization locus Haplotype D.
SEQ ID NO. 69 is a nucleotide sequence that is associated with water optimization locus Haplotype E.
SEQ ID NO. 70 is a nucleotide sequence that is associated with water optimization locus Haplotype F.
SEQ ID NO. 71 is a nucleotide sequence that is associated with water optimization locus Haplotype G.
SEQ ID NO. 72 is a nucleotide sequence that is associated with water optimization locus Haplotype H.
SEQ ID NO. 73 is a nucleotide sequence that is associated with water optimization locus Haplotype I.
SEQ ID NO. 74 is a nucleotide sequence that is associated with water optimization locus Haplotype J.
SEQ ID NO. 75 is a nucleotide sequence that is associated with water optimization locus Haplotype K.
SEQ ID NO. 76 is a nucleotide sequence that is associated with water optimization locus Haplotype L.
SEQ ID NO. 77 is a nucleotide sequence that is associated with water optimization locus Haplotype M.

### DETAILED DESCRIPTION

The presently disclosed subject matter provides compositions and methods for identifying, selecting, and/or producing maize plants with increased drought tolerance (also referred to herein as water optimization), as well as maize plants identified, selected and/or produced by a method of this invention. In addition, the presently disclosed subject matter provides maize plants and/or germplasms having within their genomes one or more markers associated with increased drought tolerance.

To assess the value of chromosomal intervals, loci, genes or markers under drought stress, diverse germplasm was screened in controlled field-experiments comprising a full irrigation control treatment and a limited irrigation treatment. A goal of the full irrigation treatment was to ensure that water did not limit the productivity of the crop. In contrast, a goal of the limited irrigation treatment was to ensure that water became the major limiting constraint to grain yield. Main effects (e.g., treatment and genotype) and interactions (e.g., genotype×treatment) could be determined when the two treatments were applied adjacent to one another in the field. Moreover, drought related phenotypes could be quantified for each genotype in the panel thereby allowing for marker trait associations to be conducted.

In practice, the method for the limited irrigation treatment can vary widely depending upon the germplasm being screened, the soil type, and climatic conditions at the site, pre-season water supply, and in-season water supply, to name just a few variables. Initially, a site is identified where in-season precipitation is low (to minimize the chance of unintended water application) and is suitable for cropping. In addition, determining the timing of the stress can be important, such that a target is defined to ensure that year-to-year, or location-to-location, screening consistency is in place. An understanding of the treatment intensity, or in some cases the yield loss desired from the limited irrigation treatment, can also be considered. Selection of a treatment intensity that is too light can fail to reveal genotypic variation. Selection of a treatment intensity that is too heavy can create large experimental error. Once the timing of stress is identified and treatment intensity is described, irrigation can be managed in a manner that is consistent with these targets. For the data generated in this application, well established trial sites were used that have been monitored for many years including such variables as weather trends, soil types, nutrient levels, etc. This allows for greater efficiencies in detecting phenotypes and subsequently genotypes for increased yield and/or drought tolerance

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods. Accordingly, unless indicated to the contrary, the numerical parameters set forth in this specification and attached claims are approximations that can vary depending upon the desired properties sought to be obtained by the presently disclosed subject matter.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the term “allele” refers to one of two or more different nucleotides or nucleotide sequences that occur at a specific chromosome locus.

As used herein, the term “anthesis silk interval” (ASI) refers to the difference between when a plant starts shedding pollen (anthesis) and when it begins producing silk (female). Data are collected on a per plot basis. In some embodiments, this interval is expressed in days.

A “locus” is a position on a chromosome where a gene or marker or allele is located. In some embodiments, a locus may encompass one or more nucleotides.

As used herein, the terms “desired allele,” “target allele”, “causative allele” and/or “allele of interest” are used interchangeably to refer to an allele associated with a desired trait (for e.g. any of the alleles listed in Tables 1-7 or closely associated alleles thereof).

As used herein, the phrase “associated with” refers to a recognizable and/or assayable relationship between two entities. For example, the phrase “associated with a water optimization trait” refers to a trait, locus, gene, allele, marker, phenotype, etc., or the expression thereof, the presence or absence of which can influence an extent, degree, and/or rate at which a plant or a part of interest thereof that has the water optimization trait grows. As such, a marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with increased drought tolerance” refers to a marker whose presence or absence can be used to predict whether and/or to what extent a plant will display a drought tolerant phenotype (e.g. markers identified in Tables 1-7 are all closely associated with increased maize yield under both drought and non-drought conditions).

As used herein, the terms “backcross” and “backcrossing” refer to the process whereby a progeny plant is crossed back to one of its parents one or more times (e.g., 1, 2, 3, 4, 5, 6, 7, 8, 9, 10, or more.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on. In some embodiments, the number of backcrosses can be about 1 to about 10 (e.g., 1, 2, 3, 4, 5, 6, 7, 8, 9, and 10). In some embodiments, the number of backcrosses is about 7.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “cultivar” and “variety” refer to a group of similar plants that by structural or genetic features and/or performance can be distinguished from other varieties within the same species.

As used herein, the terms “elite” and/or “elite line” refer to any line that is substantially homozygous and has resulted from breeding and selection for desirable agronomic performance.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration (e.g. introgression) which resulted in the subject plant or cell; (b) a plant or plant cell of the same genotype as the starting material but which has been transformed with a null construct (i.e., with a construct which does not express the transfer cell-specific protein and sugar transporter as described herein); (c) a plant or plant cell which is a non-transformed segregant among progeny of a subject plant or plant cell or; (d) a plant that essentially identical in most aspects to the subject plant or plant cell however differ in genotype, specifically a SNP, haplotype, having an insertion/deletion (e.g. a maize control plant having a unfavorable allele at a specific chromosome position versus a subject (experimental) maize plant having a favorable allele at the same position).

As used herein, the term “chromosome” is used in its art-recognized meaning of the self-replicating genetic structure in the cellular nucleus containing the cellular DNA and bearing in its nucleotide sequence the linear array of genes. The Zea mays chromosome numbers disclosed herein refer to those as set forth in Perin et al., 2002, which relates to a reference nomenclature system adopted by L'institut National da Ia Recherché Agronomique (INRA; Paris, France).

As used herein, the phrase “consensus sequence” refers to a sequence of DNA built to identify nucleotide differences (e.g., SNP and Indel polymorphisms) in alleles at a locus. A consensus sequence can be either strand of DNA at the locus and states the nucleotide(s) at one or more positions (e.g., at one or more SNPs and/or at one or more Indels) in the locus. In some embodiments, a consensus sequence is used to design oligonucleotides and probes for detecting polymorphisms in the locus.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make up for all the genes in its genome. Genotypes can be indirectly characterized, e.g., using markers and/or directly characterized by, e.g., nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.). In some embodiments, germplasm includes but is not limited to tissue culture.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment (e.g. a haplotype could consist of any combination of at least two alleles listed respectively in Table 1, 2, 3, 4, 5, 6, or 7).

As used herein, the term “heterozygous” refers to a genetic status wherein different alleles reside at corresponding loci on homologous chromosomes. In some embodiments a maize parent line or progeny plant is heterozygous for any one of yield alleles 1-7

As used herein, the term “homozygous” refers to a genetic status wherein identical alleles reside at corresponding loci on homologous chromosomes. In some embodiments a maize parent line or progeny plant is homozygous for any one of yield alleles 1-7

As used herein, the term “hybrid” in the context of plant breeding refers to a plant that is the offspring of genetically dissimilar parents produced by crossing plants of different lines or breeds or species, including but not limited to a cross between two inbred lines.

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, 5, 6, 7, 8, 9, 10 or more times) to a line having a desired genetic background, selecting for the desired allele, with the result being that the desired allele becomes fixed in the desired genetic background. For example, a marker associated with drought tolerance (e.g. any markers shown in Tables 1-7) may be introgressed from a donor into a recurrent parent that is drought susceptible. The resulting offspring could then be backcrossed one or more times and selected until the progeny comprises the genetic marker(s) associated with drought tolerance in the recurrent parent background.

As used herein, the term “linkage” refers to a phenomenon wherein alleles on the same chromosome tend to be transmitted together more often than expected by chance if their transmission were independent. Thus, two alleles on the same chromosome are said to be “linked” when they segregate from each other in the next generation in some embodiments less than 50% of the time, in some embodiments less than 25% of the time, in some embodiments less than 20% of the time, in some embodiments less than 15% of the time, in some embodiments less than 10% of the time, in some embodiments less than 9% of the time, in some embodiments less than 8% of the time, in some embodiments less than 7% of the time, in some embodiments less than 6% of the time, in some embodiments less than 5% of the time, in some embodiments less than 4% of the time, in some embodiments less than 3% of the time, in some embodiments less than 2% of the time, and in some embodiments less than 1% of the time.

As such, “linkage” typically implies and can also refer to physical proximity on a chromosome. Thus, two loci are linked if they are within in some embodiments 20 centiMorgans (cM), in some embodiments 15 cM, in some embodiments 12 cM, in some embodiments 10 cM, in some embodiments 9 cM, in some embodiments 8 cM, in some embodiments 7 cM, in some embodiments 6 cM, in some embodiments 5 cM, in some embodiments 4 cM, in some embodiments 3 cM, in some embodiments 2 cM, and in some embodiments 1 cM of each other. Similarly, a yield locus (e.g. yield alleles 1-8) of the presently disclosed subject matter is linked to a marker (e.g., a genetic marker) if it is in some embodiments within 20, 15, 12, 10, 9, 8, 7, 6, 5, 4, 3, 2, or 1 cM of the marker. Thus, a marker linked to any one of yield alleles 1-8 may be utilized to select, identify or produce maize plants having increased tolerance to drought and/or increased yield.

In some embodiments of the presently disclosed subject matter, it is advantageous to define a bracketed range of linkage, for example, from about 10 cM and about 20 cM, from about 10 cM and about 30 cM, or from about 10 cM and about 40 cM. The more closely a marker is linked to a second locus (e.g. yield alleles 1-8), the better an indicator for the second locus that marker becomes. Thus, “closely linked” or interchangeably “closely associated” loci or markers such as a marker locus and a second locus display an inter-locus recombination frequency of about 10%, 9%, 8%, 7%, 6%, 5%, 4%, 3%, or 2% or less. In some embodiments, the relevant loci display a recombination frequency of about 1% or less, e.g., about 0.75%, 0.5%, 0.25% or less. Two loci that are localized to the same chromosome, and at such a distance that recombination between the two loci occurs at a frequency of less than about 10% (e.g., about 9%, 8%, 7%, 6%, 5%, 4%, 3%, 2%, 1%, 0.75%, 0.5%, or 0.25%, or less) can also be said to be “proximal to” each other. Since one cM is the distance between two markers that show a 1% recombination frequency, any marker is closely linked (genetically and physically) to any other marker that is in close proximity, e.g., at or less than about 10 cM distant. Two closely linked markers on the same chromosome can be positioned about 9, 8, 7, 6, 5, 4, 3, 2, 1, 0.75, 0.5 or 0.25 cM or less from each other. A centimorgan (“cM”) or a genetic map unit (m.u.) is a unit of measure of recombination frequency and is defined as the distance between genes for which one product of meiosis in 100 is recombinant. One cM is equal to a 1% chance that a marker at one genetic locus will be separated from a marker at a second locus due to crossing over in a single generation. Thus, a recombinant frequency (RF) of 1% is equivalent to 1 m.u.

As used herein, the phrase “linkage group” refers to all of the genes or genetic traits that are located on the same chromosome. Within the linkage group, those loci that are close enough together can exhibit linkage in genetic crosses. Since the probability of crossover increases with the physical distance between loci on a chromosome, loci for which the locations are far removed from each other within a linkage group might not exhibit any detectable linkage in direct genetic tests. The term “linkage group” is mostly used to refer to genetic loci that exhibit linked behavior in genetic systems where chromosomal assignments have not yet been made. Thus, the term “linkage group” is synonymous with the physical entity of a chromosome, although one of ordinary skill in the art will understand that a linkage group can also be defined as corresponding to a region of (i.e., less than the entirety) of a given chromosome or for example any of intervals 1-15 as defined herein).

As used herein, the term “linkage disequilibrium” or “LD” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Markers that show linkage disequilibrium are considered linked. Linked loci co-segregate more than 50% of the time, e.g., from about 51% to about 100% of the time. In other words, two markers that co-segregate have a recombination frequency of less than 50% (and, by definition, are separated by less than 50 cM on the same chromosome). As used herein, linkage can be between two markers, or alternatively between a marker and a phenotype. A marker locus can be “associated with” (linked to) a trait, e.g., drought tolerance. The degree of linkage of a genetic marker to a phenotypic trait is measured, e.g., as a statistical probability of co-segregation of that marker with the phenotype.

Linkage disequilibrium is most commonly assessed using the measure r2, which is calculated using the formula described by Hill and Robertson, Theor. Appl. Genet. 38:226 (1968). When r2=1, complete linkage disequilibrium exists between the two marker loci, meaning that the markers have not been separated by recombination and have the same allele frequency. Values for r2 above ⅓ indicate sufficiently strong linkage disequilibrium to be useful for mapping. Ardlie et al., Nature Reviews Genetics 3:299 (2002). Hence, alleles are in linkage disequilibrium when r2 values between pairwise marker loci are greater than or equal to about 0.33, 0.4, 0.5, 0.6, 0.7, 0.8, 0.9, or 1.0.

As used herein, the term “linkage equilibrium” describes a situation where two markers independently segregate, i.e., sort among progeny randomly. Markers that show linkage equilibrium are considered unlinked (whether or not they lie on the same chromosome).

As used herein, the terms “marker”, “genetic marker” “nucleic acid marker”, and “molecular marker” are used interchangeably to refer to an identifiable position on a chromosome the inheritance of which can be monitored and/or a reagent that is used in methods for visualizing differences in nucleic acid sequences present at such identifiable positions on chromosomes. Thus, in some embodiments a marker comprises a known or detectable nucleic acid sequence. Examples of markers include, but are not limited to genetic markers, protein composition, peptide levels, protein levels, oil composition, oil levels, carbohydrate composition, carbohydrate levels, fatty acid composition, fatty acid levels, amino acid composition, amino acid levels, biopolymers, starch composition, starch levels, fermentable starch, fermentation yield, fermentation efficiency (e.g., captured as digestibility at 24, 48, and/or 72 hours), energy yield, secondary compounds, metabolites, morphological characteristics, and agronomic characteristics. As such, a marker can comprise a nucleotide sequence that has been associated with an allele or alleles of interest and that is indicative of the presence or absence of the allele or alleles of interest in a cell or organism and/or to a reagent that is used to visualize differences in the nucleotide sequence at such an identifiable position or positions. A marker can be, but is not limited to, an allele, a gene, a haplotype, a restriction fragment length polymorphism (RFLP), a simple sequence repeat (SSR), random amplified polymorphic DNA (RAPD), cleaved amplified polymorphic sequences (CAPS) (Rafalski and Tingey, Trends in Genetics 9:275 (1993)), an amplified fragment length polymorphism (AFLP) (Vos et al., Nucleic Acids Res. 23:4407 (1995)), a single nucleotide polymorphism (SNP) (Brookes, Gene 234:177 (1993)), a sequence-characterized amplified region (SCAR) (Paran and Michelmore, Theor. Appl. Genet. 85:985 (1993)), a sequence-tagged site (STS) (Onozaki et al., Euphytica 138:255 (2004)), a single-stranded conformation polymorphism (SSCP) (Orita et al., Proc. Natl. Acad. Sci. USA 86:2766 (1989)), an inter-simple sequence repeat (ISSR) (Blair et al., Theor. Appl. Genet. 98:780 (1999)), an inter-retrotransposon amplified polymorphism (IRAP), a retrotransposon-microsatellite amplified polymorphism (REMAP) (Kalendar et al., Theor. Appl. Genet. 98:704 (1999)) or an RNA cleavage product (such as a Lynx tag). A marker can be present in genomic or expressed nucleic acids (e.g., ESTs). The term marker can also refer to nucleic acids used as probes or primers (e.g., primer pairs) for use in amplifying, hybridizing to and/or detecting nucleic acid molecules according to methods well known in the art. A large number of maize molecular markers are known in the art, and are published or available from various sources, such as the Maize GDB internet resource and the Arizona Genomics Institute internet resource run by the University of Arizona.

In some embodiments, a marker corresponds to an amplification product generated by amplifying a Zea mays nucleic acid with one or more oligonucleotides, for example, by the polymerase chain reaction (PCR). As used herein, the phrase “corresponds to an amplification product” in the context of a marker refers to a marker that has a nucleotide sequence that is the same (allowing for mutations introduced by the amplification reaction itself and/or naturally occurring and/or artificial allelic differences) as an amplification product that is generated by amplifying Zea mays genomic DNA with a particular set of oligonucleotides. In some embodiments, the amplifying is by PCR, and the oligonucleotides are PCR primers that are designed to hybridize to opposite strands of the Zea mays genomic DNA in order to amplify a Zea mays genomic DNA sequence present between the sequences to which the PCR primers hybridize in the Zea mays genomic DNA. The amplified fragment that results from one or more rounds of amplification using such an arrangement of primers is a double stranded nucleic acid, one strand of which has a nucleotide sequence that comprises, in 5′ to 3′ order, the sequence of one of the primers, the sequence of the Zea mays genomic DNA located between the primers, and the reverse-complement of the second primer. Typically, the “forward” primer is assigned to be the primer that has the same sequence as a subsequence of the (arbitrarily assigned) “top” strand of a double-stranded nucleic acid to be amplified, such that the “top” strand of the amplified fragment includes a nucleotide sequence that is, in 5′ to 3′ direction, equal to the sequence of the forward primer—the sequence located between the forward and reverse primers of the top strand of the genomic fragment—the reverse-complement of the reverse primer. Accordingly, a marker that “corresponds to” an amplified fragment is a marker that has the same sequence of one of the strands of the amplified fragment.

Markers corresponding to genetic polymorphisms between members of a population can be detected by methods well-established in the art. These include, e.g., nucleic acid sequencing, hybridization methods, amplification methods (e.g., PCR-based sequence specific amplification methods), detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), and/or detection of amplified fragment length polymorphisms (AFLPs). Well established methods are also known for the detection of expressed sequence tags (ESTs) and SSR markers derived from EST sequences and randomly amplified polymorphic DNA (RAPD).

As used herein, the phrase “marker assay” refers to a method for detecting a polymorphism at a particular locus using a particular method such as but not limited to measurement of at least one phenotype (such as seed color, oil content, or a visually detectable trait); nucleic acid-based assays including, but not limited to restriction fragment length polymorphism (RFLP), single base extension, electrophoresis, sequence alignment, allelic specific oligonucleotide hybridization (ASO), random amplified polymorphic DNA (RAPD), microarray-based technologies, TAQMAN® Assays, ILLUMINA® GOLDENGATE® Assay analysis, nucleic acid sequencing technologies; peptide and/or polypeptide analyses; or any other technique that can be employed to detect a polymorphism in an organism at a locus of interest. Accordingly, in some embodiments of this invention, a marker is detected by amplifying a Zea mays nucleic acid with two oligonucleotide primers by, for example, an amplification reaction such as the polymerase chain reaction (PCR).

A “marker allele”, “allele” also described as an “allele of a marker locus,” can refer to one of a plurality of polymorphic nucleotide sequences found at a marker locus in a population that is polymorphic for the marker locus.

“Marker-assisted selection” (MAS) is a process by which phenotypes are selected based on marker genotypes. Marker assisted selection includes the use of marker genotypes for identifying plants for inclusion in and/or removal from a breeding program or planting.

“Marker-assisted counter-selection” is a process by which marker genotypes are used to identify plants that will not be selected, allowing them to be removed from a breeding program or planting. Thus maize plant breeding programs may use any of the information listed in Tables 1-7 to make marker-assisted counter-selection to eliminate maize lines or germplasm that do not have increased drought tolerance.

As used herein, the terms “marker locus”, “locus”, “loci” and “marker loci” refer to a specific chromosome location or locations in the genome of an organism where a specific marker or markers can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

As used herein, the term “probe” or “molecular probe” refers to a single-stranded oligonucleotide sequence that will form a hydrogen-bonded duplex with a complementary sequence in a target nucleic acid sequence analyte or its cDNA derivative. Thus, a “marker probe” and “probe” refers to a nucleotide sequence or nucleic acid molecule that can be used to detect the presence of one or more particular alleles within a marker locus (e.g., a nucleic acid probe that is complementary to all of or a portion of the marker or marker locus, through nucleic acid hybridization). Marker probes comprising about 8, 10, 15, 20, 30, 40, 50, 60, 70, 80, 90, 100 or more contiguous nucleotides may be used for nucleic acid hybridization. Alternatively, in some aspects, a marker probe refers to a probe of any type that is able to distinguish (i.e., genotype) the particular allele that is present at a marker locus. Non-limiting examples of a probe of this invention includes SEQ ID NO:27, SEQ ID NO:28, SEQ ID NO:31, SEQ ID NO:32, SEQ ID NO:35, SEQ ID NO:36, SEQ ID NO:39, SEQ ID NO:40, SEQ ID NO:43, SEQ ID NO:44, SEQ ID NO:47, SEQ ID NO:48, SEQ ID NO:51, SEQ ID NO:52, SEQ ID NO:55, and/or SEQ ID NO:56, as well as the sequences found in Tables 1-7.

As used herein, the term “molecular marker” may be used to refer to a genetic marker, as defined above, or an encoded product thereof (e.g., a protein) used as a point of reference when identifying a linked locus. A molecular marker can be derived from genomic nucleotide sequences or from expressed nucleotide sequences (e.g., from a spliced RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution, e.g., according to Watson-Crick base pairing rules. Some of the markers described herein can also be referred to as hybridization markers when located on an indel region. This is because the insertion region is, by definition, a polymorphism vis-ã-vis a plant without the insertion. Thus, the marker need only indicate whether the indel region is present or absent. Any suitable marker detection technology may be used to identify such a hybridization marker, e.g., technology for SNP detection.

As used herein, the term “primer” refers to an oligonucleotide which is capable of annealing to a nucleic acid target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some embodiments an extension primer and in some embodiments an amplification primer) is in some embodiments single stranded for maximum efficiency in extension and/or amplification. In some embodiments, the primer is an oligodeoxyribonucleotide. A primer is typically sufficiently long to prime the synthesis of extension and/or amplification products in the presence of the agent for polymerization. The minimum length of the primer can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification. As such, it will be understood that the term “primer,” as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing.

Primers can be prepared by any suitable method. Methods for preparing oligonucleotides of specific sequence are known in the art, and include, for example, cloning and restriction of appropriate sequences and direct chemical synthesis. Chemical synthesis methods can include, for example, the phospho di- or tri-ester method, the diethylphosphoramidate method and the solid support method disclosed in U.S. Pat. No. 4,458,066. Primers can be labeled, if desired, by incorporating detectable moieties by for instance spectroscopic, fluorescence, photochemical, biochemical, immunochemical, or chemical moieties.

Non-limiting examples of primers of the invention include SEQ ID NO:25, SEQ ID NO:26, SEQ ID NO:29, SEQ ID NO:30, SEQ ID NO:33, SEQ ID NO:34, SEQ ID NO:37, SEQ ID NO:38, SEQ ID NO:41, SEQ ID NO:42, SEQ ID NO:45, SEQ ID NO:46, SEQ ID NO:49, SEQ ID NO:50, SEQ ID NO:53, and/or SEQ ID NO:54. The PCR method is well described in handbooks and known to the skilled person. After amplification by PCR, target polynucleotides can be detected by hybridization with a probe polynucleotide, which forms a stable hybrid with the target sequence under stringent to moderately stringent hybridization and wash conditions. If it is expected that the probes are essentially completely complementary (i.e., about 99% or greater) to the target sequence, stringent conditions can be used. If some mismatching is expected, for example if variant strains are expected with the result that the probe will not be completely complementary, the stringency of hybridization can be reduced. In some embodiments, conditions are chosen to rule out non-specific/adventitious binding. Conditions that affect hybridization, and that select against non-specific binding are known in the art, and are described in, for example, Sambrook & Russell (2001). Molecular Cloning: A Laboratory Manual, Third Edition, Cold Spring Harbor Laboratory Press, Cold Spring Harbor, N.Y., United States of America. Generally, lower salt concentration and higher temperature hybridization and/or washes increase the stringency of hybridization conditions.

Different nucleotide sequences or polypeptide sequences having homology are referred to herein as “homologues” or “homolog” The term homologue includes homologous sequences from the same and other species and orthologous sequences from the same and other species. “Homology” refers to the level of similarity between two or more nucleotide sequences and/or amino acid sequences in terms of percent of positional identity (i.e., sequence similarity or identity). Homology also refers to the concept of similar functional properties among different nucleic acids, amino acids, and/or proteins.

As used herein, the phrase “nucleotide sequence homology” refers to the presence of homology between two polynucleotides. Polynucleotides have “homologous” sequences if the sequence of nucleotides in the two sequences is the same when aligned for maximum correspondence. The “percentage of sequence homology” for polynucleotides, such as 50, 55, 60, 65, 70, 75, 80, 85, 90, 95, 96, 97, 98, 99 or 100 percent sequence homology, can be determined by comparing two optimally aligned sequences over a comparison window (e.g., about 20-200 contiguous nucleotides), wherein the portion of the polynucleotide sequence in the comparison window can include additions or deletions (i.e., gaps) as compared to a reference sequence for optimal alignment of the two sequences. Optimal alignment of sequences for comparison can be conducted by computerized implementations of known algorithms, or by visual inspection. Readily available sequence comparison and multiple sequence alignment algorithms are, respectively, the Basic Local Alignment Search Tool (BLAST; Altschul et al. (1990) J Mol Biol 215:403-10; Altschul et al. (1997) Nucleic Acids Res 25:3389-3402) and ClustalX (Chenna et al. (2003) Nucleic Acids Res 31:3497-3500) programs, both available on the Internet. Other suitable programs include, but are not limited to, GAP, BestFit, PlotSimilarity, and FASTA, which are part of the Accelrys GCG Package available from Accelrys Software, Inc. of San Diego, Calif., United States of America.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., Ed.) Oxford University Press, New York (1988); Biocomputing: Informatics and Genome Projects (Smith, D. W., Ed.) Academic Press, New York (1993); Computer Analysis of Sequence Data, Part I (Griffin, A. M., and Griffin, H. G., Eds.) Humana Press, New Jersey (1994); Sequence Analysis in Molecular Biology (von Heinje, G., Ed.) Academic Press (1987); and Sequence Analysis Primer (Gribskov, M. and Devereux, J., Eds.) Stockton Press, New York (1991).

As used herein, the term “substantially identical” means that two nucleotide sequences have at least about 50%, 60%, 70%, 75%, 80%, 85%, 90% or 95% sequence identity. In some embodiments, two nucleotide sequences can have at least about 75%, 80%, 85%, 90%, 95%, or 100% sequence identity, and any range or value therein. In representative embodiments, two nucleotide sequences can have at least about 95%, 96%, 97%, 98%, 99% or 100% sequence identity, and any range or value therein.

An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window is well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo et al. (Applied Math 48:1073 (1988)). More particularly, preferred computer programs for determining sequence identity include but are not limited to the Basic Local Alignment Search Tool (BLAST) programs, which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence, BLASTX can be used to determine sequence identity; and for polynucleotide sequence, BLASTN can be used to determine sequence identity.

A “heterotic group” comprises a set of genotypes that perform well when crossed with genotypes from a different heterotic group. Hallauer et al., Corn breeding, in CORN AND CORN IMPROVEMENT p. 463-564 (1998). Inbred lines are classified into heterotic groups, and are further subdivided into families within a heterotic group, based on several criteria such as pedigree, molecular marker-based associations, and performance in hybrid combinations. Smith et al., Theor. Appl. Gen. 80:833 (1990).

As used herein, the terms “phenotype,” or “phenotypic trait” refer to one or more traits of an organism. The phenotype can be observable to the naked eye, or by any other means of evaluation known in the art, e.g., microscopy, biochemical analysis, and/or an electromechanical assay. In some cases, a phenotype is directly controlled by a single gene or genetic locus, i.e., a “single gene trait.” In other cases, a phenotype is the result of several genes.

As used herein, the terms “drought tolerance” and “drought tolerant” refer to a plant's ability to endure and/or thrive under drought stress or water deficit conditions. When used in reference to germplasm or plant, the terms refer to the ability of a plant that arises from that germplasm or plant to endure and/or thrive under drought conditions. In general, a plant or germplasm is labeled as “drought tolerant” if it displays “increased drought tolerance.”

As used herein, the term “increased drought tolerance” refers to an improvement, enhancement, or increase in one or more water optimization phenotypes as compared to one or more control plants (e.g., one or both of the parents, or a plant lacking a marker associated with increased drought tolerance). Exemplary drought tolerant phenotypes include, but are not limited to, increased yield in bushels per acre, grain yield at standard moisture percentage (YGSMN), grain moisture at harvest (GMSTP), grain weight per plot (GWTPN), percent yield recovery (PYREC), yield reduction (YRED), anthesis silk interval (ASI) and percent barren (PB) (all scenarios may be compare to increases relative to those of a control plant). Thus, a plant that demonstrates higher YGSMN than one or both of its parents when each is grown under drought stress conditions displays increased drought tolerance and can be labeled as “drought tolerant.”

The phrase “abiotic stress” as used herein refers to any adverse effect on metabolism, growth, reproduction and/or viability of a plant by abiotic factors (i.e. water availability, heat, cold, etc.). Accordingly, abiotic stress can be induced by suboptimal environmental growth conditions such as, for example, salinity, water deprivation, water deficit, drought, flooding, freezing, low or high temperature (e.g., chilling or excessive heat), toxic chemical pollution, heavy metal toxicity, anaerobiosis, nutrient deficiency, nutrient excess, atmospheric pollution or UV irradiation.

The phrase “abiotic stress tolerance” as used herein refers to the ability of a plant to endure an abiotic stress better than a control plant.

As used herein “water deficit” or “drought” means a period when water available to a plant is not replenished at the rate at which it is consumed by the plant. A long period of water deficit is colloquially called drought. Lack of rain or irrigation may not produce immediate water stress if there is an available reservoir of ground water to support the growth rate of plants. Plants grown in soil with ample groundwater can survive days without rain or irrigation without adverse effects on yield. Plants grown in dry soil are likely to suffer adverse effects with minimal periods of water deficit. Severe water deficit stress can cause wilt and plant death; moderate drought can reduce yield, stunt growth or retard development. Plants can recover from some periods of water deficit stress without significantly affecting yield. However, water deficit at the time of pollination can lower or reduce yield. Thus, a useful period in the life cycle of corn, for example, for observing response or tolerance to water deficit is the late vegetative stage of growth before tassel emergence or the transition to reproductive development. Tolerance to water deficit/drought is determined by comparison to control plants. For instance, plants of this invention can produce a higher yield than control plants when exposed to water deficit. In the laboratory and in field trials drought can be simulated by giving plants of this invention and control plants less water than is given to sufficiently-watered control plants and measuring differences in traits.

Water Use Efficiency (WUE) is a parameter frequently used to estimate the tradeoff between water consumption and CO2 uptake/growth (Kramer, 1983, Water Relations of Plants, Academic Press p. 405). WUE has been defined and measured in multiple ways. One approach is to calculate the ratio of whole plant dry weight, to the weight of water consumed by the plant throughout its life (Chu et al., 1992, Oecologia 89:580). Another variation is to use a shorter time interval when biomass accumulation and water use are measured (Mian et al., 1998, Crop Sci. 38:390). Another approach is to utilize measurements from restricted parts of the plant, for example, measuring only aerial growth and water use (Nienhuis et al 1994 Amer J Bot 81:943). WUE also has been defined as the ratio of CO2 uptake to water vapor loss from a leaf or portion of a leaf, often measured over a very short time period (e.g. seconds/minutes) (Kramer, 1983, p. 406). The ratio of 13C/12C fixed in plant tissue, and measured with an isotope ratio mass-spectrometer, also has been used to estimate WUE in plants using C-3 photosynthesis (Martin et al., 1999, Crop Sci. 1775). As used herein, the term “water use efficiency” refers to the amount of organic matter produced by a plant divided by the amount of water used by the plant in producing it, i.e. the dry weight of a plant in relation to the plant's water use. As used herein, the term “dry weight” refers to everything in the plant other than water, and includes, for example, carbohydrates, proteins, oils, and mineral nutrients.

As used herein, the term “gene” refers to a hereditary unit including a sequence of DNA that occupies a specific location on a chromosome and that contains the genetic instruction for a particular characteristic or trait in an organism.

The term “chromosome interval” designates a contiguous linear span of genomic DNA that resides in planta on a single chromosome. The term also designates any and all genomic intervals defined by any of the markers set forth in this invention. The genetic elements located on a single chromosome interval are physically linked and the size of a chromosome interval is not particularly limited. In some aspects, the genetic elements located within a single chromosome interval are physically linked, typically with a distance of, for example, less than or equal to 20 Mb, or alternatively, less than or equal to 10 Mb. An interval described by the terminal markers that define the endpoints of the interval will include the terminal markers and any marker localizing within that chromosome domain, whether those markers are currently known or unknown. Although it is anticipated that one skilled in the art may describe additional polymorphic sites at marker loci in and around the markers identified herein, any marker within the chromosome intervals described herein that are associated with drought tolerance fall within the scope of this claimed invention. The boundaries of chromosome intervals comprise markers that will be linked to the gene, genes, or loci providing the trait of interest, i.e. any marker that lies within a given interval, including the terminal markers that define the boundaries of the interval, can be used as a marker for drought tolerance. The intervals described herein encompass marker clusters that co-segregate with drought tolerance water optimization. The clustering of markers occurs in relatively small domains on the chromosomes, indicating the presence of a genetic locus controlling the trait of interest in those chromosome regions. The interval encompasses markers that map within the interval as well as the markers that define the terminal.

“Quantitative trait loci” or a “quantitative trait locus” (QTL) is a genetic domain that effects a phenotype that can be described in quantitative terms and can be assigned a “phenotypic value” which corresponds to a quantitative value for the phenotypic trait. A QTL can act through a single gene mechanism or by a polygenic mechanism. The boundaries of chromosome intervals are drawn to encompass markers that will be linked to one or more QTL. In other words, the chromosome interval is drawn such that any marker that lies within that interval (including the terminal markers that define the boundaries of the interval) can be used as markers for drought tolerance. Each interval comprises at least one QTL, and furthermore, may indeed comprise more than one QTL. Close proximity of multiple QTL in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifying the same QTL or two different QTL. Regardless, knowledge of how many QTL are in a particular interval is not necessary to make or practice the invention.

As used herein, the phrase “ILLUMINA® GOLDENGATE® Assay” refers to a high throughput genotyping assay sold by Illumina Inc. of San Diego, Calif., United States of America that can generate SNP-specific PCR products. This assay is described in detail at the website of Illumina Inc. and in Fan et al., 2006.

As used herein, the phrase “immediately adjacent”, when used to describe a nucleic acid molecule that hybridizes to DNA containing a polymorphism, refers to a nucleic acid that hybridizes to a DNA sequence that directly abuts the polymorphic nucleotide base position. For example, a nucleic acid molecule that can be used in a single base extension assay is “immediately adjacent” to the polymorphism.

As used herein, the term “improved”, and grammatical variants thereof, refers to a plant or a part, progeny, or tissue culture thereof, that as a consequence of having (or lacking) a particular water optimization associated allele (such as, but not limited to those water optimization associated alleles disclosed herein) is characterized by a higher or lower content of a water optimization associated trait, depending on whether the higher or lower content is desired for a particular purpose.

As used herein, the term “INDEL” (also spelled “indel”) refers to an insertion or deletion in a pair of nucleotide sequences, wherein a first sequence can be referred to as having an insertion relative to a second sequence or the second sequence can be referred to as having a deletion relative to the first sequence.

As used herein, the term “informative fragment” refers to a nucleotide sequence comprising a fragment of a larger nucleotide sequence, wherein the fragment allows for the identification of one or more alleles within the larger nucleotide sequence. For example, an informative fragment of the nucleotide sequence of SEQ ID NO: 17 comprises a fragment of the nucleotide sequence of SEQ ID NO: 1 and allows for the identification of one or more alleles (e.g., a G nucleotide at position 401 of SEQ ID NO: 17), the nucleotide sequence of SEQ ID NO: 18 comprises a fragment of the nucleotide sequence of SEQ ID NO: 2 and allows for the identification of one or more alleles (e.g., a G nucleotide at position 401 of SEQ ID NO: 18), the nucleotide sequence of SEQ ID NO: 19 comprises a fragment of the nucleotide sequence of SEQ ID NO: 3 and allows for the identification of one or more alleles (e.g., an A nucleotide at position 401 of SEQ ID NO: 19), the nucleotide sequence of SEQ ID NO: 20 comprises a fragment of the nucleotide sequence of SEQ ID NO: 4 and allows for the identification of one or more alleles (e.g., an A nucleotide at position 401 of SEQ ID NO: 20), the nucleotide sequence of SEQ ID NO: 21 comprises a fragment of the nucleotide sequence of SEQ ID NO: 5 and allows for the identification of one or more alleles (e.g., a G nucleotide at position 401 of SEQ ID NO: 21), the nucleotide sequence of SEQ ID NO: 22 comprises a fragment of the nucleotide sequence of SEQ ID NO: 6 and allows for the identification of one or more alleles (e.g., a C nucleotide at position 401 of SEQ ID NO: 22), the nucleotide sequence of SEQ ID NO: 23 comprises a fragment of the nucleotide sequence of SEQ ID NO: 7 and allows for the identification of one or more alleles (e.g., an A nucleotide at position 401 of SEQ ID NO: 23), and the nucleotide sequence of SEQ ID NO: 24 comprises a fragment of the nucleotide sequence of SEQ ID NO: 8 and allows for the identification of one or more alleles (e.g., a G nucleotide at position 401 of SEQ ID NO: 24).

As used herein, the phrase “interrogation position” refers to a physical position on a solid support that can be queried to obtain genotyping data for one or more predetermined genomic polymorphisms.

As used herein, the term “polymorphism” refers to a variation in the nucleotide sequence at a locus, where said variation is too common to be due merely to a spontaneous mutation. A polymorphism must have a frequency of at least about 1% in a population. A polymorphism can be a single nucleotide polymorphism (SNP), or an insertion/deletion polymorphism, also referred to herein as an “indel.” Additionally, the variation can be in a transcriptional profile or a methylation pattern. The polymorphic site or sites of a nucleotide sequence can be determined by comparing the nucleotide sequences at one or more loci in two or more germplasm entries.

As used herein, the phrase “recombination” refers to an exchange of DNA fragments between two DNA molecules or chromatids of paired chromosomes (a “crossover”) over in a region of similar or identical nucleotide sequences. A “recombination event” is herein understood to refer to a meiotic crossover.

As used herein, the term “plant” can refer to a whole plant, any part thereof, or a cell or tissue culture derived from a plant. Thus, the term “plant” can refer to a whole plant, a plant part or a plant organ (e.g., leaves, stems, roots, etc.), a plant tissue, a seed and/or a plant cell. A plant cell is a cell of a plant, taken from a plant, or derived through culture from a cell taken from a plant.

As used herein, the term “maize” refers to a plant of the Zea mays L. ssp. mays and is also known as “corn.”

As used herein, the term “maize plant” includes whole maize plants, maize plant cells, maize plant protoplast, maize plant cell or maize tissue cultures from which maize plants can be regenerated, maize plant calli, and maize plant cells that are intact in maize plants or parts of maize plants, such as maize seeds, maize cobs, maize flowers, maize cotyledons, maize leaves, maize stems, maize buds, maize roots, maize root tips, and the like.

As used herein, the phrase “native trait” refers to any existing monogenic or oligogenic trait in a certain crop's germplasm. When identified through molecular marker(s), the information obtained can be used for the improvement of germplasm through marker assisted breeding of the water optimization associated traits disclosed herein.

A “non-naturally occurring variety of maize” is any variety of maize that does not naturally exist in nature. A “non-naturally occurring variety of maize” can be produced by any method known in the art, including, but not limited to, transforming a maize plant or germplasm, transfecting a maize plant or germplasm and crossing a naturally occurring variety of maize with a non-naturally occurring variety of maize, through genome editing (e.g. CRISPR or TALEN), or through creating breeding stacks of desired alleles not present in nature. In some embodiments, a “non-naturally occurring variety of maize” can comprise one of more heterologous nucleotide sequences. In some embodiments, a “non-naturally occurring variety of maize” can comprise one or more non-naturally occurring copies of a naturally occurring nucleotide sequence (i.e., extraneous copies of a gene that naturally occurs in maize).

The “non-Stiff Stalk” heterotic group represents a major heterotic group in the northern U.S. and Canadian corn growing regions. It can also be referred to as the “Lancaster” or “Lancaster Sure Crop” heterotic group.

The “Stiff Stalk” heterotic group represents a major heterotic group in the northern U.S. and Canadian corn growing regions. It can also be referred to as the “Iowa Stiff Stalk Synthetic” or “BSSS” heterotic group.

As used herein, the term “percent barren” (PB) refers to the percentage of plants in a given area (e.g., plot) with no grain. It is typically expressed in terms of the percentage of plants per plot and can be calculated as:

$$\frac{number of plants in the plot with no grain}{total number of plants in the plot} \times 100$$

As used herein, the term “percent yield recovery” (PYREC) refers to the effect an allele and/or combination of alleles has on the yield of a plant grown under drought stress conditions as compared to that of a plant that is genetically identical except insofar as it lacks the allele and/or combination of alleles. PYREC is calculated as:

$$1 - \frac{\begin{matrix}yield under full irrigation ( w \text{/} allele ( s ) of interest ) - \\ yield under drought conditions ( w \text{/} allele ( s ) of interest )\end{matrix}}{\begin{matrix}yield under full irrigation ( w \text{/} out allelle ( s ) of interest ) - \\ yield under drought conditions ( w \text{/} out allele ( s ) of interest )\end{matrix}} \times 100$$

By way of example and not limitation, if a control plant yields 200 bushels under full irrigation conditions, but yields only 100 bushels under drought stress conditions, then its percentage yield loss would be calculated at 50%. If an otherwise genetically identical hybrid that contains the allele(s) of interest yields 125 bushels under drought stress conditions and 200 bushels under full irrigation conditions, then the percentage yield loss would be calculated as 37.5% and the PYREC would be calculated as 25% [1.00−(200−125)(400−100)×100)].

As used herein, the phrase “Grain Yield—Well Watered” refers to yield from an area that obtained enough irrigation to prevent plants from being water stressed during their growth cycle. In some embodiments, this trait is expressed in bushels per acre.

As used herein, the phrase “Yield Reduction—Hybrid” refers to a calculated trait obtained from a hybrid yield trial grown under stress and non-stress conditions. For a given hybrid, it equals:

$$\frac{non \text{-} stress yield - yield under stress}{non \text{-} stressed yield} \times 100.$$

In some embodiments, this trait is expressed as percent bushels per acre.

As used herein, the phrase “Yield Reduction—Inbred” refers to a calculated trait obtained from an inbred yield trial grown under stress and non-stress conditions. For a given inbred, it equals:

$$\frac{non \text{-} stress yield - yield under stress}{non \text{-} stressed yield} \times 100.$$

In some embodiments, this trait is expressed as percent bushels per acre.

As used herein, the terms “nucleotide sequence,” “polynucleotide,” “nucleic acid sequence,” “nucleic acid molecule” and “nucleic acid fragment” refer to a polymer of RNA or DNA that is single- or double-stranded, optionally containing synthetic, non-natural and/or altered nucleotide bases. A “nucleotide” is a monomeric unit from which DNA or RNA polymers are constructed and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, the term “plant part” includes but is not limited to embryos, pollen, seeds, leaves, flowers (including but not limited to anthers, ovules and the like), fruit, stems or branches, roots, root tips, cells including cells that are intact in plants and/or parts of plants, protoplasts, plant cell tissue cultures, plant calli, plant clumps, and the like. Thus, a plant part includes soybean tissue culture from which soybean plants can be regenerated. Further, as used herein, “plant cell” refers to a structural and physiological unit of the plant, which comprises a cell wall and also may refer to a protoplast. A plant cell of the present invention can be in the form of an isolated single cell or can be a cultured cell or can be a part of a higher-organized unit such as, for example, a plant tissue or a plant organ.

As used herein, the term “population” refers to a genetically heterogeneous collection of plants sharing a common genetic derivation.

As used herein, the terms “progeny,” “progeny plant,” and/or “offspring” refer to a plant generated from a vegetative or sexual reproduction from one or more parent plants. A progeny plant may be obtained by cloning or selfing a single parent plant, or by crossing two parental plants and includes selfings as well as the F1 or F2 or still further generations. An F1 is a first-generation offspring produced from parents at least one of which is used for the first time as donor of a trait, while offspring of second generation (F2) or subsequent generations (F3, F4, and the like) are specimens produced from selfings or crossings of F1 s, F2s and the like. An F1 can thus be (and in some embodiments is) a hybrid resulting from a cross between two true breeding parents (the phrase “true-breeding” refers to an individual that is homozygous for one or more traits), while an F2 can be an offspring resulting from self-pollination of the F1 hybrids.

As used herein, the term “reference sequence” refers to a defined nucleotide sequence used as a basis for nucleotide sequence comparison (e.g., Chromosome 1 or Chromosome 3 of Zea mays cultivar B73). The reference sequence for a marker, for example, can be obtained by genotyping a number of lines at the locus or loci of interest, aligning the nucleotide sequences in a sequence alignment program, and then obtaining the consensus sequence of the alignment. Hence, a reference sequence identifies the polymorphisms in alleles at a locus. A reference sequence may not be a copy of an actual nucleic acid sequence from any particular organism; however, it is useful for designing primers and probes for actual polymorphisms in the locus or loci.

As used herein, the term “isolated” refers to a nucleotide sequence (e.g., a genetic marker) that is free of sequences that normally flank one or both sides of the nucleotide sequence in a plant genome. As such, the phrase “isolated and purified genetic marker associated with a water optimization trait in Zea mays” can be, for example, a recombinant DNA molecule, provided one of the nucleic acid sequences normally found flanking that recombinant DNA molecule in a naturally-occurring genome is removed or absent. Thus, isolated nucleic acids include, without limitation, a recombinant DNA that exists as a separate molecule (including, but not limited to genomic DNA fragments produced by PCR or restriction endonuclease treatment) with no flanking sequences present, as well as a recombinant DNA that is incorporated into a vector, an autonomously replicating plasmid, or into the genomic DNA of a plant as part of a hybrid or fusion nucleic acid molecule.

As used herein, the phrase “TAQMAN® Assay” refers to real-time sequence detection using PCR based on the TAQMAN® Assay sold by Applied Biosystems, Inc. of Foster City, Calif., United States of America. For an identified marker, a TAQMAN® Assay can be developed for application in a breeding program.

As used herein, the term “tester” refers to a line used in a testcross with one or more other lines wherein the tester and the lines tested are genetically dissimilar. A tester can be an isogenic line to the crossed line.

As used herein, the term “trait” refers to a phenotype of interest, a gene that contributes to a phenotype of interest, as well as a nucleic acid sequence associated with a gene that contributes to a phenotype of interest. For example, a “water optimization trait” refers to a water optimization phenotype as well as a gene that contributes to a water optimization phenotype and a nucleic acid sequence (e.g., an SNP or other marker) that is associated with a water optimization phenotype.

As used herein, the term “transgene” refers to a nucleic acid molecule introduced into an organism or its ancestors by some form of artificial transfer technique. The artificial transfer technique thus creates a “transgenic organism” or a “transgenic cell”. It is understood that the artificial transfer technique can occur in an ancestor organism (or a cell therein and/or that can develop into the ancestor organism) and yet any progeny individual that has the artificially transferred nucleic acid molecule or a fragment thereof is still considered transgenic even if one or more natural and/or assisted breedings result in the artificially transferred nucleic acid molecule being present in the progeny individual.

An “unfavorable allele” of a marker is a marker allele that segregates with the unfavorable plant phenotype, therefore providing the benefit of identifying plants that can be removed from a breeding program or planting.

As used herein, the term “water optimization” refers to any measure of a plant, its parts, or its structure that can be measured and/or quantitated in order to assess an extent of or a rate of plant growth and development under conditions of sufficient water availability as compared to conditions of suboptimal water availability (e.g., drought). As such, a “water optimization trait” is any trait that can be shown to influence yield in a plant under different sets of growth conditions related to water availability. As used herein, the phrase “water optimization” refers to any measure of a plant, its parts, or its structure that can be measured and/or quantified in order to assess an extent of or a rate of plant growth and development under different conditions of water availability. (E.g. All marker alleles identified in Tables 1-7 or closely linked markers thereof may be used to identify, select or produce maize plants having increase water optimization). Similarly, “water optimization” can be considered a “phenotype”, which as used herein refers to a detectable, observable, and/or measurable characteristic of a cell or organism. In some embodiments, a phenotype is based at least in part on the genetic make-up of the cell or the organism (referred to herein as the cell or the organism's “genotype”). Exemplary water optimization phenotypes are grain yield at standard moisture percentage (YGSMN), grain moisture at harvest (GMSTP), grain weight per plot (GWTPN), and percent yield recovery (PYREC). It is noted that as used herein, the term “phenotype” takes into account how the environment (e.g., environmental conditions) might affect water optimization such that the water optimization effect is real and reproducible. As used herein, the term “yield reduction” (YD) refers to the degree to which yield is reduced in plants grown under stress conditions. YD is calculated as:

$$\frac{\begin{matrix}yield under non \text{-} stress conditions - \\ yield under stress conditions\end{matrix}}{yield under non \text{-} stress conditions} \times 100$$

Genetic loci correlating with particular phenotypes, such as drought tolerance, can be mapped in an organism's genome. By identifying a marker or cluster of markers that co-segregate with a trait of interest, the breeder is able to rapidly select a desired phenotype by selecting for the proper marker (a process called marker-assisted selection, or MAS). Such markers may also be used by breeders to design genotypes in silico and to practice whole genome selection.

The present invention provides chromosome intervals, QTL, Loci and genes associated with improved drought tolerance in plants (e.g. maize) and/or improved/increased yield in a plant (e.g. maize). Detection of these markers and/or other linked markers can be used to identify, select and/or produce maize plants having increased drought tolerance and/or to eliminate maize plants from breeding programs or from planting that do not have increased drought tolerance.

Molecular markers are used for the visualization of differences in nucleic acid sequences. This visualization can be due to DNA-DNA hybridization techniques after digestion with a restriction enzyme (e.g., an RFLP) and/or due to techniques using the polymerase chain reaction (e.g., SNP, STS, SSR/microsatellites, AFLP, and the like). In some embodiments, all differences between two parental genotypes segregate in a mapping population based on the cross of these parental genotypes. The segregation of the different markers can be compared and recombination frequencies can be calculated. Methods for mapping markers in plants are disclosed in, for example, Glick & Thompson (1993) Methods in Plant Molecular Biology and Biotechnology, CRC Press, Boca Raton, Fla., United States of America; Zietkiewicz et al. (1994) Genomics 20:176-183.

Tables 1-8 provides the names of Zea maize genomic regions (i.e. chromosome intervals, gene, QTLs, alleles or loci) the physical genetic locations of each marker on the respective maize chromosome or linkage group, and the target allele(s) that are associated with increased drought tolerance, water optimization, and/or maize yield under either drought or non-drought conditions. Markers of the present invention are described herein with respect to the positions of marker loci mapped to physical locations as they are reported on the B73 RefGen_v2 sequence public assembly by the Arizona Genomics Institute. The maize genome physical sequence can be found at the internet resources: maizeGDB (maizegdb.org/assembly) or Gramene at (gramene.org).

Thus, in some embodiments of this invention, the marker alleles, chromosome intervals and/or QTLs associated with increased drought tolerance or increased yield under drought or non-drought conditions are set forth in Tables 1-7.

In some embodiments of this invention, the marker allele(s) and closely linked markers thereof, associated with increased drought tolerance as set forth in Tables 1-7 can be located in a chromosomal interval including, but not limited to (a) a chromosome interval on chromosome 1 defined by and including base pair (bp) position 272937470 to base pair (bp) position 272938270 (PZE01271951242); (b) a chromosome interval on chromosome 2 defined by and including base pair (bp) position 12023306 to base pair (bp) position 12024104 (PZE0211924330); (c) a chromosome interval on chromosome 3 defined by and including base pair (bp) position 225037202 to base pair (bp) position 225038002 (PZE03223368820); (d) a chromosome interval on chromosome 3 defined by and including base pair (bp) position 225340531 to base pair (bp) position 225341331 (PZE03223703236); (e) a chromosome interval on chromosome 5 defined by and including base pair (bp) position 159,120,801 to base pair (bp) position 159,121,601 (PZE05158466685); (f) a chromosome interval on chromosome 9 defined by and including base pair (bp) position 12104536 to base pair (bp) position 12105336 (PZE0911973339); (g) a chromosome interval on chromosome 9 defined by and including base pair (bp) position 225343590 to base pair (bp) position 225340433 (S_18791654); (h) a chromosome interval on chromosome 10 defined by and including base pair (bp) position 14764415 to base pair (bp) position 14765098 (S_20808011); or any combination thereof. As would be understood by one of skill in the art, additional chromosomal intervals can be defined by the SNP markers provided herein in Table 1. Additionally, SNP markers within the chromosome intervals of (a)-(h) other than those provided in Table 1 may be derived by methods well known in the art.

The present invention further provides that the detecting of a molecular marker can comprise the use of a nucleic acid probe having a nucleotide base sequence that is substantially complementary to a nucleic acid sequence defining the molecular marker and which nucleic acid probe specifically hybridizes under stringent conditions with a nucleic acid sequence defining the molecular marker. A suitable nucleic acid probe can for instance be a single strand of the amplification product corresponding to the marker. In some embodiments, the detecting of a marker is designed to determine whether a particular allele of an SNP is present or absent in a particular plant.

Additionally, the methods of this invention include detecting an amplified DNA fragment associated with the presence of a particular allele of a SNP. In some embodiments, the amplified fragment associated with a particular allele of a SNP has a predicted length or nucleic acid sequence, and detecting an amplified DNA fragment having the predicted length or the predicted nucleic acid sequence is performed such that the amplified DNA fragment has a length that corresponds (plus or minus a few bases; e.g., a length of one, two or three bases more or less) to the expected length based on a similar reaction with the same primers with the DNA from the plant in which the marker was first detected or the nucleic acid sequence that corresponds (e.g., a homology of at least about 80%, 90%, 95%, 96%, 97%, 98%, 99% or more) to the expected sequence based on the sequence of the marker associated with that SNP in the plant in which the marker was first detected.

The detecting of an amplified DNA fragment having the predicted length or the predicted nucleic acid sequence can be performed by any of a number or techniques, including, but not limited to, standard gel-electrophoresis techniques or by using automated DNA sequencers. Such methods of detecting an amplified DNA fragment are not described here in detail as they are well known to those of ordinary skill in the art.

As shown in Tables 1-8, the SNP markers of this invention are associated with increased drought tolerance and/or increased yield under either drought or non-drought conditions. In some embodiments, as described herein, one marker or a combination of markers can be used to detect the presence of a drought tolerant maize plant or maize plants having increased yield under non-drought conditions as compared to a control plant. In some embodiments, a marker can be located within a chromosomal interval (QTL) or be present in the genome of the plant as a haplotype as defined herein (e.g. any one of chromosome intervals 1, 2, 3, 4, 5, 6, or 7 as defined herein).

### II. Molecular Markers, Water Optimization Associated Loci, and Compositions for Assaying Nucleic Acid Sequences

Molecular markers are used for the visualization of differences in nucleic acid sequences. This visualization can be due to DNA-DNA hybridization techniques after digestion with a restriction enzyme (e.g., an RFLP) and/or due to techniques using the polymerase chain reaction (e.g., STS, SSR/microsatellites, AFLP, and the like.). In some embodiments, all differences between two parental genotypes segregate in a mapping population based on the cross of these parental genotypes. The segregation of the different markers can be compared and recombination frequencies can be calculated. Methods for mapping markers in plants are disclosed in, for example, Glick & Thompson, 1993; Zietkiewicz et al., 1994. The recombination frequencies of molecular markers on different chromosomes are generally 50%. Between molecular markers located on the same chromosome, the recombination frequency generally depends on the distance between the markers. A low recombination frequency typically corresponds to a small genetic distance between markers on a chromosome. Comparing all recombination frequencies results in the most logical order of the molecular markers on the chromosomes. This most logical order can be depicted in a linkage map (Paterson, 1996). A group of adjacent or contiguous markers on the linkage map that is associated with increased water optimization can provide the position of an MTL associated with increased water optimization. Genetic loci correlating with particular phenotypes, such as drought tolerance, can be mapped in an organism's genome. By identifying a marker or cluster of markers that co-segregate with a trait of interest, the breeder is able to rapidly select a desired phenotype by selecting for the proper marker (a process called marker-assisted selection, or MAS). Such markers can also be used by breeders to design genotypes in silico and to practice whole genome selection.

The presently disclosed subject matter provides in some embodiments markers associated with increased drought tolerance/water optimization (e.g. markers demonstrated in Tables 1-7). Detection of these markers and/or other linked markers can be used to identify, select and/or produce drought tolerant plants and/or to eliminate plants that are not drought tolerant from breeding programs or planting.

In some embodiments, a DNA sequence within 1, 2, 3, 4, 5, 6, 7, 8, 9, 10, 15, 20, or 25 cM of a marker from Tables 1-7 of the presently disclosed subject matter displays a genetic recombination frequency of less than about 25%, 20%, 15%, 10%, 9%, 8%, 7%, 6%, 5%, 4%, 3%, 2%, or 1% with the marker of the presently disclosed subject matter. In some embodiments, the germplasm is a Zea mays line or variety.

DNA fragments associated with the presence of a water optimization associated trait, alleles, and/or haplotypes including, but not limited to SEQ ID NOs: 17-24, are also provided. In some embodiments, the DNA fragments associated with the presence of a water optimization associated trait have a predicted length and/or nucleic acid sequence, and detecting a DNA fragment having the predicted length and/or the predicted nucleic acid sequence is performed such that the amplified DNA fragment has a length that corresponds (plus or minus a few bases; e.g., a length of one, two or three bases more or less) to the predicted length. In some embodiments, a DNA fragment is an amplified fragment and the amplified fragment has a predicted length and/or nucleic acid sequence as does an amplified fragment produced by a similar reaction with the same primers with the DNA from the plant in which the marker was first detected or the nucleic acid sequence that corresponds (i.e., as a nucleotide sequence identity of more than 80%, 81%, 82%, 83%, 84%, 85%, 86%, 87%, 88%, 89%, 90%, 91%, 92%, 93%, 94%, 95%, 96%, 97%, 98%, or 99%) to the expected sequence as based on the sequence of the marker associated with that water optimization associated trait in the plant in which the marker was first detected. Upon a review of the instant disclosure, one of ordinary skill in the art would appreciate that markers that are absent in plants while they were present in at least one parent plant (so-called trans-markers), can also be useful in assays for detecting a desired trait in an progeny plant, although testing for the absence of a marker to detect the presence of a specific trait is not optimal. The detecting of an amplified DNA fragment having the predicted length or the predicted nucleic acid sequence can be performed by any of a number of techniques, including but not limited to standard gel electrophoresis techniques and/or by using automated DNA sequencers. The methods are not described here in detail as they are well known to the skilled person.

The primer (in some embodiments an extension primer and in some embodiments an amplification primer) is in some embodiments single stranded for maximum efficiency in extension and/or amplification. In some embodiments, the primer is an oligodeoxyribonucleotide. A primer is typically sufficiently long to prime the synthesis of extension and/or amplification products in the presence of the agent for polymerization. The minimum lengths of the primers can depend on many factors, including but not limited to temperature and composition (A/T vs. G/C content) of the primer.

In the context of an amplification primer, these are typically provided as one or more sets of bidirectional primers that include one or more forward and one or more reverse primers as commonly used in the art of DNA amplification such as in PCR amplification, As such, it will be understood that the term “primer”, as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing. Primers can be prepared by any suitable method. Methods for preparing oligonucleotides of specific sequence are known in the art, and include, for example, cloning, and restriction of appropriate sequences and direct chemical synthesis. Chemical synthesis methods can include, for example, the phospho di- or tri-ester method, the diethylphosphoramidate method and the solid support method disclosed in U.S. Pat. No. 4,458,068.

Primers can be labeled, if desired, by incorporating detectable moieties by for instance spectroscopic, fluorescence, photochemical, biochemical, immunochemical, or chemical moieties.

Template-dependent extension of an oligonucleotide primer is catalyzed by a polymerizing agent in the presence of adequate amounts of the four deoxyribonucleotides triphosphates (dATP, dGTP, dCTP and dTTP; i.e., dNTPs) or analogues, in a reaction medium that comprises appropriate salts, metal cations, and a pH buffering system. Suitable polymerizing agents are enzymes known to catalyze primer- and template-dependent DNA synthesis. Known DNA polymerases include, for example, E. coli DNA polymerase or its Klenow fragment, T4 DNA polymerase, and Taq DNA polymerase, as well as various modified versions thereof. The reaction conditions for catalyzing DNA synthesis with these DNA polymerases are known in the art. The products of the synthesis are duplex molecules consisting of the template strands and the primer extension strands, which include the target sequence. These products, in turn, can serve as template for another round of replication. In the second round of replication, the primer extension strand of the first cycle is annealed with its complementary primer; synthesis yields a “short” product which is bound on both the 5′- and the 3′-ends by primer sequences or their complements. Repeated cycles of denaturation, primer annealing, and extension can result in the exponential accumulation of the target region defined by the primers. Sufficient cycles are run to achieve the desired amount of polynucleotide containing the target region of nucleic acid. The desired amount can vary, and is determined by the function which the product polynucleotide is to serve.

The PCR method is well described in handbooks and known to the skilled person. After amplification by PCR, the target polynucleotides can be detected by hybridization with a probe polynucleotide which forms a stable hybrid with that of the target sequence under stringent to moderately stringent hybridization and wash conditions. If it is expected that the probes will be essentially completely complementary (i.e., about 99% or greater) to the target sequence, stringent conditions can be used. If some mismatching is expected, for example if variant strains are expected with the result that the probe will not be completely complementary, the stringency of hybridization can be reduced. In some embodiments, conditions are chosen to rule out non-specific/adventitious binding. Conditions that affect hybridization, and that select against non-specific binding are known in the art, and are described in, for example, Sambrook & Russell, 2001. Generally, lower salt concentration and higher temperature increase the stringency of hybridization conditions.

In order to detect the presence of two water optimization associated alleles on a single chromosome in a plant, chromosome painting methods can also be used. In such methods at least a first water optimization associated allele and at least a second water optimization associated allele can be detected in the same chromosome by in situ hybridization or in situ PCR techniques. More conveniently, the fact that two water optimization associated alleles are present on a single chromosome can be confirmed by determining that they are in coupling phase: i.e., that the traits show reduced segregation when compared to genes residing on separate chromosomes.

The water optimization associated alleles identified herein are located on a number of different chromosomes or linkage groups and their locations can be characterized by a number of otherwise arbitrary markers. In the present investigations, single nucleotide polymorphisms (SNPs), were used, although restriction fragment length polymorphism (RFLP) markers, amplified fragment length polymorphism (AFLP) markers, microsatellite markers (e.g., SSRs), insertion mutation markers, sequence-characterized amplified region (SCAR) markers, cleaved amplified polymorphic sequence (CAPS) markers, isozyme markers, microarray-based technologies, TAQMAN® Assays, ILLUMINA® GOLDENGATE® Assay analysis, nucleic acid sequencing technologies, or combinations of these markers might also have been used, and indeed can be used.

In general, providing complete sequence information for a water optimization associated allele and/or haplotype is unnecessary, as the way in which the water optimization associated allele and/or haplotype is first detected—through an observed correlation between the presence of one or more single nucleotide polymorphisms and the presence of a particular phenotypic trait—allows one to trace among a population of progeny plants those plants that have the genetic potential for exhibiting a particular phenotypic trait. By providing a non-limiting list of markers, the presently disclosed subject matter thus provides for the effective use of the presently disclosed water optimization associated alleles and/or haplotypes in breeding programs. In some embodiments, a marker is specific for a particular line of descent. Thus, a specific trait can be associated with a particular marker.

The markers as disclosed herein not only indicate the location of the water optimization associated allele, they also correlate with the presence of the specific phenotypic trait in a plant. It is noted that single nucleotide polymorphisms that indicate where a water optimization associated allele is present in the genome is non-limiting. In general, the location of a water optimization associated allele is indicated by a set of single nucleotide polymorphisms that exhibit statistical correlation to the phenotypic trait. Once a marker is found outside a single nucleotide polymorphism (i.e., one that has a LOD-score below a certain threshold, indicating that the marker is so remote that recombination in the region between that marker and the water optimization associated allele occurs so frequently that the presence of the marker does not correlate in a statistically significant manner to the presence of the phenotype), the boundaries of the water optimization associated allele can be considered set. Thus, it is also possible to indicate the location of the water optimization associated allele by other markers located within that specified region. It is further noted that a single nucleotide polymorphism can also be used to indicate the presence of the water optimization associated allele (and thus of the phenotype) in an individual plant, which in some embodiments means that it can be used in marker-assisted selection (MAS) procedures.

In principle, the number of potentially useful markers can be very large. Any marker that is linked to a water optimization associated allele (e.g., falling within the physically boundaries of the genomic region spanned by the markers having established LOD scores above a certain threshold thereby indicating that no or very little recombination between the marker and the water optimization associated allele occurs in crosses, as well as any marker in linkage disequilibrium to the water optimization associated allele, as well as markers that represent the actual causal mutations within the water optimization associated allele) can be used in the presently disclosed methods and compositions, and are within the scope of the presently disclosed subject matter. This means that the markers identified in the application as associated with the water optimization associated allele (e.g., markers that are present in or comprise any of SEQ ID NOs: 1-8, 17-65 as well as the alleles identified in Tables 1-7) are non-limiting examples of markers suitable for use in the presently disclosed methods and compositions. Moreover, when a water optimization associated allele, or the specific trait-conferring part thereof, is introgressed into another genetic background (i.e., into the genome of another maize or another plant species), then some markers might no longer be found in the progeny although the trait is present therein, indicating that such markers are outside the genomic region that represents the specific trait-conferring part of the water optimization associated allele in the original parent line only and that the new genetic background has a different genomic organization. Such markers of which the absence indicates the successful introduction of the genetic element in the progeny are called “trans markers” and can be equally suitable with respect to the presently disclosed subject matter.

Upon the identification of a water optimization associated allele and/or haplotype, the water optimization associated allele and/or haplotype effect (e.g., the trait) can for instance be confirmed by assessing trait in progeny segregating for the water optimization associated alleles and/or haplotypes under investigation. The assessment of the trait can suitably be performed by using phenotypic assessment as known in the art for water optimization traits. For example, (field) trials under natural and/or irrigated conditions can be conducted to assess the traits of hybrid and/or inbred maize.

The markers provided by the presently disclosed subject matter can be used for detecting the presence of one or more water optimization trait alleles and/or haplotypes at loci of the presently disclosed subject matter in a suspected water optimization trait introgressed maize plant, and can therefore be used in methods involving marker-assisted breeding and selection of such water optimization trait bearing maize plants. In some embodiments, detecting the presence of a water optimization associated allele and/or haplotype of the presently disclosed subject matter is performed with at least one of the markers for a water optimization associated allele and/or haplotype as defined herein. The presently disclosed subject matter therefore relates in another aspect to a method for detecting the presence of a water optimization associated allele and/or haplotype for at least one of the presently disclosed water optimization traits, comprising detecting the presence of a nucleic acid sequence of the water optimization associated allele and/or haplotype in a trait bearing maize plant, which presence can be detected by the use of the disclosed markers.

In some embodiments, the detecting comprises determining the nucleotide sequence of a Zea mays nucleic acid associated with a water optimization associated trait, allele and/or haplotype. The nucleotide sequence of a water optimization associated allele and/or haplotype of the presently disclosed subject matter can for instance be resolved by determining the nucleotide sequence of one or more markers associated with the water optimization associated allele and/or haplotype and designing internal primers for the marker sequences that can then be used to further determine the sequence of the water optimization associated allele and/or haplotype outside of the marker sequences.

For example, the nucleotide sequence of the SNP markers disclosed herein can be obtained by isolating the markers from the electrophoresis gel used in the determination of the presence of the markers in the genome of a subject plant, and determining the nucleotide sequence of the markers by, for example, dideoxy chain termination sequencing methods, which are well known in the art. In some embodiments of such methods for detecting the presence of a water optimization associated allele and/or haplotype in a trait bearing maize plant, the method can also comprise providing a oligonucleotide or polynucleotide capable of hybridizing under stringent hybridization conditions to a nucleic acid sequence of a marker linked to the water optimization associated allele and/or haplotype, in some embodiments selected from the markers disclosed herein, contacting the oligonucleotide or polynucleotide with digested genomic nucleic acid of a trait bearing maize plant, and determining the presence of specific hybridization of the oligonucleotide or polynucleotide to the digested genomic nucleic acid. In some embodiments, the method is performed on a nucleic acid sample obtained from the trait-bearing maize plant, although in situ hybridization methods can also be employed. Alternatively, one of ordinary skill in the art can, once the nucleotide sequence of the water optimization associated allele and/or haplotype has been determined, design specific hybridization probes or oligonucleotides capable of hybridizing under stringent hybridization conditions to the nucleic acid sequence of the water optimization associated allele and/or haplotype and can use such hybridization probes in methods for detecting the presence of a water optimization associated allele and/or haplotype disclosed herein in a trait bearing maize plant.

Particular nucleotides that are present at particular locations in the markers and nucleic acids disclosed herein can be determined using standard molecular biology techniques including, but not limited to amplification of genomic DNA from plants and subsequent sequencing. Additionally, oligonucleotide primers can be designed that would be expected to specifically hybridize to particular sequences that include the polymorphisms disclosed herein. For example, oligonucleotides can be designed to distinguish between the “A” allele and the “G” allele at a nucleotide position that corresponds to position 401 of SEQ ID NO: 17 using oligonucleotides comprising, consisting essentially of, or consisting of SEQ ID NOs: 27 and 28. The relevant difference between SEQ ID NOs: 27 and 28 is that the former has a G nucleotide at position 15 and the latter has an A nucleotide at position 16. Thus, SEQ ID NO: 27 hybridization conditions can be designed that would permit SEQ ID NO: 27 to specifically hybridize to the “G” allele, if present, but not hybridize to the “A” allele, if present. Thus, hybridization using these two primers that differ in only one nucleotide can be employed to assay for the presence of one or the other allele at a nucleotide position that corresponds to position 401 of SEQ ID NO: 17.

In some embodiments, the marker can comprise, consist essentially of, or consist of the reverse complement of any of the aforementioned markers. In some embodiments, one or more of the alleles that make up a marker haplotype is present as described above, whilst one or more of the other alleles that make up the marker haplotype is present as the reverse complement of the allele(s) described above. In some embodiments, each of the alleles that make up a marker haplotype is present as the reverse complement of the allele(s) described above.

In some embodiments, the marker can comprise, consist essentially of, or consist of an informative fragment of any of the aforementioned markers, the reverse complement of any of the aforementioned markers, or an informative fragment of the reverse complement of any of the aforementioned markers. In some embodiments, one or more of the alleles/sequences that make up a marker haplotype is present as described above, whilst one or more of the other alleles/sequences that make up the marker haplotype is present as the reverse complement of the alleles/sequences described above. In some embodiments, one or more of the alleles/sequences that make up a marker haplotype is present as described above, whilst one or more of the other alleles/sequences that make up the marker haplotype is present as an informative fragment of the alleles/sequences described above. In some embodiments, one or more of the alleles/sequences that make up a marker haplotype is present as described above, whilst one or more of the other alleles/sequences that make up the marker haplotype is present as an informative fragment of the reverse complement of the alleles/sequences described above. In some embodiments, each of the alleles/sequences that make up a marker haplotype is present as an informative fragment of the alleles/sequences described above, the reverse complement of the alleles/sequences described above, or an informative fragment of the reverse complement of the alleles/sequences described above.

In some embodiments, the marker can comprise, consist essentially of, or consist of any marker linked to the aforementioned markers. That is, any allele and/or haplotype that is in linkage disequilibrium with any of the aforementioned markers can also be used to identify, select and/or produce a maize plant with increased drought tolerance. Linked markers can be determined, for example, by using resources available on the MaizeGDB website.

Isolated and purified markers associated with increased drought tolerance are also provided. Such markers can comprise, consist essentially of, or consist of a nucleotide sequence as set forth in any of SEQ ID NOs: 1-8, AND 17-65, the alleles described in Tables 1-7 and the reverse complement thereof, or an informative fragment thereof. In some embodiments, the marker comprises a detectable moiety. In some embodiments, the marker permits the detection of one or more of the marker alleles identified herein.

Compositions comprising a primer pair capable of amplifying a nucleic acid sample isolated from a maize plant or germplasm to generate a marker associated with increased drought tolerance are also provided. In some embodiments, the marker comprises a nucleotide sequence as set forth herein, the reverse complement thereof, or an informative fragment thereof. In some embodiments, the marker comprises a nucleotide sequence that is at least about 50%, 55%, 60%, 65%, 70%, 75%, 80%, 85%, 90%, 95% 97%, 99% or 100% identical to a nucleotide sequence set forth herein, the reverse complement thereof, or an informative fragment thereof. In some embodiments, the primer pair is one of the amplification primer pairs identified in Table 8 above. One of ordinary skill in the art will understand how to select alternative primer pairs according to methods well known in the art.

The identification of plants with different alleles and/or haplotypes of interest can provide starting materials for combining alleles and/or haplotypes in progeny plants via breeding strategies designed to “stack” the alleles and/or haplotypes. As used herein, the term “stacking”, and grammatical variants thereof, refers to the intentional accumulation by breeding (including but not limited to crossing two plants, selfing a single plant, and/or creating a double haploid from a single plant) of favorable water optimization haplotypes in plants such that a plant's genome has at least one additional favorable water optimization haplotype than its immediate progenitor(s). Stacking includes in some embodiments conveying one or more water optimization traits, alleles, and/or haplotypes into a progeny maize plant such that the progeny maize plant includes higher number of water optimization traits, alleles, and/or haplotypes than does either parent from which it was derived. By way of example and not limitation, if Parent 1 has haplotypes A, B, and C, and Parent 2 has haplotypes D, E, and F, “stacking” refers to the production of a plant that has any of A, B, and C, with any combination of D, E, and F. Particularly, “stacking” refers in some embodiments to producing a plant that has A, B, and C as well as one or more of D, E, and F, or producing a plant that has D, E, and F as well as one or more of A, B, and C. In some embodiments, “stacking” refers to the production of a plant from a bi-parental cross that contains all water optimization associated haplotypes possessed by either parent.

### III. Methods for Introgressing Alleles of Interest and for Identifying Plants Comprising the Same

III.A. Marker-Assisted Selection Generally Markers can be used in a variety of plant breeding applications. See e.g., Staub et al., Hortscience 31: 729 (1996); Tanksley, Plant Molecular Biology Reporter 1: 3 (1983). One of the main areas of interest is to increase the efficiency of backcrossing and introgressing genes using marker-assisted selection (MAS). In general, MAS takes advantage of genetic markers that have been identified as having a significant likelihood of co-segregation with a desired trait. Such markers are presumed to be in/near the gene(s) that give rise to the desired phenotype, and their presence indicates that the plant will possess the desired trait. Plants which possess the marker are expected to transfer the desired phenotype to their progeny.

A marker that demonstrates linkage with a locus affecting a desired phenotypic trait provides a useful tool for the selection of the trait in a plant population. This is particularly true where the phenotype is hard to assay or occurs at a late stage in plant development. Since DNA marker assays are less laborious and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing or imparting the trait. Having flanking markers decreases the chances that false positive selection will occur. The ideal situation is to have a marker in the gene itself, so that recombination cannot occur between the marker and the gene. Such a marker is called a “perfect marker.”

When a gene is introgressed by MAS, it is not only the gene that is introduced but also the flanking regions. Gepts, Crop Sci 42:1780 (2002). This is referred to as “linkage drag.” In the case where the donor plant is highly unrelated to the recipient plant, these flanking regions carry additional genes that can code for agronomically undesirable traits. This “linkage drag” can also result in reduced yield or other negative agronomic characteristics even after multiple cycles of backcrossing into the elite maize line. This is also sometimes referred to as “yield drag.” The size of the flanking region can be decreased by additional backcrossing, although this is not always successful, as breeders do not have control over the size of the region or the recombination breakpoints. Young et al., Genetics 120:579 (1998). In classical breeding, it is usually only by chance that recombinations which contribute to a reduction in the size of the donor segment are selected. Tanksley et al., Biotechnology 7: 257 (1989). Even after 20 backcrosses, one can expect to find a sizeable piece of the donor chromosome still linked to the gene being selected. With markers, however, it is possible to select those rare individuals that have experienced recombination near the gene of interest. In 150 backcross plants, there is a 95% chance that at least one plant will have experienced a crossover within 1 cM of the gene, based on a single meiosis map distance. Markers allow for unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers. See Tanksley et al., supra. When the exact location of a gene is known, flanking markers surrounding the gene can be utilized to select for recombinations in different population sizes. For example, in smaller population sizes, recombinations can be expected further away from the gene, so more distal flanking markers would be required to detect the recombination.

The availability of integrated linkage maps of the maize genome containing increasing densities of public maize markers has facilitated maize genetic mapping and MAS. See, e.g. the IBM2 Neighbors maps, which are available online on the MaizeGDB website.

Of all the molecular marker types, SNPs are the most abundant and have the potential to provide the highest genetic map resolution. Bhattramakki et al., Plant Molec. Biol. 48:539 (2002). SNPs can be assayed in a so-called “ultra-high-throughput” fashion because they do not require large amounts of nucleic acid and automation of the assay is straight-forward. SNPs also have the benefit of being relatively low-cost systems. These three factors together make SNPs highly attractive for use in MAS. Several methods are available for SNP genotyping, including but not limited to, hybridization, primer extension, oligonucleotide ligation, nuclease cleavage, minisequencing and coded spheres. Such methods have been reviewed in various publications: Gut, Hum. Mutat. 17:475 (2001); Shi, Clin. Chem. 47:164 (2001); Kwok, Pharmacogenomics 1:95 (2000); Bhattramakki and Rafalski, Discovery and application of single nucleotide polymorphism markers in plants, in PLANT GENOTYPING: THE DNA FINGERPRINTING OF PLANTS, CABI Publishing, Wallingford (2001). A wide range of commercially available technologies utilize these and other methods to interrogate SNPs, including Masscode™ (Qiagen, Germantown, Md.), Invader® (Hologic, Madison, Wis.), SnapShot® (Applied Biosystems, Foster City, Calif.), Taqman® (Applied Biosystems, Foster City, Calif.) and Beadarrays™ (Illumina, San Diego, Calif.).

A number of SNPs together within a sequence, or across linked sequences, can be used to describe a haplotype for any particular genotype. Ching et al., BMC Genet. 3:19 (2002); Gupta et al., (2001), Rafalski, Plant Sci. 162:329 (2002b). Haplotypes can be more informative than single SNPs and can be more descriptive of any particular genotype. For example, a single SNP can be allele “T” for a specific drought tolerant line or variety, but the allele “T” might also occur in the maize breeding population being utilized for recurrent parents. In this case, a combination of alleles at linked SNPs can be more informative. Once a unique haplotype has been assigned to a donor chromosomal region, that haplotype can be used in that population or any subset thereof to determine whether an individual has a particular gene. The use of automated high throughput marker detection platforms known to those of ordinary skill in the art makes this process highly efficient and effective.

The markers of the presently disclosed subject matter can be used in marker-assisted selection protocols to identify and/or select progeny with increased drought tolerance. Such methods can comprise, consist essentially of, or consist of crossing a first maize plant or germplasm with a second maize plant or germplasm, wherein the first maize plant or germplasm comprises a marker associated with increased drought tolerance, and selecting a progeny plant that possesses the marker. Either of the first and second maize plants, or both, can be of a non-naturally occurring variety of maize.

III.B. Methods of Introgressing Alleles and/or Haplotypes of Interest

Thus, in some embodiments the presently disclosed subject matter provides methods for introgressing an allele associated with increased drought tolerance into a genetic background lacking said allele. In some embodiments, the methods comprise crossing a donor comprising said allele with a recurrent parent that lacks said allele; and repeatedly backcrossing progeny comprising said allele with the recurrent parent, wherein said progeny are identified by detecting, in their genomes, the presence of a marker within a chromosome interval the group consisting of:

(a) a chromosome interval on chromosome 1 defined by and including base pair (bp) position 272937470 to base pair (bp) position 272938270 (PZE01271951242);

(b) a chromosome interval on chromosome 2 defined by and including base pair (bp) position 12023306 to base pair (bp) position 12024104 (PZE0211924330);

(c) a chromosome interval on chromosome 3 defined by and including base pair (bp) position 225037202 to base pair (bp) position 225038002 (PZE03223368820);

(d) a chromosome interval on chromosome 3 defined by and including base pair (bp) position 225340531 to base pair (bp) position 225341331 (PZE03223703236);

(e) a chromosome interval on chromosome 5 defined by and including base pair (bp) position 159,120,801 to base pair (bp) position 159,121,601 (PZE05158466685);

(f) a chromosome interval on chromosome 9 defined by and including base pair (bp) position 12104536 to base pair (bp) position 12105336 (PZE0911973339);

(g) a chromosome interval on chromosome 9 defined by and including base pair (bp) position 225343590 to base pair (bp) position 225340433 (S_18791654);

(h) a chromosome interval on chromosome 10 defined by and including base pair (bp) position 14764415 to base pair (bp) position 14765098 (S_20808011); and thereby producing a drought tolerant maize plant or germplasm comprising said allele associated with increased drought tolerance in the genetic background of the recurrent parent, thereby introgressing the allele associated with increased drought tolerance into a genetic background lacking said allele. In some embodiments, the genome of said drought tolerant maize plant or germplasm comprising said allele associated with increased drought tolerance is at least about 95% identical to that of the recurrent parent. In some embodiments, either the donor or the recurrent parent, or both, is of a non-naturally occurring variety of maize.

Thus, in some embodiments the presently disclosed subject matter provides a method for producing a plant with increased yield comprising the steps of
a. selecting from a diverse plant population using marker selected from the group comprised of markers SM2973, SM2980, SM2982, SM2984, SM2987, SM2991, SM2995, SM2996;
b. propagating/selfing the plant.
   In further embodiments of the method the presently disclosed subject matter provides a method for producing a plant with increased yield comprising the steps of:
a. selecting from a diverse plant population using marker selected from the group comprised of markers SM2973, SM2980, SM2982, SM2984, SM2987, SM2991, SM2995, SM2996; wherein
   marker SM2973 has an “G” at nucleotide 401;
   marker SM2980 has an “C” at nucleotide 401;
   marker SM2982 has an “A” at nucleotide 401;
   marker SM2984 has an “G” at nucleotide 401;
   marker SM2987 has an “G” at nucleotide 401;
   marker SM2991 has an “G” at nucleotide 401;
   marker SM2995 has an “A” at nucleotide 401; and
   marker SM2996 has an “A” at nucleotide 401.

III.D. Methods of Stacking Alleles and/or Haplotypes of Interest

The presently disclosed subject matter relates in some embodiments to “stacking” of haplotypes associated with water optimization in order to produce plants (and parts thereof) that have multiple favorable water optimization loci. By way of example and not limitation, the presently disclosed subject matter relates in some embodiments to the identification and characterization of Zea mays loci that are each associated with one or more water optimization traits. These loci correspond to SEQ ID NOs: 1-8 and 17-65 as well has Haplotypes A-M defined herein.

For each of these loci, favorable alleles have been identified that are associated with water optimization traits. These favorable alleles are summarized herein, for example Tables 1-7 or any markers closely linked to the genes listed in Table 9. The presently disclosed subject matter provides exemplary alleles (e.g. as displayed in Tables 1-7 or Table 11) that are associated with increases and decreases of various water optimization traits as defined herein.

III.E. Methods of Identifying Plants Comprising Alleles and/or Haplotypes of Interest

Methods for identifying a drought tolerant maize plant or germplasm can comprise detecting the presence of a marker associated with increased drought tolerance. The marker can be detected in any sample taken from the plant or germplasm, including, but not limited to, the whole plant or germplasm, a portion of said plant or germplasm (e.g., a cell from said plant or germplasm) or a nucleotide sequence from said plant or germplasm. The maize plant can be of a non-naturally occurring variety of maize. In some embodiments, the genome of the maize plant or germplasm is at least about 50%, 55%, 60%, 65%, 70%, 75%, 80%, 85%, 90%, 95%, 97%, 99% or 100% identical to that of an elite variety of maize.

Methods for introgressing an allele associated with increased drought tolerance into a maize plant or germplasm can comprise crossing a first maize plant or germplasm comprising said allele (the donor) with a second maize plant or germplasm that lacks said allele (the recurrent parent) and repeatedly backcrossing progeny comprising said allele with the recurrent parent. Progeny comprising said allele can be identified by detecting, in their genomes, the presence of a marker associated with increased drought tolerance. Either the donor or the recurrent parent, or both, can be of a non-naturally occurring variety of maize.

### IV. Production of Improved Trait Carrying Maize Plants by Transgenic Methods

In some embodiments, the presently disclosed subject matter relates to the use of polymorphisms (including but not limited to SNPs) or trait-conferring parts for producing a trait carrying maize plant by introducing a nucleic acid sequence comprising a trait-associated allele and/or haplotype of the polymorphism into a recipient plant.

A donor plant, with the nucleic acid sequence that comprises a water optimization trait allele and/or haplotype can be transferred to the recipient plant lacking the allele and/or the haplotype. The nucleic acid sequence can be transferred by crossing a water optimization trait carrying donor plant with a non-trait carrying recipient plant (e.g., by introgression), by transformation, by protoplast transformation or fusion, by a doubled haploid technique, by embryo rescue, or by any other nucleic acid transfer system. Then, if desired, progeny plants comprising one or more of the presently disclosed water optimization trait alleles and/or haplotypes can be selected. A nucleic acid sequence comprising a water optimization trait allele and/or haplotype can be isolated from the donor plant using methods known in the art, and the isolated nucleic acid sequence can transform the recipient plant by transgenic methods. This can occur with a vector, in a gamete, or other suitable transfer element, such as a ballistic particle coated with the nucleic acid sequence.

Plant transformation generally involves the construction of an expression vector that will function in plant cells and includes nucleic acid sequence that comprises an allele and/or haplotype associated with the water optimization trait, which vector can comprise a water optimization trait-conferring gene. This gene usually is controlled or operatively linked to one or more regulatory element, such as a promoter. The expression vector can contain one or more such operably linked gene/regulatory element combinations, provided that at least one of the genes contained in the combinations encodes water optimization trait. The vector(s) can be in the form of a plasmid, and can be used, alone or in combination with other plasmids, to provide transgenic plants that are better water optimization plants, using transformation methods known in the art, such as the Agrobacterium transformation system. In some embodiments of the invention genes comprised in the chromosomal intervals herein may be transgenically expressed in plants to produce plants with increased drought tolerance; further, not to be limited by theory the gene models displayed in Table 9 may be transgenically expressed in plants to produce increased drought tolerant plants.

Transformed cells often contain a selectable marker to allow transformation identification. The selectable marker is typically adapted to be recovered by negative selection (by inhibiting the growth of cells that do not contain the selectable marker gene), or by positive selection (by screening for the product encoded by the selectable marker gene). Many commonly used selectable marker genes for plant transformation are known in the art, and include, for example, genes that code for enzymes that metabolically detoxify a selective chemical agent that can be an antibiotic or a herbicide, or genes that encode an altered target which is insensitive to the inhibitor. Several positive selection methods are known in the art, such as mannose selection. Alternatively, marker-less transformation can be used to obtain plants without the aforementioned marker genes, the techniques for which are also known in the art.

### Water Optimization Genes

Multiple positive associations of the assay SM2987 with increased yield under drought identify the gene GRMZM2G027059 as a water optimization gene. GRMZM2G027059 encodes 4-hydroxy-3-methylbut-2-enyl diphosphate reductase which is the last enzyme in the biosynthesis of isopentenyl diphosphate (IPP) and dimethylallyl diphosphate (DMAPP) (Arturo Guevara-Garci\acute{ } a, The Plant Cell, Vol. 17, 628-643), February 2005. In higher plants, two pathways are used for the synthesis of the basic isoprenoid units. The mevalonic (MVA) pathway occurs in the cytoplasm where sesquiterpenes (C15) and triterpenes (C30), such as phytosterols, dolichols, and farnesyl residues, for protein prenylation are produced the methyl-D-erythritol 4-phosphate (MEP) pathway operates in plastids and produces IPP and DMAPP for the synthesis of isoprenoids, such as isoprene, carotenoids, plastoquinones, phytol conjugates (such as chlorophylls and tocopherols), and hormones (gibberellins and abscisic acid). Evidence indicates that cross talk between both pathways exists (Hsieh and Goodman. Plant Physiology, June 2005). Since GRMZM2G027059 encodes 4-hydroxy-3-methylbut-2-enyl diphosphate reductase, which is an essential enzyme for the biosynthesis of photo pigments such as chlorophylls and carotenoid and hormones such as gibberellins and abscisic acid, therefore plants expressing this gene may be more tolerant to abiotic stress.

Multiple positive associations of the assay SM2991 with increased yield under drought identify the gene GRMZM2G156365 as a water optimization gene. GRMZM2G156365 belongs to PectinAcetylEsterase (PAE) family Pectin Acetyl Esterases catalyse the deacetylation of pectin, a major compound of primary cell walls. Propriatary expression array data shows that GRMZM2G156365 has very high expression in pollen and anthers and GRMZM2G156365 had higher expression in drought tolerant maize hybrid than a drought sensitive maize hybrid. Tobacco plants overexpressing a poplar PAE, PtPAE, exhibited severe male sterility hindering pollen germination and pollen tube elongation, so plants produce few or no mature seeds (Gou, J. Y., L. M. Miller, et al. (2012). “Acetylesterase-mediated deacetylation of pectin impairs cell elongation, pollen germination, and plant reproduction.” Plant Cell 24(1): 50-65). Yield loss caused by pollen sterility is one of the major drought issues. Pollen germination and pollen tube elongation require precise status of pectin acetylation in the cell wall. GRMZM2G156365 may function as an structural regulator by modulating the precise status of pectin acetylation to affect the cell wall remodeling and physiochemical properties, thereby affecting pollen cell extensibility. Plants down regulating GRMZM2G156365 gene expression in pollen might increase pollen germination under abiotic stresses such as drought.

Multiple positive associations of the assay SM2995 with increased yield under drought identify the gene GRMZM2G134234 as a water optimization gene. GRMZM2G134234 contains a domain IPR012866, protein of unknown function DUF1644. This family consists of sequences found in a number of hypothetical plant proteins of unknown function. The region of interest contains nine highly conserved cysteine residues and is approximately 160 amino acids in length, which probably represent a zinc-binding domain. An Arabidopsis DUF1644 gene, AT3G25910, respond to GA and ABA treatments (Guo, C. et al., J Integr Plant Biol (2015)). There are 9 members from rice DUF1644 family that might involve in stress response. SIDP364 localized in nucleus and was induced by ABA, high salt, drought, heat, cold and H₂O₂. Overexpression in rice increases ABA sensitivity and high salt tolerance (due to Proline accumulation and up-regulation of stress responsive genes). SIDP361 has similar function with SIDP364 in salt stress by regulating ABA dependent or independent signaling pathway. However, they have different response to different stresses (REF). Family of DUF1644-containing genes may regulate responses to abiotic stress in rice. Overexpressing OsSIDP366 in rice increased drought and salinity tolerance and reduced water loss, and RNAi plants were more sensitive to salinity and drought treatments (Guo, C., C. Luo, et al. (2015). “OsSIDP366, a DUF1644 gene, positively regulates responses to drought and salt stresses in rice.” J Integr Plant Biol). DUF1644-containing genes may regulate responses to abiotic stresses. GRMZM2G134234 might positively regulate stress responsive genes to increase maize stress tolerance. Plants overexpressing GRMZM2G134234 might be more tolerant to abiotic stresses such as drought and salt.

Multiple positive associations of the assay SM2996 with increased yield under drought identify the gene GRMZM2G094428 as a water optimization gene. GRMZM2G094428 contains a IPR003480 chloramphenicol transferase domain. Acylation is a common and biochemically significant modification of plant secondary metabolites. A large family of acyltransferases named BAHD, which utilize CoA thioesters and catalyze the formation of a diverse group of plant metabolites. The BAHD superfamily comprises a vast group of enzymes with little amino acid sequence similarity but two consensus motifs, HXXXD and DFGWG. GRMZM2G094428 is phylogenicals most similar to BAD transferases involved in cell wall feruloylation/coumaroylation. GRMZM2G094428 is predicted to involve cell wall feruloylation/coumaroylation. The cell walls of grasses such as wheat, maize, rice, and sugar cane, contains two most prominent compounds which are p-coumaric acid (pCA) and ferulic acid (FA). The pCA is almost exclusively esterified to lignin, and FA is esterified to GAX in the cell wall (Lu and Ralph, 1999). BAHD acyl-coA transferase superfamily have been identified as being responsible for the process (Hugo, et al., 2013). Overexpression or knockout of BAHD acyl-coA transferase could change cell wall composition. Knockout of BAHD acyl-coA transferase could reduce FA or p-CA content, change lignin content (Piston et al., 2010) OE of OsAT10 in rice can increase matrix polysaccharideassociated ester-linked p-CA while simultaneously decreasing matrix polysaccharide-associated FA, but no discernible phenotypic alterations in vegetative development, lignin content, or lignin composition. (Larua et al., 2013). The RNAi line of pCAT showed reduced pCA level, but lignin levels did not change (Jane, et al., 2014) Lignin and abiotic stress (reviewed by Michael, 2013). Lignification of crop tissues affects plant fitness and can confer tolerance to abiotic stresses. Transgenic tobacco plants with increased lignin levels showed improved tolerance to drought compared to the wild type. The lignin deficient maize mutants exhibited drought symptoms even in well-watered conditions and in which leaf lignin levels correlated with drought tolerance in a set of contrasting genotypes. A transgenic rice line which deposited increased levels of lignin in the roots when exposed to salt treatment was more tolerant than its wild type, which did not show such a response. GRMZM2G094428 might be responsible for p-coumaroylation of monolignols which finally involved lignin biosynthesis, and also responsible for FA esterified to GAX in the cell wall. Increased lignin content can confer plant tolerance under abiotic stresses, including drought and salt.

Multiple positive associations of the assay SM2973 with increased yield under drought identify the gene GRMZM2G416751 as a water optimization gene. The GRMZM2G416751 has 62% identity and 83% similarity to the c-terminal 450 amino acids of the Arabidopsis gene AT5G58100.1. In spot1 mutant lines (SALK_061320, SALK_041228, and SALK_079847), At5g58100 were disrupted with T-DNA insertions at different regions. Exine elements in spot1 mutant appeared to be largely disconnected, indicating possible problems with tectum formation (Dobritsa, A. A., A. Geanconteri, et al. (2011). “A large-scale genetic screen in Arabidopsis to identify genes involved in pollen exine production.” Plant Physiol 157(2): 947-970). Yield loss caused by pollen sterility is one of the major drought issues. GRMZM2G416751 might be involved in pollen exine formation to increase maize stress tolerance. Plants overexpressing this gene might avoid pollen sterility under drought stress.

Multiple positive associations of the assay SM2980 with increased yield under drought identify the gene GRMZM2G467169 as a water optimization gene. GRMZM2G467169 has a predicted conserved domain of human type polyadenylate binding protein family GRMZM2G467169 highly expressed in leaf and reproductive tissues. Arabidopsis putative ortholog AT4G01290 (RIMB3) positively regulates 2CPA (2-Cys-Peroxiredoxin A) in retrograde redox signaling from chloroplasts to the nucleus. rimb3 mutant grew slower with smaller leaves and larger rimb3 plants had chlorosis under long-day condition. RIMB3 plays a role in plant cells as sensor in response to biotic or abiotic stresses. AT4G01290 protein binds to the 5′ cap complex in Arabidopsis. AT4G01290 interacts with UBQ3 and is possibly degraded by the 26S proteasome. Under various biotic and abiotic stresses, signals such as redox imbalance in PS1 originated from chloroplast are transmitted to the nucleus to affect gene expression pattern (retrograde signaling). GRMZM2G467169 might regulate retrograde signaling to increase maize stress tolerance. Plants overexpressing this gene might be more tolerant to abiotic stresses such as drought.

Multiple positive associations of the assay SM2982 with increased yield under drought identify the gene GRMZM5G862107 as a water optimization gene. GRMZM5G862107 contains an RNA-binding domain, S1, IPR006196 and has 69% identity to the Arabidopsis protein AT5G30510. The S1 domain is very similar to that of cold shock protein (Bycroft et al., Cell, January 1997). Cold shock proteins (CSPs) contain RNA-binding sequences referred to as cold shock domains (CSDs) and are well known to act as RNA chaperones. The role of CSP in bacteria is adaptation to cold stress. Plant CSD-containing proteins share a high level of similarity with the bacterial CSPs and were shown to share in vitro and in vivo functions with bacterial CSPs (Journal of Experimental Botany, Vol. 62, No. 11, pp. 4003-4011, 2011). Plant CSD-containing proteins have generally been reported to respond to abiotic stresses. Plants overexpressing this gene might be more tolerant to abiotic stresses such as drought.

Multiple positive associations of the assay SM2984 with increased yield under drought identify the gene GRMZM2G050774 as a water optimization gene. GRMZM2G050774 encodes RING Finger domain protein Sub-type H2 (C3HC4) tentatively an E3 ligase. E3 ligases such as ATL31/6 in Arabidopsis have been reported to function in carbon and nitrogen metabolism regulation (Plant Signal Behav. 2011 October; 6(10): 1465-1468). GRMZM2G050774 could be involved in stress signaling responsible for improving drought resistance.

### Transformation

Chloramphenicol acetyltransferase gene (Callis et al. 1987, Genes Develop. 1: 1183-1200). In the same experimental system, the intron from the maize bronze 1 gene had a similar effect in enhancing expression. Intron sequences have been routinely incorporated into plant transformation vectors, typically within the non-translated leader.

“Linker” refers to a polynucleotide that comprises the connecting sequence between two other polynucleotides. The linker may be at least 1, 3, 5, 8, 10, 15, 20, 30, 50, 100, 200, 500, 1000, or 2000 polynucleotides in length. A linker may be synthetic, such that its sequence is not found in nature, or it may naturally occur, such as an intron.

“Exon” refers to a section of DNA which carries the coding sequence for a protein or part of it. Exons are separated by intervening, non-coding sequences (introns).

“Transit peptides” generally refer to peptide molecules that when linked to a protein of interest directs the protein to a particular tissue, cell, subcellular location, or cell organelle. Examples include, but are not limited to, chloroplast transit peptides, nuclear targeting signals, and vacuolar signals. To ensure localization to the plastids it is conceivable to use, but not limited to, the signal peptides of the ribulose bisphosphate carboxylase small subunit (Wolter et al. 1988, PNAS 85: 846-850; Nawrath et al., 1994, PNAS 91: 12760-12764), of the NADP malate dehydrogenase (Galiardo et al. 1995, Planta 197: 324-332), of the glutathione reductase (Creissen et al. 1995, Plant J 8: 167-175) or of the R1 protein Lorberth et al. (1998, Nature Biotechnology 16: 473-477).

The term “transformation” as used herein refers to the transfer of a nucleic acid fragment into the genome of a host cell, resulting in genetically stable inheritance. In some particular embodiments, the introduction into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of nucleic acid into the plant, plant part and/or cell thereof, or a combination thereof.

Procedures for transforming plants are well known and routine in the art and are described throughout the literature. Non-limiting examples of methods for transformation of plants include transformation via bacterial-mediated nucleic acid delivery (e.g., via bacteria from the genus Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (2002, Cell Mol Biol Lett 7:849-858 (2002)).

Thus, in some particular embodiments, the introducing into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethyleneglycol-mediated transformation, any other electrical, chemical, physical and/or biological mechanism that results in the introduction of nucleic acid into the plant, plant part and/or cell thereof, or a combination thereof.

Agrobacterium-mediated transformation is a commonly used method for transforming plants because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al 1993, Plant Cell 5:159-169). The transfer of the recombinant binary vector to Agrobacterium can be accomplished by a tri-parental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Höfgen and Willmitzer 1988, Nucleic Acids Res 16:9877).

Transformation of a plant by recombinant Agrobacterium usually involves co-cultivation of the Agrobacterium with explants from the plant and follows methods well known in the art. Transformed tissue is typically regenerated on selection medium carrying an antibiotic or herbicide resistance marker between the binary plasmid T-DNA borders.

Another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacterium or a bacteriophage, each containing one or more nucleic acids sought to be introduced) also can be propelled into plant tissue.

Thus, in particular embodiments of the present invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

By “stably introducing” or “stably introduced” in the context of a polynucleotide introduced into a cell is intended the introduced polynucleotide is stably incorporated into the genome of the cell, and thus the cell is stably transformed with the polynucleotide.

“Stable transformation” or “stably transformed” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome.

Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

The “transformation and regeneration process” refers to the process of stably introducing a transgene into a plant cell and regenerating a plant from the transgenic plant cell. As used herein, transformation and regeneration includes the selection process, whereby a transgene comprises a selectable marker and the transformed cell has incorporated and expressed the transgene, such that the transformed cell will survive and developmentally flourish in the presence of the selection agent. “Regeneration” refers to growing a whole plant from a plant cell, a group of plant cells, or a plant piece such as from a protoplast, callus, or tissue part.

A “selectable marker” or “selectable marker gene” refers to a gene whose expression in a plant cell gives the cell a selective advantage. “Positive selection” refers to a transformed cell acquiring the ability to metabolize a substrate that it previously could not use or could not use efficiently, typically by being transformed with and expressing a positive selectable marker gene. This transformed cell thereby grows out of the mass of non-transformed tissue. Positive selection can be of many types from inactive forms of plant growth regulators that are then converted to active forms by the transferred enzyme to alternative carbohydrate sources that are not utilized efficiently by the non-transformed cells, for example mannose, which then become available upon transformation with an enzyme, for example phosphomannose isomerase, that allows them to be metabolized. Non-transformed cells either grow slowly in comparison to transformed cells or not at all. Other types of selection may be due to the cells transformed with the selectable marker gene gaining the ability to grow in presence of a negative selection agent, such as an antibiotic or an herbicide, compared to the ability to grow of non-transformed cells. A selective advantage possessed by a transformed cell may also be due to the loss of a previously possessed gene in what is called “negative selection”. In this, a compound is added that is toxic only to cells that did not lose a specific gene (a negative selectable marker gene) present in the parent cell (typically a transgene).

Examples of selectable markers include, but are not limited to, genes that provide resistance or tolerance to antibiotics such as kanamycin (Dekeyser et al. 1989, Plant Phys 90: 217-23), spectinomycin (Svab and Maliga 1993, Plant Mol Biol 14: 197-205), streptomycin (Maliga et al. 1988, Mol Gen Genet 214: 456-459), hygromycin B (Waldron et al. 1985, Plant Mol Biol 5: 103-108), bleomycin (Hille et al. 1986, Plant Mol Biol 7: 171-176), sulphonamides (Guerineau et al. 1990, Plant Mol Biol 15: 127-136), streptothricin (Jelenska et al. 2000, Plant Cell Rep 19: 298-303), or chloramphenicol (De Block et al. 1984, EMBO J 3: 1681-1689). Other selectable markers include genes that provide resistance or tolerance to herbicides, such as the S4 and/or Hra mutations of acetolactate synthase (ALS) that confer resistance to herbicides including sulfonylureas, imidazolinones, triazolopyrimidines, and pyrimidinyl thiobenzoates; 5-enol-pyrovyl-shikimate-3-phosphate-synthase (EPSPS) genes, including but not limited to those described in U.S. Pat. Nos. 4,940,935, 5,188,642, 5,633,435, 6,566,587, 7,674,598 (as well as all related applications) and the glyphosate N-acetyltransferase (GAT) which confers resistance to glyphosate (Castle et al. 2004, Science 304:1151-1154, and U.S. Patent Application Publication Nos. 20070004912, 20050246798, and 20050060767); BAR which confers resistance to glufosinate (see e.g., U.S. Pat. No. 5,561,236); aryloxy alkanoate dioxygenase or AAD-1, AAD-12, or AAD-13 which confer resistance to 2,4-D; genes such as Pseudomonas HPPD which confer HPPD resistance; Sprotophorphyrinogen oxidase (PPO) mutants and variants, which confer resistance to peroxidizing herbicides including fomesafen, acifluorfen-sodium, oxyfluorfen, lactofen, fluthiacet-methyl, saflufenacil, flumioxazin, flumiclorac-pentyl, carfentrazone-ethyl, sulfentrazone); and genes conferring resistance to dicamba, such as dicamba monoxygenase (Herman et al. 2005, J Biol Chem 280: 24759-24767 and U.S. Pat. No. 7,812,224 and related applications and patents). Other examples of selectable markers can be found in Sundar and Sakthivel (2008, J Plant Physiology 165: 1698-1716), herein incorporated by reference.

Other selection systems include using drugs, metabolite analogs, metabolic intermediates, and enzymes for positive selection or conditional positive selection of transgenic plants. Examples include, but are not limited to, a gene encoding phosphomannose isomerase (PMI) where mannose is the selection agent, or a gene encoding xylose isomerase where D-xylose is the selection agent (Haldrup et al. 1998, Plant Mol Biol 37: 287-96). Finally, other selection systems may use hormone-free medium as the selection agent. One non-limiting example the maize homeobox gene kn1, whose ectopic expression results in a 3-fold increase in transformation efficiency (Luo et al. 2006, Plant Cell Rep 25: 403-409). Examples of various selectable markers and genes encoding them are disclosed in Miki and McHugh (J Biotechnol, 2004, 107: 193-232; incorporated by reference).

In some embodiments of the invention, the selectable marker may be plant derived. An example of a selectable marker which can be plant derived includes, but is not limited to, 5-enolpyruvylshikimate-3-phosphate synthase (EPSPS). The enzyme 5-enolpyruvylshikimate-3-phosphate synthase (EPSPS) catalyzes an essential step in the shikimate pathway common to aromatic amino acid biosynthesis in plants. The herbicide glyphosate inhibits EPSPS, thereby killing the plant. Transgenic glyphosate-tolerant plants can be created by the introduction of a modified EPSPS transgene which is not affected by glyphosate (for example, U.S. Pat. No. 6,040,497; incorporated by reference). Other examples of a modified plant EPSPS which can be used as a selectable marker in the presence of glyphosate includes a P106L mutant of rice EPSPS (Zhou et al 2006, Plant Physiol 140: 184-195) and a P106S mutation in goosegrass EPSPS (Baerson et al 2002, Plant Physiol 129: 1265-1275). Other sources of EPSPS which are not plant derived and can be used to confer glyphosate tolerance include but are not limited to an EPSPS P101S mutant from Salmonella typhimurium (Comai et al 1985, Nature 317: 741-744) and a mutated version of CP4 EPSPS from Agrobacterium sp. Strain CP4 (Funke et al 2006, PNAS 103: 13010-13015). Although the plant EPSPS gene is nuclear, the mature enzyme is localized in the chloroplast (Mousdale and Coggins 1985, Planta 163:241-249). EPSPS is synthesized as a preprotein containing a transit peptide, and the precursor is then transported into the chloroplast stroma and proteolytically processed to yield the mature enzyme (della-Cioppa et al. 1986, PNAS 83: 6873-6877). Therefore, to create a transgenic plant which has tolerance to glyphosate, a suitably mutated version of EPSPS which correctly translocates to the chloroplast could be introduced. Such a transgenic plant then has a native, genomic EPSPS gene as well as the mutated EPSPS transgene. Glyphosate could then be used as a selection agent during the transformation and regeneration process, whereby only those plants or plant tissue that are successfully transformed with the mutated EPSPS transgene survive.

As used herein, the terms “promoter” and “promoter sequence” refer to nucleic acid sequences involved in the regulation of transcription initiation. A “plant promoter” is a promoter capable of initiating transcription in plant cells. Exemplary plant promoters include, but are not limited to, those that are obtained from plants, from plant viruses and from bacteria that comprise genes expressed in plant cells such Agrobacterium or Rhizobium. A “tissue-specific promoter” is a promoter that preferentially initiates transcription in a certain tissue (or combination of tissues). A “stress-inducible promoter” is a promoter that preferentially initiates transcription under certain environmental conditions (or combination of environmental conditions). A “developmental stage-specific promoter” is a promoter that preferentially initiates transcription during certain developmental stages (or combination of developmental stages).

As used herein, the term “regulatory sequences” refers to nucleotide sequences located upstream (5′ non-coding sequences), within or downstream (3′ non-coding sequences) of a coding sequence, which influence the transcription, RNA processing or stability, or translation of the associated coding sequence. Regulatory sequences include, but are not limited to, promoters, enhancers, exons, introns, translation leader sequences, termination signals, and polyadenylation signal sequences. Regulatory sequences include natural and synthetic sequences as well as sequences that can be a combination of synthetic and natural sequences. An “enhancer” is a nucleotide sequence that can stimulate promoter activity and can be an innate element of the promoter or a heterologous element inserted to enhance the level or tissue specificity of a promoter. The coding sequence can be present on either strand of a double-stranded DNA molecule, and is capable of functioning even when placed either upstream or downstream from the promoter.

Some embodiments include overexpressing one or more SEQ ID NOs: 9-16, and/or decreasing the expression and/or concentration (e.g., level) of SEQ ID NOs: 9-16. In some embodiments, a method and/or composition of the present invention may be used to overexpress one or more SEQ ID NOs: 9-16, and/or decrease the expression and/or concentration of SEQ ID NOs: 9-16 in a tissue specific manner. For example, one or more SEQ ID NOs: 9-16 may be operably linked to a tissue-specific promoter sequence to provide tissue-specific expression (e.g., root- and/or green tissue-specific expression) of the one or more SEQ ID NOs: 9-16. In some embodiments, providing overexpression or tissue-specific expression of one or more SEQ ID NOs: 9-16 may increase yield, increase yield stability under drought stress conditions, and/or enhance drought stress tolerance in a plant and/or plant part in which said proteins are expressed.

In some embodiments of the invention, a plant having introduced into its genome a water optimization gene, wherein the said water optimization gene comprises a nucleotide sequence encoding at least one polypeptide comprising SEQ ID NO: 9-16 is provided.

In some embodiments, said plant has increased yield as compared to a control plant.
In some embodiments, increased yield is yield under water deficit conditions.
In some embodiments a parental line of said plant was selected by or identified by a nucleotide probe or primer that annealed to any one of SEQ ID NOs: 1-8 and said parental line conferred increased yield as compared to a plant not comprising SEQ ID NOs: 1-8.
In some embodiments said gene is introduced by heterologous expression. In some embodiments said gene is introduced by gene editing. In some embodiments said gene is introduced by breeding or trait introgression.
In some embodiments the nucleic acid sequence comprises any one of SEQ ID NOs: 1-8.
In some embodiments increased yield is yield under water deficit conditions.
In some embodiments said plant is maize.
In some embodiments said plant is an elite maize line or a hybrid.
In some embodiments said gene is a nucleotide sequence having 80-100% sequence homology with any one of SEQ ID NOs: 1-8.
In some embodiments said plant also comprises at least one Haplotypes A-M.
In some embodiments a plant cell, germplasm, pollen, seed or plant part from the plant of any one of the previous embodiments is provided.
In some embodiments a genotyped plant, plant cell, germplasm, pollen, seed or plant part selected or identified based on the detection of any one of SEQ ID NOs: 1-8 is provided.
In some embodiments of the invention, the plant, plant cell, germplasm, pollen, seed or plant part is genotyped by isolating DNA from said plant, plant cell, germplasm, pollen, seed or plant part and DNA is genotyped using either PCR or nucleotide probes that adhere to any one of SEQ ID NOs 1-8.

In another embodiment, A method of selecting a first maize plant or germplasm that displays either increased yield under drought or increased yield under non-drought conditions, the method comprising: a)isolating nucleic acids from the first maize plant or germplasm; b) detecting in the first maize plant or germplasm at least one allele of a quantitative trait locus that is associated with increased yield under drought, wherein said quantitative trait locus is localized to a chromosomal interval flanked by and including markers IIM56014 and IIM48939 on chromosome 1, IIM39140 and IIM40144 on chromosome 3, IIM6931 and IIM7657 on chromosome 9, IIM40272 and IIM41535 on chromosome 2, IIM39102 and IIM40144 on chromosome 3, IIM25303 and IIM48513 on chromosome 5, IIM4047 and IIM4978 on chromosome 9, and IIM19 and IIM818 on chromosome 10; and c) selecting said first maize plant or germplasm, or selecting a progeny of said first maize plant or germplasm, comprising at least one allele associated with increased yield under drought. Additionally the method wherein said quantitative trait locus is localized to a chromosomal interval flanked by and including IIM56705 and IIM56748 on chromosome 1; a chromosomal interval flanked by and including IIM39914 and IIM39941 on chromosome 3; a chromosomal interval flanked by and including IIM7249 and IIM7272 on chromosome 9; a chromosomal interval flanked by and including IIM40719 and IIM40771 on chromosome 2; a chromosomal interval flanked by and including IIM39900 and IIM39935 on chromosome 3; a chromosomal interval flanked by and including IIM25799 and IIM25806 on chromosome 5; a chromosomal interval flanked by and including IIM4345 and IIM4458 on chromosome 9; a chromosomal interval flanked by and including IIM46822 and IIM62316 on chromosome 10. The method of further comprising crossing said selected first maize plant or germplasm with a second maize plant or germplasm, and wherein the introgressed maize plant or germplasm displays increased yield under drought. The embodiment further wherein, the at least one allele is detected using a composition comprising a detectable label

In another embodiment, A method introgressing a water optimization locus comprising: a) providing a first population of maize plants; b) detecting the presence of a genetic marker that is associated with water optimization and is closely linked to and within 24 Mb of SM2987 in the first population; c) selecting one or more plants with the water optimization locus from the first population of maize plants; and d) producing offspring from the one or more plants with the water optimization locus, wherein the offspring exhibit improved water optimization compared to the first population. The embodiment wherein the genetic marker is detected within 10 Mb of SM2987; 5 Mb of SM2987; 1 Mb of SM2987; 0.5 Mb of SM2987. The embodiment wherein the genetic marker detected is within any one of: a chromosomal interval comprised by and flanked by IIM56014 and IIM48939; a chromosomal interval comprised by and flanked by IIM59859 and IIM57051; or a chromosomal interval comprised by and flanked by IIM56705 and IIM56748. In another aspect the embodiment wherein the genetic marker is selected from or closely associated with any one of: IIM56014, IIM56027, IIM56145, IIM56112, IIM56097, IIM56166, IIM56167, IIM56176, IIM56246, IIM56250, IIM56256, IIM56261, IIM56399, IIM59999, IIM59859, IIM59860, IIM56462, IIM56470, IIM56472, IIM56483, IIM56526, IIM56539, IIM56578, IIM56602, IIM56610, IIM56611, IIM61006, IIM56626, IIM56658, IIM56671, IIM58395, IIM48879, IIM48880, IIM56700, IIM56705, SM2987, IIM56731, IIM56746, IIM56748, IIM56759, IIM56770, IIM56772, IIM69710, IIM56795 IIM56910, IIM69670, IIM59541, IIM56918, IIM48891, IIM48892, IIM58609, IIM56962, IIM56965, IIM57051, IIM57340, IIM57586, IIM57589, IIM57605, IIM57609, IIM57611, IIM57612, IIM57620, IIM57626, and IIM48939. Another aspect is a maize plant (stiff or non-stiff stalk) generated from this embodiment.

In another embodiment, a method introgressing a water optimization locus comprising: a) providing a first population of maize plants; b) detecting the presence of a genetic marker that is associated with water optimization and is closely linked to and within 10 Mb of SM2996 in the first population; c) selecting one or more plants with the water optimization locus from the first population of maize plants; and d) producing offspring from the one or more plants with the water optimization locus, wherein the offspring exhibit improved water optimization compared to the first population. The embodiment further wherein the genetic marker detected is within 0.5 Mb, 1 Mb, 2 Mb or 5 Mb of SM2996. In a further aspect the genetic marker is within a chromosomal interval comprising any of the following: a chromosomal interval comprised by and flanked by IIM39140 and IIM40144, a chromosomal interval comprised by and flanked by IIM39732 and IIM40055. a chromosomal interval comprised by and flanked by IIM39914 and IIM39941. In another aspect of the embodiment the genetic marker detected is selected from the group comprised IIM39140, IIM39142, IIM39334, IIM39347, IIM39377, IIM39378, IIM39380, IIM39381, IIM39383, IIM39384, IIM39385, IIM39386, IIM39390, IIM39453, IIM39485, IIM39496, IIM39527, IIM39715, IIM39716, IIM39725, IIM39726, IIM39731, IIM39729, IIM39728, IIM39732, IIM39771, IIM39784, IIM39783, IIM39786, IIM39787, IIM39802, IIM39856, IIM39870, IIM39873, IIM39877, IIM39883, IIM39900, IIM39914, IIM39935, IIM39941, IIM39976, IIM39990, IIM39994, IIM40032, IIM40033, IIM40045, IIM40046, IIM40047, IIM48771, IIM40055, IIM40060, IIM40061, IIM40062, IIM40064, IIM40094, IIM40095, IIM40096, IIM40099, IIM40144 or a closely linked marker of any of the above. A further aspect of the embodiment is a maize plant cell or maize plant (stiff or non-stiff stalk) generated by the method above.

A further embodiment comprises a method introgressing a water optimization locus comprising: a) providing a first population of maize plants; b) detecting the presence of a genetic marker that is associated with water optimization and is closely linked to and within 12 Mb of SM2982 in the first population; c) selecting one or more plants with the water optimization locus from the first population of maize plants; and d) producing offspring from the one or more plants with the water optimization locus, wherein the offspring exhibit improved water optimization compared to the first population. A further aspect of the embodiment wherein the genetic marker detected is within 5 Mb, 2 Mb, 1 Mb or 0.5 Mb of SM2982. Another aspect wherein the genetic marker detected is within a chromosomal interval comprising any one of a chromosomal interval defined by and flanked by IIM6931 and IIM7657; a chromosomal interval comprised by and flanked by IIM7117 and IIM7427; a chromosomal interval comprised by and flanked by IIM7204 and IIM7273. In another aspect of the embodiment the genetic marker detected is selected from the group comprising IIM6931, IIM6934, IIM6946, IIM6961, IIM7041, IIM7054, IIM7055, IIM7086, IIM7101, IIM7104, IIM7105, IIM7109, IIM7110, IIM7114, IIM7117, IIM7141, IIM7151, IIM7151, IIM7163, IIM7168, IIM7166, IIM7178, IIM7184, IIM7183, IIM7204, IIM7231, IIM7235, IIM7249, IIM7272, IIM7273, IIM7275, IIM7284, IIM7283, IIM7285, IIM7318, IIM7319, IIM7345, IIM7351, IIM7354, IIM7384, IIM7386, IIM7388, IIM7397, IIM7417, IIM7427, IIM7463, IIM7480, IIM7481, IIM7548, IIM7613, IIM7616, IIM48034, IIM7636, IIM7653, IIM7657. A further aspect of the embodiment is a maize plant cell or maize plant (stiff or non-stiff stalk) generated by the method above.

Another embodiment comprises a method of introgressing a water optimization locus into a maize plant comprising the steps of: a) providing a first population of maize plants; b) detecting the presence of a genetic marker that is associated with water optimization and is closely linked to and within 10 Mb of SM2991 in the first population; c) selecting one or more plants with the water optimization locus from the first population of maize plants; and d) producing offspring from the one or more plants with the water optimization locus, wherein the offspring exhibit improved water optimization compared to the first population. A further aspect of the embodiment wherein the genetic marker detected is within 5 Mb, 2 Mb, 1 Mb or 0.5 Mb of SM2991. Another aspect wherein the genetic marker detected is within a chromosomal interval selected from the group consisting of: a chromosomal interval defined by and flanked by IIM40272 and IIM41535; a chromosomal interval comprised by and flanked by IIM40486 and IIM40771; a chromosomal interval comprised by and flanked by IIM40646 and IIM40768. In another aspect of the embodiment the genetic marker detected is selected from the group comprising: IIM40272, IIM40279, IIM40301, IIM40310, IIM40311, IIM40440, IIM40442, IIM40463, IIM40486, IIM40522, IIM40627, IIM40646, IIM40709, IIM40719, IIM40768, IIM40771, IIM40775, IIM40788, IIM40789, IIM40790, IIM40795, IIM40802, IIM40804, IIM40837, IIM40839, IIM40848, IIM47120, IIM40862, IIM40863, IIM40888, IIM40893, IIM40909, IIM40928, IIM40931, IIM40932, IIM40940, IIM47155, IIM40936, IIM47156, IIM40991, IIM40998, IIM41001, IIM41008, IIM41013, IIM41033, IIM41064, IIM41153, IIM41229, IIM41230, IIM41247, IIM41259, IIM41261, IIM41263, IIM41283, IIM41287, IIM41310, IIM41321, IIM41359, IIM41357, IIM41366, IIM41377, IIM46720, IIM41412, IIM41430, IIM41448, IIM41456, IIM49103, IIM41479, IIM41509, IIM41535 or a closely associated marker thereof. A further aspect of the embodiment is a maize plant cell or maize plant (stiff or non-stiff stalk) generated by the method above.

In another embodiment, a method introgressing a water optimization locus comprising the steps of: a) providing a first population of maize plants; b) detecting the presence of a genetic marker that is associated with water optimization and is closely linked to and within 10 Mb, 5 Mb, 2 Mb, 1 Mb or 0.5 Mb of SM2995 in the first population; c) selecting one or more plants with the water optimization locus from the first population of maize plants; and d) producing offspring from the one or more plants with the water optimization locus, wherein the offspring exhibit improved water optimization compared to the first population. Another aspect wherein the genetic marker detected is within a chromosomal interval selected from the group consisting of: a chromosomal interval comprised by and flanked by IIM39102 and IIM40144; a chromosomal interval comprised by and flanked by IIM39732 and IIM40064; a chromosomal interval comprised by and flanked by IIM39900 and IIM39935. In another aspect of the embodiment the genetic marker detected is selected from the group comprising: IIM39102, IIM39140, IIM39142, IIM39283, IIM39291, IIM39298, IIM39300, IIM39301, IIM39304, IIM39306, IIM39309, IIM39334, IIM39335, IIM39336, IIM39340, IIM39347, IIM39375, IIM39377, IIM39378, IM39380, IIM39381, IIM39383, IIM39384, IIM39385, IIM39386, IIM39390, IIM39401, IIM39409, IIM39447, IIM39497, IIM39715, IIM39716, IIM39731, IIM39732, IIM39830, IIM39856, IIM39870, IIM39873, IIM39877, IIM39883, IIM39900, IIM39935, IIM39989, IIM40045, IIM40062, IIM40064, IIM40144 or a closely associated marker thereof. A further aspect of the embodiment is a maize plant cell or maize plant (stiff or non-stiff stalk) generated by the method above.

In another embodiment, a method introgressing a water optimization locus into a maize plant comprising the steps of: a) providing a first population of maize plants; b) detecting the presence of a genetic marker that is associated with water optimization and is closely linked to and within 20 Mb, 10 Mb, 5 Mb, 2 Mb, 1 Mb or 0.5 Mb of SM2973 in the first population; c) selecting one or more plants with the water optimization locus from the first population of maize plants; and d) producing offspring from the one or more plants with the water optimization locus, wherein the offspring exhibit improved water optimization compared to the first population. Another aspect wherein the genetic marker detected is within a chromosomal interval selected from the group consisting of: a chromosomal interval comprised by and flanked by IIM25303 and IIM48513; a chromosomal interval comprised by and flanked by IIM25545 and IIM25938; a chromosomal interval comprised by and flanked by IIM25800 and IIM25805. In another aspect of the embodiment the genetic marker detected is selected from the group comprising: IIM25303, IIM25304, IIM25320, IIM25350, IIM25391, IIM25399, IIM25400, IIM25402, IIM25407, IIM25414, IIM25429, IIM25442, IIM25449, IIM25526, IIM25543, IIM25545, IIM25600, IIM25688, IIM25694, IIM25731, IIM25740, IIM25799, IIM25800, IIM25805, IIM25806, IIM25819, IIM25820, IIM25821, IIM25823, IIM25824, IIM25828, IIM25830, IIM25856, IIM25864, IIM25870, IIM25895, IIM25905, IIM25921, IIM25938, IIM25939, IIM25945, IIM25965, IIM25966, IIM25968, IIM25975, IIM25978, IIM25983, IIM25984, IIM25987, IIM25999, IIM25999, IIM26009, IIM26023, IIM26084, IIM26119, IIM26132, IIM26133, IIM26145, IIM26151, IIM48428, IIM26170, IIM26175, IIM26226, IIM26263, IIM26264, IIM26267, IIM26268, IIM26271, IIM26272, IIM26273, IIM26274, IIM26291, IIM26319, IIM26323, IIM26325, IIM26383, IIM26402, IIM26493, IIM26495, IIM48513 or a closely associated marker thereof. A further aspect of the embodiment is a maize plant cell or maize plant (stiff or non-stiff stalk) generated by the method above.

Another embodiment comprising a method of introgressing a water optimization locus into a maize plant comprising the steps of: a) providing a first population of maize plants; b) detecting the presence of a genetic marker that is associated with water optimization and is closely linked to and within 10 Mb, 5 Mb, 2 Mb, 1 Mb or 0.5 Mb of SM2980 in the first population; c) selecting one or more plants with the water optimization locus from the first population of maize plants; and d) producing offspring from the one or more plants with the water optimization locus, wherein the offspring exhibit improved water optimization compared to the first population. Another aspect wherein the genetic marker detected is within a chromosomal interval selected from the group consisting of: a chromosomal interval comprised by and flanked by IIM4047 and IIM4978; a chromosomal interval comprised by and flanked by IIM4231 and IIM4607; or a chromosomal interval comprised by and flanked by IIM4395 and IIM4458. In another aspect of the embodiment the genetic marker detected is selected from the group comprising: IIM4047, IIM4046, IIM4044, IIM4038, IIM4109, IIM4121, IIM4143, IIM4177, IIM4203, IIM4212, IIM4214, IIM4214, IIM4215, IIM4219, IIM4226, IIM4227, IIM4229, IIM4231, IIM4232, IIM4233, IIM4235, IIM4236, IIM4237, IIM4239, IIM4239, IIM4240, IIM4241, IIM4242, IIM4244, IIM4255, IIM4263, IIM4264, IIM4265, IIM4308, IIM4295, IIM4289, IIM4280, IIM4345, IIM4387, IIM4387, IIM4388, IIM4388, IIM4389, IIM4390, IIM4390, IIM4392, IIM4395, IIM4458, IIM4469, IIM4482, IIM4607, IIM4608, IIM4609, IIM4613, IIM4614, IIM4674, IIM4681, IIM4682, IIM4738, IIM4755, IIM4756, IIM4768, IIM4777, IIM4816, IIM4818, IIM4822, IIM4831, IIM4851, IIM4856, IIM47276, IIM4857, IIM4858, IIM4859, IIM4860, IIM4875, IIM4878, IIM4967, IIM4974, IIM4978 or a closely associated marker thereof. A further aspect of the embodiment is a maize plant cell or maize plant (stiff or non-stiff stalk) generated by the method above.

Another embodiment comprising a method of introgressing a water optimization locus into a maize plant comprising the steps of: a) providing a first population of maize plants; b) detecting the presence of a genetic marker that is associated with water optimization and is closely linked to and within 5 Mb, 4 Mb, 2 Mb, 1 Mb or 0.5 Mb of SM2984 in the first population; c) selecting one or more plants with the water optimization locus from the first population of maize plants; and d) producing offspring from the one or more plants with the water optimization locus, wherein the offspring exhibit improved water optimization compared to the first population. Another aspect wherein the genetic marker detected is within a chromosomal interval selected from the group consisting of: a chromosomal interval comprised by and flanked by IIM19 and IIM818; a chromosomal interval comprised by and flanked by IIM43 and IIM291 or a chromosomal interval comprised by and flanked by IIM121 and IIM211. In another aspect of the embodiment the genetic marker detected is selected from the group comprising: IIM19, IIM26, IIM32, IIM43, IIM66, IIM72, IIM78, IIM77, IIM84, IIM108, IIM121, IIM46822, IIM211, IIM236, IIM274, IIM275, IIM291, IIM347, IIM47190, IIM638, IIM738, IIM739, IIM818 or a closely associated marker thereof. A further aspect of the embodiment is a maize plant cell or maize plant (stiff or non-stiff stalk) generated by the method above.

In another embodiment, A method introgressing a water optimization locus comprising: a) providing a first population of maize plants; b) detecting the presence of a genetic marker that is associated with water optimization and is closely linked to and within 24 Mb of SM2987 in the first population; c) selecting one or more plants with the water optimization locus from the first population of maize plants; and d) producing offspring from the one or more plants with the water optimization locus, wherein the offspring exhibit improved water optimization compared to the first population. The embodiment wherein the genetic marker is detected within 10 Mb of SM2987; 5 Mb of SM2987; 1 Mb of SM2987; 0.5 Mb of SM2987. The embodiment wherein the genetic marker detected is within any one of: a chromosomal interval comprised by and flanked by IIM56014 and IIM48939; a chromosomal interval comprised by and flanked by IIM59859 and IIM57051; or a chromosomal interval comprised by and flanked by IIM56705 and IIM56748. In another aspect the embodiment wherein the genetic marker is selected from or closely associated with any one of: IIM56014, IIM56027, IIM56145, IIM56112, IIM56097, IIM56166, IIM56167, IIM56176, IIM56246, IIM56250, IIM56256, IIM56261, IIM56399, IIM59999, IIM59859, IIM59860, IIM56462, IIM56470, IIM56472, IIM56483, IIM56526, IIM56539, IIM56578, IIM56602, IIM56610, IIM56611, IIM61006, IIM56626, IIM56658, IIM56671, IIM58395, IIM48879, IIM48880, IIM56700, IIM56705, SM2987, IIM56731, IIM56746, IIM56748, IIM56759, IIM56770, IIM56772, IIM69710, IIM56795 IIM56910, IIM69670, IIM59541, IIM56918, IIM48891, IIM48892, IIM58609, IIM56962, IIM56965, IIM57051, IIM57340, IIM57586, IIM57589, IIM57605, IIM57609, IIM57611, IIM57612, IIM57620, IIM57626, and IIM48939. Another aspect is a maize plant (stiff or non-stiff stalk) generated from this embodiment.

In another embodiment, a method introgressing a water optimization locus comprising: a) providing a first population of maize plants; b) detecting the presence of a genetic marker that is associated with water optimization and is closely linked to and within 10 Mb of SM2996 in the first population; c) selecting one or more plants with the water optimization locus from the first population of maize plants; and d) producing offspring from the one or more plants with the water optimization locus, wherein the offspring exhibit improved water optimization compared to the first population. The embodiment further wherein the genetic marker detected is within 0.5 Mb, 1 Mb, 2 Mb or 5 Mb of SM2996. In a further aspect the genetic marker is within a chromosomal interval comprising any of the following: a chromosomal interval comprised by and flanked by IIM39140 and IIM40144, a chromosomal interval comprised by and flanked by IIM39732 and IIM40055 or a chromosomal interval comprised by and flanked by IIM39914 and IIM39941. In another aspect of the embodiment the genetic marker detected is selected from the group comprised IIM39140, IIM39142, IIM39334, IIM39347, IIM39377, IIM39378, IIM39380, IIM39381, IIM39383, IIM39384, IIM39385, IIM39386, IIM39390, IIM39453, IIM39485, IIM39496, IIM39527, IIM39715, IIM39716, IIM39725, IIM39726, IIM39731, IIM39729, IIM39728, IIM39732, IIM39771, IIM39784, IIM39783, IIM39786, IIM39787, IIM39802, IIM39856, IIM39870, IIM39873, IIM39877, IIM39883, IIM39900, IIM39914, IIM39935, IIM39941, IIM39976, IIM39990, IIM39994, IIM40032, IIM40033, IIM40045, IIM40046, IIM40047, IIM48771, IIM40055, IIM40060, IIM40061, IIM40062, IIM40064, IIM40094, IIM40095, IIM40096, IIM40099, IIM40144 or a closely linked marker of any of the above. A further aspect of the embodiment is a maize plant cell or maize plant (stiff or non-stiff stalk) generated by the method above.

A further embodiment comprises a method introgressing a water optimization locus comprising: a) providing a first population of maize plants; b) detecting the presence of a genetic marker that is associated with water optimization and is closely linked to and within 12 Mb of SM2982 in the first population; c) selecting one or more plants with the water optimization locus from the first population of maize plants; and d) producing offspring from the one or more plants with the water optimization locus, wherein the offspring exhibit improved water optimization compared to the first population. A further aspect of the embodiment wherein the genetic marker detected is within 5 Mb, 2 Mb, 1 Mb or 0.5 Mb of SM2982. Another aspect wherein the genetic marker detected is within a chromosomal interval comprising any one of a chromosomal interval defined by and flanked by IIM6931 and IIM7657; a chromosomal interval comprised by and flanked by IIM7117 and IIM7427; a chromosomal interval comprised by and flanked by IIM7204 and IIM7273. In another aspect of the embodiment the genetic marker detected is selected from the group comprising IIM6931, IIM6934, IIM6946, IIM6961, IIM7041, IIM7054, IIM7055, IIM7086, IIM7101, IIM7104, IIM7105, IIM7109, IIM7110, IIM7114, IIM7117, IIM7141, IIM7151, IIM7151, IIM7163, IIM7168, IIM7166, IIM7178, IIM7184, IIM7183, IIM7204, IIM7231, IIM7235, IIM7249, IIM7272, IIM7273, IIM7275, IIM7284, IIM7283, IIM7285, IIM7318, IIM7319, IIM7345, IIM7351, IIM7354, IIM7384, IIM7386, IIM7388, IIM7397, IIM7417, IIM7427, IIM7463, IIM7480, IIM7481, IIM7548, IIM7613, IIM7616, IIM48034, IIM7636, IIM7653, IIM7657. A further aspect of the embodiment is a maize plant cell or maize plant (stiff or non-stiff stalk) generated by the method above.

Another embodiment comprises a method of identifying or selecting a maize plant having increased yield under drought or increased yield under non-drought conditions as compared to a control plant wherein yield is increased bushels of corn per acre, the method comprising the steps of: a) isolating a nucleic acid from a plant cell; b) detecting the presence of a genetic marker in said nucleic acid that is associated with increased yield (drought or non-drought conditions) wherein said genetic marker is closely linked to and within 10 Mb, 5 Mb, 2 Mb, 1 Mb or 0.5 Mb of SM2991; c) selecting a maize plant on the basis of the genetic marker detected in b). Another aspect wherein the genetic marker detected is within a chromosomal interval selected from the group consisting of: a chromosomal interval defined by and flanked by IIM40272 and IIM41535; a chromosomal interval comprised by and flanked by IIM40486 and IIM40771; a chromosomal interval comprised by and flanked by IIM40646 and IIM40768. In another aspect of the embodiment the genetic marker detected is selected from the group comprising: IIM40272, IIM40279, IIM40301, IIM40310, IIM40311, IIM40440, IIM40442, IIM40463, IIM40486, IIM40522, IIM40627, IIM40646, IIM40709, IIM40719, IIM40768, IIM40771, IIM40775, IIM40788, IIM40789, IIM40790, IIM40795, IIM40802, IIM40804, IIM40837, IIM40839, IIM40848, IIM47120, IIM40862, IIM40863, IIM40888, IIM40893, IIM40909, IIM40928, IIM40931, IIM40932, IIM40940, IIM47155, IIM40936, IIM47156, IIM40991, IIM40998, IIM41001, IIM41008, IIM41013, IIM41033, IIM41064, IIM41153, IIM41229, IIM41230, IIM41247, IIM41259, IIM41261, IIM41263, IIM41283, IIM41287, IIM41310, IIM41321, IIM41359, IIM41357, IIM41366, IIM41377, IIM46720, IIM41412, IIM41430, IIM41448, IIM41456, IIM49103, IIM41479, IIM41509, IIM41535 or a closely associated marker thereof. A further aspect of the embodiment is a maize plant cell or maize plant (stiff or non-stiff stalk) selected by the method above.

Another embodiment comprises a method of identifying or selecting a maize plant having increased yield under drought or increased yield under non-drought conditions as compared to a control plant wherein yield is increased bushels of corn per acre, the method comprising the steps of: a) isolating a nucleic acid from a plant cell; b) detecting the presence of a genetic marker in said nucleic acid that is associated with increased yield (drought or non-drought conditions) wherein said genetic marker is closely linked to and within 10 Mb, 5 Mb, 2 Mb, 1 Mb or 0.5 Mb of SM2995 c) selecting a maize plant on the basis of the genetic marker detected in b). Another aspect wherein the genetic marker detected is within a chromosomal interval selected from the group consisting of: a chromosomal interval comprised by and flanked by IIM39102 and IIM40144; a chromosomal interval comprised by and flanked by IIM39732 and IIM40064; a chromosomal interval comprised by and flanked by IIM39900 and IIM39935. In another aspect of the embodiment the genetic marker detected is selected from the group comprising: IIM39102, IIM39140, IIM39142, IIM39283, IIM39291, IIM39298, IIM39300, IIM39301, IIM39304, IIM39306, IIM39309, IIM39334, IIM39335, IIM39336, IIM39340, IIM39347, IIM39375, IIM39377, IIM39378, IM39380, IIM39381, IIM39383, IIM39384, IIM39385, IIM39386, IIM39390, IIM39401, IIM39409, IIM39447, IIM39497, IIM39715, IIM39716, IIM39731, IIM39732, IIM39830, IIM39856, IIM39870, IIM39873, IIM39877, IIM39883, IIM39900, IIM39935, IIM39989, IIM40045, IIM40062, IIM40064, IIM40144 or a closely associated marker thereof. A further aspect of the embodiment is a maize plant cell or maize plant (stiff or non-stiff stalk) selected by the method above.

In Another embodiment comprises a method of identifying or selecting a maize plant having increased yield under drought or increased yield under non-drought conditions as compared to a control plant wherein yield is increased bushels of corn per acre, the method comprising the steps of: a) isolating a nucleic acid from a plant cell; b) detecting the presence of a genetic marker in said nucleic acid that is associated with increased yield (drought or non-drought conditions) wherein said genetic marker is closely linked to and within 20 Mb, 10 Mb, 5 Mb, 2 Mb, 1 Mb or 0.5 Mb of SM2973 c) selecting a maize plant on the basis of the genetic marker detected in b). Another aspect wherein the genetic marker detected is within a chromosomal interval selected from the group consisting of: a chromosomal interval comprised by and flanked by IIM25303 and IIM48513; a chromosomal interval comprised by and flanked by IIM25545 and IIM25938; a chromosomal interval comprised by and flanked by IIM25800 and IIM25805. In another aspect of the embodiment the genetic marker detected is selected from the group comprising: IIM25303, IIM25304, IIM25320, IIM25350, IIM25391, IIM25399, IIM25400, IIM25402, IIM25407, IIM25414, IIM25429, IIM25442, IIM25449, IIM25526, IIM25543, IIM25545, IIM25600, IIM25688, IIM25694, IIM25731, IIM25740, IIM25799, IIM25800, IIM25805, IIM25806, IIM25819, IIM25820, IIM25821, IIM25823, IIM25824, IIM25828, IIM25830, IIM25856, IIM25864, IIM25870, IIM25895, IIM25905, IIM25921, IIM25938, IIM25939, IIM25945, IIM25965, IIM25966, IIM25968, IIM25975, IIM25978, IIM25983, IIM25984, IIM25987, IIM25999, IIM25999, IIM26009, IIM26023, IIM26084, IIM26119, IIM26132, IIM26133, IIM26145, IIM26151, IIM48428, IIM26170, IIM26175, IIM26226, IIM26263, IIM26264, IIM26267, IIM26268, IIM26271, IIM26272, IIM26273, IIM26274, IIM26291, IIM26319, IIM26323, IIM26325, IIM26383, IIM26402, IIM26493, IIM26495, IIM48513 or a closely associated marker thereof. A further aspect of the embodiment is a maize plant cell or maize plant (stiff or non-stiff stalk) generated by the method above.

In another embodiment comprises a method of identifying or selecting a maize plant having increased yield under drought or increased yield under non-drought conditions as compared to a control plant wherein yield is increased bushels of corn per acre, the method comprising the steps of: a) isolating a nucleic acid from a plant cell; b) detecting the presence of a genetic marker in said nucleic acid that is associated with increased yield (drought or non-drought conditions) wherein said genetic marker is closely linked to and within 10 Mb, 5 Mb, 2 Mb, 1 Mb or 0.5 Mb of SM2980 c) selecting a maize plant on the basis of the genetic marker detected in b). Another aspect wherein the genetic marker detected is within a chromosomal interval selected from the group consisting of: a chromosomal interval comprised by and flanked by IIM4047 and IIM4978; a chromosomal interval comprised by and flanked by IIM4231 and IIM4607; or a chromosomal interval comprised by and flanked by IIM4395 and IIM4458. In another aspect of the embodiment the genetic marker detected is selected from the group comprising: IIM4047, IIM4046, IIM4044, IIM4038, IIM4109, IIM4121, IIM4143, IIM4177, IIM4203, IIM4212, IIM4214, IIM4214, IIM4215, IIM4219, IIM4226, IIM4227, IIM4229, IIM4231, IIM4232, IIM4233, IIM4235, IIM4236, IIM4237, IIM4239, IIM4239, IIM4240, IIM4241, IIM4242, IIM4244, IIM4255, IIM4263, IIM4264, IIM4265, IIM4308, IIM4295, IIM4289, IIM4280, IIM4345, IIM4387, IIM4387, IIM4388, IIM4388, IIM4389, IIM4390, IIM4390, IIM4392, IIM4395, IIM4458, IIM4469, IIM4482, IIM4607, IIM4608, IIM4609, IIM4613, IIM4614, IIM4674, IIM4681, IIM4682, IIM4738, IIM4755, IIM4756, IIM4768, IIM4777, IIM4816, IIM4818, IIM4822, IIM4831, IIM4851, IIM4856, IIM47276, IIM4857, IIM4858, IIM4859, IIM4860, IIM4875, IIM4878, IIM4967, IIM4974, IIM4978 or a closely associated marker thereof. A further aspect of the embodiment is a maize plant cell or maize plant (stiff or non-stiff stalk) generated by the method above.

In another embodiment comprises a method of identifying or selecting a maize plant having increased yield under drought or increased yield under non-drought conditions as compared to a control plant wherein yield is increased bushels of corn per acre, the method comprising the steps of: a) isolating a nucleic acid from a plant cell; b) detecting the presence of a genetic marker in said nucleic acid that is associated with increased yield (drought or non-drought conditions) wherein said genetic marker is closely linked to and within 5 Mb, 4 Mb, 2 Mb, 1 Mb or 0.5 Mb of SM2984 c) selecting a maize plant on the basis of the genetic marker detected in b). Another aspect wherein the genetic marker detected is within a chromosomal interval selected from the group consisting of: a chromosomal interval comprised by and flanked by IIM19 and IIM818; a chromosomal interval comprised by and flanked by IIM43 and IIM291 or a chromosomal interval comprised by and flanked by IIM121 and IIM211. In another aspect of the embodiment the genetic marker detected is selected from the group comprising: IIM19, IIM26, IIM32, IIM43, IIM66, IIM72, IIM78, IIM77, IIM84, IIM108, IIM121, IIM46822, IIM211, IIM236, IIM274, IIM275, IIM291, IIM347, IIM47190, IIM638, IIM738, IIM739, IIM818 or a closely associated marker thereof. A further aspect of the embodiment is a maize plant cell or maize plant (stiff or non-stiff stalk) generated by the method above.

Another embodiment comprises a method for producing a hybrid plant with increased yield under drought or non-drought conditions as compared to a control, the steps comprising: (a) providing a first plant comprising a first genotype comprising any one of haplotypes A-M: (b) providing a second plant comprising a second genotype comprising any one from the group comprised of SM2987, SM2991, SM2995, SM2996, SM2973, SM2980, SM2982, or SM2984, wherein the second plant comprises at least one marker from the group comprised of SM2987, SM2991, SM2995, SM2996, SM2973, SM2980, SM2982, or SM2984 that is not present in the first plant; (c) crossing the first plant and the second maize plant to produce an F1 generation; identifying one or more members of the F1 generation that comprises a desired genotype comprising any combination of haplotypes A-M and any markers from the group comprised of SM2987, SM2991, SM2995, SM2996, SM2973, SM2980, SM2982, or SM2984, wherein the desired genotype differs from both the first genotype of (a) and the second genotype of (b), whereby a hybrid plant with increased water optimization is produced. The embodiment further wherein the hybrid plant with increased yield comprises each of haplotypes A-M that are present in the first plant as well as at least one additional haplotype selected from the group comprised of SM2987, SM2991, SM2995, SM2996, SM2973, SM2980, SM2982, or SM2984 that is present in the second plant. A further aspect of the embodiment wherein the first plant is a recurrent parent comprising at least one of haplotypes A-M and the second plant is a donor that comprises at least one marker from the group comprised of SM2987, SM2991, SM2995, SM2996, SM2973, SM2980, SM2982, or SM2984 that is not present in the first plant. Another aspect of the embodiment wherein the first plant is homozygous for at least two, three, four, or five of haplotypes A-M. In another aspect, the hybrid plant comprises at least three, four, five, six, seven, eight, or nine of haplotypes A-M and markers from the group comprised of SM2987, SM2991, SM2995, SM2996, SM2973, SM2980, SM2982, or SM2984. In a further aspect, wherein the identifying comprises genotyping one or more members of an F1 generation produced by crossing the first plant and the second plant with respect to each of the haplotypes A-M and markers from the group comprised of SM2987, SM2991, SM2995, SM2996, SM2973, SM2980, SM2982, or SM2984 present in either the first plant or the second plant. Further aspect of the embodiment wherein the first plant and the second plant are Zea mays plants. The embodiment wherein increased yield is T increased or stabilized yield in a water stressed environment as compared to a control plant. A further aspect wherein the hybrid with increased yield can be planted at a higher crop density and/or confers no yield drag when under favorable moisture levels. Another aspect is a hybrid Zea mays plant produced by the embodiment or a cell, tissue culture, seed, or part thereof.

Another embodiment of the invention is a plant having introduced into its genome a water optimization gene, wherein the said water optimization gene comprises a nucleotide sequence encoding at least one polypeptide comprising SEQ ID NO: 9-16 and further wherein introduction of said water optimization gene increases yield in drought or non-drought conditions. Another aspect of the embodiment wherein introduction is any one of plant introgression through breeding, genome editing (TALEN, CRISPR, etc.), or transgenic expression. Another aspect of the embodiment wherein said plant has increased yield as compared to a control plant. In another aspect, wherein increased yield is yield under water deficit conditions. A further aspect wherein a parental line of said plant was selected by or identified by a nucleotide probe or primer that annealed to any one of SEQ ID NOs: 1-8 and said parental line conferred increased yield as compared to a plant not comprising SEQ ID NOs: 1-8. In another aspect the plant, wherein increased yield is yield under well-watered conditions. A further aspect where the plant is maize, a hybrid maize plant or an elite maize line. A further aspect wherein said gene is a nucleotide sequence having 90-100% sequence homology with any one of SEQ ID NOs: 1-8. Further aspect of the embodiment wherein said plant also comprises at least one Haplotypes A-M.

Another embodiment comprises a genotyped plant, plant cell, germplasm, pollen, seed or plant part selected or identified based on the detection of any one of SEQ ID NOs: 1-8 or closely associated markers thereof (e.g. those demonstrated in Tables 1-7). A further aspect of the embodiment wherein the plant, plant cell, germplasm, pollen, seed or plant part is genotyped by isolating DNA from said plant, plant cell, germplasm, pollen, seed or plant part and DNA is genotyped using either PCR or nucleotide probes that adhere to any one of SEQ ID NOs 1-8.

Another embodiment is a method for producing a plant with increased yield comprising the steps of: a) selecting from a diverse plant population using marker selected from the group comprised of markers SM2973, SM2980, SM2982, SM2984, SM2987, SM2991, SM2995, SM2996; b) propagating/selfing the plant. In another aspect the marker SM2973 has an “G” at nucleotide 401; marker SM2980 has an “C” at nucleotide 401; marker SM2982 has an “A” at nucleotide 401; marker SM2984 has an “G” at nucleotide 401; marker SM2987 has an “G” at nucleotide 401; marker SM2991 has an “G” at nucleotide 401; marker SM2995 has an “A” at nucleotide 401; and marker SM2996 has an “A” at nucleotide 401.

In another embodiment comprises a method of identifying or selecting a maize plant having increased yield under drought or increased yield under non-drought conditions as compared to a control plant wherein yield is increased bushels of corn per acre, the method comprising the steps of: a) isolating a nucleic acid from a plant cell; b) detecting the presence of a genetic marker in said nucleic acid that is associated with increased yield (drought or non-drought conditions) wherein said genetic marker is closely linked to and within 10 Mb, 5 Mb, 2 Mb, 1 Mb or 0.5 Mb of a maize gene selected from the group consisting of GRMZM5G862107_01; GRMZM2G094428_01; GRMZM2G027059_01; GRMZM2G050774_01; GRMZM2G134234_03; GRMZM2G416751_02; GRMZM2G467169_02; GRMZM2G156365_06; or any combination thereof and; c) selecting a maize plant on the basis of the genetic marker detected in b).

In another embodiment a crop plant comprising within its genome a plant expression cassette wherein said expression cassette comprises a plant promoter (constitutive or tissue/cell specific or preferred) operably linked to a gene comprising a DNA sequence having 70%, 80%, 90%, 95%, 99% or 100% sequence identity to any one of SEQ ID Nos: 1-8 wherein the term “crop plant”, herein, means monocotyledons such as cereals (wheat, millet, sorghum, rye, triticale, oats, barley, teff, spelt, buckwheat, fonio and quinoa), rice, maize (corn), and/or sugar cane; or dicotyledon crops such as beet (such as sugar beet or fodder beet); fruits (such as pomes, stone fruits or soft fruits, for example apples, pears, plums, peaches, almonds, cherries, strawberries, raspberries or blackberries); leguminous plants (such as beans, lentils, peas or soybeans); oil plants (such as rape, mustard, poppy, olives, sunflowers, coconut, castor oil plants, cocoa beans or groundnuts); cucumber plants (such as marrows, cucumbers or melons); fibre plants (such as cotton, flax, hemp or jute); citrus fruit (such as oranges, lemons, grapefruit or mandarins); vegetables (such as spinach, lettuce, cabbages, carrots, tomatoes, potatoes, cucurbits or paprika); lauraceae (such as avocados, cinnamon or camphor); tobacco; nuts; coffee; tea; vines; hops; durian; bananas; natural rubber plants; and ornamentals (such as flowers, shrubs, broad-leaved trees or evergreens, for example conifers). This list does not represent any limitation.

In another embodiment a crop plant comprising within its genome a plant expression cassette wherein said expression cassette comprises a plant promoter (constitutive or tissue/cell specific or preferred) operably linked to a gene encoding a protein having 70%, 80%, 90%, 95%, 99% or 100% sequence identity to any one of SEQ ID Nos: 9-16.

Another embodiment provides a method of producing a maize plant having increased yield under drought conditions or increased yield under non-drought conditions, wherein increased yield is increased bushels per acre as compared to a control plant, the method comprising the steps of: (a) isolating a nucleic acid from a plant cell; (b) editing the genomic sequence of the plant cell of a) to comprise a molecular marker associated with increased drought tolerance wherein the molecular marker is any molecular marker as described in Tables 1-7 and further wherein the genomic sequence did not have said molecular marker previously; and (c) producing a plant or plant callus from the plant cell of (b). In another aspect of the embodiment, a nucleic acid template can be generated to facilitate the edit(s) as described wherein one skilled in the art could use known genome editing tools to make direct edits within the target plant's genome (e.g. genome editing carried out by CRISPR, TALEN or Meganuclease genome editing methods as taught in the art). In another aspect of the embodiment, wherein the edit comprises any one of the following corresponding to:
i. SM2987 located on maize chromosome 1 corresponding to a G allele at position 272937870;
ii. SM2991 located on maize chromosome 2 corresponding to a G allele at position 12023706;
iii. SM2995 located on maize chromosome 3 corresponding to a A allele at position 225037602;
iv. SM2996 located on maize chromosome 3 corresponding to a A allele at position 225340931;
v. SM2973 located on maize chromosome 5 corresponding to a G allele at position 159121201; (6)
vi. SM2980 located on maize chromosome 9 corresponding to a C allele at position 12104936;
vii. SM2982 located on maize chromosome 9 corresponding to a A allele at position 133887717; or
viii. SM2984 located on maize chromosome 10 corresponding to a G allele at position 4987333; and

In another embodiment the plants of the invention, not to be limited by theory, comprise improved agronomical traits such as seedling vigor, yield potential, phosphate uptake, plant growth, seedling growth, phosphorus uptake, lodging, reproductive growth, or grain quality.

In another embodiment is encompassed the use of a molecular marker within a chromosomal interval to select, identify or produce a maize plant having increased drought tolerance and/or yield wherein the chromosomal interval is any one of: a interval located within 20 cM, 15 cM, 10 cM, 9 cM, 8 cM, 7 cM, 6 cM, 5 cM, 4 cM, 3 cM, 2 cM, 1 cM or closely linked to a yield allele corresponding to any one of: SM2987 located on maize chromosome 1 corresponding to a G allele at position 272937870; SM2991 located on maize chromosome 2 corresponding to a G allele at position 12023706; SM2995 located on maize chromosome 3 corresponding to a A allele at position 225037602; SM2996 located on maize chromosome 3 corresponding to a A allele at position 225340931; SM2973 located on maize chromosome 5 corresponding to a G allele at position 159121201; SM2980 located on maize chromosome 9 corresponding to a C allele at position 12104936; SM2982 located on maize chromosome 9 corresponding to a A allele at position 133887717; or SM2984 located on maize chromosome 10 corresponding to a G allele at position 4987333; or a chromosomal interval flanked by and including any one of: IIM56014 and IIM48939 on maize chromosome 1 located at physical base pair positions 248150852-296905665, IIM39140 and IIM40144 on maize chromosome 3 located at physical base pair positions 201538048-230992107, IIM6931 and IIM7657 on maize chromosome 9 located at physical base pair positions 121587239-145891243, IIM40272 and IIM41535 on maize chromosome 2 located at physical base pair positions 1317414-36929703, IIM25303 and IIM48513 on maize chromosome 5 located at physical base pair positions 139231600-183321037, IIM4047 and IIM4978 on maize chromosome 9 located at physical base pair positions 405220-34086738, or IIM19 and IIM818 on maize chromosome 10 located at physical base pair positions 1285447-29536061.

In another embodiment, the use of any allele listed in Tables 1-7 to produce a genome edit or modification to produce a plant with increased yield under drought and/or non-drought conditions.

Thus, the presently disclosed subject matter provides in some embodiments inbred Zea mays plants comprising one or more alleles associated with increased yield, increased yield under drought, or a desired water optimization trait.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Introduction to the Examples

To assess the value of various molecular markers/alleles under drought stress, diverse germplasm were screened in controlled field-experiments comprising a full irrigation control treatment and a limited irrigation treatment. The goal of the full irrigation treatment is to ensure water does not limit the productivity of the crop. In contrast, the goal of the limited irrigation treatment is to ensure that water is the major limiting constraint to grain yield. Main effects (e.g., treatment and genotype) and interactions (e.g., genotype×treatment) can be determined when the two treatments are applied adjacent to one another in the field. Moreover, drought related phenotypes can be quantified for each genotype in the panel thereby allowing for marker: trait associations to be conducted.

In practice, the method for the limited irrigation treatment can vary widely depending upon the germplasm being screened, the soil type, climatic conditions at the site, pre-season water supply, and in-season water supply, to name just a few. Initially, a site is identified where in-season precipitation is low (to minimize the chance of unintended water application) and is suitable for cropping. In addition, determining the timing of the stress can be important, such that a target is defined to ensure that year-to-year, or location-to-location, screening consistency is in place. An understanding of the treatment intensity, or in some cases the yield loss desired from the limited irrigation treatment, can also be considered. Selection of a treatment intensity that is too light can fail to reveal genotypic variation. Selection of a treatment intensity that is too heavy can create large experimental error. Once the timing of stress is identified and treatment intensity is described, irrigation can be managed in a manner that is consistent with these targets.

General methods for assessing and assessing drought tolerance can be found in Salekdeh et al., 2009 and in U.S. Pat. Nos. 6,635,803; 7,314,757; 7,332,651; and 7,432,416.

### Example 1 Identification of Maize Genetic Loci Associated with Yield Under Drought & Non-Drought Conditions

Complete genome-wide association (GWA) analysis was carried out by testing genic single nucleotide polymorphisms (SNPs) for association with drought-related traits measured on the Water Optimization (WO) association panel in maize. This work identified loci, markers, alleles and QTL associated with yield traits under drought or well-watered conditions.

### Marker Genotyping & Discovery

Approximately 1.09 million SNP markers were identified across 754 diverse maize lines using next generation sequencing technology. In order to extrapolate genome-wide marker coverage from this dataset, 21.8 million markers published in the maize HapMap2 (Chia et al. Nat. Gen. 2012 44:803-809), were remapped onto the B73 RefGen_v2 assembly (www.genome.arizona.edu/modules/publisher/item.php?itemid=16). An overlap of the 26 NAM parents (Buckler et al. Science 2009 325:714-718) were used to impute the Panzea HapMap2 markers across the panel. In order to reduce genotyping errors, an empirically derived predicted error (estimated percentage of incorrect imputed genotypes) threshold of 0.025 was used to filter the 21.8 million markers to 9.7 million markers for downstream analysis. The markers were further filtered by only considering genic SNP markers in the first phase of analysis, resulting in 1.4 million SNPs. An example of an appropriate imputation method is the software package NPUTE (Roberts et al. Bioinformatics 2007 23:i401-i407).

### Phenotypic Data

Of the 754 maize lines analyzed for SNP marker data, 512 lines had yield data available from previous drought trials. Two yield traits were measured to measure drought tolerance, specifically yield under irrigation (YGSMN_i) or yield under drought stress conditions (YGSMN_s). Measurements for each line were measured across multiple environments. The best linear predictions (BLUPs) calculated across environmental variables were correlated for YGSMN_i and YGSMN_s (r=0.63, P<0.001). All association analyses were conducted with these BLUPs for each trait separately. Maize phenotypic data and genotypic data was combined to identify chromosomal intervals, QTL and SNPs having a significant association with yield under drought or non-drought conditions.

### Association Analysis

Of the 1.4 million genic SNP markers, approximately 780,000 SNPs were originally tested for association with yield data. The remaining 620,000 markers were monomorphic across the 512 lines with yield data and therefore had no power for association analysis for yield under drought or non-drought conditions. The remaining 780,000 SNPs were parsed into sets of 10,000 adjacent markers and tested for association analysis with the yield data using a unified mixed model (Zhang et al. Nat. Gen. 2010 42:355-362). Three different unified mixed models were tested with the data all following the format:

y=Pv+Sa+Iu+e

Where y is a vector of phenotypic values, v is a vector of fixed effects regarding population structure, a is the fixed effect for the candidate marker, u is a vector of the random effects pertaining to recent co-ancestry, and e is a vector of residuals. P is a matrix of vectors defining population structure, S is the vector of genotypes at the candidate marker, and I is an identity matrix. The variances of the random effects are assumed to be Var(u)=2 KV_{g} and Var(e)=IV_{R}, where K is the kinship matrix consisting of the proportion of shared allele values, and I is an identity matrix.

Three mixed models were tested to assess three different methods for kinship matrix calculation and to determine whether membership to the breeding groups should be included as fixed effects in the model. For the first model (referred to as the QLocalK model), P was defined as membership to seven of the nine breeding groups. Only eight of the nine breeding groups were represented in our panel, which lead to the inclusion of seven vectors (the eighth was not necessary since the vector components for each individual summed to one). For each set of 10,000 adjacent markers, a unique kinship matrix was calculated and included in the model. Similarly, in the second model tested (referred to as the QGlobalK model) P was defined as membership to seven of the nine breeding groups. However, instead of a local kinship matrix calculated from a set of 10,000 adjacent markers a global kinship matrix based on 10,000 randomly selected markers from the genome was calculated. This global kinship matrix was used to test all markers. Lastly, the third model (referred to as the ChrK model) was tested which did not include a fixed effect for population structure (no P term) but simply a chromosomal kinship matrix. Kinship matrices specific to each chromosome based on 55K chip data from the MaizeSNP50 BeadChip (Illumina, San Diego, Calif.) were used in the model. These kinship matrices included information for 478 of the lines with yield under irrigation phenotypic data and 479 of the lines with yield under stress data. Each marker was then tested with the corresponding chromosomal K matrix. All associations were created using Tassel Version 3.0 (August 2012) (Bradbury et al. Bioinformatics 2007 23:2633-2635) using both the population parameters previously determined (P3D) and compressed MLM (Zhang et al. Nat. Gen. 2010 42:355-362).

### Stepwise Regression

Of the SNPs found to be significantly associated with yield under stress, only those SNPs that were observed in at least 20 of the 512 lines with phenotypic data were considered when creating the stepwise regression models to ensure the application of discovered markers across a diverse maize population. Stepwise regression was carried out using the SAS procedure GLMSelect. GLMSelect allows forward selection and backward elimination to be implemented competitively based on the adjusted R² of the models. Model optimization is stopped once the specified predicted residual sum of squares has been accounted for with the model. Within heterotic-group structure was accounted for by incorporating the breeding group membership as a fixed effect.

SNPs Associated with Yield Under Irrigation and Stress Conditions

As stated above, three different models controlling for population structure in different ways, were used to test all 780,000 SNPs for association with both yield under stress (YGSMN_s) and yield under irrigation (YGSMN_i) as measured across all locations.

In total, exactly 771,698 SNPs were tested for association with yield under irrigation (YGSMN_i) as measured across multiple locations. Subsequently the association with markers where the minor allele was only observed in three or less individuals were filtered out, resulting in 262,081 SNPs tested. Of those tested, 427 SNPs were significantly associated (P<0.001) with the yield under irrigation.

Slightly more SNPs (772,008) were tested for association with yield under stress (YGSMN_s) as measured across all locations. Once again markers where the minor allele was only observed in three or less individuals were filtered out, resulting in 262,224 SNPs tested. However, fewer (268) were significantly associated (P<0.001) with this trait in comparison to yield under irrigation. Again, six SNPs remained significantly associated with YGSMN_s when a threshold of P<10⁻⁵ was used. Similar to that observed for YGSMN_i, the LD decayed quickly among the SNPs significantly associated with YGSMN_s, thus identifying several potential causative SNP and/or gene(s).

Based on association analysis, several Genes were identified to be strongly associated with increased yield under non-drought conditions and increased yield under Drought stress, these include: GRMZM2G027059, GRMZM2G156365, GRMZM2G134234, GRMZM2G094428, GRMZM2G416751, GRMZM2G467169, GRMZM5G862107, and GRMZM2G050774. Further, markers closely associated with these respective genes were mapped and likewise associated with increased yield in drought & non-drought conditions (See Tables 1-7 for a complete listing; also Tables 10a and 10b; and Table 11 showing Maize Inbred association mapping).

Table 10a and 10b: Examples of maize alleles that associate with yield in different maize heterotic groups. Effect measured in YGSMN_i and YGSMN_s. All instances show an increase in bushels per acre under drought and non-drought conditions in both non-stiff stalk (NSS) and stiff stalk (SS) maize lines as compared to controls.

TABLE 10a

| | | | | | | SS |
|---|---|---|---|---|---|---|
| | | | | Cumula- | SS | Effect |
| | | | Favor- | tive | Effect | under |
| | | | able | Adjusted | under | Irriga- |
| SNP | Chr | Position | Allele | R²* | Stress^{§} | tion^{§} |
| SM2980 | 9 | 12104936 | C | 0.24 | 2.03 | 3.59 |
| SM2973 | 5 | 159121201 | G | 0.3 | 4.36 | 2.57 |
| SM2982 | 9 | 133887717 | A | 0.33 | 5.14 | 4.07 |
| SM2995 | 3 | 225037602 | A | 0.38 | 2.64 | 2.45 |

TABLE 10b

| | | | | | | NSS |
|---|---|---|---|---|---|---|
| | | | | Cumula- | NSS | Effect |
| | | | Favor- | tive | Effect | under |
| | | | able | Adjusted | under | Irriga- |
| SNP | Chr | Position | Allele | R²* | Stress^{§} | tion^{§} |
| SM2982 | 9 | 133887717 | A | 0.4 | 3.39 | 2.71 |
| SM2987 | 1 | 272937870 | G | 0.45 | 2.37 | 1.85 |
| SM2991 | 2 | 12023706 | G | 0.5 | 3.16 | 1.36 |
| SM2996 | 3 | 225340931 | A | 0.55 | 3.11 | 2.42 |
| SM2984 | 10 | 4987333 | G | 0.56 | 1.26 | 0.51 |

*Statistics specific to that SNP within the stepwise regression model.§ heterotic group effect sizes calculated for each marker individually.

TABLE 11
Maize Inbred panel association mapping (maize inbred
association where allele effect is a estimated statistical
contribution of the respective allele)

| | | | | | Allele |
|---|---|---|---|---|---|
| | Heterotic | | Favorable | | Effect^{~} |
| Name | Group | Marker | Allele | Trait | (bu/acre) |
| Inbred 44 | Stiff | SM2973 | G | YGSMN | 1.3 |
| Inbred 45 | Stiff | SM2973 | G | YGSMN | 1.3 |
| Inbred 46 | Stiff | SM2973 | G | YGSMN | 1.3 |
| Inbred 47 | Stiff | SM2973 | G | YGSMN | 1.3 |
| Inbred 48 | Stiff | SM2973 | G | YGSMN | 1.3 |
| Inbred 49 | Stiff | SM2973 | G | YGSMN | 1.3 |
| Inbred 50 | Stiff | SM2973 | G | YGSMN | 1.3 |
| Inbred 51 | Stiff | SM2973 | G | YGSMN | 1.3 |
| Inbred 52 | Stiff | SM2973 | G | YGSMN | 1.3 |
| Inbred 53 | Stiff | SM2973 | G | YGSMN | 1.3 |
| Inbred 54 | Stiff | SM2973 | G | YGSMN | 1.3 |
| Inbred 55 | Stiff | SM2973 | G | YGSMN | 1.3 |
| Inbred 56 | Stiff | SM2973 | G | YGSMN | 1.3 |
| Inbred 57 | Stiff | SM2973 | G | YGSMN | 1.3 |
| Inbred 58 | Stiff | SM2973 | G | YGSMN | 1.3 |
| Inbred 59 | Stiff | SM2973 | G | YGSMN | 1.3 |
| Inbred 60 | Stiff | SM2973 | G | YGSMN | 1.3 |
| Inbred 61 | Non-stiff | SM2973 | G | YGSMN | 2.4 |
| Inbred 62 | Non-stiff | SM2973 | G | YGSMN | 2.4 |
| Inbred 63 | Non-stiff | SM2973 | G | YGSMN | 2.4 |
| Inbred 64 | Non-stiff | SM2973 | G | YGSMN | 2.4 |
| Inbred 65 | Non-stiff | SM2973 | G | YGSMN | 2.4 |
| Inbred 66 | Non-stiff | SM2973 | G | YGSMN | 2.4 |
| Inbred 67 | Non-stiff | SM2973 | G | YGSMN | 2.4 |
| Inbred 68 | Non-stiff | SM2973 | G | YGSMN | 2.4 |
| Inbred 69 | Non-stiff | SM2973 | G | YGSMN | 2.4 |
| Inbred 70 | Non-stiff | SM2973 | G | YGSMN | 2.4 |
| Inbred 71 | Non-stiff | SM2973 | G | YGSMN | 2.4 |
| Inbred 72 | Non-stiff | SM2973 | G | YGSMN | 2.4 |
| Inbred 73 | Non-stiff | SM2973 | G | YGSMN | 2.4 |
| Inbred 74 | Non-stiff | SM2973 | G | YGSMN | 2.4 |
| Inbred 75 | Non-stiff | SM2973 | G | YGSMN | 2.4 |
| Inbred 76 | Non-stiff | SM2973 | G | YGSMN | 2.4 |
| Inbred 77 | Non-stiff | SM2973 | G | YGSMN | 2.4 |
| Inbred 78 | Non-stiff | SM2973 | G | YGSMN | 2.4 |
| Inbred 79 | Non-stiff | SM2973 | G | YGSMN | 2.4 |
| Inbred 80 | Non-stiff | SM2973 | G | YGSMN | 2.4 |
| Inbred 81 | Non-stiff | SM2973 | G | YGSMN | 2.4 |
| Inbred 82 | Non-stiff | SM2973 | G | YGSMN | 2.4 |
| Inbred 83 | Non-stiff | SM2973 | G | YGSMN | 2.4 |
| Inbred 84 | Non-stiff | SM2973 | G | YGSMN | 2.4 |
| Inbred 85 | Non-stiff | SM2973 | G | YGSMN | 2.4 |
| Inbred 86 | Non-stiff | SM2973 | G | YGSMN | 2.4 |
| Inbred 87 | Non-stiff | SM2973 | G | YGSMN | 2.4 |
| Inbred 88 | Non-stiff | SM2973 | G | YGSMN | 2.4 |
| Inbred 89 | Non-stiff | SM2973 | G | YGSMN | 2.4 |
| Inbred 90 | Non-stiff | SM2973 | G | YGSMN | 2.4 |
| Inbred 91 | Non-stiff | SM2973 | G | YGSMN | 2.4 |
| Inbred 92 | Non-stiff | SM2973 | G | YGSMN | 2.4 |
| Inbred 93 | Non-stiff | SM2973 | G | YGSMN | 2.4 |
| Inbred 94 | Non-stiff | SM2973 | G | YGSMN | 2.4 |
| Inbred 95 | Non-stiff | SM2973 | G | YGSMN | 2.4 |
| Inbred 96 | Non-stiff | SM2973 | G | YGSMN | 2.4 |
| Inbred 97 | Non-stiff | SM2973 | G | YGSMN | 2.4 |
| Inbred 98 | Non-stiff | SM2973 | G | YGSMN | 2.4 |
| Inbred 99 | Non-stiff | SM2973 | G | YGSMN | 2.4 |
| Inbred 100 | Non-stiff | SM2973 | G | YGSMN | 2.4 |
| Inbred 101 | Non-stiff | SM2973 | G | YGSMN | 2.4 |
| Inbred 102 | Non-stiff | SM2973 | G | YGSMN | 2.4 |
| Inbred 103 | Non-stiff | SM2973 | G | YGSMN | 2.4 |
| Inbred 104 | Non-stiff | SM2973 | G | YGSMN | 2.4 |
| Inbred 105 | Non-stiff | SM2973 | G | YGSMN | 2.4 |
| Inbred 106 | Non-stiff | SM2973 | G | YGSMN | 2.4 |
| Inbred 107 | Non-stiff | SM2973 | G | YGSMN | 2.4 |
| Inbred 108 | Non-stiff | SM2973 | G | YGSMN | 2.4 |
| Inbred 109 | Non-stiff | SM2973 | G | YGSMN | 2.4 |
| Inbred 110 | Non-stiff | SM2973 | G | YGSMN | 2.4 |
| Inbred 111 | Non-stiff | SM2973 | G | YGSMN | 2.4 |
| Inbred 112 | Non-stiff | SM2973 | G | YGSMN | 2.4 |
| Inbred 113 | Non-stiff | SM2973 | G | YGSMN | 2.4 |
| Inbred 114 | Non-stiff | SM2973 | G | YGSMN | 2.4 |
| Inbred 115 | Non-stiff | SM2973 | G | YGSMN | 2.4 |
| Inbred 116 | Non-stiff | SM2973 | G | YGSMN | 2.4 |
| Inbred 117 | Non-stiff | SM2973 | G | YGSMN | 2.4 |
| Inbred 118 | Non-stiff | SM2973 | G | YGSMN | 2.4 |
| Inbred 119 | Non-stiff | SM2973 | G | YGSMN | 2.4 |
| Inbred 120 | Non-stiff | SM2973 | G | YGSMN | 2.4 |
| Inbred 121 | Non-stiff | SM2973 | G | YGSMN | 2.4 |
| Inbred 122 | Non-stiff | SM2973 | G | YGSMN | 2.4 |
| Inbred 123 | Non-stiff | SM2973 | G | YGSMN | 2.4 |
| Inbred 124 | Non-stiff | SM2973 | G | YGSMN | 2.4 |
| Inbred 125 | Non-stiff | SM2973 | G | YGSMN | 2.4 |
| Inbred 126 | Non-stiff | SM2973 | G | YGSMN | 2.4 |
| Inbred 127 | Non-stiff | SM2973 | G | YGSMN | 2.4 |
| Inbred 128 | Non-stiff | SM2973 | G | YGSMN | 2.4 |
| Inbred 129 | Non-stiff | SM2973 | G | YGSMN | 2.4 |
| Inbred 130 | Non-stiff | SM2973 | G | YGSMN | 2.4 |
| Inbred 131 | Non-stiff | SM2973 | G | YGSMN | 2.4 |
| Inbred 132 | Non-stiff | SM2973 | G | YGSMN | 2.4 |
| Inbred 133 | Non-stiff | SM2973 | G | YGSMN | 2.4 |
| Inbred 134 | Non-stiff | SM2973 | G | YGSMN | 2.4 |
| Inbred 135 | Non-stiff | SM2973 | G | YGSMN | 2.4 |
| Inbred 136 | Non-stiff | SM2973 | G | YGSMN | 2.4 |
| Inbred 137 | Non-stiff | SM2973 | G | YGSMN | 2.4 |
| Inbred 138 | Non-stiff | SM2973 | G | YGSMN | 2.4 |
| Inbred 139 | Non-stiff | SM2973 | G | YGSMN | 2.4 |
| Inbred 140 | Non-stiff | SM2973 | G | YGSMN | 2.4 |
| Inbred 141 | Non-stiff | SM2973 | G | YGSMN | 2.4 |
| Inbred 142 | Non-stiff | SM2973 | G | YGSMN | 2.4 |
| Inbred 143 | Non-stiff | SM2973 | G | YGSMN | 2.4 |
| Inbred 144 | Non-stiff | SM2973 | G | YGSMN | 2.4 |
| Inbred 145 | Non-stiff | SM2973 | G | YGSMN | 2.4 |
| Inbred 146 | Non-stiff | SM2973 | G | YGSMN | 2.4 |
| Inbred 147 | Non-stiff | SM2973 | G | YGSMN | 2.4 |
| Inbred 148 | Non-stiff | SM2973 | G | YGSMN | 2.4 |
| Inbred 149 | Non-stiff | SM2973 | G | YGSMN | 2.4 |
| Inbred 150 | Non-stiff | SM2973 | G | YGSMN | 2.4 |
| Inbred 151 | Non-stiff | SM2973 | G | YGSMN | 2.4 |
| Inbred 152 | Non-stiff | SM2973 | G | YGSMN | 2.4 |
| Inbred 153 | Non-stiff | SM2973 | G | YGSMN | 2.4 |
| Inbred 154 | Non-stiff | SM2973 | G | YGSMN | 2.4 |
| Inbred 155 | Non-stiff | SM2973 | G | YGSMN | 2.4 |
| Inbred 156 | Non-stiff | SM2973 | G | YGSMN | 2.4 |
| Inbred 157 | Non-stiff | SM2973 | G | YGSMN | 2.4 |
| Inbred 158 | Non-stiff | SM2973 | G | YGSMN | 2.4 |
| Inbred 159 | Non-stiff | SM2973 | G | YGSMN | 2.4 |
| Inbred 160 | Non-stiff | SM2973 | G | YGSMN | 2.4 |
| Inbred 161 | Non-stiff | SM2973 | G | YGSMN | 2.4 |
| Inbred 162 | Non-stiff | SM2973 | G | YGSMN | 2.4 |
| Inbred 163 | Non-stiff | SM2973 | G | YGSMN | 2.4 |
| Inbred 164 | Non-stiff | SM2973 | G | YGSMN | 2.4 |
| Inbred 165 | Non-stiff | SM2973 | G | YGSMN | 2.4 |
| Inbred 166 | Non-stiff | SM2973 | G | YGSMN | 2.4 |
| Inbred 167 | Non-stiff | SM2973 | G | YGSMN | 2.4 |
| Inbred 168 | Non-stiff | SM2973 | G | YGSMN | 2.4 |
| Inbred 169 | Non-stiff | SM2973 | G | YGSMN | 2.4 |
| Inbred 170 | Non-stiff | SM2973 | G | YGSMN | 2.4 |
| Inbred 171 | Non-stiff | SM2973 | G | YGSMN | 2.4 |
| Inbred 172 | Non-stiff | SM2973 | G | YGSMN | 2.4 |
| Inbred 173 | Non-stiff | SM2973 | G | YGSMN | 2.4 |
| Inbred 174 | Non-stiff | SM2973 | G | YGSMN | 2.4 |
| Inbred 175 | Non-stiff | SM2973 | G | YGSMN | 2.4 |
| Inbred 176 | Non-stiff | SM2973 | G | YGSMN | 2.4 |
| Inbred 177 | Non-stiff | SM2973 | G | YGSMN | 2.4 |
| Inbred 178 | Non-stiff | SM2973 | G | YGSMN | 2.4 |
| Inbred 179 | Non-stiff | SM2973 | G | YGSMN | 2.4 |
| Inbred 180 | Non-stiff | SM2973 | G | YGSMN | 2.4 |
| Inbred 181 | Non-stiff | SM2973 | G | YGSMN | 2.4 |
| Inbred 182 | Non-stiff | SM2973 | G | YGSMN | 2.4 |
| Inbred 183 | Non-stiff | SM2973 | G | YGSMN | 2.4 |
| Inbred 184 | Non-stiff | SM2973 | G | YGSMN | 2.4 |
| Inbred 185 | Non-stiff | SM2973 | G | YGSMN | 2.4 |
| Inbred 186 | Non-stiff | SM2973 | G | YGSMN | 2.4 |
| Inbred 187 | Non-stiff | SM2973 | G | YGSMN | 2.4 |
| Inbred 188 | Non-stiff | SM2973 | G | YGSMN | 2.4 |
| Inbred 189 | Non-stiff | SM2973 | G | YGSMN | 2.4 |
| Inbred 190 | Non-stiff | SM2973 | G | YGSMN | 2.4 |
| Inbred 191 | Non-stiff | SM2973 | G | YGSMN | 2.4 |
| Inbred 192 | Non-stiff | SM2973 | G | YGSMN | 2.4 |
| Inbred 193 | Non-stiff | SM2973 | G | YGSMN | 2.4 |
| Inbred 194 | Non-stiff | SM2973 | G | YGSMN | 2.4 |
| Inbred 195 | Non-stiff | SM2973 | G | YGSMN | 2.4 |
| Inbred 196 | Non-stiff | SM2973 | G | YGSMN | 2.4 |
| Inbred 197 | Non-stiff | SM2973 | G | YGSMN | 2.4 |
| Inbred 198 | Non-stiff | SM2973 | G | YGSMN | 2.4 |
| Inbred 199 | Non-stiff | SM2973 | G | YGSMN | 2.4 |
| Inbred 200 | Non-stiff | SM2973 | G | YGSMN | 2.4 |
| Inbred 201 | Non-stiff | SM2973 | G | YGSMN | 2.4 |
| Inbred 202 | Non-stiff | SM2973 | G | YGSMN | 2.4 |
| Inbred 559 | Stiff | SM2980 | C | YGSMN | 0.88 |
| Inbred 560 | Stiff | SM2980 | C | YGSMN | 0.88 |
| Inbred 561 | Stiff | SM2980 | C | YGSMN | 0.88 |
| Inbred 562 | Stiff | SM2980 | C | YGSMN | 0.88 |
| Inbred 563 | Stiff | SM2980 | C | YGSMN | 0.88 |
| Inbred 564 | Stiff | SM2980 | C | YGSMN | 0.88 |
| Inbred 565 | Stiff | SM2980 | C | YGSMN | 0.88 |
| Inbred 566 | Stiff | SM2980 | C | YGSMN | 0.88 |
| Inbred 567 | Stiff | SM2980 | C | YGSMN | 0.88 |
| Inbred 568 | Stiff | SM2980 | C | YGSMN | 0.88 |
| Inbred 569 | Stiff | SM2980 | C | YGSMN | 0.88 |
| Inbred 570 | Stiff | SM2980 | C | YGSMN | 0.88 |
| Inbred 571 | Stiff | SM2980 | C | YGSMN | 0.88 |
| Inbred 572 | Stiff | SM2980 | C | YGSMN | 0.88 |
| Inbred 573 | Stiff | SM2980 | C | YGSMN | 0.88 |
| Inbred 574 | Stiff | SM2980 | C | YGSMN | 0.88 |
| Inbred 575 | Stiff | SM2980 | C | YGSMN | 0.88 |
| Inbred 576 | Stiff | SM2980 | C | YGSMN | 0.88 |
| Inbred 577 | Stiff | SM2980 | C | YGSMN | 0.88 |
| Inbred 578 | Stiff | SM2980 | C | YGSMN | 0.88 |
| Inbred 579 | Stiff | SM2980 | C | YGSMN | 0.88 |
| Inbred 580 | Stiff | SM2980 | C | YGSMN | 0.88 |
| Inbred 581 | Stiff | SM2980 | C | YGSMN | 0.88 |
| Inbred 582 | Stiff | SM2980 | C | YGSMN | 0.88 |
| Inbred 583 | Stiff | SM2980 | C | YGSMN | 0.88 |
| Inbred 584 | Stiff | SM2980 | C | YGSMN | 0.88 |
| Inbred 585 | Stiff | SM2980 | C | YGSMN | 0.88 |
| Inbred 586 | Stiff | SM2980 | C | YGSMN | 0.88 |
| Inbred 587 | Stiff | SM2980 | C | YGSMN | 0.88 |
| Inbred 588 | Stiff | SM2980 | C | YGSMN | 0.88 |
| Inbred 589 | Stiff | SM2980 | C | YGSMN | 0.88 |
| Inbred 590 | Stiff | SM2980 | C | YGSMN | 0.88 |
| Inbred 591 | Non-stiff | SM2982 | A | YGSMN | 0.8886 |
| Inbred 592 | Non-stiff | SM2982 | A | YGSMN | 0.8886 |
| Inbred 593 | Non-stiff | SM2984 | G | YGSMN | 1.0331 |
| Inbred 594 | Non-stiff | SM2984 | G | YGSMN | 1.0331 |
| Inbred 595 | Non-stiff | SM2984 | G | YGSMN | 1.0331 |
| Inbred 596 | Non-stiff | SM2984 | G | YGSMN | 1.0331 |
| Inbred 597 | Non-stiff | SM2985 | G | YGSMN | 0.9079 |
| Inbred 598 | Non-stiff | SM2985 | G | YGSMN | 0.9079 |
| Inbred 599 | Non-stiff | SM2985 | G | YGSMN | 0.9079 |
| Inbred 600 | Non-stiff | SM2985 | G | YGSMN | 0.9079 |
| Inbred 601 | Non-stiff | SM2987 | G | YGSMN | 1.0163 |
| Inbred 602 | Non-stiff | SM2987 | G | YGSMN | 1.0163 |
| Inbred 603 | Non-stiff | SM2987 | G | YGSMN | 1.0163 |
| Inbred 604 | Non-stiff | SM2987 | G | YGSMN | 1.0163 |
| Inbred 605 | Non-stiff | SM2987 | G | YGSMN | 1.0163 |
| Inbred 606 | Non-stiff | SM2987 | G | YGSMN | 1.0163 |
| Inbred 607 | Non-stiff | SM2987 | G | YGSMN | 1.0163 |
| Inbred 608 | Non-stiff | SM2987 | G | YGSMN | 1.0163 |
| Inbred 609 | Non-stiff | SM2987 | G | YGSMN | 1.0163 |
| Inbred 610 | Non-stiff | SM2987 | G | YGSMN | 1.0163 |
| Inbred 611 | Non-stiff | SM2987 | G | YGSMN | 1.0163 |
| Inbred 612 | Non-stiff | SM2987 | G | YGSMN | 1.0163 |
| Inbred 613 | Non-stiff | SM2987 | G | YGSMN | 1.0163 |
| Inbred 614 | Non-stiff | SM2987 | G | YGSMN | 1.0163 |
| Inbred 615 | Non-stiff | SM2987 | G | YGSMN | 1.0163 |
| Inbred 616 | Non-stiff | SM2987 | G | YGSMN | 1.0163 |
| Inbred 617 | Non-stiff | SM2987 | G | YGSMN | 1.0163 |
| Inbred 618 | Non-stiff | SM2990 | G | YGSMN | 2.21 |
| Inbred 619 | Non-stiff | SM2990 | G | YGSMN | 2.21 |
| Inbred 620 | Non-stiff | SM2990 | G | YGSMN | 2.21 |
| Inbred 621 | Non-stiff | SM2990 | G | YGSMN | 2.21 |
| Inbred 622 | Non-stiff | SM2990 | G | YGSMN | 2.21 |
| Inbred 623 | Non-stiff | SM2990 | G | YGSMN | 2.21 |
| Inbred 624 | Non-stiff | SM2990 | G | YGSMN | 2.21 |
| Inbred 625 | Non-stiff | SM2990 | G | YGSMN | 2.21 |
| Inbred 626 | Non-stiff | SM2990 | G | YGSMN | 2.21 |
| Inbred 627 | Non-stiff | SM2990 | G | YGSMN | 2.21 |
| Inbred 628 | Non-stiff | SM2990 | G | YGSMN | 2.21 |
| Inbred 629 | Non-stiff | SM2990 | G | YGSMN | 2.21 |
| Inbred 630 | Non-stiff | SM2990 | G | YGSMN | 2.21 |
| Inbred 631 | Non-stiff | SM2990 | G | YGSMN | 2.21 |
| Inbred 632 | Non-stiff | SM2990 | G | YGSMN | 2.21 |
| Inbred 633 | Non-stiff | SM2990 | G | YGSMN | 2.21 |
| Inbred 634 | Non-stiff | SM2990 | G | YGSMN | 2.21 |
| Inbred 635 | Non-stiff | SM2990 | G | YGSMN | 2.21 |
| Inbred 636 | Non-stiff | SM2990 | G | YGSMN | 2.21 |
| Inbred 637 | Non-stiff | SM2990 | G | YGSMN | 2.21 |
| Inbred 638 | Non-stiff | SM2990 | G | YGSMN | 2.21 |
| Inbred 639 | Non-stiff | SM2990 | G | YGSMN | 2.21 |
| Inbred 640 | Non-stiff | SM2990 | G | YGSMN | 2.21 |
| Inbred 641 | Non-stiff | SM2990 | G | YGSMN | 2.21 |
| Inbred 642 | Non-stiff | SM2990 | G | YGSMN | 2.21 |
| Inbred 643 | Non-stiff | SM2990 | G | YGSMN | 2.21 |
| Inbred 644 | Non-stiff | SM2990 | G | YGSMN | 2.21 |
| Inbred 645 | Non-stiff | SM2990 | G | YGSMN | 2.21 |
| Inbred 646 | Non-stiff | SM2990 | G | YGSMN | 2.21 |
| Inbred 647 | Non-stiff | SM2990 | G | YGSMN | 2.21 |
| Inbred 648 | Non-stiff | SM2990 | G | YGSMN | 2.21 |
| Inbred 649 | Non-stiff | SM2990 | G | YGSMN | 2.21 |
| Inbred 650 | Non-stiff | SM2990 | G | YGSMN | 2.21 |
| Inbred 651 | Non-stiff | SM2990 | G | YGSMN | 2.21 |
| Inbred 652 | Non-stiff | SM2990 | G | YGSMN | 2.21 |
| Inbred 653 | Non-stiff | SM2990 | G | YGSMN | 2.21 |
| Inbred 654 | Non-stiff | SM2990 | G | YGSMN | 2.21 |
| Inbred 655 | Non-stiff | SM2990 | G | YGSMN | 2.21 |
| Inbred 656 | Non-stiff | SM2990 | G | YGSMN | 2.21 |
| Inbred 657 | Non-stiff | SM2990 | G | YGSMN | 2.21 |
| Inbred 658 | Non-stiff | SM2990 | G | YGSMN | 2.21 |
| Inbred 659 | Non-stiff | SM2990 | G | YGSMN | 2.21 |
| Inbred 660 | Non-stiff | SM2990 | G | YGSMN | 2.21 |
| Inbred 661 | Non-stiff | SM2990 | G | YGSMN | 2.21 |
| Inbred 662 | Non-stiff | SM2990 | G | YGSMN | 2.21 |
| Inbred 663 | Non-stiff | SM2990 | G | YGSMN | 2.21 |
| Inbred 664 | Non-stiff | SM2990 | G | YGSMN | 2.21 |
| Inbred 665 | Non-stiff | SM2990 | G | YGSMN | 2.21 |
| Inbred 666 | Non-stiff | SM2990 | G | YGSMN | 2.21 |
| Inbred 667 | Non-stiff | SM2990 | G | YGSMN | 2.21 |
| Inbred 668 | Non-stiff | SM2990 | G | YGSMN | 2.21 |
| Inbred 669 | Non-stiff | SM2990 | G | YGSMN | 2.21 |
| Inbred 670 | Non-stiff | SM2990 | G | YGSMN | 2.21 |
| Inbred 671 | Non-stiff | SM2990 | G | YGSMN | 2.21 |
| Inbred 672 | Non-stiff | SM2990 | G | YGSMN | 2.21 |
| Inbred 673 | Non-stiff | SM2990 | G | YGSMN | 2.21 |
| Inbred 674 | Non-stiff | SM2990 | G | YGSMN | 2.21 |
| Inbred 675 | Non-stiff | SM2990 | G | YGSMN | 2.21 |
| Inbred 676 | Non-stiff | SM2990 | G | YGSMN | 2.21 |
| Inbred 677 | Non-stiff | SM2990 | G | YGSMN | 2.21 |
| Inbred 678 | Non-stiff | SM2990 | G | YGSMN | 2.21 |
| Inbred 679 | Non-stiff | SM2990 | G | YGSMN | 2.21 |
| Inbred 680 | Non-stiff | SM2990 | G | YGSMN | 2.21 |
| Inbred 681 | Non-stiff | SM2990 | G | YGSMN | 2.21 |
| Inbred 682 | Non-stiff | SM2990 | G | YGSMN | 2.21 |
| Inbred 683 | Non-stiff | SM2990 | G | YGSMN | 2.21 |
| Inbred 684 | Non-stiff | SM2990 | G | YGSMN | 2.21 |
| Inbred 685 | Non-stiff | SM2990 | G | YGSMN | 2.21 |
| Inbred 686 | Non-stiff | SM2990 | G | YGSMN | 2.21 |
| Inbred 687 | Non-stiff | SM2990 | G | YGSMN | 2.21 |
| Inbred 688 | Non-stiff | SM2990 | G | YGSMN | 2.21 |
| Inbred 689 | Non-stiff | SM2990 | G | YGSMN | 2.21 |
| Inbred 690 | Non-stiff | SM2990 | G | YGSMN | 2.21 |
| Inbred 691 | Non-stiff | SM2990 | G | YGSMN | 2.21 |
| Inbred 692 | Non-stiff | SM2990 | G | YGSMN | 2.21 |
| Inbred 693 | Non-stiff | SM2990 | G | YGSMN | 2.21 |
| Inbred 694 | Non-stiff | SM2990 | G | YGSMN | 2.21 |
| Inbred 695 | Non-stiff | SM2990 | G | YGSMN | 2.21 |
| Inbred 696 | Non-stiff | SM2990 | G | YGSMN | 2.21 |
| Inbred 697 | Non-stiff | SM2990 | G | YGSMN | 2.21 |
| Inbred 698 | Non-stiff | SM2990 | G | YGSMN | 2.21 |
| Inbred 699 | Non-stiff | SM2990 | G | YGSMN | 2.21 |
| Inbred 700 | Non-stiff | SM2990 | G | YGSMN | 2.21 |
| Inbred 701 | Non-stiff | SM2990 | G | YGSMN | 2.21 |
| Inbred 702 | Non-stiff | SM2990 | G | YGSMN | 2.21 |
| Inbred 703 | Non-stiff | SM2990 | G | YGSMN | 2.21 |
| Inbred 704 | Non-stiff | SM2990 | G | YGSMN | 2.21 |
| Inbred 705 | Non-stiff | SM2990 | G | YGSMN | 2.21 |
| Inbred 706 | Non-stiff | SM2990 | G | YGSMN | 2.21 |
| Inbred 707 | Non-stiff | SM2990 | G | YGSMN | 2.21 |
| Inbred 708 | Non-stiff | SM2990 | G | YGSMN | 2.21 |
| Inbred 709 | Non-stiff | SM2990 | G | YGSMN | 2.21 |
| Inbred 710 | Non-stiff | SM2990 | G | YGSMN | 2.21 |
| Inbred 711 | Non-stiff | SM2990 | G | YGSMN | 2.21 |
| Inbred 712 | Non-stiff | SM2990 | G | YGSMN | 2.21 |
| Inbred 713 | Non-stiff | SM2990 | G | YGSMN | 2.21 |
| Inbred 714 | Non-stiff | SM2990 | G | YGSMN | 2.21 |
| Inbred 715 | Non-stiff | SM2990 | G | YGSMN | 2.21 |
| Inbred 716 | Non-stiff | SM2990 | G | YGSMN | 2.21 |
| Inbred 717 | Non-stiff | SM2990 | G | YGSMN | 2.21 |
| Inbred 718 | Non-stiff | SM2990 | G | YGSMN | 2.21 |
| Inbred 719 | Non-stiff | SM2990 | G | YGSMN | 2.21 |
| Inbred 720 | Non-stiff | SM2990 | G | YGSMN | 2.21 |
| Inbred 721 | Non-stiff | SM2990 | G | YGSMN | 2.21 |
| Inbred 722 | Non-stiff | SM2990 | G | YGSMN | 2.21 |
| Inbred 723 | Non-stiff | SM2990 | G | YGSMN | 2.21 |
| Inbred 724 | Non-stiff | SM2990 | G | YGSMN | 2.21 |
| Inbred 725 | Non-stiff | SM2990 | G | YGSMN | 2.21 |
| Inbred 726 | Non-stiff | SM2990 | G | YGSMN | 2.21 |
| Inbred 727 | Non-stiff | SM2990 | G | YGSMN | 2.21 |
| Inbred 728 | Non-stiff | SM2990 | G | YGSMN | 2.21 |
| Inbred 729 | Non-stiff | SM2990 | G | YGSMN | 2.21 |
| Inbred 730 | Non-stiff | SM2990 | G | YGSMN | 2.21 |
| Inbred 731 | Non-stiff | SM2990 | G | YGSMN | 2.21 |
| Inbred 732 | Non-stiff | SM2990 | G | YGSMN | 2.21 |
| Inbred 733 | Non-stiff | SM2990 | G | YGSMN | 2.21 |
| Inbred 734 | Non-stiff | SM2990 | G | YGSMN | 2.21 |
| Inbred 735 | Non-stiff | SM2990 | G | YGSMN | 2.21 |
| Inbred 736 | Non-stiff | SM2990 | G | YGSMN | 2.21 |
| Inbred 737 | Non-stiff | SM2990 | G | YGSMN | 2.21 |
| Inbred 738 | Non-stiff | SM2990 | G | YGSMN | 2.21 |
| Inbred 739 | Non-stiff | SM2990 | G | YGSMN | 2.21 |
| Inbred 740 | Non-stiff | SM2990 | G | YGSMN | 2.21 |
| Inbred 741 | Non-stiff | SM2990 | G | YGSMN | 2.21 |
| Inbred 742 | Non-stiff | SM2990 | G | YGSMN | 2.21 |
| Inbred 743 | Non-stiff | SM2990 | G | YGSMN | 2.21 |
| Inbred 744 | Non-stiff | SM2990 | G | YGSMN | 2.21 |
| Inbred 745 | Non-stiff | SM2990 | G | YGSMN | 2.21 |
| Inbred 746 | Non-stiff | SM2990 | G | YGSMN | 2.21 |
| Inbred 747 | Non-stiff | SM2990 | G | YGSMN | 2.21 |
| Inbred 748 | Non-stiff | SM2990 | G | YGSMN | 2.21 |
| Inbred 749 | Non-stiff | SM2990 | G | YGSMN | 2.21 |
| Inbred 750 | Non-stiff | SM2990 | G | YGSMN | 2.21 |
| Inbred 751 | Non-stiff | SM2990 | G | YGSMN | 2.21 |
| Inbred 752 | Non-stiff | SM2990 | G | YGSMN | 2.21 |
| Inbred 753 | Non-stiff | SM2990 | G | YGSMN | 2.21 |
| Inbred 754 | Non-stiff | SM2990 | G | YGSMN | 2.21 |
| Inbred 755 | Non-stiff | SM2990 | G | YGSMN | 2.21 |
| Inbred 756 | Non-stiff | SM2990 | G | YGSMN | 2.21 |
| Inbred 757 | Non-stiff | SM2990 | G | YGSMN | 2.21 |
| Inbred 758 | Non-stiff | SM2990 | G | YGSMN | 2.21 |
| Inbred 759 | Non-stiff | SM2990 | G | YGSMN | 2.21 |
| Inbred 760 | Non-stiff | SM2990 | G | YGSMN | 2.21 |
| Inbred 761 | Non-stiff | SM2990 | G | YGSMN | 2.21 |
| Inbred 762 | Non-stiff | SM2990 | G | YGSMN | 2.21 |
| Inbred 763 | Non-stiff | SM2990 | G | YGSMN | 2.21 |
| Inbred 764 | Non-stiff | SM2990 | G | YGSMN | 2.21 |
| Inbred 765 | Non-stiff | SM2990 | G | YGSMN | 2.21 |
| Inbred 766 | Non-stiff | SM2990 | G | YGSMN | 2.21 |
| Inbred 767 | Non-stiff | SM2990 | G | YGSMN | 2.21 |
| Inbred 768 | Non-stiff | SM2990 | G | YGSMN | 2.21 |
| Inbred 769 | Non-stiff | SM2990 | G | YGSMN | 2.21 |
| Inbred 770 | Non-stiff | SM2990 | G | YGSMN | 2.21 |
| Inbred 771 | Non-stiff | SM2990 | G | YGSMN | 2.21 |
| Inbred 772 | Non-stiff | SM2990 | G | YGSMN | 2.21 |
| Inbred 773 | Non-stiff | SM2990 | G | YGSMN | 2.21 |
| Inbred 774 | Non-stiff | SM2990 | G | YGSMN | 2.21 |
| Inbred 775 | Non-stiff | SM2990 | G | YGSMN | 2.21 |
| Inbred 776 | Non-stiff | SM2990 | G | YGSMN | 2.21 |
| Inbred 777 | Non-stiff | SM2990 | G | YGSMN | 2.21 |
| Inbred 778 | Non-stiff | SM2990 | G | YGSMN | 2.21 |
| Inbred 779 | Non-stiff | SM2990 | G | YGSMN | 2.21 |
| Inbred 780 | Non-stiff | SM2990 | G | YGSMN | 2.21 |
| Inbred 781 | Non-stiff | SM2990 | G | YGSMN | 2.21 |
| Inbred 782 | Non-stiff | SM2990 | G | YGSMN | 2.21 |
| Inbred 783 | Non-stiff | SM2990 | G | YGSMN | 2.21 |
| Inbred 784 | Non-stiff | SM2990 | G | YGSMN | 2.21 |
| Inbred 785 | Non-stiff | SM2990 | G | YGSMN | 2.21 |
| Inbred 786 | Non-stiff | SM2990 | G | YGSMN | 2.21 |
| Inbred 787 | Non-stiff | SM2990 | G | YGSMN | 2.21 |
| Inbred 788 | Non-stiff | SM2990 | G | YGSMN | 2.21 |
| Inbred 789 | Non-stiff | SM2990 | G | YGSMN | 2.21 |
| Inbred 790 | Non-stiff | SM2990 | G | YGSMN | 2.21 |
| Inbred 791 | Non-stiff | SM2990 | G | YGSMN | 2.21 |
| Inbred 792 | Non-stiff | SM2990 | G | YGSMN | 2.21 |
| Inbred 793 | Non-stiff | SM2990 | G | YGSMN | 2.21 |
| Inbred 794 | Non-stiff | SM2990 | G | YGSMN | 2.21 |
| Inbred 795 | Non-stiff | SM2990 | G | YGSMN | 2.21 |
| Inbred 796 | Non-stiff | SM2990 | G | YGSMN | 2.21 |
| Inbred 797 | Non-stiff | SM2990 | G | YGSMN | 2.21 |
| Inbred 798 | Non-stiff | SM2990 | G | YGSMN | 2.21 |
| Inbred 799 | Non-stiff | SM2990 | G | YGSMN | 2.21 |
| Inbred 800 | Non-stiff | SM2990 | G | YGSMN | 2.21 |
| Inbred 801 | Non-stiff | SM2990 | G | YGSMN | 2.21 |
| Inbred 802 | Non-stiff | SM2990 | G | YGSMN | 2.21 |
| Inbred 803 | Non-stiff | SM2990 | G | YGSMN | 2.21 |
| Inbred 804 | Non-stiff | SM2990 | G | YGSMN | 2.21 |
| Inbred 805 | Non-stiff | SM2990 | G | YGSMN | 2.21 |
| Inbred 806 | Non-stiff | SM2990 | G | YGSMN | 2.21 |
| Inbred 807 | Non-stiff | SM2990 | G | YGSMN | 2.21 |
| Inbred 808 | Non-stiff | SM2990 | G | YGSMN | 2.21 |
| Inbred 809 | Non-stiff | SM2990 | G | YGSMN | 2.21 |
| Inbred 810 | Non-stiff | SM2990 | G | YGSMN | 2.21 |
| Inbred 811 | Non-stiff | SM2990 | G | YGSMN | 2.21 |
| Inbred 812 | Non-stiff | SM2990 | G | YGSMN | 2.21 |
| Inbred 813 | Non-stiff | SM2990 | G | YGSMN | 2.21 |
| Inbred 814 | Non-stiff | SM2990 | G | YGSMN | 2.21 |
| Inbred 815 | Non-stiff | SM2990 | G | YGSMN | 2.21 |
| Inbred 816 | Non-stiff | SM2990 | G | YGSMN | 2.21 |
| Inbred 817 | Non-stiff | SM2990 | G | YGSMN | 2.21 |
| Inbred 818 | Non-stiff | SM2990 | G | YGSMN | 2.21 |
| Inbred 819 | Non-stiff | SM2990 | G | YGSMN | 2.21 |
| Inbred 820 | Non-stiff | SM2990 | G | YGSMN | 2.21 |
| Inbred 821 | Non-stiff | SM2990 | G | YGSMN | 2.21 |
| Inbred 822 | Non-stiff | SM2990 | G | YGSMN | 2.21 |
| Inbred 823 | Non-stiff | SM2990 | G | YGSMN | 2.21 |
| Inbred 824 | Non-stiff | SM2990 | G | YGSMN | 2.21 |
| Inbred 825 | Non-stiff | SM2990 | G | YGSMN | 2.21 |
| Inbred 826 | Non-stiff | SM2990 | G | YGSMN | 2.21 |
| Inbred 827 | Non-stiff | SM2990 | G | YGSMN | 2.21 |
| Inbred 828 | Non-stiff | SM2990 | G | YGSMN | 2.21 |
| Inbred 829 | Non-stiff | SM2990 | G | YGSMN | 2.21 |
| Inbred 830 | Non-stiff | SM2990 | G | YGSMN | 2.21 |
| Inbred 831 | Non-stiff | SM2990 | G | YGSMN | 2.21 |
| Inbred 832 | Non-stiff | SM2990 | G | YGSMN | 2.21 |
| Inbred 833 | Non-stiff | SM2990 | G | YGSMN | 2.21 |
| Inbred 834 | Non-stiff | SM2990 | G | YGSMN | 2.21 |
| Inbred 835 | Non-stiff | SM2990 | G | YGSMN | 2.21 |
| Inbred 836 | Non-stiff | SM2990 | G | YGSMN | 2.21 |
| Inbred 837 | Non-stiff | SM2990 | G | YGSMN | 2.21 |
| Inbred 838 | Non-stiff | SM2990 | G | YGSMN | 2.21 |
| Inbred 839 | Non-stiff | SM2990 | G | YGSMN | 2.21 |
| Inbred 840 | Non-stiff | SM2990 | G | YGSMN | 2.21 |
| Inbred 841 | Non-stiff | SM2990 | G | YGSMN | 2.21 |
| Inbred 842 | Non-stiff | SM2991 | G | YGSMN | 2.36 |
| Inbred 843 | Non-stiff | SM2991 | G | YGSMN | 2.36 |
| Inbred 844 | Non-stiff | SM2991 | G | YGSMN | 2.36 |
| Inbred 845 | Non-stiff | SM2991 | G | YGSMN | 2.36 |
| Inbred 846 | Non-stiff | SM2991 | G | YGSMN | 2.36 |
| Inbred 847 | Non-stiff | SM2991 | G | YGSMN | 2.36 |
| Inbred 848 | Non-stiff | SM2991 | G | YGSMN | 2.36 |
| Inbred 849 | Non-stiff | SM2991 | G | YGSMN | 2.36 |
| Inbred 850 | Non-stiff | SM2991 | G | YGSMN | 2.36 |
| Inbred 851 | Non-stiff | SM2991 | G | YGSMN | 2.36 |
| Inbred 852 | Non-stiff | SM2991 | G | YGSMN | 2.36 |
| Inbred 853 | Non-stiff | SM2991 | G | YGSMN | 2.36 |
| Inbred 854 | Non-stiff | SM2991 | G | YGSMN | 2.36 |
| Inbred 855 | Non-stiff | SM2991 | G | YGSMN | 2.36 |
| Inbred 856 | Non-stiff | SM2991 | G | YGSMN | 2.36 |
| Inbred 857 | Non-stiff | SM2991 | G | YGSMN | 2.36 |
| Inbred 858 | Non-stiff | SM2991 | G | YGSMN | 2.36 |
| Inbred 859 | Non-stiff | SM2991 | G | YGSMN | 2.36 |
| Inbred 860 | Non-stiff | SM2991 | G | YGSMN | 2.36 |
| Inbred 861 | Non-stiff | SM2991 | G | YGSMN | 2.36 |
| Inbred 862 | Non-stiff | SM2991 | G | YGSMN | 2.36 |
| Inbred 863 | Non-stiff | SM2991 | G | YGSMN | 2.36 |
| Inbred 864 | Non-stiff | SM2991 | G | YGSMN | 2.36 |
| Inbred 865 | Non-stiff | SM2991 | G | YGSMN | 2.36 |
| Inbred 866 | Non-stiff | SM2991 | G | YGSMN | 2.36 |
| Inbred 867 | Non-stiff | SM2991 | G | YGSMN | 2.36 |
| Inbred 868 | Non-stiff | SM2991 | G | YGSMN | 2.36 |
| Inbred 869 | Non-stiff | SM2991 | G | YGSMN | 2.36 |
| Inbred 870 | Non-stiff | SM2991 | G | YGSMN | 2.36 |
| Inbred 871 | Non-stiff | SM2991 | G | YGSMN | 2.36 |
| Inbred 872 | Non-stiff | SM2991 | G | YGSMN | 2.36 |
| Inbred 873 | Non-stiff | SM2991 | G | YGSMN | 2.36 |
| Inbred 874 | Non-stiff | SM2991 | G | YGSMN | 2.36 |
| Inbred 875 | Non-stiff | SM2991 | G | YGSMN | 2.36 |
| Inbred 876 | Non-stiff | SM2991 | G | YGSMN | 2.36 |
| Inbred 877 | Non-stiff | SM2991 | G | YGSMN | 2.36 |
| Inbred 878 | Non-stiff | SM2991 | G | YGSMN | 2.36 |
| Inbred 879 | Non-stiff | SM2991 | G | YGSMN | 2.36 |
| Inbred 880 | Non-stiff | SM2991 | G | YGSMN | 2.36 |
| Inbred 881 | Non-stiff | SM2991 | G | YGSMN | 2.36 |
| Inbred 882 | Non-stiff | SM2991 | G | YGSMN | 2.36 |
| Inbred 883 | Non-stiff | SM2991 | G | YGSMN | 2.36 |
| Inbred 884 | Non-stiff | SM2991 | G | YGSMN | 2.36 |
| Inbred 885 | Non-stiff | SM2991 | G | YGSMN | 2.36 |
| Inbred 886 | Non-stiff | SM2991 | G | YGSMN | 2.36 |
| Inbred 887 | Non-stiff | SM2991 | G | YGSMN | 2.36 |
| Inbred 888 | Non-stiff | SM2991 | G | YGSMN | 2.36 |
| Inbred 889 | Non-stiff | SM2991 | G | YGSMN | 2.36 |
| Inbred 890 | Non-stiff | SM2991 | G | YGSMN | 2.36 |
| Inbred 891 | Non-stiff | SM2991 | G | YGSMN | 2.36 |
| Inbred 892 | Non-stiff | SM2991 | G | YGSMN | 2.36 |
| Inbred 893 | Non-stiff | SM2991 | G | YGSMN | 2.36 |
| Inbred 894 | Non-stiff | SM2991 | G | YGSMN | 2.36 |
| Inbred 895 | Non-stiff | SM2991 | G | YGSMN | 2.36 |
| Inbred 896 | Non-stiff | SM2991 | G | YGSMN | 2.36 |
| Inbred 897 | Non-stiff | SM2991 | G | YGSMN | 2.36 |
| Inbred 898 | Non-stiff | SM2991 | G | YGSMN | 2.36 |
| Inbred 899 | Non-stiff | SM2991 | G | YGSMN | 2.36 |
| Inbred 900 | Non-stiff | SM2991 | G | YGSMN | 2.36 |
| Inbred 901 | Non-stiff | SM2991 | G | YGSMN | 2.36 |
| Inbred 902 | Non-stiff | SM2991 | G | YGSMN | 2.36 |
| Inbred 903 | Non-stiff | SM2991 | G | YGSMN | 2.36 |
| Inbred 904 | Non-stiff | SM2991 | G | YGSMN | 2.36 |
| Inbred 905 | Non-stiff | SM2991 | G | YGSMN | 2.36 |
| Inbred 906 | Non-stiff | SM2991 | G | YGSMN | 2.36 |
| Inbred 907 | Non-stiff | SM2991 | G | YGSMN | 2.36 |
| Inbred 908 | Non-stiff | SM2991 | G | YGSMN | 2.36 |
| Inbred 909 | Non-stiff | SM2991 | G | YGSMN | 2.36 |
| Inbred 910 | Non-stiff | SM2991 | G | YGSMN | 2.36 |
| Inbred 1073 | Stiff | SM2994 | A | YGSMN | 1.7038 |
| Inbred 1074 | Stiff | SM2994 | A | YGSMN | 1.7038 |
| Inbred 1075 | Stiff | SM2994 | A | YGSMN | 1.7038 |
| Inbred 1076 | Stiff | SM2994 | A | YGSMN | 1.7038 |
| Inbred 1077 | Stiff | SM2994 | A | YGSMN | 1.7038 |
| Inbred 1078 | Stiff | SM2994 | A | YGSMN | 1.7038 |
| Inbred 1079 | Stiff | SM2994 | A | YGSMN | 1.7038 |
| Inbred 1080 | Stiff | SM2995 | A | YGSMN | 1.5 |
| Inbred 1081 | Stiff | SM2995 | A | YGSMN | 1.5 |
| Inbred 1082 | Stiff | SM2995 | A | YGSMN | 1.5 |
| Inbred 1083 | Stiff | SM2995 | A | YGSMN | 1.5 |
| Inbred 1084 | Stiff | SM2995 | A | YGSMN | 1.5 |
| Inbred 1085 | Stiff | SM2995 | A | YGSMN | 1.5 |
| Inbred 1086 | Stiff | SM2995 | A | YGSMN | 1.5 |
| Inbred 1087 | Stiff | SM2995 | A | YGSMN | 1.5 |
| Inbred 1088 | Stiff | SM2995 | A | YGSMN | 1.5 |
| Inbred 1089 | Stiff | SM2995 | A | YGSMN | 1.5 |
| Inbred 1090 | Stiff | SM2995 | A | YGSMN | 1.5 |
| Inbred 1091 | Stiff | SM2995 | A | YGSMN | 1.5 |
| Inbred 1092 | Stiff | SM2995 | A | YGSMN | 1.5 |
| Inbred 1093 | Stiff | SM2995 | A | YGSMN | 1.5 |
| Inbred 1094 | Stiff | SM2995 | A | YGSMN | 1.5 |
| Inbred 1095 | Stiff | SM2995 | A | YGSMN | 1.5 |
| Inbred 1096 | Stiff | SM2995 | A | YGSMN | 1.5 |
| Inbred 1097 | Stiff | SM2995 | A | YGSMN | 1.5 |
| Inbred 1098 | Stiff | SM2995 | A | YGSMN | 1.5 |
| Inbred 1099 | Stiff | SM2995 | A | YGSMN | 1.5 |
| Inbred 1100 | Stiff | SM2995 | A | YGSMN | 1.5 |
| Inbred 1101 | Stiff | SM2995 | A | YGSMN | 1.5 |
| Inbred 1102 | Stiff | SM2995 | A | YGSMN | 1.5 |
| Inbred 1103 | Stiff | SM2995 | A | YGSMN | 1.5 |
| Inbred 1104 | Stiff | SM2995 | A | YGSMN | 1.5 |
| Inbred 1105 | Stiff | SM2995 | A | YGSMN | 1.5 |
| Inbred 1106 | Stiff | SM2995 | A | YGSMN | 1.5 |
| Inbred 1107 | Stiff | SM2995 | A | YGSMN | 1.5 |
| Inbred 1108 | Stiff | SM2995 | A | YGSMN | 1.5 |
| Inbred 1109 | Stiff | SM2995 | A | YGSMN | 1.5 |
| Inbred 1110 | Stiff | SM2995 | A | YGSMN | 1.5 |
| Inbred 1111 | Stiff | SM2995 | A | YGSMN | 1.5 |
| Inbred 1112 | Stiff | SM2995 | A | YGSMN | 1.5 |
| Inbred 1113 | Stiff | SM2995 | A | YGSMN | 1.5 |
| Inbred 1114 | Stiff | SM2995 | A | YGSMN | 1.5 |
| Inbred 1115 | Stiff | SM2995 | A | YGSMN | 1.5 |
| Inbred 1116 | Non-stiff | SM2996 | A | YGSMN | 0.8168 |
| Inbred 1117 | Non-stiff | SM2996 | A | YGSMN | 0.8168 |

### Example 2 Hybrid Maize Association Studies

In order to assess the repeatability of these results in a hybrid background, hybrid genotypic and phenotypic (yield under drought conditions) data was used to look for associations using the identified SNPs (See Tables 12-13).

Two heterotic groups, Non-stiff stalk (NSS) and Stiff Stalk (SS), were analyzed separately. For each heterotic group two different phenotypic datasets were analyzed for, 1) yield under drought stress in bu/acre as measured at managed stress environment (MSE) trials; and 2) yield under drought stress in bu/acre as measured at target stress environment (TSE) trials. In MSE trials, water exposure of the plant is tightly regulated in order to ensure that drought stress occurs during flowering as opposed to TSE trials where plants are grown in sites with low rainfall and water exposure is partially regulated resulting in moderate drought stress throughout the entire growing season. Populations from 24 parental lines were used to generate the families (progeny lines) used in the NSS analyses. In total these parents had 167,854 variants segregating among them. The 24 parental lines were sequenced using a reduced genomic next generation sequencing approach. Merging the genotypic and phenotypic data from the NSS-MSE analysis resulted in 24 parental lines crossed to generate 45 populations which had a grand total of 1040 families among them. These families were then crossed to two testers. Populations with less than 10 families were excluded from the analysis since they would provide little additional value. Similarly, after merging genotypic and phenotypic data for the NSS-TSE analysis there were 24 parental lines, 46 populations and 1138 families Again, replicates from these families were then crossed to two testers to generate the hybrids that were phenotyped. Twenty parental lines were used to generate the populations and families for the SS datasets. Across these twenty parents 112,466 variants were segregating. Similar to the NSS datasets, parental lines were sequenced using a reduced genomic next generation sequencing approach. Upon merging this genotypic data with the phenotypic data there were 23 populations and a grand total of 553 families that had genotypic and phenotypic data available. Replicates from these families were then crossed to two testers to generate the hybrids that were phenotyped. When merging the genotypic data with the phenotypic data we had 23 populations and a grand total of 631 families (progeny lines) represented. Again, individuals from each family were crossed to two testers to generate the hybrids phenotyped.

### Models Tested

The two initial models tested were the fixed effect model with interaction term (1) tested using PROC GLM in SAS and a random effect model with interaction term (2) tested using PROC Mixed REML in SAS.

y=Population(fixed)+SNP(fixed)+Population×SNP(fixed)+ε (1)

y=Population(random)+SNP(fixed)+Population×SNP(random)+ε (2)

The difference between these models is whether or not the population and corresponding interaction term are treated as fixed or random. If the population term is designated as fixed, then the results are specific to the populations sampled. If the population term is designated as random, then the populations included in the analysis are assumed to be a random sampling from a larger group of populations.

The MaizeSNP50 BeadChip (Illumina, San Diego, Calif.) was also used to genotype the Family Based Association Panel. Markers linked to water optimization loci SM2987, SM2996, SM2982, SM2991, SM2995, SM2973, SM2980, and SM2984 with significant associations to increased yield under drought conditions were identified (Markers and Negative log of the P value of the association can be found in Tables 1-7).

TABLE 12
Markers associated with yield (YGSMNs) in maize hybrid backgrounds over a two year field trials
(results for each marker effect averaged for the respective year and relative to controls).

| | | | Gene Info | ID Hybrid | | | Max Marker |
|---|---|---|---|---|---|---|---|
| | | | Gene | Panel | | Favrorable | Effect |
| Chr. | Position | Marker | model | Analysis | Max_NGS_NegLogPvalue | Allele | (bu/ac) |
| 1 | 272937870 | SM2987 | GRMZM2G027059 | YGSMNSSS_YEAR1 | 1.484524 | G | 5.6273 |
| 2 | 12023706 | SM2991 | GRMZM2G156365 | YGSMNSNSS_YEAR1 | 1.31903 | G | 2.136 |
| 3 | 225037602 | SM2995 | GRMZM2G134234 | YGSMNSSS_YEAR1 | 2.441291 | A | 4.3143 |
| 3 | 225340931 | SM2996 | GRMZM2G094428 | YGSMNSNSSYEAR1 | 1.633204 | A | 2.4524 |
| 5 | 159121201 | SM2973 | GRMZM2G416751 | YGSMNSNSS_YEAR2 | 1.1143 | G | 1.6222 |
| 5 | 159121201 | SM2973 | GRMZM2G416751 | YGSMNSSS_YEAR2 | 1.649364 | G | 1.2837 |
| 9 | 12104936 | SM2980 | GRMZM2G467169 | YGSMNSSS_YEAR2 | 1.033764 | C | 0.7753 |
| 9 | 133887717 | SM2982 | GRMZM5G862107 | YGSMNSNSS_YEAR1 | 1.805486 | A | 4.4902 |
| 10 | 4987333 | SM2984 | GRMZM2G050774 | YGSMNSSS_YEAR2 | 1.140021558 | G | 2.3224 |

TABLE 12
Further Hybrid Maize Association Data:

| | | Allele (in brackets = | YGSMN effect size |
|---|---|---|---|
| Hybrid [symbol] | Locus [symbol] | favorable) [symbol] | (bu/acres) [symbol] |
| Hybrid 1 | SM2987 | (GG) | 1.0163 |
| Hybrid 1 | SM2991 | AA | 0 |
| Hybrid 1 | SM2973 | (GG) | 2.4 |
| Hybrid 1 | SM2990 | (GG) | 2.21 |
| Hybrid 1 | SM2995 | (AA) | 1.5 |
| Hybrid 1 | SM2980 | GG | 0 |
| Hybrid 1 | SM2994 | GG | 0 |
| Hybrid 2 | SM2995 | CC | 0 |
| Hybrid 2 | SM2973 | CC | 0 |
| Hybrid 2 | SM2980 | GG | 0 |
| Hybrid 2 | SM2994 | GG | 0 |
| Hybrid 2 | SM2991 | (GG) | 2.36 |
| Hybrid 2 | SM2973 | (GG) | 2.4 |
| Hybrid 2 | SM2990 | (GG) | 2.21 |
| Hybrid 2 | SM2985 | CC | 0 |
| Hybrid 3 | SM2995 | CC | 0 |
| Hybrid 3 | SM2973 | CC | 0 |
| Hybrid 3 | SM2980 | (CC) | 0.88 |
| Hybrid 3 | SM2990 | (GG) | 2.21 |
| Hybrid 3 | SM2994 | (AA) | 1.7038 |
| Hybrid 3 | SM2987 | (GG) | 1.0163 |
| Hybrid 3 | SM2996 | (AA) | 0.8168 |
| Hybrid 3 | SM2991 | (GG) | 2.36 |
| Hybrid 3 | SM2973 | (GG) | 2.4 |
| Hybrid 3 | SM2990 | (GG) | 2.21 |
| Hybrid 4 | SM2995 | CC | 0 |
| Hybrid 4 | SM2973 | CC | 0 |
| Hybrid 4 | SM2980 | GG | 0 |
| Hybrid 4 | SM2994 | GG | 0 |
| Hybrid 4 | SM2987 | (GG) | 1.0163 |
| Hybrid 4 | SM2991 | (GG) | 2.36 |
| Hybrid 4 | SM2973 | (GG) | 2.4 |
| Hybrid 4 | SM2990 | (GG) | 2.21 |

### Example 3 Transgenic Expression of Maize Yield Genes

Transgenic Arabidopsis plants were created constitutively expressing the following maize genes: GRMZM2G027059 (SEQ ID NO: 1); GRMZM2G156365 (SEQ ID NO: 2); GRMZM2G134234 (SEQ ID NO: 3); GRMZM2G094428 (SEQ ID NO: 4); GRMZM2G416751 (SEQ ID NO: 5); GRMZM2G467169 (SEQ ID NO: 6); GRMZM5G862107 (SEQ ID NO: 7); GRMZM2G050774 (SEQ ID NO: 8). The experiments and results are summarized below.

### Methodology

The predicted coding sequence for each of the maize genes were synthesized and cloned into a binary vector driven by a 35s promoter without codon optimization.

Arabidopsis transformation was carried out as described by Zhang et al. (2006) using the Agrobacterium strain GV3101. The Agrobacterium carrying the construct were then transformed into Arabidopsis ecotype Col-0. T0 seeds were screened on MS medium containing 0.6% PAT. PAT resistant T0 events were confirmed by Taqman© assay and then transferred to the green house to generate T1 seed.

Greenhouse conditions were maintained using a 10 hour daylight photoperiod for the first four weeks and 16 hour daylight photoperiod during flowering. Light intensity was maintained at approximately 6000 Lux and Temperature at around 24° C. during daytime and 20° C. during nighttime. Humidity was maintained at around 40-60%. The plants were grown in nutrition soil and vermiculite mixture 1:1.

Protein Expression

For protein expression studies all genes of interest were fused with GST on their N-terminal and cloned into a expression vector. The expression vectors were transformed into E. coli using standard transformation procedures and cells were grown in LB medium to an OD600 of 0.8. Expression was induced by addition of IPTG (isopropyl Beta-D-1-thiogalactopyranoside) to 0.4 mM final concentration. Cells were incubated at 16° C. while shaking for 16 hours. The cells were pelleted via centrifugation and resuspended in 20 mM Tris-HCL, pH 8.0, 500 mM NaCl and supplemented with complete Protease Inhibitor mixture (Roche). Cells were lysed via sonification and clarified lysate was bound in batch to GST agarose (GE Life Sciences). The resin was washed extensively with 20 mM Tris-HCL, pH 8.0, 500 mM NaCL, and bound protein was eluted in wash buffer containing 10 mM Glutathione (Sigma). Eluted protein was diluted into 20% (vol/vol) glycerol and stored at −20° C.

Chlorophyll Content Measurement

Sample leaf tissue of Arabidopsis transgenic events and wild-type controls was taken at 0.01 g with 3 replicates each. Leaf samples were ground and 800 μl of acetone added. This was then put in the dark for two hours then pelleted via centrifugation. The liquid portion was then analyzed in a spectrophotometer at 663 nm and 645 nm. Total chlorophyll (μg/mL) was calculated according to the following formula:

Total chlorophyll(μg/mL)=chlorophyll a+chlorophyll b=(20.2×A645)+(8.02×A663)

Esterase Assay

Esterase activity was assayed as described by Brick et al. (1995). The assay mixture was incubated in microtiter wells at room temperature for 50 minutes. The hydrolysis of p-nitrophenyl-acetate (pNP-Ac, Sigma, Cat # N8130) and formation of p-nitrophenol was monitored spectrophotometrically by the increase in absorbance at 400 nm. Assay mixtures without Assay mixtures without substrate or enzyme were included as controls. Substrate control (substrate incubated without enzyme) was also used because of the spontaneous deacetylation of pNP-Ac.

Metabolite Analysis

Plants were grown in soil for 4 weeks under a 10 hour daylight photoperiod. Leaf samples were collected and measured for total fresh weight (˜1 g). Next, leaf samples were ground to a powder with a mortar and pestle under liquid nitrogen. The powdered material was then lyophilized by Freeze Dryer EPSILON 2-4 LSC with the following procedure: Main drying (−10° C., 0.4 mbar for 2 days) followed by Final drying (40° C., 0.1 mbar for 6 hours). Transfer the powder to a polypropylene tube for shipping. Metabolites analysis was carried out by Metabolon, Inc., US.

### A. GRMZM2G027059 (SEQ ID NO: 1) Gene Putatively Involved in Controlling Chlorophyll Content

GRMZM2G027059 is believed to encode a 4-hydroxy-3-methylbut-2-enyl diphosphate reductase which is an essential enzyme for the biosynthesis of photo pigments (such as chlorophylls and carotenoids), and hormones (gibberellins and ABA). Not to be limited by theory it is believed that plants overexpressing or harboring this gene may be more tolerant to abiotic stress (e.g. drought) as compared to a control gene.

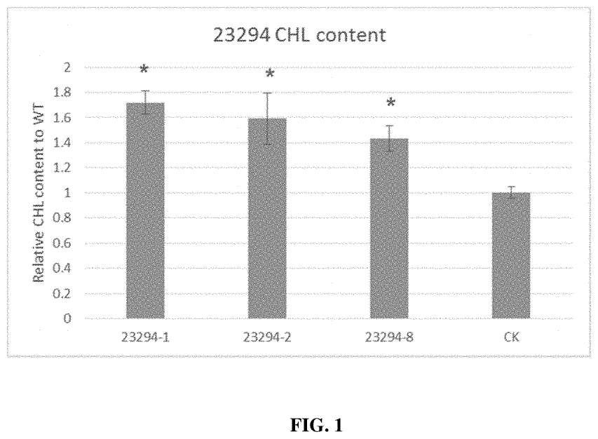

GRMZM2G027059 was expressed in Arabidopsis (construct 23294) and chlorophyll content measured as described previously. As seen in FIG. 1 chlorophyll content of transgenic plants was significantly higher than that of the control (CK) plant (See FIG. 1). This study confirms that GRMZM2G027059 does play a role in increased chlorophyll content and this in turn may be a possible mode of action for creating plants with increased yield under drought and non-drought conditions. Another possibility, not to be limited by theory, is that the overexpression of GRMZM2G027059 may also increase hormone production of sensitivity, for example increased ABA response to stress.

### B. GRMZM2G156365 (SEQ ID NO: 2) Gene Putatively Involved in Cell Wall Development & Structure

GRMZM2G156365 possibly functions as a structural regulator by modulating the precise status of pectin acetylation (i.e. a possible pectinacetylesterase). This acetylation would affect the cell wall remodeling and physiochemical properties, thereby affecting pollen cell extensibility. Not to be limited by theory, it is possible that downregulation of this gene might increase pollen germination under abiotic stress conditions (e.g. drought).

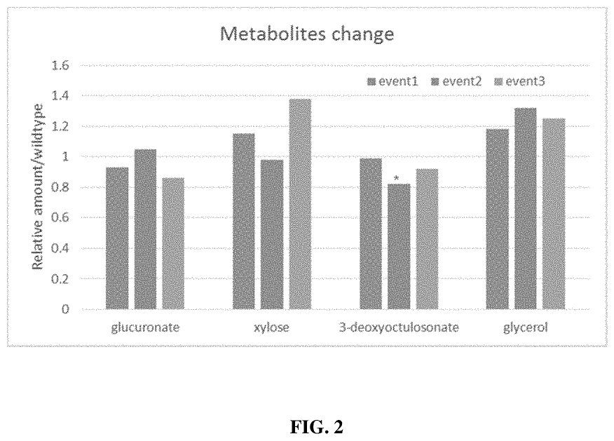

GRMZM2G156365 overexpression changed glucoronate, xylose, and 3-deoxyotulosonate contents in transgenic plants (see FIG. 2). These are all sugar residues involved with pectin formation. A little more glycerol was detected in transgenic events than wildtype control, this may be due to the esterase activity which releases glycerol.

### C. GRMZM2G134234 (SEQ ID NO: 3) Gene Involved in Abiotic Stress Regulation

Maize gene GRMZM2G134234 encodes a putative DUF1644 family transcription factor based on amino acid sequence analysis. These gene types are known to enhance drought and salinity tolerance in other crops such as rice. It is believed that GRMZM2G134234 might positively regulate stress responsive genes to increase maize stress tolerance during times of stress. Not to be limited by theory, Plants overexpressing GRMZM2G134234 might be more tolerant to abiotic stresses such as drought and salt.

### D. GRMZM2G094428 (SEQ ID NO: 4) Gene Putatively Involved in Lignin Biosynthesis and Cell Wall Structure

Maize gene GRMZM2G094428 encodes a putative BAHD acyltransferase based on amino acid sequence analysis. Thus the gene might be responsible for p-coumaroylation of monolignols in lignin biosynthesis, and ferulic acid (FA) esterification to glucuronoarabinoxylan (GAX) in the cell wall. Overexpression of the gene may increase lignin content that can confer plant tolerance under abiotic stresses, including drought and salt. Not to be limited by theory, downregulation of BAHD acyl-coA transferase could reduce FA or pCA content and change lignin content.

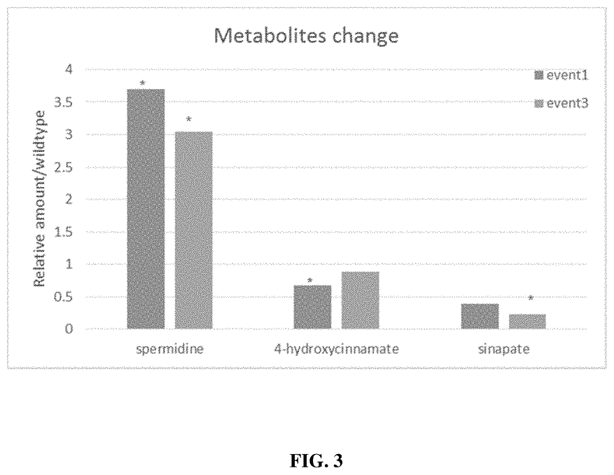

Results indicate p-coumaric acid (pCA) and sinapatic acid (SA) decreased and spermidine increased in T1 transgenic plants (see FIG. 3). GRMZM2G094428 protein appears to likely be involved in cell wall formation. Overexpression of the gene in transgenic plant changed cell wall related components.

### E. GRMZM2G416751 (SEQ ID NO: 5) Gene Putatively Involved in Pollen Exine Formation

Yield loss caused by pollen sterility caused by drought is a major factor in commercial agriculture. GRMZM2G416751 might be involved in pollen exine formation and plants overexpressing this gene might avoid pollen sterility under drought stress.

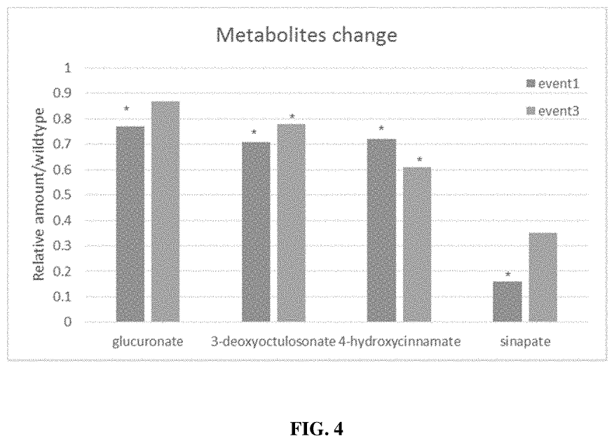

Results indicate that overexpression of GRMZM2G416751 showed decrease metabolites for cell wall formation (see FIG. 4). Metabolite profiles indicate that several metabolites for cell wall formation decreased in transgenic events such as glucoronate and 3-deoxyotulosonate for pectin, p-CA for cutin and lignin, and sinapate for lignin biosynthesis. Further analysis with male reproductive tissues like pollen or anther are needed to evaluate the genes role in pollen exine formation.

### F. GRMZM2G467169 (SEQ ID NO: 6) Gene Putatively Involved in Regulation of Retrograde Signaling

Under various biotic and abiotic stresses, signals such as redox imbalance in PS1 originate from chloroplast and are transmitted to the nucleus to control gene expression patterns (retrograde signaling). GRMZM2G467169 encodes a putative polyadenylate binding protein that might regulate retrograde signaling to increase maize stress tolerance. Plants overexpressing this gene might be more tolerant to abiotic stresses such as drought.

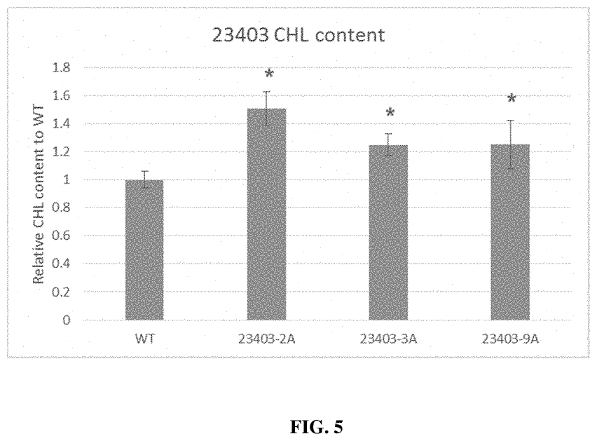

Data indicates that overexpression of GRMZM2G467169 increases chlorophyll content as compared to controls (see FIG. 5).

G. GRMZM5G862107 (SEQ ID NO: 7) Gene Putatively Involved in Modulating Gene Expression of Heat Responsive Gens and/or Target Genes.

Maize gene GRMZM5G862107 encodes a putative 30S ribosomal RNA-binding protein 51 based on amino acid sequence analysis. GRMZM5G862107 might be responsible for cold and heat stress through modulating gene expression of heat-responsive gene and/or its target genes.

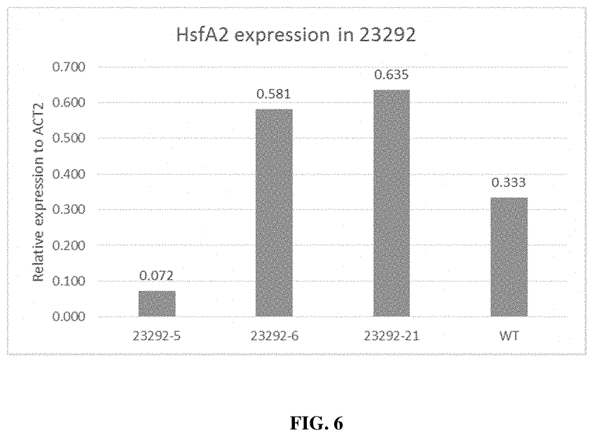

Data indicates that GRMZM5G862107 protein is involved in HsfA2 expression regulation. HsfA2 had relatively higher expression in 23292 as compared to wild type control plants (see FIG. 6).

### H. GRMZM2G050774 (SEQ ID NO: 8) Gene Putatively Involved in Plant Defense Responses

Maize gene GRMZM2G050774 encodes a putative ATL6-like RING-finger E3 ligase based on amino acid sequence analysis. In Arabidopsis it was found that ATL6/ATL31 plays a critical role in the C/N status response and plant defense response as well. Overexpressing ATL6/ATL31 can allow plant to grow well under low N supply condition and display increased resistance to Pst. DC3000. 14-3-3χ (also known as GRF1) was identified as target of ATL31. Not to be limited by theory it is possible that GRMZM2G050774 may play a role in plant nitrogen utilization/efficiency and overexpression of said gene allows a plant to better adapt to high stress conditions (e.g. such as drought or heat stress).

It will be understood that various details of the presently disclosed subject matter can be changed without departing from the scope of the presently disclosed subject matter. Furthermore, the foregoing description is for the purpose of illustration only, and not for the purpose of limitation.

1-16. (canceled)

17. A method of producing a maize plant having increased yield under drought conditions or increased yield under non-drought conditions, wherein increased yield is increased bushels per acre as compared to a control plant, the method comprising the steps of:
a) isolating a nucleic acid from the a first maize plant;
b) detecting in the nucleic acid of a) at least one molecular marker that is associated with increased yield under drought conditions or increased yield under non-drought conditions, wherein said allele is localized within 20 cM, 15 cM, 10 cM, 9 cM, 8 cM, 7 cM, 6 cM, 5 cM, 4 cM, 3 cM, 2 cM, 1 cM or genetically linked to a yield allele corresponding to any one of:
(i) SM2987 located on maize chromosome 1 corresponding to a G allele at position 272937870;
(ii) SM2991 located on maize chromosome 2 corresponding to a G allele at position 12023706;
(iii) SM2995 located on maize chromosome 3 corresponding to a A allele at position 225037602;
(iv) SM2996 located on maize chromosome 3 corresponding to a A allele at position 225340931;
(v) SM2973 located on maize chromosome 5 corresponding to a G allele at position 159121201;
(vi) SM2980 located on maize chromosome 9 corresponding to a C allele at position 12104936;
(vii) SM2982 located on maize chromosome 9 corresponding to a A allele at position 133887717; or
(viii) SM2984 located on maize chromosome 10 corresponding to a G allele at position 4987333; and
c) selecting a first maize plant based on the presence of the molecular marker detected in b);
d) crossing the maize plant of c) with a second maize plant not comprising in its genome the molecular marker detected in the first maize plant; and
e) producing a progeny plant from the cross of d) resulting in a maize plant having increased yield under drought conditions or increased yield under non-drought conditions as compared to a control plant.

18. The method of claim 17, wherein said molecular marker is localized within a chromosomal interval flanked by and including any one of:
a. IIM56014 and IIM48939 on maize chromosome 1 located at physical base pair positions 248150852-296905665,
b. IIM39140 and IIM40144 on maize chromosome 3 located at physical base pair positions 201538048-230992107,
c. IIM6931 and IIM7657 on maize chromosome 9 located at physical base pair positions 121587239-145891243,
d. IIM40272 and IIM41535 on maize chromosome 2 located at physical base pair positions 1317414-36929703,
e. IIM25303 and IIM48513 on maize chromosome 5 located at physical base pair positions 139231600-183321037,
f. IIM4047 and IIM4978 on maize chromosome 9 located at physical base pair positions 405220-34086738, or
g. IIM19 and IIM818 on maize chromosome 10 located at physical base pair positions 1285447-29536061.

19. The method of claim 17, wherein said molecular marker is localized within a chromosomal interval including any one of:
a. a chromosome interval on maize chromosome 1 defined by and including base pair position 272937470 to base pair position 272938270;
b. a chromosome interval on maize chromosome 2 defined by and including base pair position 12023306 to base pair position 12024104;
c. a chromosome interval on maize chromosome 3 defined by and including base pair position 225037202 to base pair position 225038002;
d. a chromosome interval on maize chromosome 3 defined by and including base pair position 225340531 to base pair position 225341331;
e. a chromosome interval on maize chromosome 5 defined by and including base pair position 159,120,801 to base pair position 159,121,601;
f. a chromosome interval on maize chromosome 9 defined by and including base pair position 12104536 to base pair position 12105336;
g. a chromosome interval on maize chromosome 9 defined by and including base pair position 225343590 to base pair position 225340433; or
h. a chromosome interval on maize chromosome 10 defined by and including base pair position 14764415 to base pair position 14765098.

20. The method of claim 17, wherein the detected molecular marker is closely associated with the presence of any one of the following genes encoding a protein comprising any one of SEQ ID Nos: 9-16.

21. The method of claim 20, wherein the gene comprises any one of the nucleotide sequences SEQ ID Nos: 1-8.

22. The method of claim 17, wherein the detected molecular marker is any allele or closely associated allele listed in Tables 1-7.

23. The method of claim 17, wherein detecting comprises: a) admixing an amplification primer or amplification primer pair with a nucleic acid isolated from a maize plant or maize germplasm, wherein the primer or primer pair is complementary or partially complementary to at least a portion of the marker locus, and is capable of initiating DNA polymerization by a DNA polymerase using the maize nucleic acid as a template; and, b) extending the primer or primer pair in a DNA polymerization reaction comprising a DNA polymerase and a template nucleic acid to generate at least one informative fragment wherein the informative fragment comprises any of the markers listed in Tables 1-7.

24. The method of claim 23, wherein the informative fragment comprises any of the following SEQ ID Nos: 17-24.

25. The method of claim 23, wherein the informative fragment allows for the identification of any of the following alleles:
a. a G nucleotide at position 401 of SEQ ID NO: 17;
b. a G nucleotide at position 401 of SEQ ID NO: 18;
c. a A nucleotide at position 401 of SEQ ID NO: 19;
d. a A nucleotide at position 401 of SEQ ID NO: 20;
e. a G nucleotide at position 401 of SEQ ID NO: 21;
f. a C nucleotide at position 401 of SEQ ID NO: 22;
g. a A nucleotide at position 401 of SEQ ID NO: 23; and
h. a G nucleotide at position 401 of SEQ ID NO: 24.

26. The method of claim 17, wherein the progeny plant is a hybrid maize plant.

27. The method of claim 17, wherein the first and second maize plants are inbred maize plants.

28. The method of claim 17, wherein the progeny maize plant further comprises in its genome a transgenic gene or is a non-naturally occurring maize plant.

29. The method of claim 17, wherein the plant is an elite maize plant.

30. The method of claim 17, wherein the progeny maize plant further comprises in its genome any one of SEQ ID Nos: 65-77.

31. The method of claim 17, wherein the detection is carried out comprising a primer pair or molecular probe selected from the group consisting of SEQ ID Nos: 25-56.

32. The method of claim 17, wherein the molecular marker is a single nucleotide polymorphism (SNP), a quantitative trait locus (QTL), an amplified fragment length polymorphism (AFLP), randomly amplified polymorphic DNA (RAPD), a restriction fragment length polymorphism (RFLP) or a microsatellite.

33-42. (canceled)

43. A maize plant, maize seed, maize germplasm or maize plant cell produced using the method as described in claim 17.

44. A primer or molecular probe comprising any one of SEQ ID Nos: 25-56.

45. A composition comprising the primer or molecular probe of claim 44.

46-49. (canceled)

50. The method of claim 17, wherein increased yield under drought conditions is increased drought tolerance and is any one of: increased grain yield at standard moisture percentage (YGSMN); decreased grain moisture at harvest (GMSTP); increased grain weight per plot (GWTPN); increased percent yield recovery (PYREC); decreased yield reduction (YRED); or decreased percent barren (PB).

51-53. (canceled)
